(19) United States
(12) Patent Application Publication
(10) Pub. No.: US 2023/0348545 A1
(43) Pub. Date: Nov. 2, 2023

(54) HOST CELLS OVEREXPRESSING TRANSLATIONAL FACTORS

(71) Applicant: LONZA LTD, Visp (CH)

(72) Inventors: Brigitte GASSER, Vienna (AT); Jennifer STAUDACHER, Visp (CH); Diethard MATTANOVICH, Vienna (AT)

(21) Appl. No.: 18/245,687

(22) PCT Filed: Sep. 30, 2021
(86) PCT No.: PCT/EP2021/076910

§ 371 Date: Mar. 16, 2023

(30) Foreign Application Priority Data
Sep. 30, 2020 (EP) 20199354.0

(51) Int. Cl. C07K 14/47; C12N 15/79

(52) U.S. Cl. CPC C07K 14/4705; C12N 15/79

(57) ABSTRACT

A recombinant eukaryotic host cell expressing a gene of interest (GOI) which is engineered by genetic modifications to increase expression of two or more genes encoding translation initiation factors (TIF genes) of the messenger ribonucleoprotein (mRNP), compared to the host cell prior to said one or more genetic modifications, wherein said TIF genes comprise at least a gene encoding eIF4A and a gene encoding eIF4G, and wherein expression of at least one of said TIF genes is under transcriptional control of a promoter different from the promoter controlling expression of said GOI.

### TECHNICAL FIELD

The invention refers to improving the yield of recombinant protein production and host cells engineered to increase expression of one or more translational factors.

### BACKGROUND

Proteins produced in recombinant host cell culture have become increasingly important as diagnostic and therapeutic agents. For this purpose, cells are engineered and/or selected to produce unusually high levels of a recombinant or heterologous protein of interest.

Successful production of proteins of interest (POI) has been accomplished with eukaryotic host cells in cell culture. Eukaryotic host cells, in particular mammalian host cells, yeasts or filamentous fungi, or bacteria are commonly used as production hosts for biopharmaceutical proteins as well as for bulk chemicals. The most prominent examples are methylotrophic yeasts such as Pichia pastoris, which is well reputed for efficient secretion of heterologous proteins. In 2005, P. pastoris has been reclassified into a new genus, Komagataella, and split into three species, K. pastoris, K. phaffii, and K. pseudopastoris. Strains commonly used for biotechnological applications belong to two proposed species, K. pastoris and K. phaffii. The strains GS115, X-33, CBS2612, and CBS7435 are K. phaffii, while the strain DSMZ70382 is classified into the type species, K. pastoris, which is the reference strain for all the available P. pastoris strains (Kurtzman 2009, J Ind Microbiol Biotechnol. 36(11):1435-8). Mattanovich et al. (Microbial Cell Factories 2009, 8:29 doi:10.1186/1475-2859-8-29) describe the genome sequencing of the type strain DSMZ70382 of K. pastoris, and analyzed its secretome and sugar transporters.

The ribosome is a complex ribonucleoprotein assembly that carries out the protein synthesis. The messenger ribonucleoprotein (mRNP) is an mRNA-protein complex, where a transcript is bound by a changing set of proteins that mediate the co-transcriptional and post-transcriptional events that make up a transcript's lifecycle. Transcripts first undergo 5′ end capping, splicing in many cases, 3′ cleavage and polyadenylation, mRNA quality control by the nuclear exosome, and export factor recruitment. They are then exported to the cytoplasm, where some undergo specific subcellular localization. Transcripts are eventually translated, often in a regulated manner, and degraded.

Translation initiation is on the critical pathway for the production of recombinant proteins. Formation of a closed loop structure comprised of mRNA, a number of eukaryotic initiation factors (eIFs) and ribosomal proteins is under discussion to aid initiation of translation and therefore increase global translational efficiency.

Mead et al. (Biochem. J. 2015, 472:261-273) describe mRNA and protein levels of key components of the closed loop, eIFs (eIF3a, eIF3b, eIF3c, eIF3h, eIF3i and eIF4G1), poly(A)-binding protein (PABP) 1 and PABP-interacting protein 1 (PAIP1), across a panel of 30 recombinant CHO cell lines producing monoclonal antibodies (mAb). High-producing cell lines were found to maintain amounts of the translation initiation factors involved in the formation of the closed loop mRNA, maintaining these proteins at appropriate levels to deliver enhanced recombinant protein production.

The eIF4F complex is comprised of the cap-binding protein eIF4E, eIF4G, and the RNA helicase eIF4A. eIF4G is a scaffold protein that harbors binding domains for PABP (PAB1), eIF4E, eIF4A, and (in mammals) eIF3. Both yeast and human eIF4G also bind RNA. The binding domains for eIF4E and PABP in eIF4G, along with its RNA-binding activity, enable eIF4G to coordinate independent interactions with mRNA via the cap, poly(A) tail, and sequences in the mRNA body to assemble a stable, circular messenger ribonucleoprotein (mRNP), referred to as the “closed-loop” structure.

The closed loop model proposes the interaction of the 5′- and 3′-ends of the mRNA via a bridging mechanism mediated by a number of proteins, including several translation initiation factors. The core bridge of the closed loop is formed between the 5′-cap, eIF4F (composed of eIF4A, eIF4E and eIF4G), eIF3, poly(A)-binding protein (PABP)-interacting protein 1 (PAIP1), PABP1 and the poly(A) tail. It is largely accepted that this circularization of mRNA enhances translation rates by enhanced recycling of ribosomes and/or by ensuring eIF4F remains tethered to the mRNA and does not have to be re-recruited from the free eIF4F pool for every round of translation initiation. The elongation, termination and recycling phases of translation in eukaryotes are reviewed by Dever et al. (Cold Spring Harb Perspect Biol 2012; 4:a013706) and Hinnebusch et al. (Cold Spring Harb Perspect Biol 2012; 4:a011544).

Roobol et al. (Metabolic Engineering 2020, 59:98-105) examine the effect of transient and stable overexpression of eIF3i and eIF3v subunits of the large eIF3 complex in the mammalian cell lines HEK and CHO cell lines, respectively, on increased growth rate, increased protein synthetic capacity and delayed apoptosis. eIF3i is a component of the eukaryotic initiation factor 3 (eIF3) complex comprising a single copy of 12 different subunits, 5 of which, a, b, c, g and i, are conserved and essential in vivo from yeasts to mammals.

Archer et al. (RNA Biol. 2015 March; 12(3): 248-254) investigated the mRNA closed-loop formed through interactions between the cap structure, poly(A) tail, eIF4E, eIF4G and PAB, in yeast.

Chan et al. (eLife 2018; 7:e32536) describe that inhibiting translation initiation destabilizes individual transcripts and leads to accelerated mRNA decay in yeast. Overexpression of a 5′cap-binding mutant of eIF4E caused a subtle inhibition of growth. Upon simultaneously downregulation of eIF4E and eIF4G, a strong synthetic growth defect was observed.

The translation initiation factors eIF4E, eIF4G1, and eIF4G2 present in 39S and 57S translation complexes co-purify with PAB1. Such complexes contain the closed-loop factors, eIF4E, eIF4G, and PAB1, apparently associated with an mRNA through eIF4E binding to the mRNA cap and PAB1 binding to the polyadenylated tail (cf. Denis et al. Nature Scientific Reports 2018, 8:11468).

RLI1 is known to be important for ribosome recycling and required for efficient stop codon recognition, thus stimulating translation termination. However, RLI1 has dual functions in translation initiation and ribosome biogenesis. Yarunin et al. (The EMBO Journal 2005, 24:580-588) describe RLI1 with functions in ribosome formation associated with pre-40S particles and mature 40S subunits. RLI1 is specifically associated with MFC components and 40S ribosomes (Dong et al. THE JOURNAL OF BIOLOGICAL CHEMISTRY 2004, 279(40):42157-42168).

Liao et al. (Biotechnol Lett (2020). https://doi.org/10.1007/s10529-020-02977-z) discloses expression profiles of eGFP under methanol induction in translation-related factor-overexpressing strains and identified Bcy1, a ribosome biogenesis factor, as a factor that significantly increased eGFP expression when overexpressed under methanol induction. eIF4A and eIF4G overexpressors did not have a significant effect in such expression system. Bcy1 is a regulatory subunit of the cyclic AMP-dependent protein kinase (PKA) and regulates ribosome protein genes, postdiauxic shift genes and stress response element genes, leading to improved cell growth and heterologous protein expression.

WO2019173204A1 discloses yeast overexpressing PAB1 thereby reducing acetate formation, for use in large-scale ethanol production. U.S. Pat. No. 5,646,009 discloses a hybrid vector including in one open reading frame a DNA segment encoding eIF4E, and another DNA segment encoding a protein of interest, thereby increasing expression of the protein in a eukaryotic host cell, in particular HEK cells.

CN110551750 refers to improving efficiency of yeast mRNA expression by overexpressing RLI1.

EP3663319 discloses expressing a fusion protein comprising PAB1 and eIF4G, and expressing a protein of interest in yeast.

### SUMMARY OF THE INVENTION

This summary is provided to introduce a selection of concepts in a simplified form that are further described below in the detailed description. This summary is not intended to identify key or essential features of the claimed subject matter, nor is it intended to be used to limit the scope of the claimed subject matter. Other features, details, utilities, and advantages of the claimed subject matter will be apparent from the following written detailed description, including those aspects illustrated in the accompanying drawings and defined in the appended claims.

It is the objective of the invention to improve recombinant protein production in production host cells. It is a particular object to increase the yield of recombinant proteins by increasing translational efficiency.

The objective is solved by the subject of the claims and as further described herein.

The invention provides for a recombinant eukaryotic host cell expressing a gene of interest (GOI) which is engineered by genetic modifications to increase expression of two or more genes encoding translation initiation factors (TIF genes) of the messenger ribonucleoprotein (mRNP), compared to the host cell prior to said one or more genetic modifications.

Specifically, said two of more TIF genes are TIF genes which comprise at least a gene encoding eIF4A and a gene encoding eIF4G.

According to a specific aspect, the TIF genes further comprise any one or more of genes encoding eIF4E, PAB1 or RLI1.

According to specific embodiments, said TIF genes encode TIFs comprising or consisting of the following TIFs, in particular comprising or consisting of the following combinations of TIFs:
a) eIF4A and eIF4G,
b) eIF4A, eIF4G, and eIF4E,
c) eIF4A, eIF4G, eIF4E, and PAB1;
d) eIF4A, eIF4G, and PAB1.

Specifically, any of the combinations of TIFs of embodiments a) to d) above may optionally further comprise RLI1.

According to a specific aspect, the host cell is engineered to overexpress at least
a) genes encoding eIF4A and eIF4G,
b) genes encoding eIF4A, eIF4G, and eIF4E,
c) genes encoding eIF4A, eIF4G, eIF4E, and PAB1;
d) genes encoding eIF4A, eIF4G, and PAB1.

Specifically, the host cell is engineered to overexpress any of the combinations of genes recited in a) to d) above, and may optionally further be engineered to engineered to overexpress RLI1. Specifically, expression of at least one of said TIF genes is under transcriptional control of a promoter that is different from the promoter controlling expression of said GOI. Specifically, the GOI is expressed by a GOI expression cassette (GOIEC) and the respective TIF gene is expressed by a TIF gene expression cassette (TIFEC). The expression cassette comprises or consists of at least a promoter operably linked to the gene to be expressed.

According to a specific aspect, the GOIEC promoter is different from any one or more or all of the TIFEC promoters.

The promoters are specifically comprised in respective separate expression cassettes to express the TIF gene(s) and the GOI.

Specifically, the TIFs of the mRNP are TIFs which are present in the mRNP complex or activated mRNP, such as before binding to the 43S preinitiation complex (PIC). Among such TIFs are particularly one or more of the closed-loop factors, such as eIF4E, eIF4A, eIF4G, PAB1, and/or RLI1, which is understood to be associated to the closed loop structure, and in particularly one or more of the factors of the eIF4F complex, such as eIF4E, eIF4A, or eIF4G.

Specifically, said eIF4G is eIF4G2 (TIF4632, Eukaryotic initiation factor 4F subunit p130), preferably of yeast, such as Pichia or Saccharomyces.

According to a specific embodiment, the host cell is genetically engineered to overexpress at least two, at least three, at least four, or at least five of said TIF genes, wherein said TIF genes at least comprise genes encoding eIF4A and eIF4G.

Specifically, at least two of said TIFs are of the eIF4F complex, in particular eIF4A, eIF4G, and optionally eIF4E. Specifically, two, three, or more, or all of the TIF genes of the eIF4F complex are overexpressed, in particular eIF4A, eIF4G, and optionally eIF4E.

Specifically, at least one of said TIFs may be a closed-loop factor, in particular eIF4G, and optionally any one or both of eIF4E or PAB1. Specifically, one, two, three, or more, or all of the closed-loop factors are overexpressed, in particular eIF4G, and optionally any one or both of eIF4E or PAB1.

Specifically, the TIF genes are of eukaryotic or prokaryotic origin, in particular of yeast or mammalian origin, including naturally-occurring genes or artificial variants thereof, in particular those encoding naturally-occurring TIFs (including naturally-occurring isoforms), or functionally active variants with high sequence identity and about the same or increased function as TIF in the activated mRNP and/or translation initiation in a production host cell.

Specifically, the TIF genes are of eukaryotic origin, such as originating from any of the host cell species that are further described herein, such as originating from:
a) a yeast cell of a genus selected from the group consisting of Pichia, Hansenula, Komagataella, Saccharomyces, Kluyveromyces, Candida, Ogataea, Yarrowia, and Geotrichum, preferably Pichia pastoris, Komagataella phaffii, Komagataella pastoris, Komagataella pseudopastoris, Saccharomyces cerevisiae, Ogataea minuta, Kluyveromces lactis, Kluyveromes marxianus, Yarrowia lipolytica or Hansenula polymorphs; or
b) a cell of filamentous fungi, such as Aspergillus awamori or Trichoderma reesei; or
c) a non-human primate, human, rodent or bovine cell, such as mouse myeloma (NSO)-cell lines, Chinese hamster ovary (CHO)-cell lines, HT1080, H9, HepG2, MCF7, MDBK Jurkat, MDCK, NIH3T3, PC12, BHK (baby hamster kidney cell), VERO, SP2/0, YB2/0, Y0, C127, L cell, COS, e.g., COS1 and COS7, QC1-3, HEK-293, VERO, PER.C6, HeLA, EBI, EB2, EB3, oncolytic or hybridoma-cell lines; or
d) an insect cell, such as Sf9, Mimic™ Sf9, Sf21, High Five (BT1-TN-5B1-4), or BT1-Ea88 cells; or
e) an algae cell, such as of the genus Amphora, Bacillariophyceae, Dunaliella, Chlorella, Chlamydomonas, Cyanophyta (cyanobacteria), Nannochloropsis, Spirulina, or Ochromonas), or
f) a plant cell, such as cells from monocotyledonous plants, preferably maize, rice, wheat, or Setaria, or from a dicotyledonous plant, preferably cassava, potato, soybean, tomato, tobacco, alfalfa, Physcomitrella patens or Arabidopsis.

According to a specific aspect, the TIF genes are of eukaryotic origin, such as originating from the same species origin as the POI. In particular, the TIF gene(s) may be of yeast origin, such as Pichia or Saccharomyces; or of mammalian origin, such as of human or non-human animal origin. According to a specific aspect, any one, two, three, four or five of the TIFs is a yeast or mammalian (such as for example human, mouse, hamster, or ape) protein, including naturally-occurring isoforms.

According to specific embodiments, any of the yeast or human TIF gene(s) are selected for overexpression in the host cell.

Specifically, a TIF as used for the purposes provided herein, comprises or consists of an amino acid sequence which is at least any one of 60%, 70%, 80%, 85%, 90%, 95%, 96%, 97%, 98%, 99% or 100% identical to the sequence of the respective naturally-occurring (also referred to as native, or wild-type) TIF, in particular to any one of the TIFs identified by the sequences provided herein.

According to a specific aspect,
a) said TIF gene is encoding el F4E that comprises or consists of at least any one of 60%, 70%, 80%, 85%, 90%, 95%, 96%, 97%, 98%, 99% or 100% sequence identity to any one of SEQ ID NO:1-11;
b) said TIF gene is encoding el F4A that comprises or consists of at least any one of 60%, 70%, 80%, 85%, 90%, 95%, 96%, 97%, 98%, 99% or 100% sequence identity to any one of SEQ ID NO:12-33;
c) said TIF gene is encoding eIF4G that comprises or consists of at least any one of 60%, 70%, 80%, 85%, 90%, 95%, 96%, 97%, 98%, 99% or 100% sequence identity to any one of SEQ ID NO:34-44;
d) said TIF gene is encoding PAB1 that comprises or consists of at least any one of 60%, 70%, 80%, 85%, 90%, 95%, 96%, 97%, 98%, 99% or 100% sequence identity to any one of SEQ ID NO:45-55; and
e) said TIF gene is encoding RLI1 that comprises or consists of at least any one of 60%, 70%, 80%, 85%, 90%, 95%, 96%, 97%, 98%, 99% or 100% sequence identity to any one of SEQ ID NO:56-65.

According to a specific aspect,
a) the eIF4E protein comprises or consists of at least any one of 60%, 70%, 80%, 85%, 90%, 95%, 96%, 97%, 98%, 99% or 100% sequence identity to any one of SEQ ID NO:1-11;
b) the eIF4A protein comprises or consists of at least any one of 60%, 70%, 80%, 85%, 90%, 95%, 96%, 97%, 98%, 99% or 100% sequence identity to any one of SEQ ID NO:12-33;
c) the eIF4G protein comprises or consists of at least any one of 60%, 70%, 80%, 85%, 90%, 95%, 96%, 97%, 98%, 99% or 100% sequence identity to any one of SEQ ID NO:34-44;
d) the PAB1 protein comprises or consists of at least any one of 60%, 70%, 80%, 85%, 90%, 95%, 96%, 97%, 98%, 99% or 100% sequence identity to any one of SEQ ID NO:45-55; and
e) the RLI1 protein comprises or consists of at least any one of 60%, 70%, 80%, 85%, 90%, 95%, 96%, 97%, 98%, 99% or 100% sequence identity to any one of SEQ ID NO:56-65.

Specifically, a TIF gene as used for the purposes provided herein, is a nucleic acid molecule comprising or consisting of the nucleotide sequence encoding the respective TIF. A TIF gene may comprise or consist of a naturally-occurring (also referred to as native, or wild-type) nucleotide sequence, or be mutated e.g., optimized for expressing said TIF gene in a host cell., e.g., a codon-optimized sequence, or a Golden Gate optimized sequence.

Specifically, a TIF gene as used for the purposes provided herein, comprises or consists of a nucleotide sequence which is at least any one of 60%, 70%, 80%, 85%, 90%, 95%, 96%, 97%, 98%, 99% or 100% identical to the sequence of the respective naturally-occurring (also referred to as native, or wild-type) TIF gene, in particular to any one of the TIF genes identified by the sequences provided herein.

According to a specific aspect, said one or more genetic modifications comprise a knockin, substitution, disruption, deletion or knockout of (i) one or more polynucleotides, or a part thereof; or (ii) an expression control sequence, preferably an expression control sequence selected from the group consisting of a promoter, a ribosomal binding site, transcriptional or translational start and stop sequences, an enhancer and activator sequence, preferably wherein said one or more genetic modifications comprise the integration of a heterologous polynucleotide or expression cassette into the host cell genome.

According to a specific aspect, said one or more genetic modifications include an increase in the number of said TIF gene(s) or the number of expression cassettes comprising said TIF gene(s), and/or a gain-of-function alteration in said TIF gene(s), resulting in increasing the level or activity of said TIF gene(s).

According to a specific aspect, said one or more genetic modifications include a gain-of-function alteration in the respective TIF gene resulting in increasing the level or activity of the TIF, e.g., by overexpressing the respective TIF gene(s), and/or by reducing degradation, or increasing stability of the respective TIF gene(s) or TIF mRNA.

Specifically, said gain-of-function alteration includes a knockin of the respective TIF gene.

Specifically, said gain-of-function alteration up-regulates the respective TIF gene expression in said cell.

Specifically, said gain-of-function alteration includes an insertion of a heterologous expression cassette to overexpress the respective TIF gene in said cell.

Gain-of-function alterations are specifically to increase expression of a TIF gene, including e.g., introducing a polynucleotide encoding the TIF (or a TIFEC comprising such polynucleotide) into the host cell genome, and optionally disrupting the promoter which is operably linked to such polynucleotide, replacing such promoter with another promoter which has higher promoter activity.

Specific methods of modifying gene expression employ modulating (e.g., activating, up-regulating, inactivating, inhibiting, or down-regulating) regulatory sequences which modulate the expression of a polynucleotide (a gene), such as using respective transcription regulators targeted to the relevant sequences by an RNA guided ribonuclease used in a CRISPR based method of modifying a host cell, e.g., regulatory sequences selected from the group consisting of promoter, ribosomal binding sites, transcriptional start or stop sequences, translational start or stop sequences, enhancer or activator sequences, repressor or inhibitor sequences, signal or leader sequences, in particular those which control the expression and/or secretion of a protein.

Specifically, said one or more genetic modifications to increase expression of a TIF gene include one or more genomic mutations including insertion or activation of a respective gene or genomic sequence which increases expression of a gene or part of a gene by at least 50%, 60%, 70%, 80%, 90%, or 95%, or even more e.g., by a knockin of a heterologous gene, or increasing the copy number of the endogenous gene, as compared to the respective host without such genetic modification.

Specifically, the one or more genetic modifications increasing expression comprise genomic mutations which constitutively improve or otherwise increase the expression of one or more endogenous polynucleotides.

Specifically, the one or more genetic modifications increasing expression comprise genomic mutations introducing one or more inducible or repressible regulatory sequences which conditionally improve or otherwise increase the expression of one or more endogenous polynucleotides. Such conditionally active modifications are particularly targeting those regulatory elements and genes which are active and/or expressed dependent on cell culture conditions.

Specifically, the expression of the polynucleotide encoding the respective TIF is increased when using the host cell in a method of producing a protein of interest (POI).

Specifically, upon genetic modification, expression of the respective TIF gene is increased under conditions of the host cell culture during which the POI is produced.

Specifically, the host cell is genetically modified to increase the amount (e.g., the level, activity or concentration) of the respective TIF(s), by at least any one of 50%, 60%, 70%, 80%, 90%, or 95%, (mol/mol), or even more, compared to the host cell without said modification, e.g., by a knockin of one or more respective TIF genes. According to a specific embodiment, the host cell is genetically modified to comprise one or more insertions of (one or more) genomic sequences, in particular genomic sequences encoding the respective TIF(s), which are integrated in the host cell genome. Such host cell is typically provided as a knockin strain.

According to a specific embodiment, once the host cell described herein is cultured in a cell culture, the total amount of the respective overexpressed TIF(s) in the host cell or host cell culture is increased by at least any one of 50%, 60%, 70%, 80%, 90%, or 95%, (activity % or mol/mol), or even by 100% or more, compared to a reference amount expressed or produced by the host cell prior to or without such genetic modification, or compared to a reference amount produced in a respective host cell culture, or compared to the host cell prior to or without said modification.

According to a specific aspect, one or more of said TIF genes are endogenous or heterologous to the host cell. Specifically, said TIF gene(s) are comprised in respective TIF expressing cassettes (TIFECs). Specifically, said TIF gene(s) are expressed in one or more TIFECs.

Specifically, said TIF gene(s) are comprised in one or more heterologous expression cassette(s), in particular comprising a heterologous expression construct containing one or more expression control sequences such as e.g., a promoter, operably linked to a TIF gene. Specifically, said expression construct is not naturally-occurring in said host cell, or integrated within the host cell's genome or chromosome at a site that is different from the site where the respective endogenous TIF gene or expression construct naturally occurs, or provided on an episomal plasmid.

Specifically, any one, two three, four or five, or more, or all TIFECs comprise a promoter referred to as TIF expression cassette (TIFEC) promoter.

Specifically, the TIFEC promoter is a constitutive promoter, or a regulatable promoter such as inducible or de-repressible promoter, which TIFEC promoter is operably linked to the TIF gene to be expressed.

Specifically, at least one, such as any one or more, or all TIFEC promoters used in TIF expression cassettes within the same host cell, are not pAOX1 of P. pastoris, in particular K. pastoris or K. phaffii, or not methanol-inducible. The pAOX1 promoter is understood as the native promoter of the “AOX1” gene which is referred to as the native gene encoding alcohol oxidase 1 of P. pastoris alcohol oxidase 1 identified by UniProtKB-F2QY27.

According to a specific aspect, the host cell further comprises an expression cassette comprising a GOI and one or more expression control sequences operably linked to said GOI to express said GOI in a host cell culture.

Specifically, said GOI is expressed in a GOI expression cassette (GOIEC), which is separate from the TIF expression cassette(s).

Specifically, the GOIEC comprises a promoter referred to as GOI expression cassette (GOIEC) promoter.

Specifically, the GOIEC promoter is a regulatable promoter such as an inducible or de-repressible promoter, or a constitutive promoter, which GOIEC promoter is operably linked to the GOI to be expressed.

Preferably, at least one, such as any one or more, or all TIFEC promoters used in TIF expression cassettes within the same host cell, is any other than the GOIEC promoter.

Specifically,
a) any one or more, or all TIFEC promoters are constitutive, and the GOIEC promoter is an inducible or (de)repressible promoter;
b) any one or more, or all TIFEC promoters are inducible or (de)repressible, and the GOIEC promoter is a constitutive promoter;
c) any one or more, or all TIFEC promoters are constitutive, and the GOIEC promoter is a constitutive promoter of a type or strength that differs from any one or more of such TIFEC promoter(s);
d) any one or more, or all TIFEC promoters are inducible or (de)repressible, and the GOIEC promoter is an inducible or (de)repressible promoter of a type or strength that differs from any one or more of such TIFEC promoter(s).

According to a specific aspect, the GOIEC promoter has a higher promoter strength as compared to any of the TIFEC promoters.

In a preferred embodiment, expression of the polynucleotide encoding a respective TIF is driven by a constitutive promoter and expression of the polynucleotide (gene) encoding the POI is driven by an inducible promoter. In yet another preferred embodiment, expression of the polynucleotide encoding a respective TIF is driven by an inducible promoter and expression of the polynucleotide (gene) encoding the POI is driven by a constitutive promoter.

As an example, expression of the polynucleotide encoding a TIF may be driven by a constitutive GAP promoter and expression of the polynucleotide encoding the POI may be driven by a methanol-inducible promoter, such as the A0X1 or AOX2 promoter.

As another example, expression of the polynucleotide encoding a TIF may be driven by a constitutive promoter such as MDH3, POR1, PDC1, FBA1-1, or GPM1 (Prielhofer et al. 2017, BMC Sys Biol. 11: 123), and expression of the polynucleotide encoding the POI may be driven by a methanol-inducible promoter, such as the A0X1 or AOX2 promoter.

As another example, expression of the polynucleotide encoding a TIF may be driven by a constitutive promoter such as a GAP promoter, and expression of the polynucleotide encoding the POI may be driven by a by a de-repressible promoter, such as those further described herein.

As another example, expression of the polynucleotide encoding a TIF may be driven by a constitutive promoter and expression of the polynucleotide encoding the POI may be driven by a de-repressible promoter, such as those further described herein.

As another example, expression of the polynucleotide encoding a TIF may be driven by a de-repressible promoter, and expression of the polynucleotide encoding the POI may be driven by a de-repressible promoter, such as those further described herein.

Specifically, the expression cassette(s) referred to herein include at least one promoter and the polynucleotide (or gene) to be expressed under transcriptional control of said promoter, and optionally further regulatory sequences, such as selected from the group consisting of ribosomal binding sites, transcriptional start or stop sequences, translational start or stop sequences, enhancer or activator sequences, repressor or inhibitor sequences, signal or leader sequences, in particular those which control the expression and/or secretion of a protein.

Specifically, an expression cassette is used which is heterologous to the host cell, in particular wherein the expression cassette comprises a promoter operably linked to a polynucleotide, wherein the promoter and the polynucleotide are heterologous to each other, meaning that they are not occurring in such combination in nature e.g., wherein either one (or only one) of the promoter and polynucleotide is artificial or heterologous to the other and/or to the host cell described herein; the promoter is an endogenous promoter and the polynucleotide is a heterologous polynucleotide; or the promoter is an artificial or heterologous promoter and the polynucleotide is an endogenous polynucleotide; wherein both, the promoter and polynucleotide, are artificial, heterologous or from different origin, such as from a different species or type (strain) of cells compared to the host cell described herein. Specifically, the promoter is not naturally associated with and/or not operably linked to said polynucleotide in the cell which is used as a host cell described herein.

According to a specific aspect, the heterologous expression cassette is comprised in an autonomously replicating vector or plasmid, or integrated within a chromosome of said host cell.

The GOI-expressing construct may comprise or be composed of the expression control sequence(s) such as e.g., a promoter, operably linked to the Gal, as necessary to express said GOI from said expression construct in the host cell. The GOI-expressing construct may be comprised in a separate expression cassette, or in an expression cassette that additionally expresses one or more of said TIF gene(s).

According to a specific aspect, the host cell is a recombinant host cell comprising at least one heterologous GOIEC, wherein at least one component or combination of components comprised in the GOIEC is heterologous to the host cell. Specifically, an artificial expression cassette is used, in particular wherein the promoter and GOI are heterologous to each other, not occurring in such combination in nature e.g., wherein either one (or only one) of the promoter and GOI is artificial or heterologous to the other and/or to the host cell described herein; the promoter is an endogenous promoter and the GOI is a heterologous GOI; or the promoter is an artificial or heterologous promoter and the GOI is an endogenous GOI; wherein both, the promoter and GOI, are artificial, heterologous or from different origin, such as from a different species or type (strain) of cells compared to the host cell described herein. Specifically, the GOIEC promoter is not naturally associated with and/or not operably linked to said GOI in the cell which is used as a host cell described herein.

Specifically, the host cell comprises:
a) an expression system to express one or more of said TIF genes in one or more heterologous TIF expression cassettes, each comprising one or more expression control sequences operably linked to said TIF gene(s); and
b) a GOI expression cassette comprising a GOI and one or more expression control sequences operably linked to said GOI;
wherein the expression system of a) and the expression cassette of b) are engineered to express the respective TIF gene(s) and GOI when culturing the host cell in a cell culture.

According to a specific aspect, the host cell comprises an expression system to express one, two, three, four or five, or more of said TIF genes in one or more heterologous expression cassettes, each comprising one or more expression control sequences operably linked to said TIF gene(s). In specific embodiments, each TIF gene is operably linked to a TIFEC promoter.

The number of GOIECs or TIEFECs per cell typically determines the amount of the respective expression products. The host cell specifically comprises at least one GOIEC and at least one TIFEC copy per cell. One expression cassette is typically referred to as “one copy”.

According to a specific aspect, the number of one type of GOIEC or GOIEC copies per host cell is at least (or up to) any one of 1, 2, 3, 4, 5, 6, 7, 8, 9, or 10, or even a higher number up to 20, 30, 40, or 50 can be used.

According to a specific aspect, the number of one type of TIFEC or TIFEC copies per host cell is at least (or up to) any one of 1, 2, 3, 4, or 5.

According to a specific aspect, the number of heterologous TIFECs per host cell is at least (or up to) 1, 2, 3, 4, or 5.

While the TIFECs are preferably heterologous to the host cell, the GOIEC may be heterologous or endogenous.

According to a specific aspect, the host cell comprises one or more (e.g. multiple) heterologous expression cassettes, including e.g., one or more expression cassette(s) expressing the TIF(s), and one or more (multiple) copies of an expression cassette expressing the GOI, such as at least 1, 2, 3, 4, or 5 copies (gene copy number, GCN) of a TIFEC or GOIEC. Each of the copies may comprise or consist of the same or different sequences, including the expression control sequences operably linked to to the respective gene to be expressed.

Specifically, for each of the TIFs overexpressed in a host cell, the number of the TIF coding polynucleotides per cell is about (+/−1) the same as for the other overexpressed TIFs, to ensure about the same level of all overexpressed TIFs.

According to a specific aspect,
a) at least one, i.e. any one or more, or all of the TIF expression cassettes comprises a constitutive promoter; and/or
b) the GOI expression cassette comprises an inducible, de-repressible or otherwise regulatable promoter, or a constitutive promoter.

Specifically, the GOI is a polynucleotide or gene that is different from said TIF genes. Specifically, the GOI is expressing a protein of interest (POI). Specifically, the POI is a polypeptide or protein different from said TIFs.

According to a specific aspect, the POI is heterologous to the host cell species.

According to a specific aspect, the POI is a therapeutic or diagnostic product. Preferably, the POI is a therapeutic protein functioning in mammals.

Specifically, the POI is a peptide, polypeptide or protein selected from the group consisting of an antigen-binding protein, a therapeutic protein, an enzyme, a peptide, a protein antibiotic, a toxin fusion protein, a carbohydrate—protein conjugate, a structural protein, a regulatory protein, a vaccine antigen, a growth factor, a hormone, a cytokine, a process enzyme, and a metabolic enzyme.

Specifically, the POI is a eukaryotic protein, preferably a mammalian derived or related protein such as a human protein or a protein comprising a human protein sequence, or a bacterial protein or bacterial derived protein. Any such mammalian, bacterial or artificial protein not naturally-occurring in the yeast host cell is understood to be heterologous to the host cell.

In specific cases, the POI is a multimeric protein, specifically a dimer or tetramer.

Specifically, the antigen-binding protein is selected from the group consisting of
a) antibodies or antibody fragments, such as any of chimeric antibodies, humanized antibodies, bi-specific antibodies, Fab, Fd, scFv, diabodies, triabodies, Fv tetramers, minibodies, single-domain antibodies like VH, VHH, IgNARs, or V-NAR;
b) antibody mimetics, such as Adnectins, Affibodies, Affilins, Affimers, Affitins, Alphabodies, Anticalins, Avimers, DARPins, Fynomers, Kunitz domain peptides, Monobodies, or NanoCLAMPS; or
c) fusion proteins comprising one or more immunoglobulin-fold domains, antibody domains or antibody mimetics.

A specific POI is an antigen-binding molecule such as an antibody, or a fragment thereof, in particular an antibody fragment comprising an antigen-binding domain. Among specific POIs are antibodies such as monoclonal antibodies (mAbs), immunoglobulin (Ig) or immunoglobulin class G (IgG), heavy-chain antibodies (HcAb's), or fragments thereof such as fragment-antigen binding (Fab), Fd, single-chain variable fragment (scFv), or engineered variants thereof such as for example Fv dimers (diabodies), Fv trimers (triabodies), Fv tetramers, or minibodies and single-domain antibodies like VH, VHH, IgNARs, or V-NAR, or any protein comprising an immunoglobulin-fold domain. Further antigen-binding molecules may be selected from antibody mimetics, or (alternative) scaffold proteins such as e.g., engineered Kunitz domains, Adnectins, Affibodies, Affiline, Anticalins, or DARPins.

According to a specific aspect, the POI is e.g., BOTOX, Myobloc, Neurobloc, Dysport (or other serotypes of botulinum neurotoxins), alglucosidase alpha, daptomycin, YH-16, choriogonadotropin alpha, filgrastim, cetrorelix, interleukin-2, aldesleukin, teceleulin, denileukin diftitox, interferon alpha-n3 (injection), interferon alpha-nl, DL-8234, interferon, Suntory (gamma-1a), interferon gamma, thymosin alpha 1, tasonermin, DigiFab, ViperaTAb, EchiTAb, CroFab, nesiritide, abatacept, alefacept, Rebif, eptoterminalfa, teriparatide (osteoporosis), calcitonin injectable (bone disease), calcitonin (nasal, osteoporosis), etanercept, hemoglobin glutamer 250 (bovine), drotrecogin alpha, collagenase, carperitide, recombinant human epidermal growth factor (topical gel, wound healing), DWP401, darbepoetin alpha, epoetin omega, epoetin beta, epoetin alpha, desirudin, lepirudin, bivalirudin, nonacog alpha, Mononine, eptacog alpha (activated), recombinant Factor VIII+VWF, Recombinate, recombinant Factor VIII, Factor VIII (recombinant), Alphnmate, octocog alpha, Factor VIII, palifermin, indikinase, tenecteplase, alteplase, pamiteplase, reteplase, nateplase, monteplase, follitropin alpha, rFSH, hpFSH, micafungin, pegfilgrastim, lenograstim, nartograstim, sermorelin, glucagon, exenatide, pramlintide, iniglucerase, galsulfase, Leucotropin, molgramostirn, triptorelin acetate, histrelin (subcutaneous implant, Hydron), deslorelin, histrelin, nafarelin, leuprolide sustained release depot (ATRIGEL), leuprolide implant (DUROS), goserelin, Eutropin, KP-102 program, somatropin, mecasermin (growth failure), enlfavirtide, Org-33408, insulin glargine, insulin glulisine, insulin (inhaled), insulin lispro, insulin deternir, insulin (buccal, RapidMist), mecasermin rinfabate, anakinra, celmoleukin, 99 mTc-apcitide injection, myelopid, Betaseron, glatiramer acetate, Gepon, sargramostim, oprelvekin, human leukocyte-derived alpha interferons, Bilive, insulin (recombinant), recombinant human insulin, insulin aspart, mecasenin, Roferon-A, interferon-alpha 2, Alfaferone, interferon alfacon-1, interferon alpha, Avonex′ recombinant human luteinizing hormone, dornase alpha, trafermin, ziconotide, taltirelin, diboterminalfa, atosiban, becaplermin, eptifibatide, Zemaira, CTC-111, Shanvac-B, HPV vaccine (quadrivalent), octreotide, lanreotide, ancestirn, agalsidase beta, agalsidase alpha, laronidase, prezatide copper acetate (topical gel), rasburicase, ranibizumab, Actimmune, PEG-Intron, Tricomin, recombinant house dust mite allergy desensitization injection, recombinant human parathyroid hormone (PTH) 1-84 (sc, osteoporosis), epoetin delta, transgenic antithrombin III, Granditropin, Vitrase, recombinant insulin, interferon-alpha (oral lozenge), GEM-21S, vapreotide, idursulfase, omnapatrilat, recombinant serum albumin, certolizumab pegol, glucarpidase, human recombinant Cl esterase inhibitor (angioedema), Ianoteplase, recombinant human growth hormone, enfuvirtide (needle-free injection, Biojector 2000), VGV-1, interferon (alpha), lucinactant, aviptadil (inhaled, pulmonary disease), icatibant, ecallantide, omiganan, Aurograb, pexigananacetate, ADI-PEG-20, LDI-200, degarelix, cintredelinbesudotox, FavId, MDX-1379, ISAtx-247, liraglutide, teriparatide (osteoporosis), tifacogin, AA4500, T4N5 liposome lotion, catumaxomab, DWP413, ART-123, Chrysalin, desmoteplase, amediplase, corifollitropinalpha, TH-9507, teduglutide, Diamyd, DWP-412, growth hormone (sustained release injection), recombinant G-CSF, insulin (inhaled, AIR), insulin (inhaled, Technosphere), insulin (inhaled, AERx), RGN-303, DiaPep277, interferon beta (hepatitis C viral infection (HCV)), interferon alpha-n3 (oral), belatacept, transdermal insulin patches, AMG-531, MBP-8298, Xerecept, opebacan, AIDSVAX, GV-1001, LymphoScan, ranpirnase, Lipoxysan, lusupultide, MP52 (beta-tricalciumphosphate carrier, bone regeneration), melanoma vaccine, sipuleucel-T, CTP-37, Insegia, vitespen, human thrombin (frozen, surgical bleeding), thrombin, TransMlD, alfimeprase, Puricase, terlipressin (intravenous, hepatorenal syndrome), EUR-1008M, recombinant FGF-I (injectable, vascular disease), BDM-E, rotigaptide, ETC-216, P-113, MBI-594AN, duramycin (inhaled, cystic fibrosis), SCV-07, OPI-45, Endostatin, Angiostatin, ABT-510, Bowman Birk Inhibitor Concentrate, XMP-629, 99 mTc-Hynic-Annexin V, kahalalide F, CTCE-9908, teverelix (extended release), ozarelix, rornidepsin, BAY-504798, interleukin4, PRX-321, Pepscan, iboctadekin, rhlactoferrin, TRU-015, IL-21, ATN-161, cilengitide, Albuferon, Biphasix, IRX-2, omega interferon, PCK-3145, CAP-232, pasireotide, huN901-DMI, ovarian cancer immunotherapeutic vaccine, SB-249553, Oncovax-CL, OncoVax-P, BLP-25, CerVax-16, multi-epitope peptide melanoma vaccine (MART-1, gp100, tyrosinase), nemifitide, rAAT (inhaled), rAAT (dermatological), CGRP (inhaled, asthma), pegsunercept, thymosinbeta4, plitidepsin, GTP-200, ramoplanin, GRASPA, OBI-1, AC-100, salmon calcitonin (oral, eligen), calcitonin (oral, osteoporosis), examorelin, capromorelin, Cardeva, velafermin, 131I-TM-601, KK-220, T-10, ularitide, depelestat, hematide, Chrysalin (topical), rNAPc2, recombinant Factor V111 (PEGylated liposomal), bFGF, PEGylated recombinant staphylokinase variant, V-10153, SonoLysis Prolyse, NeuroVax, CZEN-002, islet cell neogenesis therapy, rGLP-1, BIM-51077, LY-548806, exenatide (controlled release, Medisorb), AVE-0010, GA-GCB, avorelin, ACM-9604, linaclotid eacetate, CETi-1, Hemospan, VAL (injectable), fast-acting insulin (injectable, Viadel), intranasal insulin, insulin (inhaled), insulin (oral, eligen), recombinant methionyl human leptin, pitrakinra subcutancous injection, eczema), pitrakinra (inhaled dry powder, asthma), Multikine, RG-1068, MM-093, NBI-6024, AT-001, PI-0824, Org-39141, Cpn10 (autoimmune diseases/inflammation), talactoferrin (topical), rEV-131 (ophthalmic), rEV-131 (respiratory disease), oral recombinant human insulin (diabetes), RPI-78M, oprelvekin (oral), CYT-99007 CTLA4-Ig, DTY-001, valategrast, interferon alpha-n3 (topical), IRX-3, RDP-58, Tauferon, bile salt stimulated lipase, Merispase, alaline phosphatase, EP-2104R, Melanotan-II, bremelanotide, ATL-104, recombinant human microplasmin, AX-200, SEMAX, ACV-1, Xen-2174, CJC-1008, dynorphin A, SI-6603, LAB GHRH, AER-002, BGC-728, malaria vaccine (virosomes, PeviPRO), ALTU-135, parvovirus B19 vaccine, influenza vaccine (recombinant neuraminidase), malaria/HBV vaccine, anthrax vaccine, Vacc-5q, Vacc-4x, HIV vaccine (oral), HPV vaccine, Tat Toxoid, YSPSL, CHS-13340, PTH(1-34) liposomal cream (Novasome), Ostabolin-C, PTH analog (topical, psoriasis), MBRI-93.02, MTB72F vaccine (tuberculosis), MVA-Ag85A vaccine (tuberculosis), FARA04, BA-210, recombinant plague FIV vaccine, AG-702, OxSODrol, rBetV1, Der-p1/Der-p2/Der-p7 allergen-targeting vaccine (dust mite allergy), PR1 peptide antigen (leukemia), mutant ras vaccine, HPV-16 E7 lipopeptide vaccine, labyrinthin vaccine (adenocarcinoma), CML vaccine, WT1-peptide vaccine (cancer), IDD-5, CDX-110, Pentrys, Norelin, CytoFab, P-9808, VT-111, icrocaptide, telbermin (dermatological, diabetic foot ulcer), rupintrivir, reticulose, rGRF, HA, alpha-galactosidase A, ACE-011, ALTU-140, CGX-1160, angiotensin therapeutic vaccine, D-4F, ETC-642, APP-018, rhMBL, SCV-07 (oral, tuberculosis), DRF-7295, ABT-828, ErbB2-specific immunotoxin (anticancer), DT3SSIL-3, TST-10088, PRO-1762, Combotox, cholecystokinin-B/gastrin-receptor binding peptides, 111In-hEGF, AE-37, trasnizumab-DM1, Antagonist G, IL-12 (recombinant), PM-02734, IMP-321, rhIGF-BP3, BLX-883, CUV-1647 (topical), L-19 based radioimmunotherapeutics (cancer), Re-188-P-2045, AMG-386, DC/1540/KLH vaccine (cancer), VX-001, AVE-9633, AC-9301, NY-ESO-1 vaccine (peptides), NA17.A2 peptides, melanoma vaccine (pulsed antigen therapeutic), prostate cancer vaccine, CBP-501, recombinant human lactoferrin (dry eye), FX-06, AP-214, WAP-8294A (injectable), ACP-HIP, SUN-11031, peptide YY [3-36] (obesity, intranasal), FGLL, atacicept, BR3-Fc, BN-003, BA-058, human parathyroid hormone 1-34 (nasal, osteoporosis), F-18-CCR1, AT-1100 (celiac disease/diabetes), JPD-003, PTH(7-34) liposomal cream (Novasome), duramycin (ophthalmic, dry eye), CAB-2, CTCE-0214, GlycoPEGylated erythropoietin, EPO-Fc, CNTO-528, AMG-114, JR-013, Factor XIII, aminocandin, PN-951, 716155, SUN-E7001, TH-0318, BAY-73-7977, teverelix (immediate release), EP-51216, hGH (controlled release, Biosphere), OGP-I, sifuvirtide, TV4710, ALG-889, Org-41259, rhCC10, F-991, thymopentin (pulmonary diseases), r(m)CRP, hepatoselective insulin, subalin, L19-IL-2 fusion protein, elafin, NMK-150, ALTU-139, EN-122004, rhTPO, thrombopoietin receptor agonist (thrombocytopenic disorders), AL-108, AL-208, nerve growth factor antagonists (pain), SLV-317, CGX-1007, INNO-105, oral teriparatide (eligen), GEM-OS1, AC-162352, PRX-302, LFn-p24 fusion vaccine (Therapore), EP-1043, S pneumoniae pediatric vaccine, malaria vaccine, Neisseria meningitidis Group B vaccine, neonatal group B streptococcal vaccine, anthrax vaccine, HCV vaccine (gpE1+gpE2+MF-59), otitis media therapy, HCV vaccine (core antigen+ISCOMATRIX), hPTH(1-34) (transdermal, ViaDerm), 768974, SYN-101, PGN-0052, aviscumnine, BIM-23190, tuberculosis vaccine, multi-epitope tyrosinase peptide, cancer vaccine, enkastim, APC-8024, GI-5005, ACC-001, TTS-CD3, vascular-targeted TNF (solid tumors), desmopressin (buccal controlled-release), onercept, or TP-9201, adalimumab (HUMIRA), infliximab (REMICADE™), rituximab (RITUXAN™/MAB THERA™), etanercept (ENBREL™), bevacizumab (AVASTIN™), trastuzumab (HERCEPTIN™), pegrilgrastim (NEULASTA™), or any other suitable POI including biosimilars and biobetters.

Specifically, the POI is heterologous to the host cell species.

Specifically, the POI is a secreted peptide, polypeptide, or protein, i.e. secreted from the host cell into the cell culture supernatant.

Specifically, the GOI is expressed with a secretion signal sequence, preferably wherein the secretion signal peptide (or a leader comprising a secretion signal peptide) is fused to the N-terminus of the POI.

The invention further provides for a method for producing a host cell as described herein. Specifically, such method comprises genetically engineering a host cell to comprise within one or more heterologous expression cassettes one or more of said TIF genes and a gene of interest (GOI).

According to a further specific aspect, the invention provides for a method for producing a host cell described herein which is capable of producing a protein of interest (POI) in a host cell culture, by genetic engineering the host cell to introduce within one or more expression cassettes, two or more heterologous nucleic acid molecules and expression control sequences operably linked to each of the heterologous nucleic acid molecules, wherein one of the nucleic acid molecules comprises a gene of interest (GOI) encoding the POI, and further one or more nucleic acid molecules encode TIFs such as TIF gene(s), as further described herein.

Specifically, the host cell is provided by genetic engineering of a wild-type host cell.

According to a specific example, the host cell may be produced by first modifying to introduce one or more expression cassettes to express said TIF gene(s). Such modified host cell may then be further engineered to comprise the expression cassette for POI production.

According to another specific example, the host cell may be produced by first engineering to comprise the expression cassette for POI production. Such engineered host cell may be further modified to introduce one or more expression cassettes to express the TIF gene(s).

The invention further provides for a method for producing a protein of interest (POI) encoded by a gene of interest (GOI) by culturing the host cell described herein under conditions to produce said POI.

The invention further provides for a method for producing a protein of interest (POI) in a host cell, comprising the steps:
(i) genetically engineering the host cell to comprise within one or more heterologous expression cassettes said TIF gene(s) as described herein, and a gene of interest (GOI) encoding the POI,
(ii) culturing said host cell in a culture medium under conditions to co-express said TIF gene(s) and said GOI thereby obtaining a POI, and
(iii) recovering the POI from the host cell or culture medium.

Specifically, step i) of the method described herein is carried out before step (ii).

According to a specific example, a wild-type host cell is genetically modified according to step i) of the method described herein.

Specifically, the host cell is provided upon introducing said genetic modifications into a wild-type host cell strain for expressing the heterologous expression cassettes. Yet, according to a specific embodiment, the host cell may have undergone one or more further genetic modifications of a wild-type host cell e.g., to improve the cell's capability of expressing and/or secreting proteins, or to reduce undesired by-products, such as host cell proteins, before genetically modifying according to step i).

Specifically, suitable method steps are employed to produce the recombinant host cell as further described herein.

Specifically, the POI can be produced by culturing the host cell in an appropriate medium, isolating the expressed POI from the cell culture, in particular from the cell culture supernatant or medium upon separating the cells, and purifying it by a method appropriate for the expressed product, in particular upon separating the POI from the cell and purifying by suitable means. Thereby, a purified POI preparation can be produced.

Specifically, the methods described herein are characterized by the features further described herein, in particular by the recombinant host cell and/or expression system as further described herein.

According to a specific aspect, the invention further provides for the use of the host cell described herein for the production of a POI.

Specifically, the POI is produced by expressing said GOI while culturing the host cells under conditions to co-express or overexpress one or more of said TIF genes. Specifically, by such method, expression of said GOI and the production yield of said POI is increased.

Specifically, the host cell is cultured in a culture medium under conditions to co-express one or more of said TIF genes and to secrete said POI into the host cell culture, and the POI is recovered from the host cell culture.

Specifically, the host cell is a cell line cultured in a cell culture, in particular a production host cell line.

According to a specific embodiment, the cell line is cultured under suitable batch, fed-batch or continuous culture conditions. The culture may be performed in microtiter plates, shake-flasks, or a bioreactor, and optionally starting with a batch phase as the first step, followed by a fed-batch phase or a continuous culture phase as the second step.

Specifically, said cell culture employs growing the cells in a batch phase; and culturing the cells to produce said POI in a fed-batch or a continuous cultivation phase, optionally starting with a batch phase as the first step, followed by a fed-batch phase or a continuous culture phase as the second step.

According to a specific aspect, the method described herein comprises a growing phase and a production phase.

Specifically, the method comprises the steps:
a) culturing the host cell under growing conditions (growing phase, or “growth phase”); and a further step
b) culturing the host cell under growth-limiting conditions (production phase), during which the GOI is expressed to produce said POI.

Specifically, the second step b) follows the first step a).

Specifically, the host cell is modified to co-express one or more of said TIF genes at a level that increases the host cell's specific productivity for said POI (μg/g yeast dry mass (YDM) per hour and/or volumetric productivity for said POI (μg/L per hour).

Specifically, by such co-expression the productivity or yield is increased by of at least any one of 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, 3 fold, 3.5 fold, 4 fold, 5 fold, 5.5 fold, 6 fold, 6.5 fold, 7 fold, 7.5 fold, 8 fold, 8.5 fold, 9 fold, 9.5 fold, 10 fold, 10.5 fold, 11 fold, 11.5 fold, or 12 fold, as compared to the comparable host cell expressing said Gal, which is not engineered to co-express said TIFs.

When comparing the host cell described herein for the effect of the genetic modification(s) to produce said TIF(s), it is typically compared to the comparable host cell prior to or without such genetic modification. Comparison is typically made with the same host cell species or type without such genetic modification, which is engineered to produce the POI, in particular when cultured under conditions to produce said POI. However, a comparison can also be made with the same host cell species or type which is not further engineered to produce the POI. The production of said TIF(s) upon expression of the respective coding sequences can be determined by the amount (e.g., the level or concentration) of said TIF(s) produced by the host cell. Specifically, the amount can be determined by a suitable method, such as employing a Western Blot, immunofluorescence imaging, flow cytometry or mass spectrometry, in particular wherein mass spectrometry is liquid chromatography-mass spectrometry (LC-MS), or liquid chromatography tandem-mass spectrometry (LC-MS/MS).

According to a specific aspect, the host cell described herein may undergo one or more further genetic modifications e.g., for improving protein production.

Specifically, the host cell can be further engineered to modify one or more genes influencing proteolytic activity used to generate protease deficient strains, in particular a strain deficient in carboxypeptidase Y activity. Particular examples are described in WO1992017595A1. Further examples of a protease deficient Pichia strain with a functional deficiency in a vacuolar protease, such as proteinase A or proteinase B, are described in U.S. Pat. No. 6,153,424A. Further examples are Pichia strains which have an ade2 deletion, and/or deletions of one or both of the protease genes, PEP4 and PRB1, are provided by e.g., ThermoFisher Scientific.

Specifically, the host cell can be engineered to modify at least one nucleic acid sequence encoding a functional gene product, in particular a protease, selected from the group consisting of PEP4, PRB1, YPS1, YPS2, YMP1, YMP2, YMP1, DAP2, GRHI, PRD1, YSP3, and PRB3, as disclosed in WO2010099195A1.

Overexpression or underexpression of genes encoding helper factors can be applied to enhance expression of a Gal, e.g. as described in WO2015158800A1.

According to a specific aspect, the host cell is a eukaryotic host cell.

Specifically, the host cell is:
a) a yeast cell of a genus selected from the group consisting of Pichia, Hansenula, Komagataella, Saccharomyces, Kluyveromyces, Candida, Ogataea, Yarrowia, and Geotrichum, preferably Pichia pastoris, Komagataella phaffii, Komagataella pastoris, Komagataella pseudopastoris, Saccharomyces cerevisiae, Ogataea minuta, Kluyveromces lactis, Kluyveromes marxianus, Yarrowia lipolytica or Hansenula polymorpha, or
b) a cell of filamentous fungi, such as Aspergillus awamori or Trichoderma reesei; or
c) a non-human primate, human, rodent or bovine cell, such as mouse myeloma (NSO)-cell lines, Chinese hamster ovary (CHO)-cell lines, HT1080, H9, HepG2, MCF7, MDBK Jurkat, MDCK, NIH3T3, PC12, BHK (baby hamster kidney cell), VERO, SP2/0, YB2/0, Y0, C127, L cell, COS, e.g., COS1 and COS7, QC1-3, HEK-293, VERO, PER.C6, HeLA, EBI, EB2, EB3, oncolytic or hybridoma-cell lines; or
d) an insect cell, such as Sf9, Mimic™ Sf9, Sf21, High Five (BT1-TN-5B1-4), or BT1-Ea88 cells; or
e) an algae cell, such as of the genus Amphora, Bacillariophyceae, Dunaliella, Chlorella, Chlamydomonas, Cyanophyta (cyanobacteria), Nannochloropsis, Spirulina, or Ochromonas); or
f) a plant cell, such as cells from monocotyledonous plants, preferably maize, rice, wheat, or Setaria, or from a dicotyledonous plant, preferably cassava, potato, soybean, tomato, tobacco, alfalfa, Physcomitrella patens or Arabidopsis.

According to a specific aspect, the host cell can be any yeast cell. Specifically the host cell is a cell of a genus selected from the group consisting of Pichia, Hansenula, Komagataella, Saccharomyces, Kluyveromyces, Candida, Ogataea, Yarrowia, and

Geotrichum, specifically Saccharomyces cerevisiae, Pichia pastoris, Ogataea minuta, Kluyveromces lactis, Kluyveromes marxianus, Yarrowia lipolytica or Hansenula polymorpha, or of filamentous fungi like Aspergillus awamori or Trichoderma reesei. Preferably, the host cell is a methylotrophic yeast, preferably Pichia pastoris. Herein Pichia pastoris is used synonymously for all, Komagataella pastoris, Komagataella phaffii and Komagataella pseudopastoris.

Specific examples refer to a yeast cell of a a Pichia genus (e.g. Pichia pastoris, Pichia methanolica, Pichia kluyveri, and Pichia angusta), Komagataella genus (e.g., Komagataella pastoris, Komagataella pseudopastoris or Komagataella phaffii), Saccharomyces genus (e.g. Saccharomyces cerevisae, Saccharomyces kluyveri, Saccharomyces uvarum), Kluyveromyces genus (e.g. Kluyveromyces lactis, Kluyveromyces marxianus), the Candida genus (e.g. Candida utilis, Candida cacaoi, Candida bolding, the Geotrichum genus (e.g. Geotrichum fermentans), Hansenula polymorpha, Yarrowia lipolytica, or Schizosaccharomyces pombe.

Preferred is the species Pichia pastoris. Specifically, the host cell is a Pichia pastoris strain selected from the group consisting of CBS704, CBS2612, CBS7435, CBS9173-9189, DSMZ 70877, X-33, GS115, KM71, KM71H and SMD1168.

Sources: CBS704 (=NRRL Y-1603=DSMZ 70382), CBS2612 (=NRRL Y-7556), CBS7435 (=NRRL Y-11430), CBS9173-9189 (CBS strains: CBS-KNAW Fungal Biodiversity Centre, Centraalbureau voor Schimmelculturen, Utrecht, The Netherlands), and DSMZ 70877 (German Collection of Microorganisms and Cell Cultures); strains from Thermo Fisher, such as X-33, GS115, KM71, KM71H and SMD1168.

Examples of preferred S. cerevisiae strains include W303, CEN.PK and the BY-series (EUROSCARF collection). All of the strains described above have been successfully used to produce transformants and express heterologous genes.

The invention further provides for a method of increasing the yield of a protein of interest (POI) when produced by a host cell expressing a gene of interest (GOI) encoding said POI, by co-expressing one or more heterologous expression cassettes expressing one or more TIF gene(s) of the messenger ribonucleoprotein (mRNP) in a cell culture.

The invention further provides for a polypeptide expression system comprising one or more heterologous expression cassettes expressing one or more TIF gene(s) of the messenger ribonucleoprotein (mRNP), such as the TIF gene(s) as further described herein. Such expression cassette is herein also referred to as TIF-expressing construct, or TIF (TIF gene) expression cassette (TIFEC). Specifically, a heterologous expression cassette comprises one or more expression control sequences operably linked to said TIF gene to express said TIF gene, in particular wherein at least one of said expression control sequences such as e.g., a promoter, a signal peptide or a leader, is not naturally operably linked to said TIF gene. Specifically, the TIF expression cassette is characterized as further described herein.

Specifically, the expression system further comprises an expression cassette comprising a GOI encoding a protein of interest (POI) and one or more expression control sequences operably linked to said GOI. Such expression cassette is herein also referred to as GOI-expressing construct (GOIEC), or GOI expression cassette. Specifically, the GOI expression cassette is characterized as further described herein. Specifically, the expression cassette comprising the GOI is separate from the other expression cassettes expressing TIF gene(s).

Specifically, the expression system described herein is characterized by the features of the expression cassettes and recombinant expression constructs as further described herein.

Specifically, the TIF gene(s) which are overexpressed by said genetic engineering are each comprised in separate expression cassettes. Yet, an expression cassette may be used comprising at least two or three of the TIF gene(s), and optionally further comprising the GOI.

The invention further provides for a host cell, in particular a host cell, such as described herein, comprising the expression system described herein, in particular the expression system comprising expression cassettes to express the TIF gene(s) and the expression cassette to express the GOI.

According to a specific aspect, the host cell is a recombinant host cell comprising at least one heterologous GOIEC, which comprises an expression cassette promoter operably linked to the GOI, wherein at least one component or combination of components comprised in the GOIEC is heterologous to the host cell.

Specifically, an artificial expression cassette is used, in particular wherein the promoter and gene to be expressed under the control of said promoter are heterologous to each other, not occurring in such combination in nature e.g., wherein either one (or only one) of the promoter and the gene is artificial or heterologous to the other and/or to the host cell described herein; the promoter is an endogenous promoter and the gene to be expressed is a heterologous gene; or the promoter is an artificial or heterologous promoter and the gene is an endogenous gene; wherein both, the promoter and gene, are artificial, heterologous or from different origin, such as from a different species or type (strain) of cells compared to the host cell described herein.

According to a specific aspect, any one or more (or all) of the heterologous expression cassettes is comprised in one or more autonomously replicating vectors or plasmids, or integrated within a chromosome of said host cell.

An expression cassette may be introduced into the host cell and integrated into the host cell genome (or any of its chromosomes) as intrachromosomal element e.g., at a specific site of integration or randomly integrated, whereupon a high producer host cell line is selected. Alternatively, an expression cassette may be integrated within an extrachromosomal genetic element, such as a plasmid or an artificial chromosome e.g., a yeast artificial chromosome (YAC). According to a specific example, an expression cassette is introduced into the host cell by a vector, in particular an expression vector, which is introduced into the host cell by a suitable transformation or transfection technique. For this purpose, the heterologous polynucleotide(s) to be expressed (in particular the GOI) may be ligated into an expression vector.

A preferred yeast expression vector (which is preferably used for expression in yeast) is selected from the group consisting of plasmids derived from pPICZ, pGAPZ, pPIC9, pPICZalfa, pGAPZalfa, pPIC9K, pGAPHis, pPUZZLE or GoldenPiCS.

Techniques for transfecting or transforming host cells for introducing a vector or plasmid are well known in the art. These can include electroporation, spheroplasting, lipid vesicle mediated uptake, heat shock mediated uptake, calcium phosphate mediated transfection (calcium phosphate/DNA co-precipitation), viral infection, and particularly using modified viruses such as, for example, modified adenoviruses, microinjection and electroporation.

As used herein, the term “transforming” a yeast cell is understood to encompass “transfecting” the same.

Transformants as described herein can be obtained by introducing the expression cassette, vector or plasmid DNA into a host and selecting transformants which express the relevant protein or selection marker. Host cells can be treated to introduce heterologous or foreign DNA by methods conventionally used for transformation of host cells, such as the electric pulse method, the protoplast method, the lithium acetate method, and modified methods thereof. Preferred methods of transformation for the uptake of the recombinant DNA fragment by the microorganism include chemical transformation, electroporation or transformation by protoplastation.

According to a specific aspect, an expression cassette is used comprising or consisting of an artificial fusion of polynucleotides, including a promoter operably linked to the heterologous polynucleotide, and optionally further sequences, such as a signal, leader, or a terminator sequence.

Specifically, the TIFEC expresses said TIF(s) as intracellularly protein(s).

Specifically, the GOIEC comprises signal and leader sequences, as necessary to express and produce the POI as secreted proteins.

According to a specific aspect, the GOI is fused at the 5′-end to a nucleotide sequence encoding a secretion signal sequence, preferably a heterologous secretion signal sequence.

According to a specific aspect, the GOIEC comprises a nucleotide sequence encoding a signal peptide enabling the secretion of the POI. Specifically, the nucleotide sequence encoding the signal peptide is fused adjacent to the 5′-end of the GOI.

The signal sequence may be of a native signal sequence, herein understood as the signal sequence which is co-expressed, fused or otherwise associated with the naturally-occurring protein, to secrete such protein upon expression. For example, a native secretion signal sequence is typically a signal sequence co-expressed, fused or otherwise associated with the respective protein to be secreted. Specifically, a native secretion signal sequence is used which is heterologous to (or not natively associated with) said POI, such as a signal sequence that is originating from a secreted protein that differs from said POI.

Alternatively, an artificial secretion signal sequence, in particular a signal sequence which is of at least any one of 85%, 90%, or 95% sequence identity to a naturally-occurring one, can be used.

Specifically, the signal sequence is selected from the group consisting of signal sequences from S. cerevisiae alpha-mating factor prepro-peptide, the signal sequences from the P. pastoris acid phosphatase gene (PHO1) and the extracellular protein X (EPX1) (Heiss, S., V. Puxbaum, C. Gruber, F. Altmann, D. Mattanovich & B. Gasser, Microbiology 2015; 161(7): 1356-68).

Specifically, any of the signal and/or leader sequences as described in WO2014067926 A1 or WO2012152823 A1 can be used.

### FIGURES

FIG. 1: Sequences referred to herein

### DETAILED DESCRIPTION OF THE INVENTION

Unless indicated or defined otherwise, all terms used herein have their usual meaning in the art, which will be clear to the skilled person. Reference is for example made to the standard handbooks, such as Sambrook et al, “Molecular Cloning: A Laboratory Manual” (2nd Ed.), Vols. 1-3, Cold Spring Harbor Laboratory Press (1989); Lewin, “Genes IV”, Oxford University Press, New York, (1990), and Janeway et al., “Immunobiology” (5th Ed., or more recent editions), Garland Science, New York, 2001.

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−10% or +/−5% of the given value.

Specific terms as used throughout the specification have the following meaning.

The term “cell” with respect to a “host cell” as used herein shall refer to a single cell, a single cell clone, or a cell line of a host cell.

The term “cell line” as used herein refers to an established clone of a particular cell type that has acquired the ability to proliferate over a prolonged period of time. A cell line is typically used for expressing an endogenous or recombinant nucleic acid molecule or gene, or products of a metabolic pathway to produce polypeptides or cell metabolites mediated by such polypeptides. A “production host cell line” or “production cell line” is commonly understood to be a cell line ready-to-use for cell culture in a bioreactor to obtain the product of a production process, such as a POI.

Specific embodiments described herein refer to a production host cell line which is engineered to co-express at least two different polynucleotides (nucleic acid molecules or genes), at least one TIF gene encoding a TIF as described herein, and at least one gene of interest (GOI) encoding a POI, in particular wherein a POI is produced in a high yield by co-expressing the respective polynucleotides.

The host cell producing the POI as described herein is also referred to as “production host cell”, and a respective cell line a “production cell line”. Specific embodiments described herein refer to such POI production host cell line which is engineered to co-express said TIF(s), and which is characterized by a high yield of POI production.

The term “host cell” as used herein shall particularly apply to any cell, which is suitably used for recombination purposes to produce a POI or a host cell metabolite. It is well understood that the term “host cell” does not include human beings. Specifically, recombinant host cells as described herein are artificial organisms and derivatives of native (wild-type) host cells. It is well understood that the host cells, methods and uses described herein, e.g., specifically referring to those comprising one or more genetic modifications, heterologous expression cassettes or artificial expression constructs, said transfected or transformed host cells and recombinant proteins, are non-naturally occurring, are “man-made” or synthetic, and are therefore not considered as a result of “law of nature”. Genetic modifications described herein may employ tools, methods and techniques known in the art, such as described by J. Sambrook et al., Molecular Cloning: A Laboratory Manual (3rd edition), Cold Spring Harbor Laboratory, Cold Spring Harbor Laboratory Press, New York (2001).

The term “cell culture” or “culturing” or “cultivation” as used herein with respect to a host cell refers to the maintenance of cells in an artificial, e.g., an in vitro environment, under conditions favoring growth, differentiation or continued viability, in an active or quiescent state, of the cells, specifically in a controlled bioreactor according to methods known in the industry.

When culturing a cell culture using appropriate culture media, the cells are brought into contact with the media in a culture vessel or with substrate under conditions suitable to support culturing the cells in the cell culture. Standard cell culture media and techniques are well-known in the art.

The cell cultures as described herein particularly employ techniques which provide for the production of a secreted POI, such as to obtain the POI in the cell culture medium, which is separable from the cellular biomass, herein referred to as “cell culture supernatant”, and may be purified to obtain the POI at a higher degree of purity. When a protein (such as e.g., a POI) is produced and secreted by the host cell in a cell culture, it is herein understood that such proteins are secreted into the cell culture supernatant, and can be obtained by separating the cell culture supernatant from the host cell biomass, and optionally further purifying the protein to produce a purified protein preparation.

Cell culture media provide the nutrients necessary to maintain and grow cells in a controlled, artificial and in vitro environment. Characteristics and compositions of the cell culture media vary depending on the particular cellular requirements. Important parameters include osmolality, pH, and nutrient formulations. Feeding of nutrients may be done in a continuous or discontinuous mode according to methods known in the art.

Whereas a batch process is a cell culture mode in which all the nutrients necessary for culturing the cells are contained in the initial culture medium, without additional supply of further nutrients during fermentation, in a fed-batch or continuous process, after a batch phase, a feeding phase takes place in which one or more nutrients are supplied to the culture by feeding. Although in most processes the mode of feeding is critical and important, the host cell and methods described herein are not restricted with regard to a certain mode of cell culture.

A recombinant POI can be produced using the host cell and the respective cell line described herein, by culturing in an appropriate medium, isolating the expressed product or metabolite from the culture, and optionally purifying it by a suitable method.

Several different approaches for the production of the POI as described herein are preferred. A POI may be expressed, processed and optionally secreted by transforming or transfecting a host cell with an expression vector harboring recombinant DNA encoding the relevant protein, preparing a culture of the transformed or transfected cell, growing the culture, inducing transcription and POI production, and recovering the POI.

In certain embodiments, the cell culture process is a fed-batch process. Specifically, a host cell transfected with a nucleic acid construct encoding a desired recombinant POI, is cultured in a growth phase and transitioned to a production phase in order to produce a desired recombinant POI.

In another embodiment, host cells described herein are cultured in a continuous mode, e.g., employing a chemostat. A continuous fermentation process is characterized by a defined, constant and continuous rate of feeding of fresh culture medium into a bioreactor, whereby culture broth is at the same time removed from the bioreactor at the same defined, constant and continuous removal rate. By keeping culture medium, feeding rate and removal rate at the same constant level, the cell culture parameters and conditions in the bioreactor remain constant.

In another embodiment, host cells described herein are cultured in a perfusion mode, e.g., culturing cells within a device while supplying fresh medium and removing the supernatant.

A stable cell culture as described herein is specifically understood to refer to a cell culture maintaining the genetic properties, specifically keeping the POI production level high, e.g. at least at a pg level, even after about 20 generations of cultivation, preferably at least 30 generations, more preferably at least 40 generations, most preferred of at least 50 generations. Specifically, a stable recombinant host cell line is provided which is considered a great advantage when used for industrial scale production.

The cell culture described herein is particularly advantageous for methods on an industrial manufacturing scale, e.g. with respect to both the volume and the technical system, in combination with a cultivation mode that is based on feeding of nutrients, in particular a fed-batch or batch process, or a continuous or semi-continuous process (e.g. chemostat).

The host cell described herein is typically tested for its capacity to express the GOI for POI production, tested for the POI yield by any of the following tests: ELISA, activity assay, capillary electrophoresis, HPLC, or other suitable tests, such as SDS-PAGE and Western Blotting techniques, or mass spectrometry.

To determine the effect of co-expressing one or more TIF(s), e.g., the effect on POI production, the host cell line may be cultured in microtiter plates, shake flask, or bioreactor using fed-batch or chemostat fermentations in comparison with strains without such genetic modification for co-expression in the respective cell.

The production method described herein specifically allows for the fermentation on a pilot or industrial scale. The industrial process scale would preferably employ volumes of at least 10 L, specifically at least 50 L, preferably at least 1 m³, preferably at least 10 m³, most preferably at least 100 m³.

Production conditions in industrial scale are preferred, which refer to e.g., fed batch culture in reactor volumes of 100 L to 10 m³ or larger, employing typical process times of several days, or continuous processes in fermenter volumes of approximately 50-1000 L or larger, with dilution rates of approximately 0.001-0.15 h⁻¹.

The devices, facilities and methods used for the purpose described herein are specifically suitable for use in and with culturing any desired cell line. Further, the devices, facilities and methods are suitable for culturing any yeast host cell type, and are particularly suitable for production operations configured for production of pharmaceutical and biopharmaceutical products—such as polypeptide products (POI), nucleic acid products (for example DNA or RNA), or cells and/or viruses such as those used in cellular and/or viral therapies.

In certain embodiments, the cells express or produce a product, such as a recombinant therapeutic or diagnostic product. As described in more detail herein, examples of products produced by cells include, but are not limited to, POIs such as exemplified herein including antibody molecules (e.g., monoclonal antibodies, bispecific antibodies), antibody mimetics (polypeptide molecules that bind specifically to antigens but that are not structurally related to antibodies such as e.g. DARPins, affibodies, adnectins, or IgNARs), fusion proteins (e.g., Fc fusion proteins, chimeric cytokines), other recombinant proteins (e.g., glycosylated proteins, enzymes, hormones), or viral therapeutics (e.g., anti-cancer oncolytic viruses, viral vectors for gene therapy and viral immunotherapy), cell therapeutics (e.g., pluripotent stem cells, mesenchymal stem cells and adult stem cells), vaccines or lipid-encapsulated particles (e.g., exosomes, virus-like particles), RNA (such as e.g. siRNA) or DNA (such as e.g. plasmid DNA), antibiotics or amino acids. In embodiments, the devices, facilities and methods can be used for producing biosimilars.

As mentioned, in certain embodiments, devices, facilities and methods allow for the production of eukaryotic cells, such as for example yeast cells, and/or products of said cells, e.g., POIs including proteins, peptides, or antibiotics, amino acids, nucleic acids (such as DNA or RNA), synthesized by said cells in a large-scale manner. Unless stated otherwise herein, the devices, facilities, and methods can include any desired volume or production capacity including but not limited to bench-scale, pilot-scale, and full production scale capacities.

Moreover, and unless stated otherwise herein, the devices, facilities, and methods can include any suitable reactor(s) including but not limited to stirred tank, airlift, fiber, microfiber, hollow fiber, ceramic matrix, fluidized bed, fixed bed, and/or spouted bed bioreactors. As used herein, “reactor” can include a fermenter or fermentation unit, or any other reaction vessel and the term “reactor” is used interchangeably with “fermenter.” For example, in some aspects, an example bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO₂ levels, maintenance of pH level, agitation (e.g., stirring), and/or cleaning/sterilizing. Example reactor units, such as a fermentation unit, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. In various embodiments, the bioreactor can be suitable for batch, semi fed-batch, fed-batch, perfusion, and/or a continuous fermentation process. Any suitable reactor diameter can be used. In embodiments, the bioreactor can have a volume between about 100 mL and about 50,000 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable reactors can be multi-use, single-use, disposable, or non-disposable and can be formed of any suitable material including metal alloys such as stainless steel (e.g., 316L or any other suitable stainless steel) and Inconel, plastics, and/or glass.

In embodiments and unless stated otherwise herein, the devices, facilities, and methods described herein can also include any suitable unit operation and/or equipment not otherwise mentioned, such as operations and/or equipment for separation, purification, and isolation of such products. Any suitable facility and environment can be used, such as traditional stick-built facilities, modular, mobile and temporary facilities, or any other suitable construction, facility, and/or layout. For example, in some embodiments modular clean-rooms can be used. Additionally, and unless otherwise stated, the devices, systems, and methods described herein can be housed and/or performed in a single location or facility or alternatively be housed and/or performed at separate or multiple locations and/or facilities.

Suitable techniques may encompass culturing in a bioreactor starting with a batch phase, followed by a short exponential fed batch phase at high specific growth rate, further followed by a fed batch phase at a low specific growth rate. Another suitable culture technique may encompass a batch phase followed by a fed-batch phase at any suitable specific growth rate or combinations of specific growth rates such as going from high to low growth rate over POI production time, or from low to high growth rate over POI production time. Another suitable culture technique may encompass a batch phase followed by a continuous culturing phase at a low dilution rate.

A preferred embodiment includes a batch culture to provide biomass followed by a fed-batch culture for high yield POI production.

It is preferred to culture a host cell as described herein in a bioreactor under growth conditions to obtain a cell density of at least 1 g/L cell dry weight, more preferably at least 10 g/L cell dry weight, preferably at least 20 g/L cell dry weight, preferably at least any one of 30, 40, 50, 60, 70, or 80 g/L cell dry weight. It is advantageous to provide for such yields of biomass production on a pilot or industrial scale.

A growth medium allowing the accumulation of biomass, specifically a basal growth medium, typically comprises a carbon source, a nitrogen source, a source for sulphur and a source for phosphate. Typically, such a medium comprises furthermore trace elements and vitamins, and may further comprise amino acids, peptone or yeast extract.

Preferred nitrogen sources include NH₄H₂PO₄, or NH₃ or (NH₄)₂SO₄, Preferred sulphur sources include MgSO₄, or (NH₄)₂SO₄ or K₂SO₄, Preferred phosphate sources include NH₄H₂PO₄, or H₃PO₄, or NaH₂PO₄, KH₂PO₄, Na₂HPO₄ or K₂HPO₄;

Further typical medium components include KCl, CaCl₂), and Trace elements such as: Fe, Co, Cu, Ni, Zn, Mo, Mn, I, B;

Preferably the medium is supplemented with vitamins essential for growth, e.g., B vitamins such as B7; A typical growth medium for P. pastoris comprises glycerol, sorbitol or glucose, NH₄H₂PO₄, MgSO₄, KCl, CaCl₂), biotin, and trace elements.

In the production phase a production medium is specifically used with only a limited amount of a supplemental carbon source.

Preferably the host cell line is cultured in a mineral medium with a suitable carbon source, thereby further simplifying the isolation process significantly. An example of a preferred mineral medium is one containing an utilizable carbon source (e.g., glucose, glycerol, sorbitol, methanol, ethanol, or combinations thereof), salts containing the macro elements (potassium, magnesium, calcium, ammonium, chloride, sulphate, phosphate) and trace elements (copper, iodide, manganese, molybdate, cobalt, zinc, and iron salts, and boric acid), and optionally vitamins or amino acids, e.g., to complement auxotrophies.

Specifically, the cells are cultured under conditions suitable to effect expression of the desired POI, which can be purified from the cells or culture medium, depending on the nature of the expression system and the expressed protein, e.g., whether the protein is fused to a signal peptide and whether the protein is soluble or membrane-bound. As will be understood by the skilled artisan, culture conditions will vary according to factors that include the type of host cell and particular expression vector employed.

A typical production medium comprises a supplemental carbon source, and further NH₄H₂PO₄, MgSO₄, KCl, CaCl₂), biotin, and trace elements.

For example, the feed of the supplemental carbon source added to the fermentation may comprise a carbon source with up to 50 wt % utilizable sugars, or up to 100% utilizable alcohols.

The fermentation preferably is carried out at a pH ranging from 3 to 8.

Typical fermentation times are about 24 to 120 hours with temperatures in the range of 20° C. to 35° C., preferably 22-30° C.

The POI is preferably expressed employing conditions to produce yields of at least 1 mg/L, preferably at least 10 mg/L, preferably at least 100 mg/L, most preferred at least 1 g/L.

The term “expression” or “expression cassette” is herein understood to refer to nucleic acid molecules (herein also referred to as polynucleotides), which contain a desired coding sequence (herein referred to as a gene), and control sequences in operable linkage, so that hosts transformed or transfected with these molecules incorporate the respective sequences and are capable of producing the encoded proteins or host cell metabolites. The term “expression” as used herein refers to expression of a polynucleotide or gene, or to the expression of the respective polypeptide or protein.

One or more expression cassettes are herein also understood as “expression system”. The expression system may be included in an expression construct, such as a vector; however, the relevant DNA may also be integrated into a host cell chromosome. Expression may refer to secreted or non-secreted expression products, including polypeptides or metabolites.

Expression cassettes are conveniently provided as expression constructs e.g., in the form of “vectors” or “plasmids”, which are typically DNA sequences that are required for the transcription of cloned recombinant nucleotide sequences, i.e. of recombinant genes and the translation of their mRNA in a suitable host organism. Expression vectors or plasmids usually comprise an origin for autonomous replication or a locus for genome integration in the host cells, selectable markers (e.g., an amino acid synthesis gene or a gene conferring resistance to antibiotics such as zeocin, kanamycin, G418 or hygromycin, nourseothricin), a number of restriction enzyme cleavage sites, a suitable promoter sequence and a transcription terminator, which components are operably linked together. The terms “plasmid” and “vector” as used herein include autonomously replicating nucleotide sequences as well as genome integrating nucleotide sequences, such as artificial chromosomes e.g., a yeast artificial chromosome (YAC).

Expression vectors may include but are not limited to cloning vectors, modified cloning vectors and specifically designed plasmids. Preferred expression vectors described herein are expression vectors suitable for expressing of a recombinant gene in a eukaryotic host cell and are selected depending on the host organism. Appropriate expression vectors typically comprise regulatory sequences suitable for expressing DNA encoding a POI in a eukaryotic host cell. Examples of regulatory sequences include promoter, operators, enhancers, ribosomal binding sites, and sequences that control transcription and translation initiation and termination. The regulatory sequences are typically operably linked to the DNA sequence to be expressed.

To allow expression of a recombinant nucleotide sequence in a host cell, a promoter sequence is typically regulating and initiating transcription of the downstream nucleotide sequence, with which it is operably linked. An expression cassette or vector typically comprises a promoter nucleotide sequence which is adjacent to the 5′ end of a coding sequence, e.g., upstream from and adjacent to the coding sequence (e.g., encoding a helper factor) or gene of interest (GOI), or if a signal or leader sequence is used, upstream from and adjacent to said signal and leader sequence, respectively, to facilitate translation initiation and expression of coding sequences to obtain the expression product (e.g., TIF(s) or the POI).

Specific expression constructs described herein comprise a promoter operably linked to a nucleotide sequence encoding a TIF or POI under the transcriptional control of said promoter. Specifically, the promoter can be used which is not natively associated with said coding sequence.

Specific expression constructs described herein comprise a polynucleotide encoding the POI linked with a leader sequence (e.g., a secretion signal peptide sequence (pre-sequence), or a pro-sequence), which causes transport of the POI into the secretory pathway and/or secretion of the POI from the host cell. The presence of such a secretion leader sequence in the expression vector is typically required when the POI intended for recombinant expression and secretion is a protein which is not naturally secreted and therefore lacks a natural secretion leader sequence, or its nucleotide sequence has been cloned without its natural secretion leader sequence. In general, any secretion leader sequence effective to cause secretion of the POI from the host cell may be used. The secretion leader sequence may originate from yeast source, e.g. from yeast alpha-factor such as MFα of Saccharomyces cerevisiae, or yeast phosphatase, from mammalian or plant source, or others.

In specific embodiments, multicloning vectors may be used, which are vectors having a multicloning site. Specifically, a desired heterologous polynucleotide can be integrated or incorporated at a multicloning site to prepare an expression vector. In the case of multicloning vectors, a promoter is typically placed upstream of the multicloning site.

The term “gene expression”, or “expressing a polynucleotide” or “expressing a nucleic acid molecule” as used herein, is meant to encompass at least one step selected from the group consisting of DNA transcription into mRNA, mRNA translation and processing, mRNA maturation, mRNA export, protein folding and/or protein transport.

The term “polynucleotide” “nucleic acid molecule(s)” or “nucleic acid sequence(s)” as interchangeably used herein, refers to nucleotides, either ribonucleotides or deoxyribonucleotides or a combination of both, in a polymeric unbranched form of any length. Preferably, a polynucleotide refers to deoxyribonucleotides in a polymeric unbranched form of any length. Here, nucleotides consist of a pentose sugar (deoxyribose), a nitrogenous base (adenine, guanine, cytosine or thymine) and a phosphate group.

The term “co-express” or “co-expression” as used herein shall mean the concomitant or consecutive (yet, while culturing the cell in the same cell culture or containment) or simultaneous expression of at least two or multiple polynucleotides (nucleic acid molecules, such as genes) in a host cell, cell line or cell culture e.g., at about the same or different amounts or ratios.

As described herein polynucleotides (like TIF gene(s)) may be co-expressed such that at least one of the polynucleotides (like a GOI) is overexpressed.

A host cell co-expressing TIF gene(s) such as described herein is specifically genetically engineered and modified to increase expression of said TIF gene(s) in the host cell culture, which is herein also referred to as “overexpression” or “co-overexpression”.

The term “overexpress” or “overexpression” as used herein shall refer to expression of an expression product, such as a polypeptide or protein, at a level greater than the expression of the same expression product prior to a genetic modification of the host cell or in a comparable host which has not been genetically modified at defined conditions. TIFs being heterologous to a host cell are always understood to be overexpressed, if such host cell is expressing such TIFs. For example, where a host cell as described herein does not natively express any of said TIFs, heterologous polynucleotides encoding such TIFs proteins are newly introduced into the host cell for expression; in such case, any detectable expression of such TIFs is encompassed by the term “overexpression.”

Overexpression of a gene encoding a protein (such as a TIF gene) is also referred to as overexpression of a protein (such as a TIF). Overexpression can be achieved in any ways known to a skilled person in the art. In general, it can be achieved by increasing transcription/translation of the gene, e.g. by increasing the copy number of the gene or altering or modifying regulatory sequences or sites associated with expression of a gene. For example, the gene can be operably linked to a strong promoter in order to reach high expression levels. Such promoters can be endogenous promoters or heterologous, in particular recombinant promoters. One can substitute a promoter with a heterologous promoter which increases expression of the gene. Using inducible promoters additionally makes it possible to increase the expression in the course of cultivation. Furthermore, overexpression can also be achieved by, for example, modifying the chromosomal location of a particular gene, altering nucleic acid sequences adjacent to a particular gene such as a ribosome binding site or transcription terminator, introducing a frame-shift in the open reading frame, modifying proteins (e.g., regulatory proteins, suppressors, enhancers, transcriptional activators and the like) involved in transcription of the gene and/or translation of the gene product, or any other conventional means of deregulating expression of a particular gene routine in the art (including but not limited to use of antisense nucleic acid molecules, for example, to block expression of repressor proteins or deleting or mutating the gene for a transcriptional factor which normally represses expression of the gene desired to be overexpressed. Prolonging the life of the mRNA may also improve the level of expression. For example, certain terminator regions may be used to extend the half-lives of mRNA. If multiple copies of genes are included, the genes can either be located in plasmids of variable copy number or integrated and amplified in the chromosome. It is possible to introduce one or more genes or genomic sequences into the host cell for expression.

According to a specific embodiment, a polynucleotide encoding the respective TIF can be presented in a single copy or in multiple copies per cell. The copies may be adjacent to or distant from each other. According to another specific embodiment, overexpression of the respective TIF employs recombinant nucleotide sequences encoding the TIF provided on one or more plasmids suitable for integration into the genome (i.e., knockin) of the host cell, in a single copy or in multiple copies per cell. The copies may be adjacent to or distant from each other. Overexpression can be achieved by expressing multiple copies of the polynucleotide, such as 2, 3, 4, 5, 6 or more copies of said polynucleotide per host cell.

A recombinant nucleotide sequence comprising a GOI and a polynucleotide (gene) encoding the respective TIF may be provided on one or more autonomously replicating plasmids, and introduced in a single copy or in multiple copies per cell.

Alternatively, the recombinant nucleotide sequence comprising a GOI and a polynucleotide (gene) encoding the TIF may be present on the same plasmid, and introduced in a single copy or multiple copies per cell.

A heterologous polynucleotide (gene) encoding the respective TIF or a heterologous recombinant expression construct comprising the polynucleotide (gene) encoding the TIF is preferably integrated into the genome of the host cell.

The term “genome” generally refers to the whole hereditary information of an organism that is encoded in the DNA (or RNA). It may be present in the chromosome, on a plasmid or vector, or both. Preferably, a polynucleotide (gene) encoding the respective TIF is integrated into the chromosome of said cell.

The polynucleotide (gene) encoding the respective TIF may be integrated in its natural locus. “Natural locus” means the location on a specific chromosome, where the polynucleotide (gene) encoding the TIF is located in a naturally-occurring wild-type cell. However, in another embodiment, the polynucleotide (gene) encoding the TIF is present in the genome of the host cell not at the natural locus, but integrated ectopically. The term “ectopic integration” means the insertion of a nucleic acid into the genome of a microorganism at a site other than its usual chromosomal locus, i.e., predetermined or random integration. In another embodiment, the polynucleotide (gene) encoding the TIF is integrated into the natural locus and ectopically. Heterologous recombination can be used to achieve random or non-targeted integration. Heterologous recombination refers to recombination between DNA molecules with significantly different sequences.

In specific embodiments, the polynucleotide (gene) encoding the respective TIF and/or the GOI can be integrated in a plasmid or vector. Preferably, the plasmid is a eukaryotic expression vector, preferably a yeast expression vector. Suitable plasmids or vectors are further described herein.

Overexpression of an endogenous or heterologous polynucleotide in a recombinant host cell can be achieved by modifying expression control sequences. Expression control sequences are known in the art and include, for example, promoters, enhancers, polyadenylation signals, transcription terminators, internal ribosome entry sites (IRES), and the like, that provide for the expression of the polynucleotide sequence in a host cell. Expression control sequences interact specifically with cellular proteins involved in transcription. Exemplary expression control sequences are described in, for example, Goeddel, Gene Expression Technology: Methods in Enzymology, Vol. 185, Academic Press, San Diego, Calif. (1990).

In a preferred embodiment, the overexpression is achieved by using an enhancer to express the polynucleotide. Transcriptional enhancers are relatively orientation and position independent, having been found 5′ and 3′ to the transcription unit, within an intron, as well as within the coding sequence itself. The enhancer may be spliced into the expression vector at a position 5′ or 3′ to the coding sequence, but is preferably located at a site 5′ from the promoter. Most yeast genes contain only one UAS, which generally lies within a few hundred base pairs of the cap site and most yeast enhancers (UASs) cannot function when located 3′ of the promoter, but enhancers in higher eukaryotes can function both 5′ and 3′ of the promoter.

Many enhancer sequences are known from mammalian genes (globin, RSV, SV40, EMC, elastase, albumin, a-fetoprotein and insulin). One may also use an enhancer from a eukaryotic cell virus, such as the SV40 late enhancer, the cytomegalovirus early promoter enhancer, the polyoma enhancer on the late side of the replication origin, and adenovirus enhancers.

Specifically, the GOI and/or the TIF encoding polynucleotide (gene) as described herein, are operably linked to transcriptional and translational regulatory sequences that provide for expression in the host cells. The term “translational regulatory sequences” as used herein refers to nucleotide sequences that are associated with a gene nucleic acid sequence and which regulate the translation of the gene. Transcriptional and/or translational regulatory sequences can either be located in plasmids or vectors or integrated in the chromosome of the host cell. Transcriptional and/or translational regulatory sequences are located in the same nucleic acid molecule of the gene which it regulates.

Specifically, the overexpression of the respective TIF can be achieved by methods known in the art, for example by genetically modifying their endogenous regulatory regions, as described by Marx et al., 2008 (Marx, H., Mattanovich, D. and Sauer, M. Microb Cell Fact 7 (2008): 23), such methods include, for example, integration of a recombinant promoter that increases expression of a gene.

For example, overexpression of an endogenous or heterologous polynucleotide in a recombinant host cell can be achieved by modifying the promoters controlling such expression, for example, by replacing a promoter (e.g., an endogenous promoter or a promoter which is natively linked to said polynucleotide in a wild-type organism) which is operably linked to said polynucleotide with another, stronger promoter in order to reach high expression levels. Such promoter may be inductive or constitutive. Modification of a promoter may also be performed by mutagenesis methods known in the art.

Specific embodiments refer to co-expression of TIFs (or TIF genes) along with expressing a GOI. In some embodiments described herein, a vector or nucleic acid sequence may include one or more expression cassettes for co-expressing at least one TIF molecule and a GOI. The vector or nucleic acid sequence may be constructed to allow for the co-expression of two or more polynucleotides using a multitude of techniques including co-transfection of two or more plasmids, the use of multiple or bidirectional promoters, or the creation of bicistronic or multicistronic vectors.

Specific embodiments refer to genetic modifications to stably co-express at least one, two, three, four or five TIFs, e.g., upon introducing the respective expression cassette(s) for stable integration within the host cell genome or chromosome.

The term “functionally active variant” also referred to as “functional variant” as used herein, means anything other than a native sequence (“native” being understood as a sequence naturally-occurring in a wild-type cell), e.g., derived from or relates to a TIF or nucleotide sequence or amino acid sequence of a TIF. Herein described are specific functional variants of any of the (parent) TIFs or the respective TIF genes (such as comprising or consisting of any one of SEQ ID NO:1-65; in particular any of SEQ ID NO:1, 12, 23, 34, 45, or 56) with a certain sequence identity to the parent sequence.

According to a specific embodiment, the functional variant is originating from a native sequence and comprises or consists of a predetermined sequence with proven function in the host cell which is about the same and/or even improved as compared to the native sequence from which it originates.

According to a specific embodiment, the functional variant is an isoform or orthologue of a naturally-occurring parent molecule, which orthologue is naturally-occurring in a species other than the species which comprises the naturally-occurring parent molecule e.g., a mammalian or fungal species.

In some embodiments, the functional variant of a polynucleotide or nucleic acid molecule comprises a nucleotide sequence which is sequence optimized e.g., for improving nucleic acid stability, increasing translation efficacy in the host cell, reducing the number of truncated proteins expressed, improving the folding or prevent misfolding of the expressed proteins, reducing toxicity of the expressed products, reducing cell death caused by the expressed products, or increasing and/or decreasing protein aggregation. According to a specific embodiment, the functional variant of a parent nucleotide sequence is a codon-optimized variant of said parent nucleotide sequence to be expressed in a host cell, which is obtainable by one or more genetic modifications of the parent nucleotide sequence for improved expression in the cellular environment of the host cell.

Functional variants of TIFs as described herein are considered functionally active, if having substantially the same or improved activity of the native sequence, in particular to improve the POI production when co-expressed in a host cell.

A functionally active variant of a TIF can be prepared by mutagenesis of a respective native (wild-type) TIF gene to produce a variant thereof, expressing the variant in the host cell concomitantly or simultaneously with a heterologous POI encoding gene, and assessing the activity of the variant to improve the host cell productivity to produce a POI.

The activity of a TIF may be determined as described by well-known methods using e.g., in vitro and in vivo approaches.

Suitable methods to analyse translational activity are summarized in Dermit et al. (Mol Biosyst. 2017 Nov. 21; 13(12):2477-24882017).

Some further suitable methods employ radioactive labelling of actively translated proteins (incorporation of radiolabelled amino acids) as described by Martin R (1998; Protein synthesis: methods and protocols. Methods in Molecular Biology, Volume 77, Totowa, N.J.: Humana Press).

A specific test measuring translational activity is described in the Examples section below.

Functional variants of a parent protein include, for instance, proteins wherein one or more amino acid residues are added, or deleted, at the N-or C-terminus, as well as within one or more internal domains. Specific functionally active variants comprise additional amino acids at the N-terminal and/or at the C-terminal end, to prolong a parent sequence, e.g. by less than 100 amino acids, specifically less than 75 amino acids, more specifically less than 50 amino acids, more specifically less than 25 amino acids, or else less than 10 amino acids. Further specific functionally active variants may be fusion proteins, wherein a sequence of the invention is prolonged by additional amino acid residues of another polypeptide or protein.

Specific functional variants are fragments of a parent protein or nucleic acid molecule.

Functional variants which are fragments of a polynucleotide or nucleic acid molecule may range from at least 20 nucleotides, preferably at least 100 nucleotides, up to the full-length nucleotide sequence encoding a TIF as described herein. Functionally active fragments of a polynucleotide or nucleic acid molecule may comprise at least 50% of the respective nucleotide sequence, preferably at least any of 60, 70, 80, 85,90%, or 95%.

Functional variants which are fragments of a polypeptide or protein may comprise or consist of at least 10 amino acids, specifically at least 25 amino acids, more specifically at least 50 amino acids, more specifically at least 75 amino acids, or at least 100 contiguous amino acids, or up to the total number of amino acids present in a full-length protein.

The term “endogenous” as used herein is meant to include those molecules and sequences, in particular endogenous genes or proteins, which are present in the wild-type (native) host cell, prior to its modification to reduce expression of the respective endogenous genes and/or reduce the production of the endogenous proteins. In particular, an endogenous nucleic acid molecule (e.g., a gene) or protein that does occur in (and can be obtained from) a particular host cell as it is found in nature, is understood to be “host cell endogenous” or “endogenous to the host cell”. Moreover, a cell “endogenously expressing” a nucleic acid or protein expresses that nucleic acid or protein as does a host of the same particular type as it is found in nature. Moreover, a host cell “endogenously producing” or that “endogenously produces” a nucleic acid, protein, or other compound produces that nucleic acid, protein, or compound as does a host cell of the same particular type as it is found in nature.

Thus, even if an endogenous protein is no more produced by a host cell, such as in a knockout mutant of the host cell, where the protein encoding gene is inactivated or deleted, the protein is herein still referred to as “endogenous”.

The term “heterologous” as used herein with respect to a nucleotide sequence, construct such as an expression cassette, amino acid sequence or protein, refers to a compound which is either foreign to a given host cell, i.e. “exogenous”, such as not found in nature in said host cell; or that is naturally found in a given host cell, e.g., is “endogenous”, however, in the context of a heterologous construct or integrated in such heterologous construct, e.g., employing a heterologous nucleic acid fused or in conjunction with an endogenous nucleic acid, thereby rendering the construct heterologous. The heterologous nucleotide sequence as found endogenously may also be produced in an unnatural, e.g., greater than expected or greater than naturally found, amount in the cell. The heterologous nucleotide sequence, or a nucleic acid comprising the heterologous nucleotide sequence, possibly differs in sequence from the endogenous nucleotide sequence but encodes the same protein as found endogenously. Specifically, heterologous nucleotide sequences are those not found in the same relationship to a host cell in nature. Any recombinant or artificial nucleotide sequence is understood to be heterologous. An example of a heterologous polynucleotide is a nucleotide sequence not natively associated with a promoter, e.g., to obtain a hybrid promoter, or operably linked to a coding sequence, as described herein. As a result, a hybrid or chimeric polynucleotide may be obtained. A further example of a heterologous compound is a POI encoding polynucleotide operably linked to a transcriptional control element, e.g., a promoter, to which an endogenous, naturally-occurring POI coding sequence is not normally operably linked.

The term “translation initiation factor” abbreviated “TIF” as used herein shall refer to the translation initiation factor protein or the polynucleotide (a nucleic acid molecule) encoding the translation initiation factor.

Specifically, neither of the TIFs described herein is the protein of interest (POI). It is specifically understood that the recombinant host cell described herein comprises an expression system to express the TIF(s) and additionally express another polynucleotide (different from said TIF(s) coding polynucleotides), herein referred to as gene of interest (GOI).

The term “translation initiation factor” as used herein particularly refers to any of the factors comprised in the mRNP.

Specific TIF encoding nucleotide sequences are naturally-occurring, or functionally active variants thereof, such as a variant nucleotide sequence that differs from the parent (naturally-occurring) one by one or more, e.g., up to any one of 50, 40, 30, 20, or 10 point mutations to optimize the sequences, such as by a nucleotide sequence optimization algorithm or by codon-optimization techniques, to improve its expression in recombinant host cells.

Specific optimization techniques are improving expression of the nucleotide sequence in the host cell, such as by a nucleotide sequence optimization algorithm or by codon-optimization techniques.

Specific optimization techniques are improving cloning, such as optimization for Golden Gate cloning or Golden Gate assembly.

Specific optimized nucleotide sequences comprise “silent” mutations such as e.g. to avoid the presence of the recognition sites of any restriction enzymes used (e.g. BsaI and BpiI).

The optimized nucleotide sequences described herein typically allow one or more, e.g. a few point mutations in the encoded amino acid sequence e.g., up to 10, 9, 8, 7, 6, 5, 4, or 3 point mutations.

“eIF4E”, also known as Eukaryotic translation initiation factor 4E, is a TIF involved in the formation of the closed loop mRNA, which is a closed-loop factor and part of the el F4F cap-binding complex and part of the mRNP. It is characterized by any one of SEQ ID NO:1-11, or orthologs in other eukaryotic species. eIF4E is encoded by an eIF4E coding nucleotide sequence, which may be a naturally-occurring eIF4E gene, or a respective functional variant thereof encoding eIF4E that has a certain sequence identity to the naturally occurring eIF4E. Exemplary eIF4E coding nucleotide sequences are identified by SEQ ID NO:66 of Komagataella phaffii encoding SEQ ID NO:1, or a functionally active variant thereof. SEQ ID NO:67 identifies an example of an optimized coding nucleotide sequence, which has been produced by Golden gate optimization, and differs from SEQ ID NO:66 by three nucleotide substitutions: A276G, T354C, G492A.

eIF4A, also known as Eukaryotic translation initiation factor 4A, is a TIF involved in the formation of the closed loop mRNA, which is a closed-loop factor and part of the eIF4F cap-binding complex and part of the mRNP. It is characterized by any one of SEQ ID NO:12-33, or orthologs in other eukaryotic species. The term “eIF4A” includes TIF2a and Tif2b, with TIF2b being a 249 nucleotides (corresponding to 83 amino acids) longer variant of TIF2a on the 5′ end. Both sequences were amplified directly from the P. pastoris genome, and present two variants of eIF4A that have alternative start positions.

eIF4A is encoded by an eIF4A coding nucleotide sequence, which may be a naturally-occurring eIF4A gene, or a respective functional variant thereof encoding eIF4A that has a certain sequence identity to the naturally occurring eIF4A. Exemplary eIF4A coding nucleotide sequences are identified by SEQ ID NO:68 or SEQ ID NO:70 of Komagataella phaffii encoding SEQ ID NO:12 and SEQ ID NO:23, respectively, or a functionally active variant thereof. SEQ ID NO:69 identifies an example of an optimized coding nucleotide sequence, which has been produced by Golden gate optimization, and differs from SEQ ID NO:68 by one nucleotide substitution: C45A. SEQ ID NO:71 identifies an example of an optimized coding nucleotide sequence, which has been produced by Golden gate optimization, and differs from SEQ ID NO:70 by one nucleotide substitution: C294A.

eIF4G, also known as Eukaryotic translation initiation factor 4G, is a TIF involved in the formation of the closed loop mRNA, which is a closed-loop factor and part of the eIF4F cap-binding complex and part of the mRNP. It is characterized by any one of SEQ ID NO:34-44, or orthologs in other eukaryotic species. eIF4G is encoded by an eIF4G coding nucleotide sequence, which may be a naturally-occurring eIF4G gene, or a respective functional variant thereof encoding eIF4G that has a certain sequence identity to the naturally occurring eIF4G. Exemplary eIF4G coding nucleotide sequences are identified by SEQ ID NO:72 of Komagataella phaffii encoding SEQ ID NO:34, or a functionally active variant thereof. SEQ ID NO:73 identifies an example of an optimized coding nucleotide sequence, which has been produced by Golden gate optimization, and differs from SEQ ID NO:72 by four nucleotide substitutions: A564G, A1923G, G2037C, T2100C.

PAB1, also known as Polyadenylate-binding protein 1, is a TIF involved in the formation of the closed loop mRNA and part of the mRNP. It is characterized by any one of SEQ ID NO:45-55, or orthologs in other eukaryotic species. PAB1 is encoded by a PAB1 coding nucleotide sequence, which may be a naturally-occurring PAB1 gene, or a respective functional variant thereof encoding PAB1 that has a certain sequence identity to the naturally occurring PAB1. Exemplary PAB1 coding nucleotide sequences are identified by SEQ ID NO:74 of Komagataella phaffii encoding SEQ ID NO:45, or a functionally active variant thereof. SEQ ID NO:75 identifies an example of an optimized coding nucleotide sequence, which has been produced by Golden gate optimization, and differs from SEQ ID NO:74 by three nucleotide substitutions C150A, T384C, C707T.

RLI1, also known as ATP-binding cassette sub-family E member 1 (ABCE1) also known as RNase L inhibitor (RLI) is an ATP-binding cassette (ABC) protein that in humans is encoded by the ABCE1 gene. It is a TIF with a dual role in translation initiation and ribosome biogenesis as well as in translation termination and part of the mRNP. It is characterized by any one of SEQ ID NO:56-65, or orthologs in other eukaryotic species. RLI1 is encoded by a RLI1 coding nucleotide sequence, which may be a naturally-occurring RLI1 gene, or a respective functional variant thereof encoding RLI1 that has a certain sequence identity to the naturally occurring RLI1. Exemplary RLI1 coding nucleotide sequences are identified by SEQ ID NO:76 of Komagataella phaffii encoding SEQ ID NO:56, or a functionally active variant thereof.

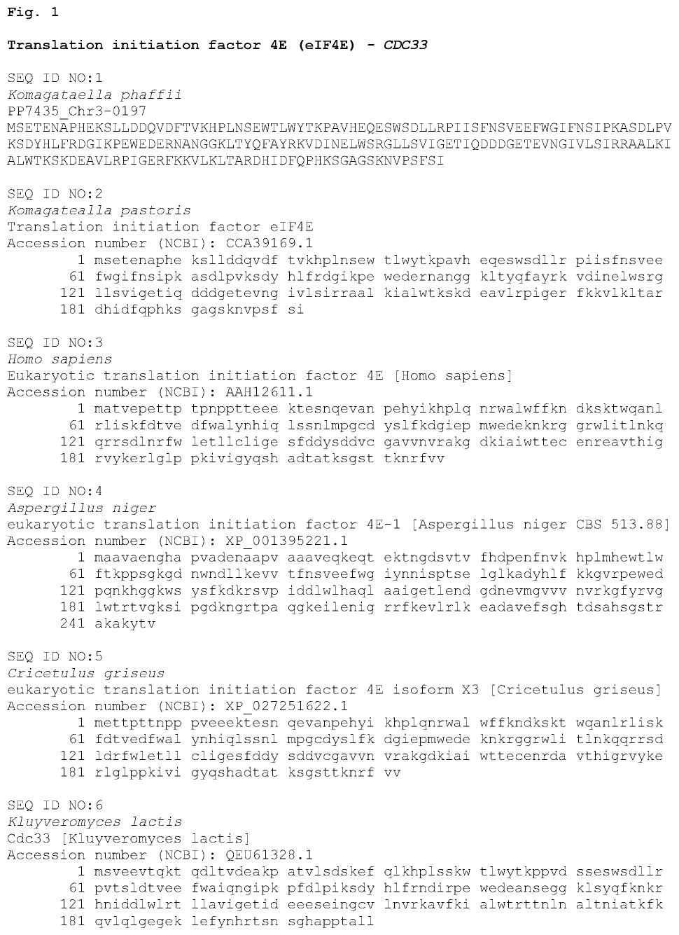

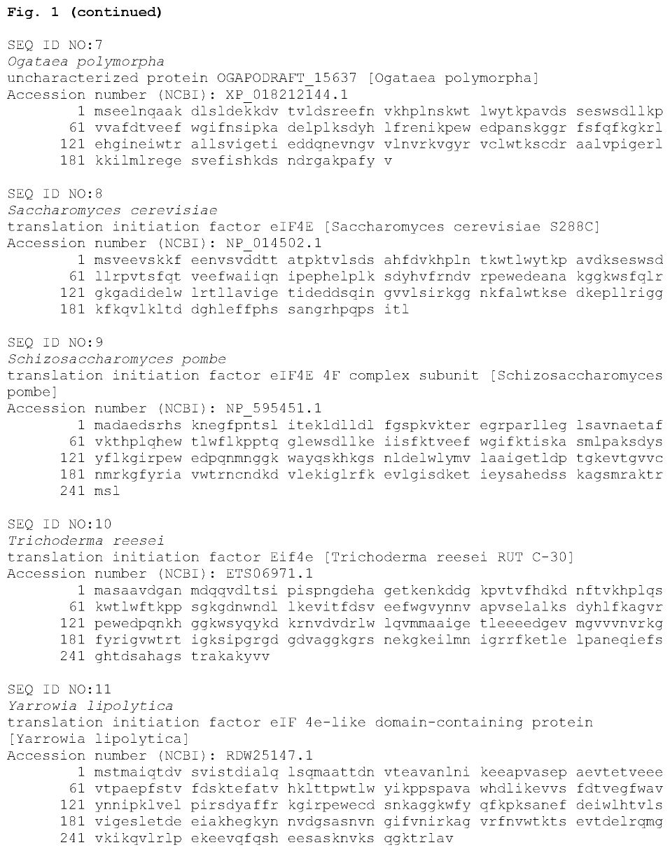

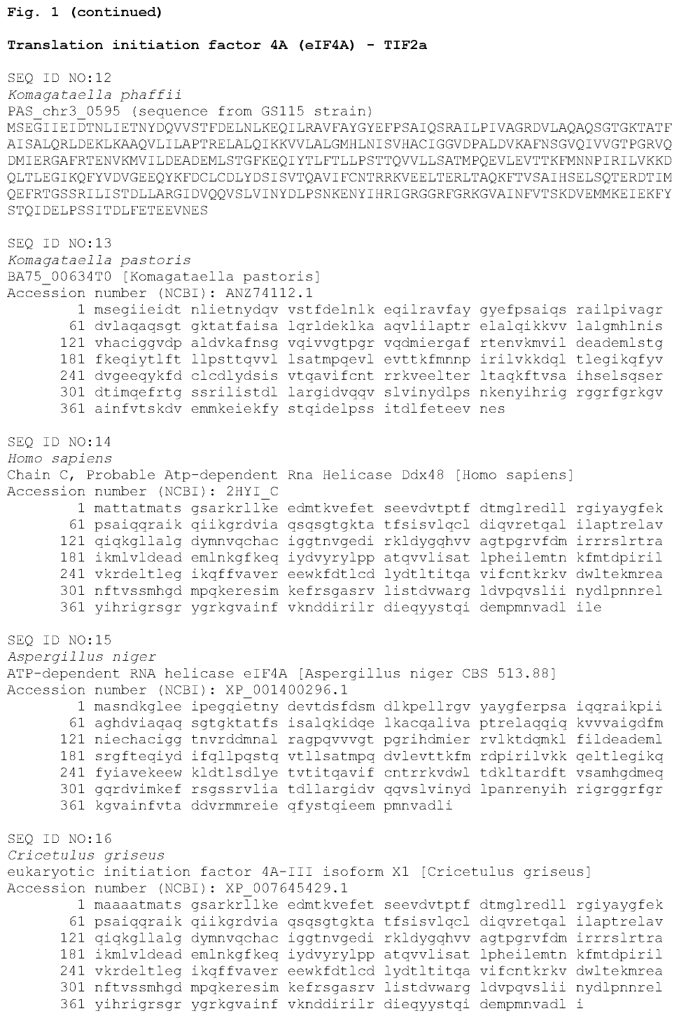

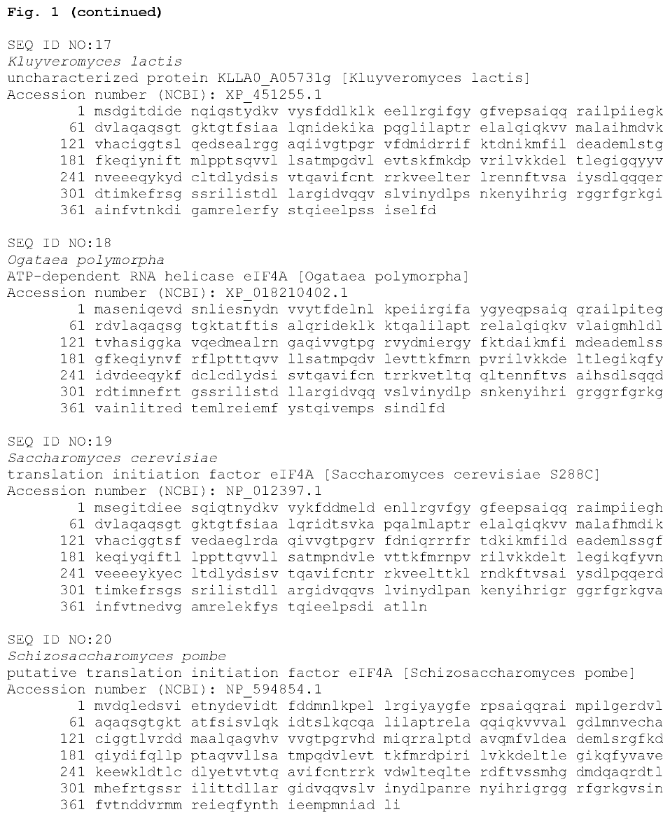

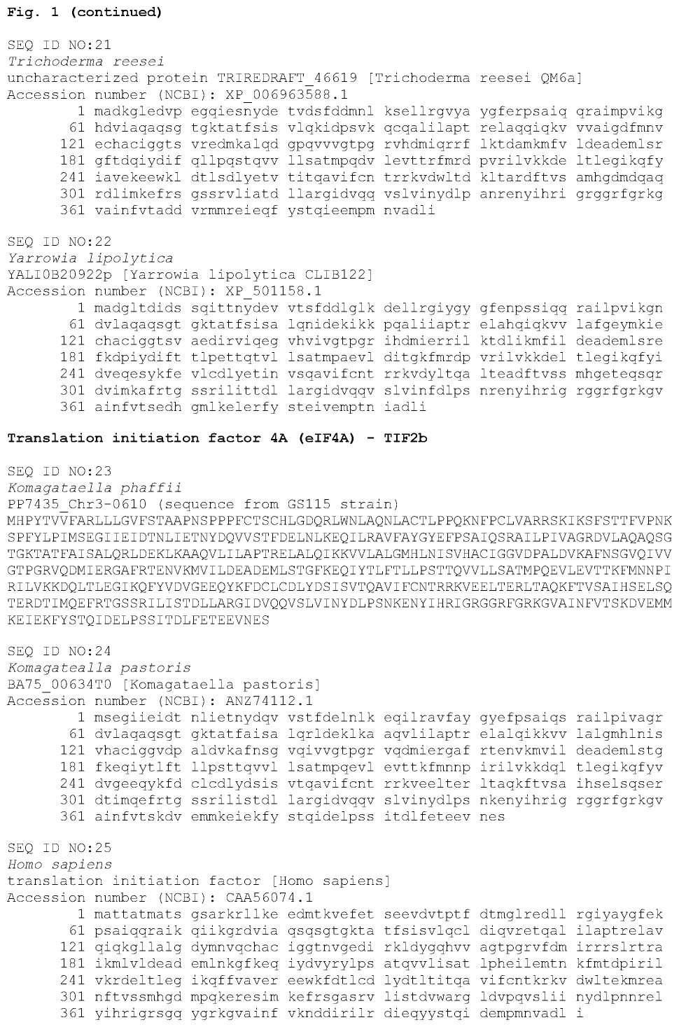

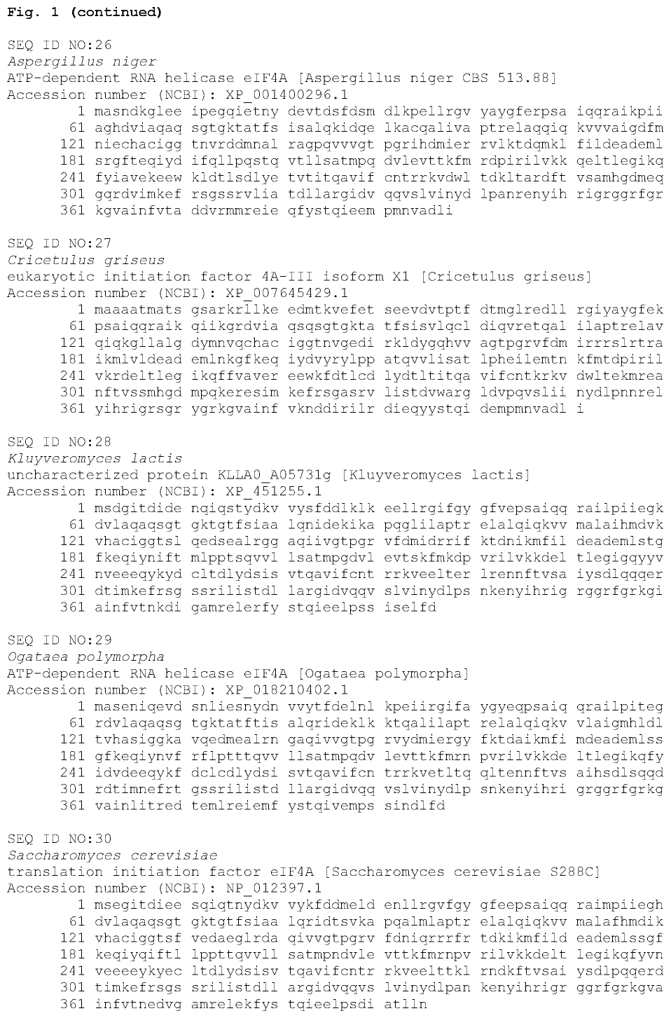

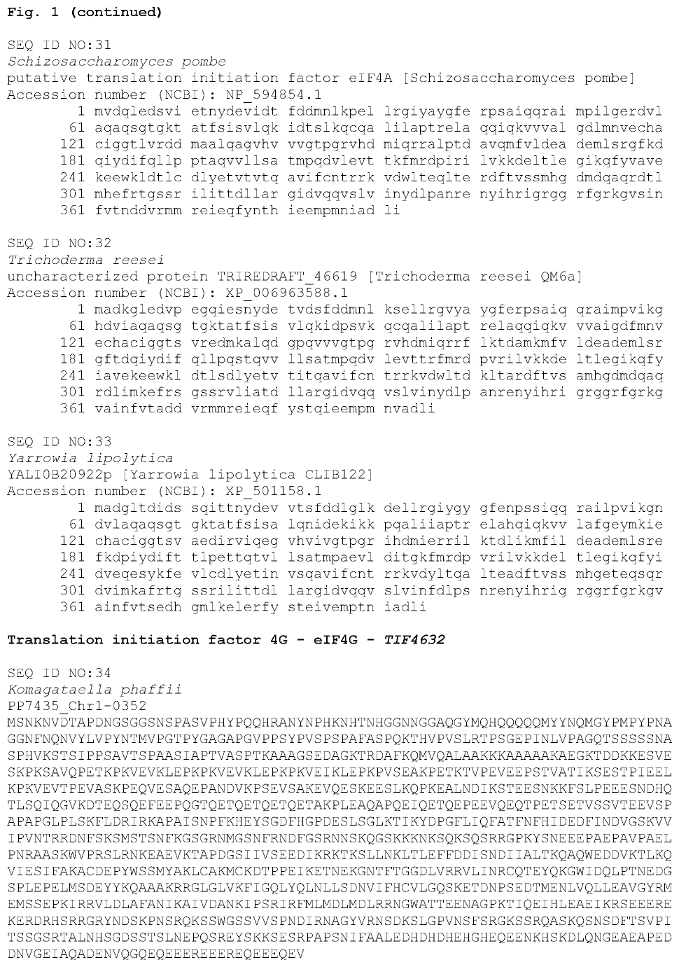

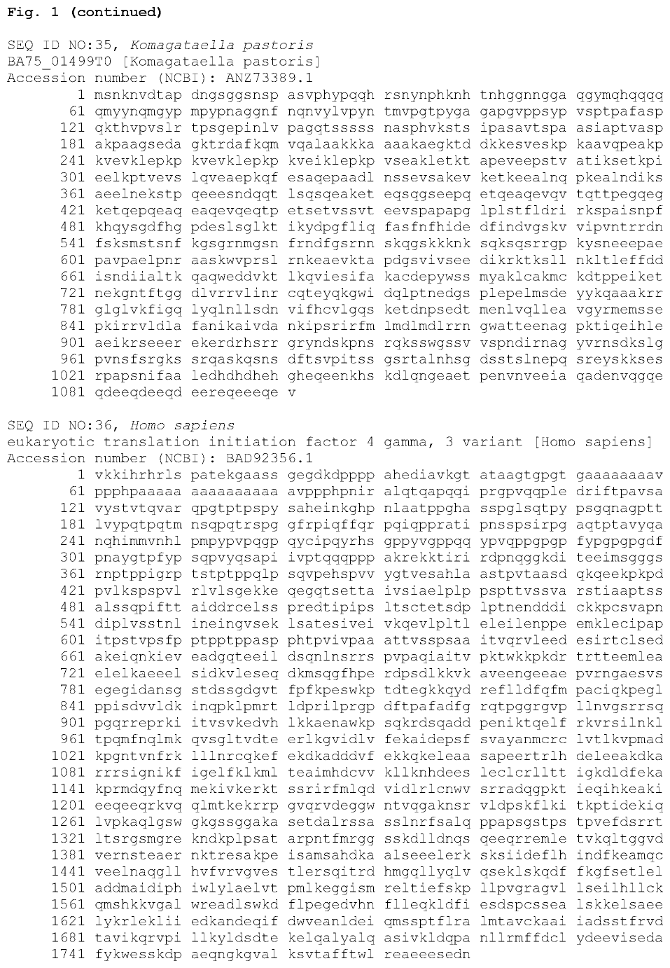

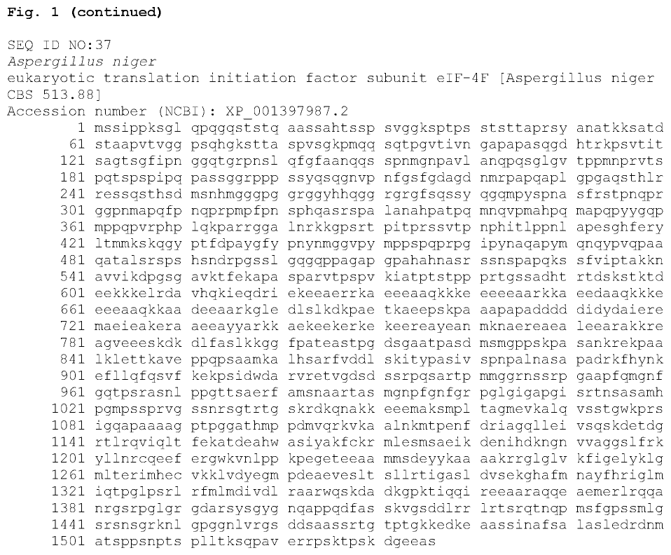

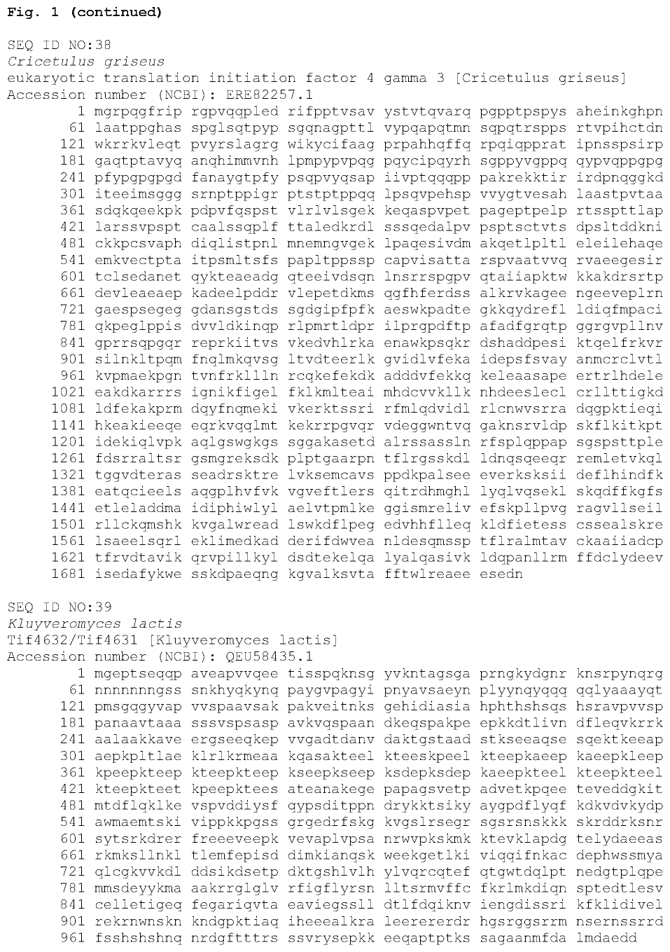

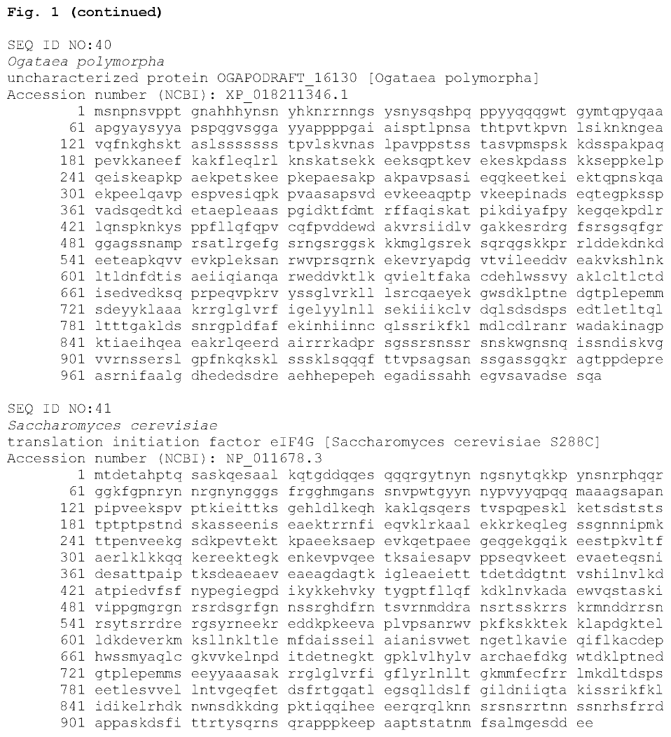

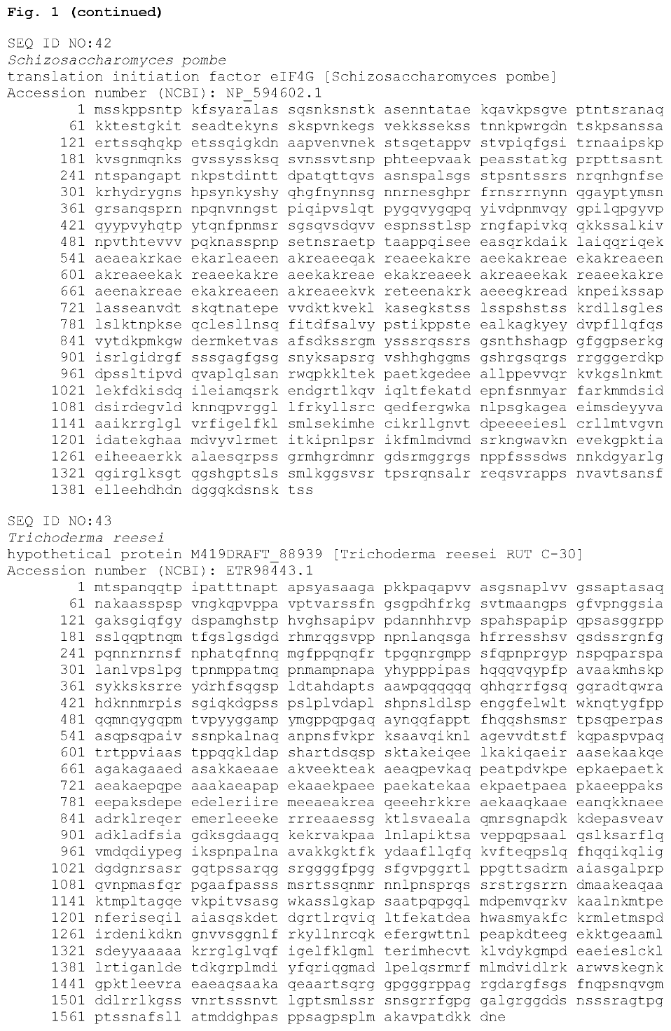

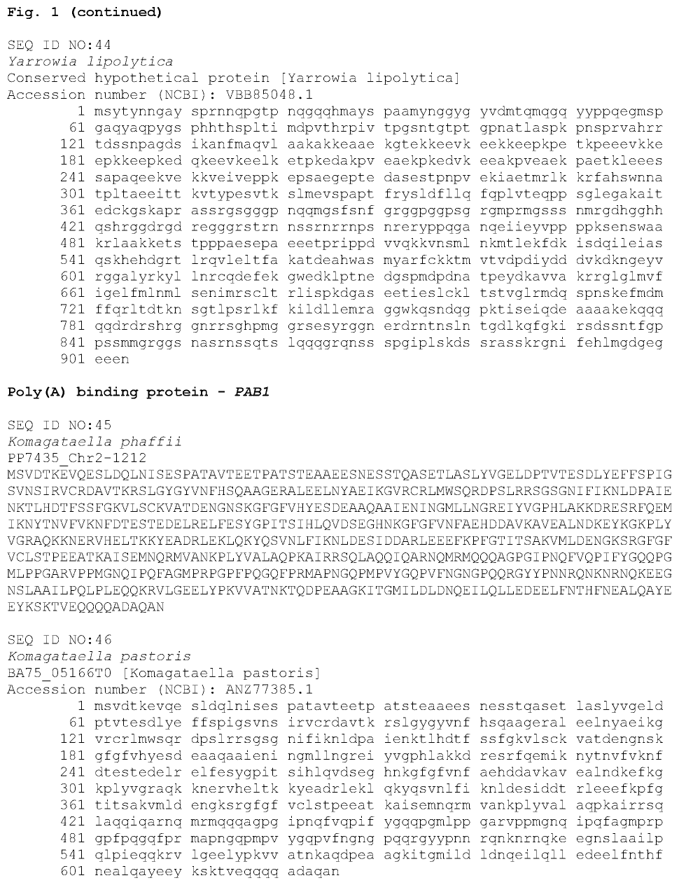

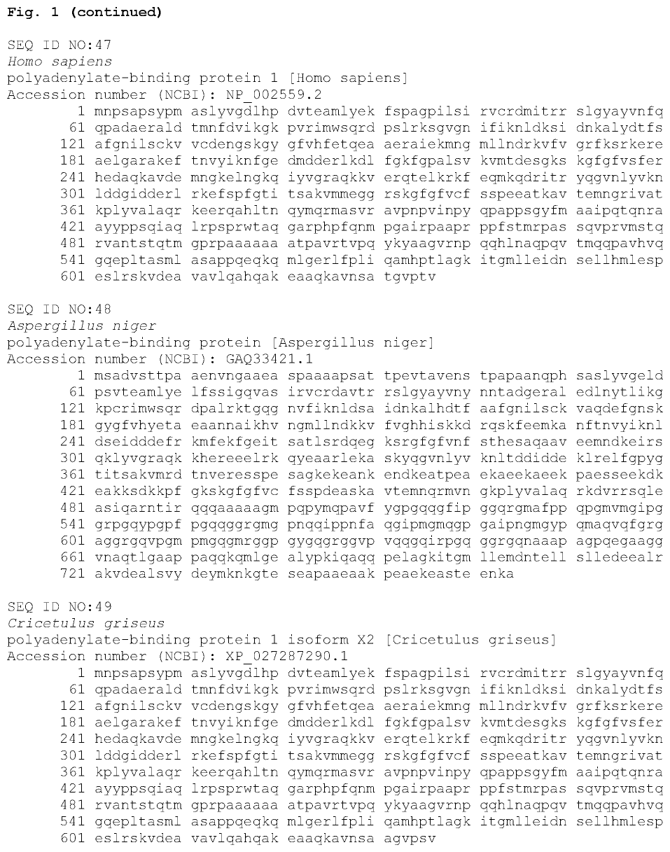

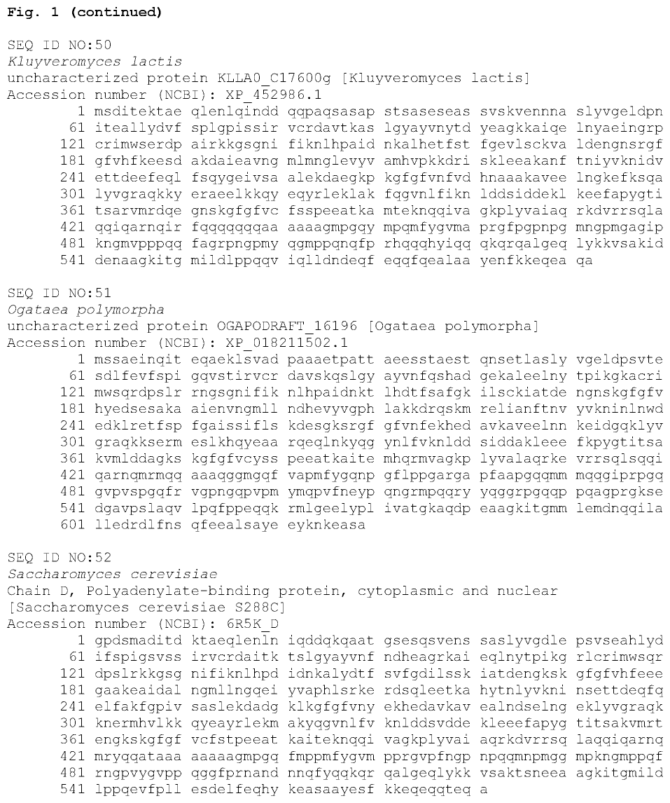

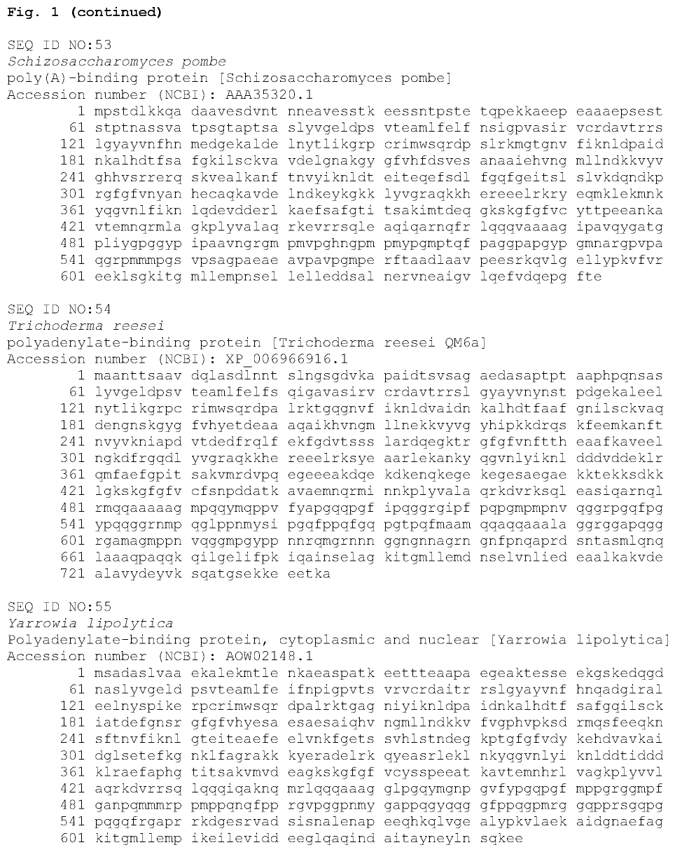

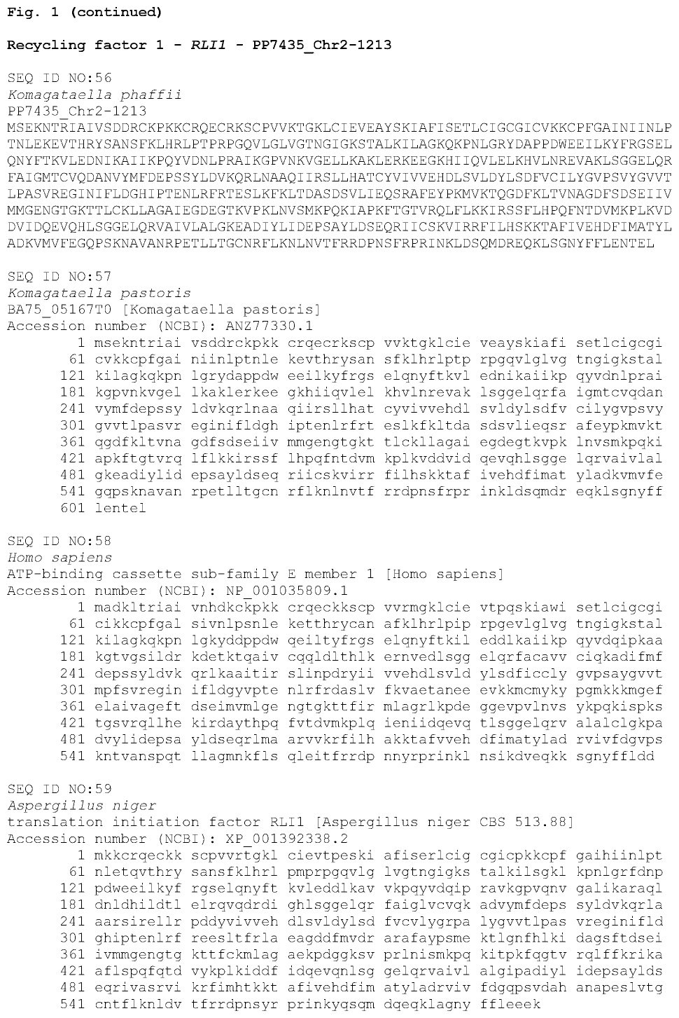

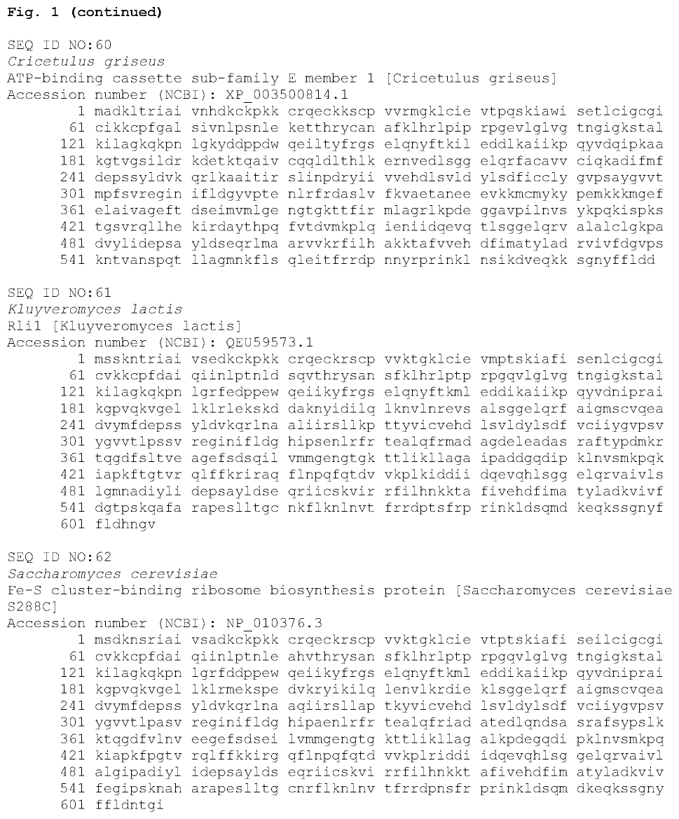

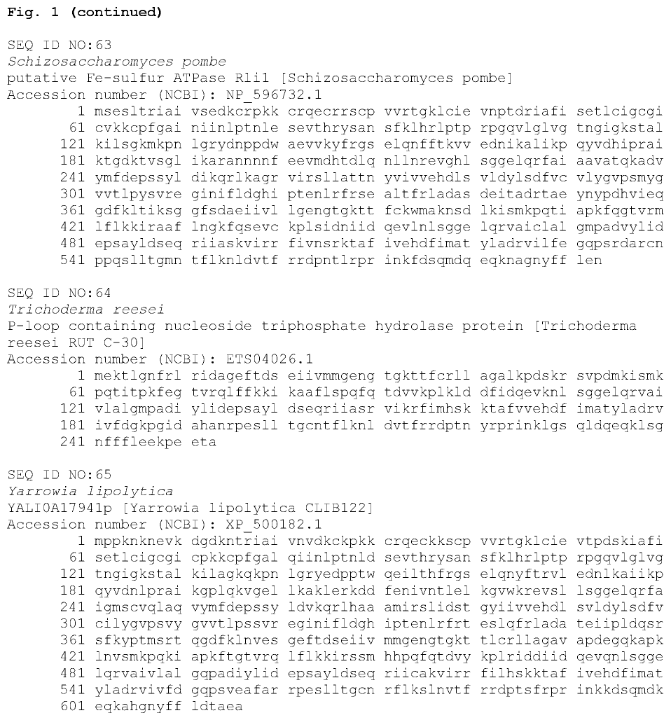

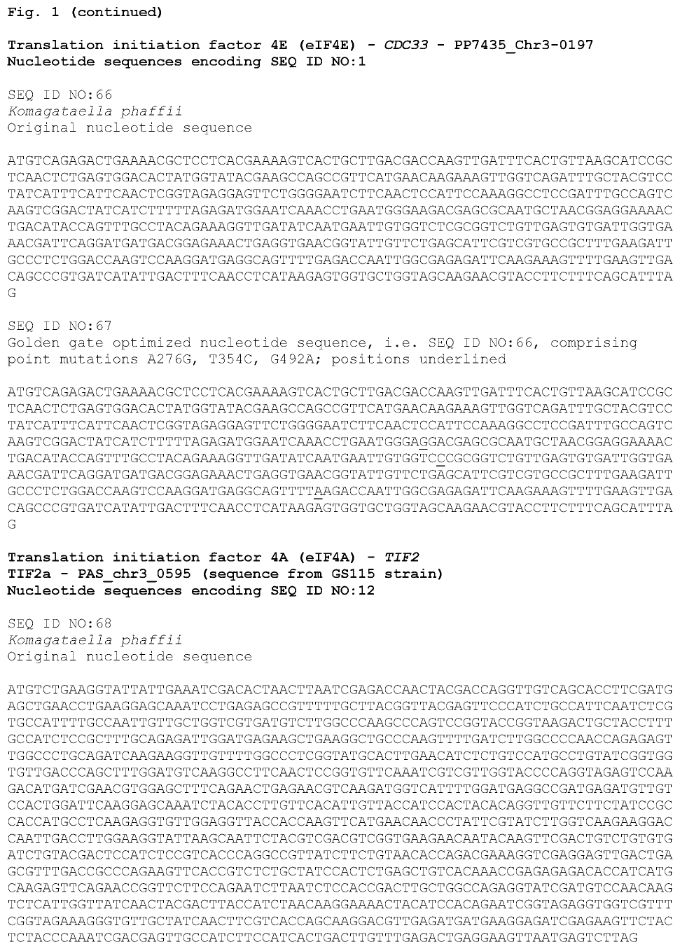

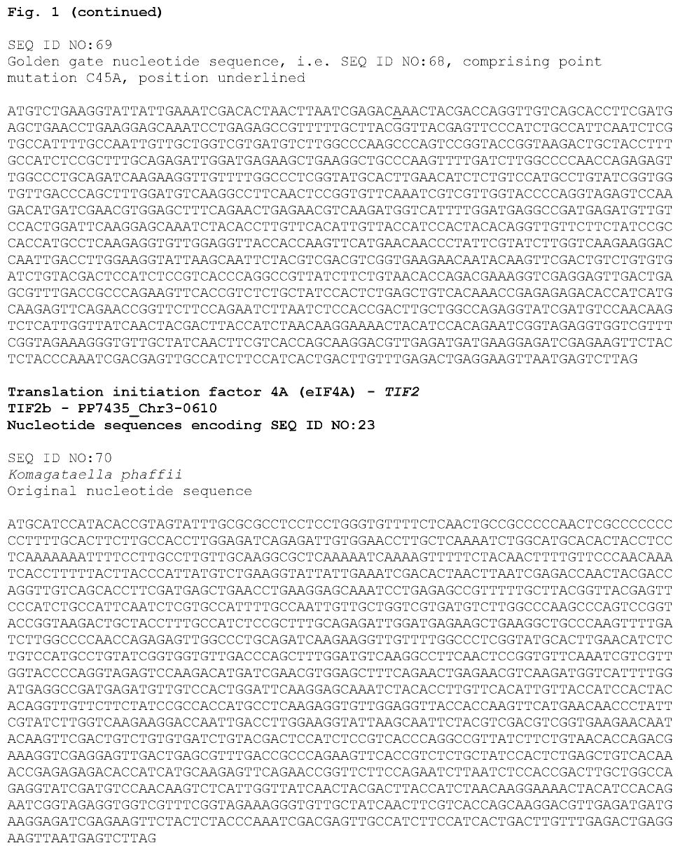

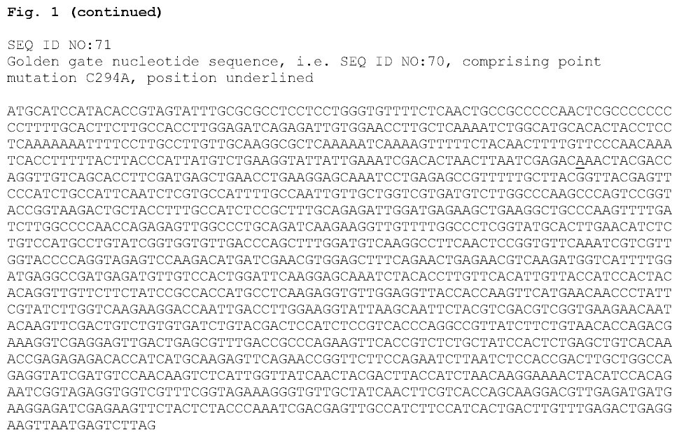

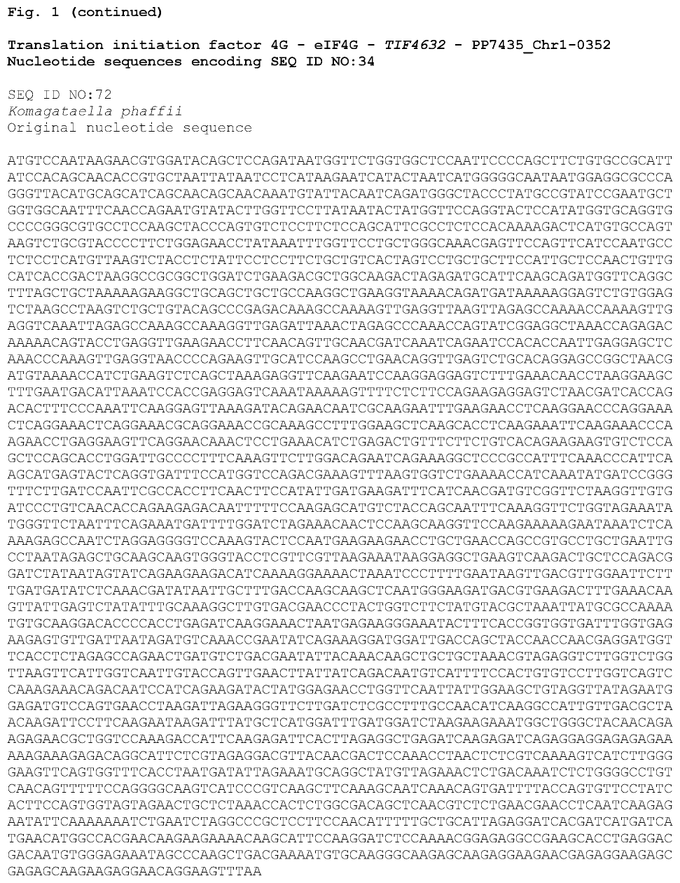

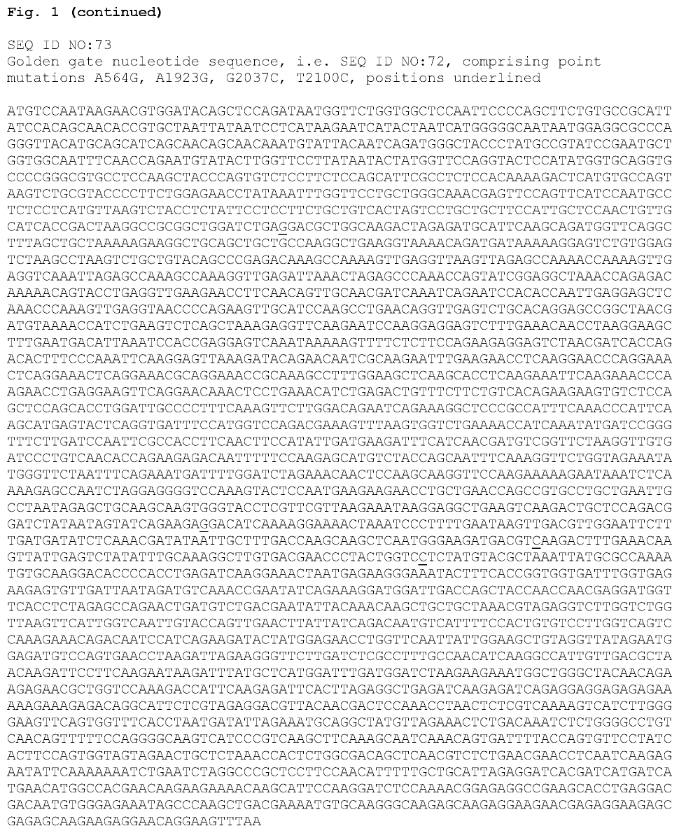

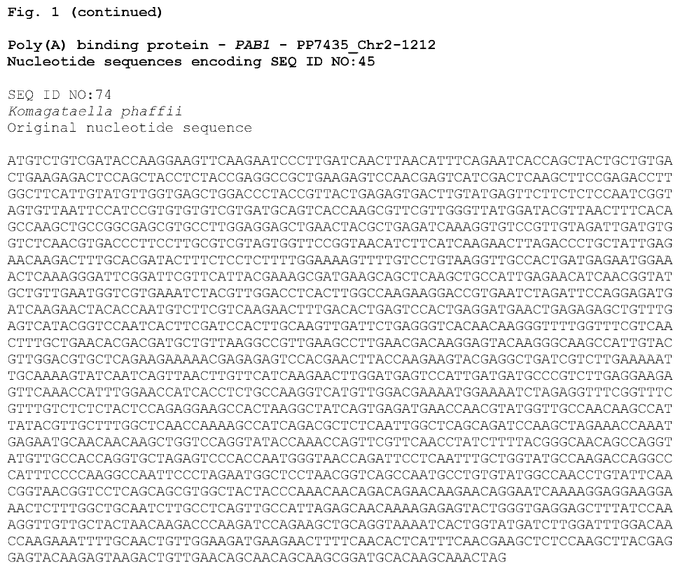

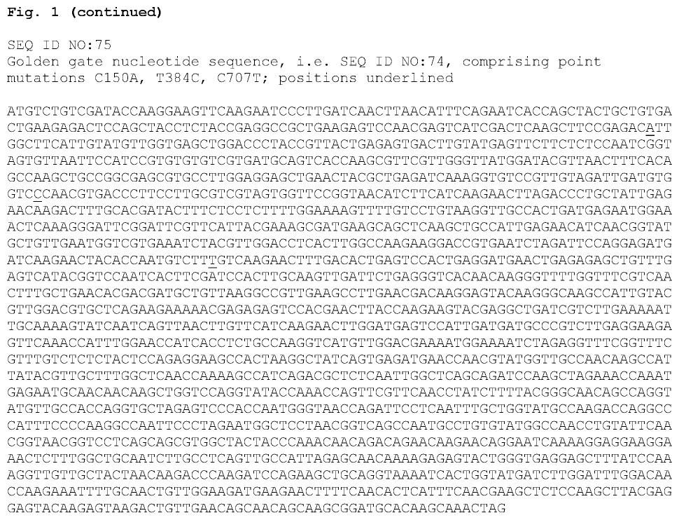

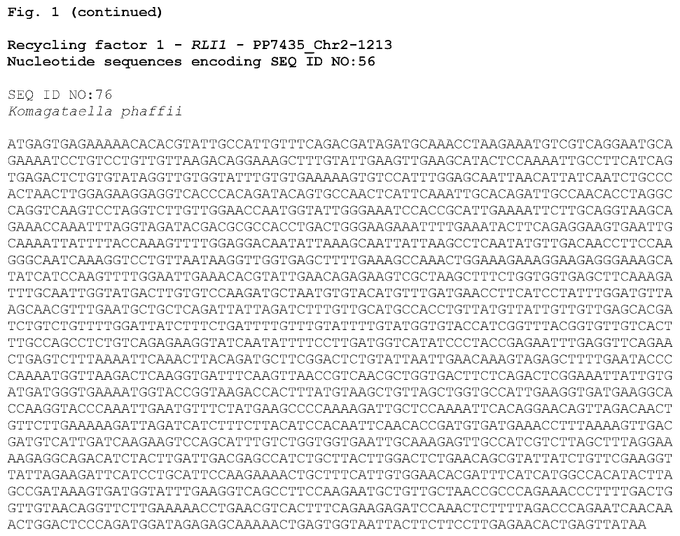

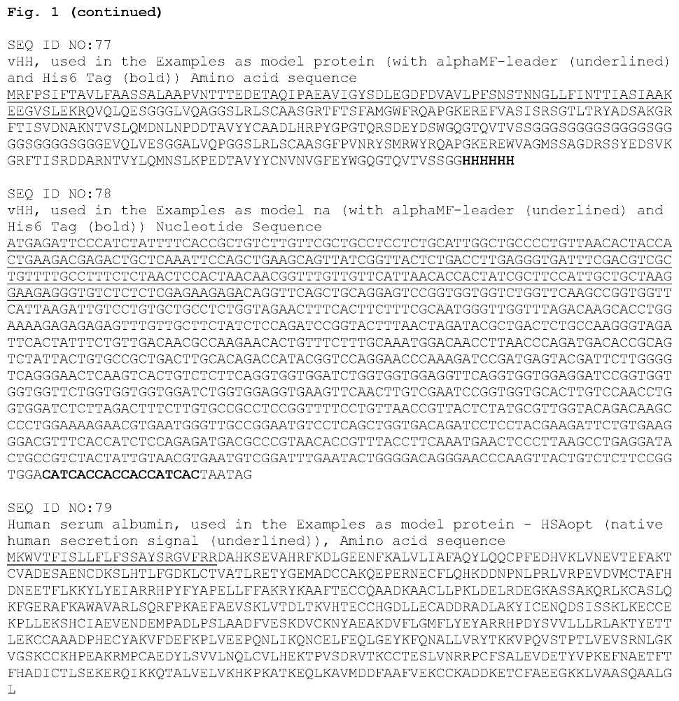

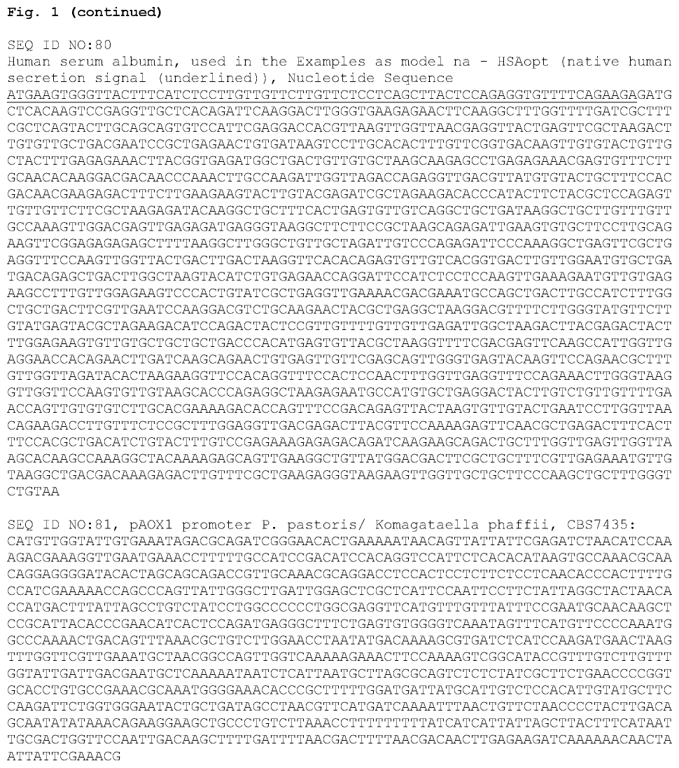

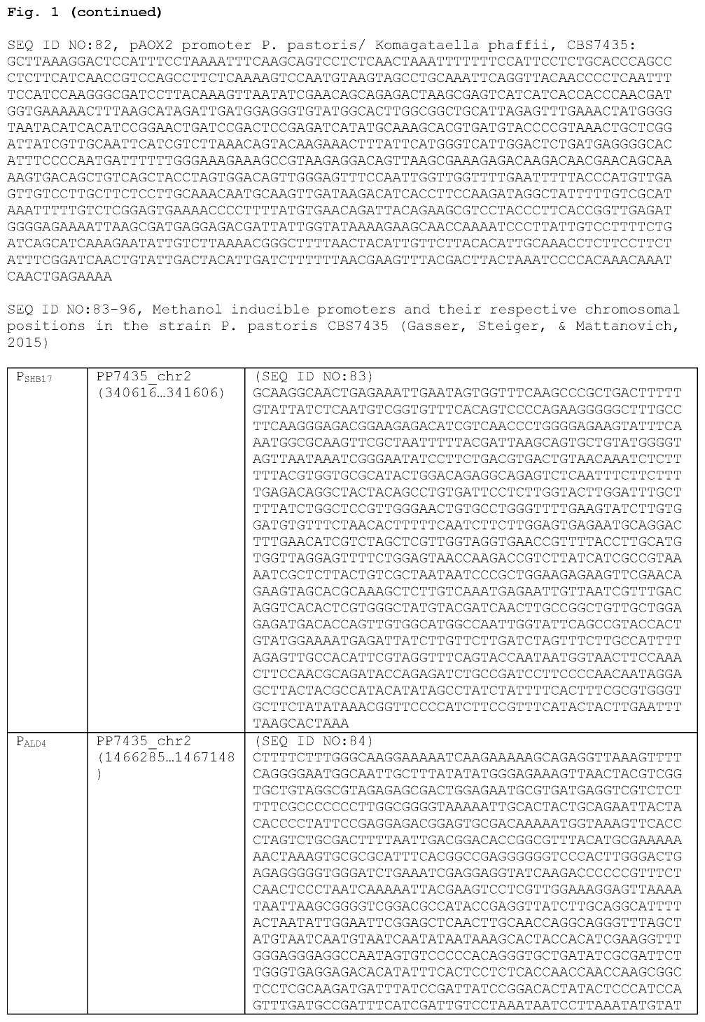

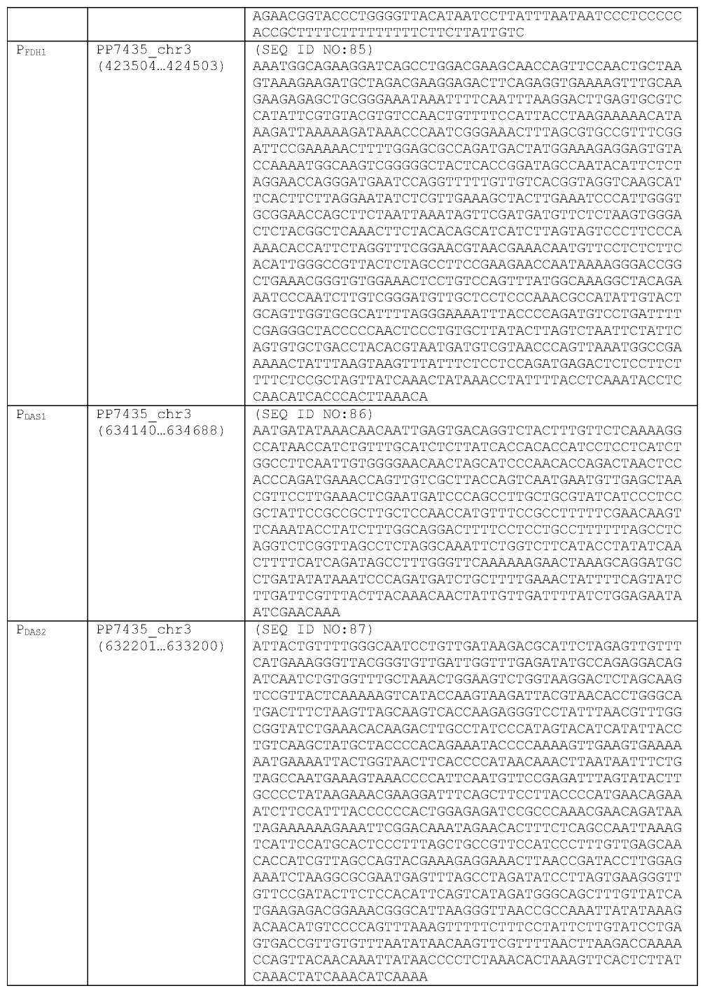

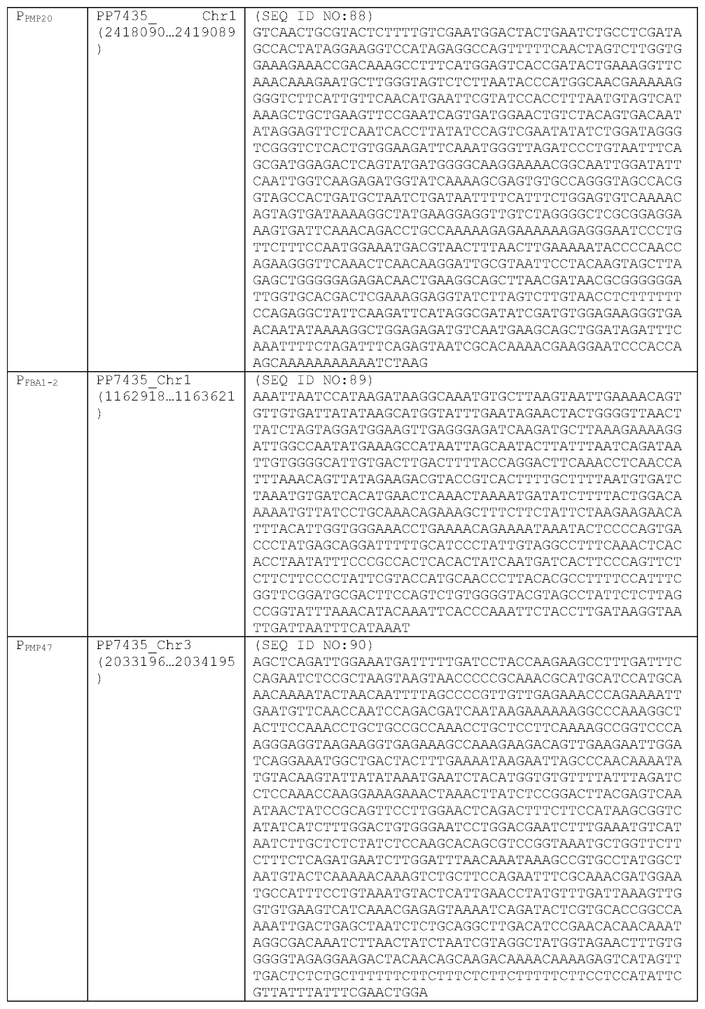

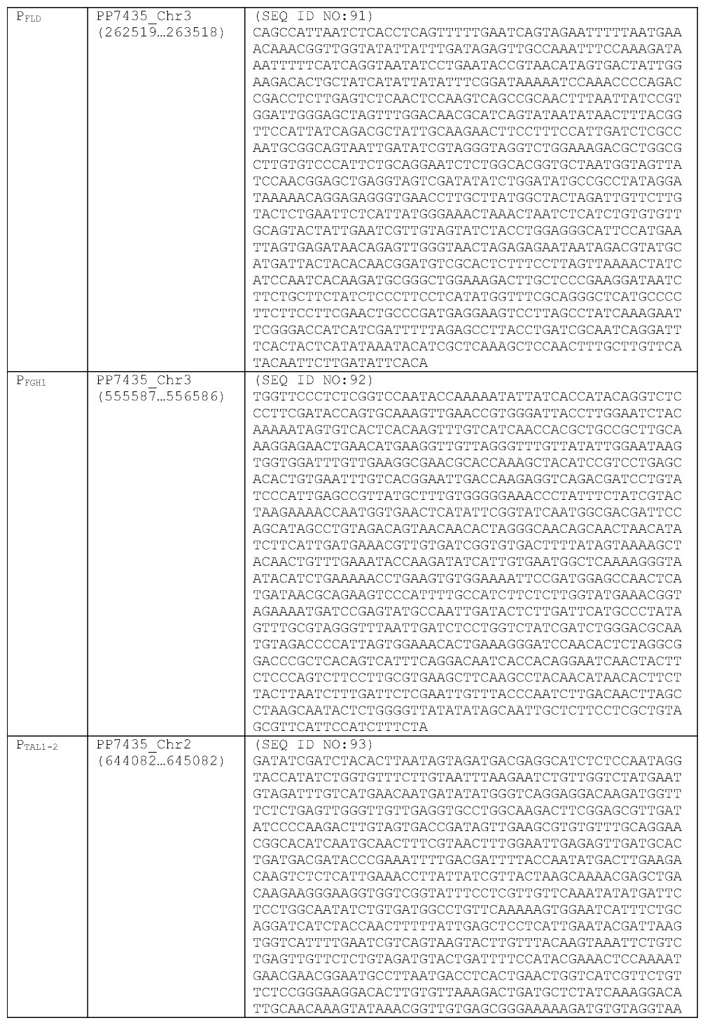

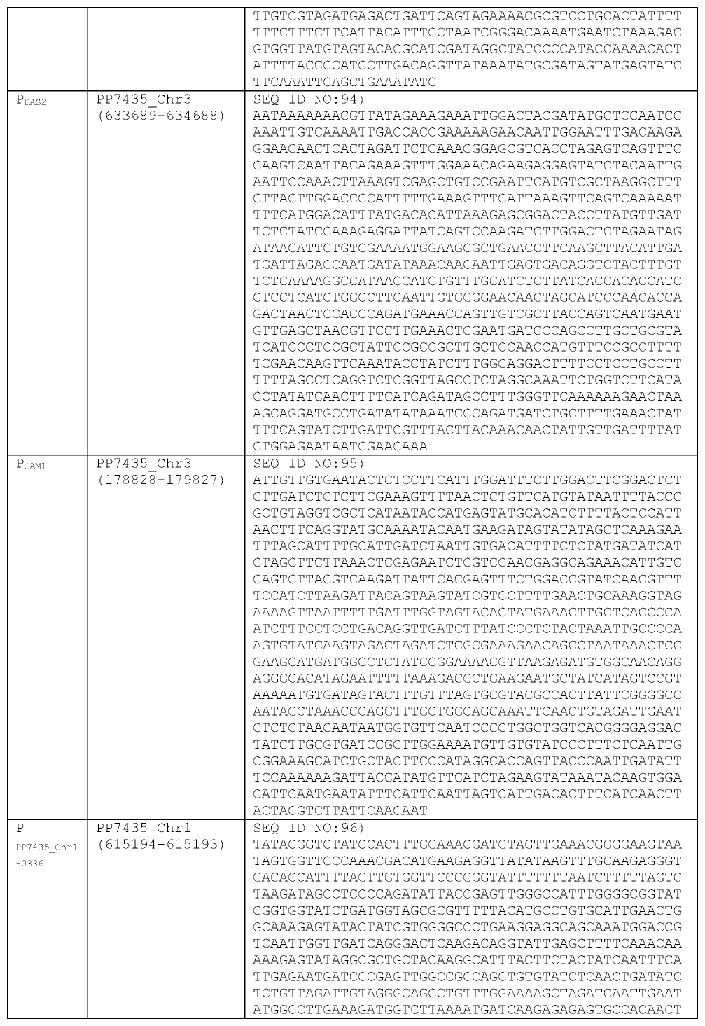

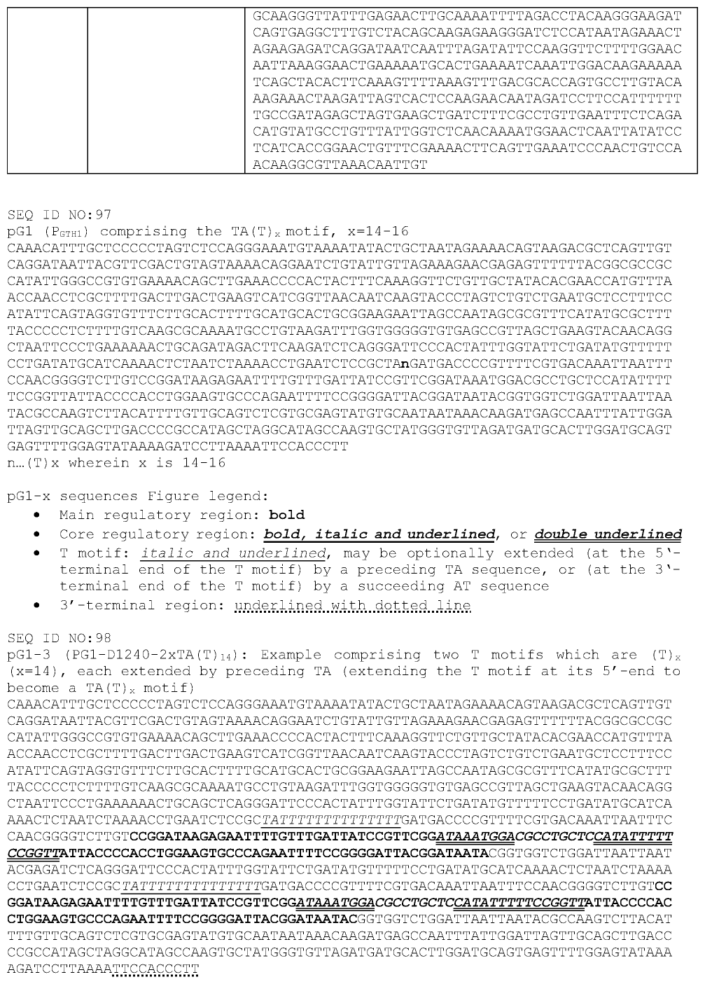

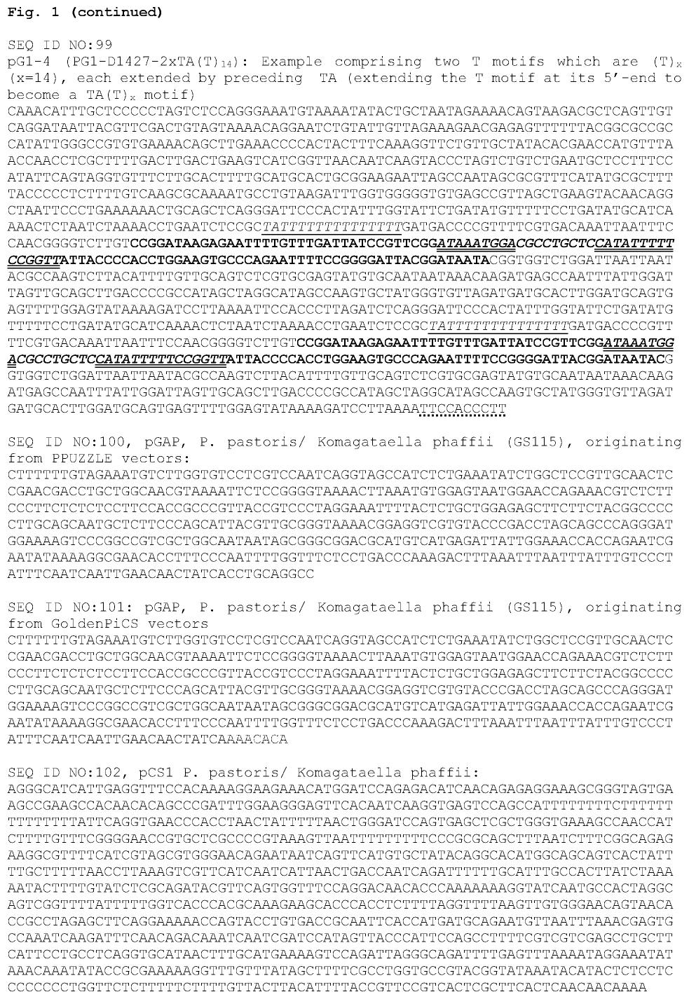

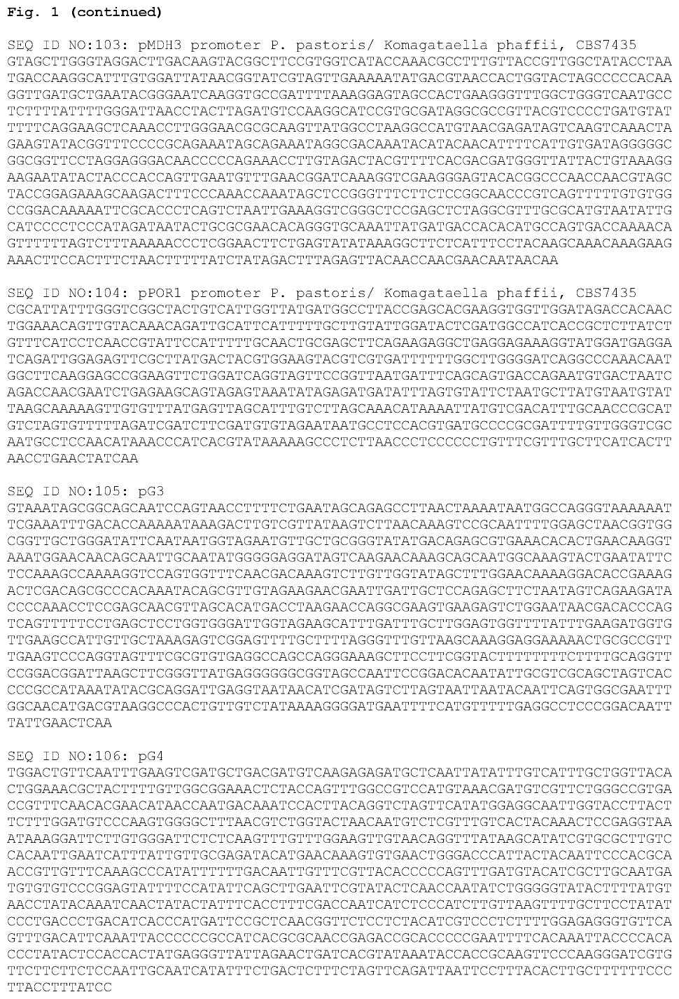

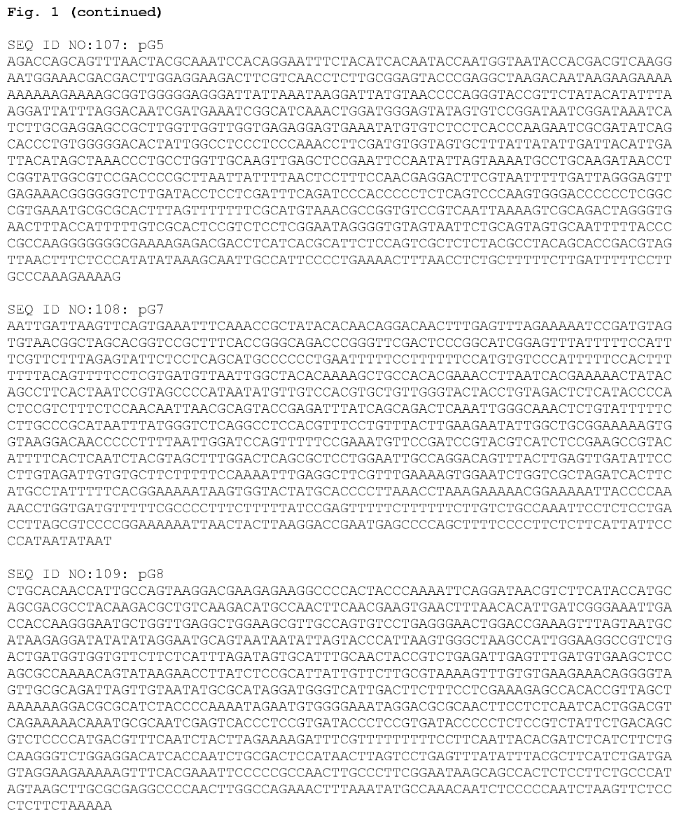

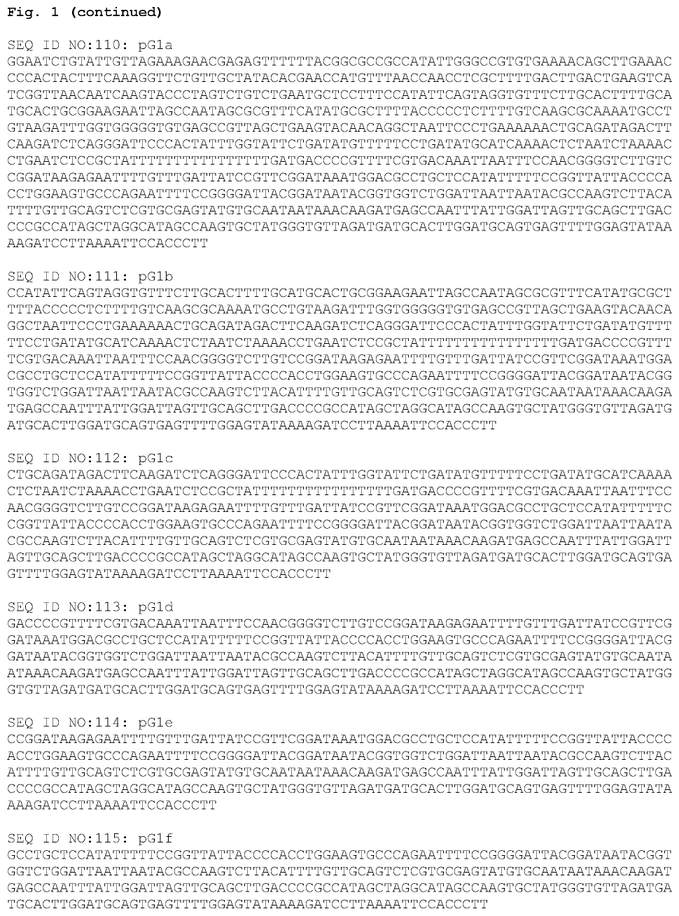

The TIFs comprising or consisting of the amino acid sequence identified by SEQ ID NO:1, 12, 23, 34, 45 and 56 as provided herein, and as used in the Examples section (including the respective (optimized) nucleotide sequences), are of K. phaffii origin. The TIFs comprising or consisting of the amino acid sequence identified by SEQ ID NO:2, 13, 24, 35, 46 and 57 as provided herein are of K. pastoris origin. It is well understood that there are homologous sequences present in other yeast host cells, in particular in methylotrophic yeast, such as those provided in FIG. 1, which can be used as described herein. For example, yeast of Pichia pastoris comprise the respective homologous sequences. Pichia pastoris has been reclassified into the genus, Komagataella, and split into three species, K. pastoris, K. phaffii, and K. pseudopastoris.

For example, any homologous sequence of a respective TIF with a certain sequence identity described herein, can be used, in particular any such protein which is an ortholog of the respective P. pastoris TIF, such as of K. phaffii, K. pastoris, or K. pseudopastoris.

The term “mRNP” as used herein shall refer to messenger RNP (messenger ribonucleoprotein) which is understood as a particle or complex consisting of mRNA with bound proteins. mRNA is bound by various proteins while being synthesized, spliced, exported, and translated in the cytoplasm.

The term “operably linked” as used herein refers to the association of nucleotide sequences on a single nucleic acid molecule, e.g., a vector, or an expression cassette, in a way such that the function of one or more nucleotide sequences is affected by at least one other nucleotide sequence present on said nucleic acid molecule. By operably linking, a nucleic acid sequence is placed into a functional relationship with another nucleic acid sequence on the same nucleic acid molecule. For example, a promoter is operably linked with a coding sequence of a recombinant gene, when it is capable of effecting the expression of that coding sequence. As a further example, a nucleic acid encoding a signal peptide is operably linked to a nucleic acid sequence encoding a POI, when it is capable of expressing a protein in the secreted form, such as a preform of a mature protein or the mature protein. Specifically, such nucleic acids operably linked to each other may be immediately linked, i.e. without further elements or nucleic acid sequences in between the nucleic acid encoding the signal peptide and the nucleic acid sequence encoding a POI. Alternatively, a suitable linking sequence can be used such as e.g., a cloning site positioned between the promoter and the GOI.

A “promoter” sequence is typically understood to be operably linked to a coding sequence, if the promoter controls the transcription of the coding sequence. If a promoter sequence is not natively associated with the coding sequence, its transcription is either not controlled by the promoter in native (wild-type) cells or the sequences are recombined with different contiguous sequences.

A promoter is herein described to initiate, regulate, or otherwise mediate or control the expression of a protein coding polynucleotide (DNA), such as a POI coding DNA. Promoter DNA and coding DNA may be from the same gene or from different genes, and may be from the same or different organisms.

The strength of a promoter specifically refers to its transcription strength, represented by the efficiency of initiation of transcription occurring at that promoter with high or low frequency. The higher the transcription strength, the more frequently transcription will occur at that promoter. Promoter strength is a typical feature of a promoter, because it determines how often a given mRNA sequence is transcribed, effectively giving higher priority for transcription to some genes over others, leading to a higher concentration of the transcript. A gene that codes for a protein that is required in large quantities, for example, typically requires a relatively strong promoter. The RNA polymerase can only perform one transcription task at a time and so must prioritize its work to be efficient. Differences in promoter strength are selected to allow for this prioritization.

The promoter strength may also refer to the frequency of transcription which is commonly understood as the transcription rate, e.g. as determined by the amount of a transcript in a suitable assay, e.g. RT-PCR or Northern blotting. For example, the transcription strength of a promoter described herein is determined in the host cell which is P. pastoris and compared to the native pGAP promoter of P. pastoris.

The strength of a promoter to express a gene of interest is commonly understood as the expression strength or the capability of supporting a high expression level/rate. For example, the expression and/or transcription strength of a promoter of the invention is determined in the host cell which is P. pastoris and compared to the native pGAP promoter of P. pastoris, e.g. measured upon being fully induced or derepressed.

According to a specific aspect, the GOIEC promoter is stronger than the TIFEC promoter. Preferably, a promoter is used, which has a transcription rate or strength is at least any one of 20%, 30%, 40%, 50%, 60%, 70%, 80%, 90% or 100%, or even higher, such as at least any one of 110%, 120%, 130%, 140%, 150%, 160%, 170%, 180%, 190%, or 200%, or even higher, as compared to the native pGAP promoter, such as determined in the (e.g., eukaryotic) host cell selected as a host cell for recombination purpose to produce the POI. The expression rate may, for example, be determined by the amount of expression of a reporter gene, such as eGFP.

The native pGAP promoter typically initiates expression of the gap gene encoding glyceraldehyde-3-phosphate dehydrogenase (GAPDH), which is a constitutive promoter present in most living organisms. GAPDH (EC 1.2.1.12), a key enzyme of glycolysis and gluconeogenesis, plays a crucial role in catabolic and anabolic carbohydrate metabolism.

The comparative transcription strength compared to a reference promoter may be determined by standard methods, such as by measuring the quantity of transcripts, e.g. employing a microarray, or else in a cell culture, such as by measuring the quantity of respective gene expression products in recombinant cells. In particular, the transcription rate may be determined by the transcription strength on a microarray, Northern blot or with quantitative real time PCR (qRT-PCR) or with RNA sequencing (RNA-seq).

As described herein, a heterologous promoter can be used in respective TIFECs to express any one or more, or all of the TIFs, and/or in a GOIEC to express the GOI. The heterologous promoter may be heterologous to the polynucleotide to be expressed and/or an artificial promoter, or a promoter that is originating from the wild-type host cell, but positioned in the host cell genome within a heterologous expression cassette or positioned at a location where it is not naturally-occurring in the wild-type host cell.

As described herein, according to specific embodiments, any of the TIF expression cassettes or the GOIEC may comprise and employ a constitutive promoter, such as any of the promoters further described herein.

Specific examples of constitutive promoter include e.g., the pGAP (e.g. SEQ ID NO:100, SEQ ID NO:101) and functional variants thereof, any of the constitutive promoter such as pCS1 (e.g. SEQ ID NO:102, or functional variants thereof such as published in WO2014139608), pMDH3 (e.g., SEQ ID NO:103), pPOR1 (e.g., SEQ ID NO:104), pRPP1B, pPDC1, pGPM1, pFBA1-1, or a functional variant of any of the foregoing.

Specific examples of inducible or repressible promoter include e.g., the native pAOX1 or pAOX2 and functional variants thereof, any of the regulatory promoter, such as pG1-pG8, and fragments thereof, published in WO2013050551; any of the regulatory promoter, such as pG1 and pG1-x, published in WO2017021541 A1.

In particular, a regulatable promoter, such as a de-repressible or repressible (herein referred to as (de)repressible), or inducible promoter may be used e.g., the native methanol-inducible promoters pAOX1 (SEQ ID NO:81) or pAOX2 (SEQ ID NO:82), or any of the native methanol-inducible promoters of P. pastoris (e.g., SEQ ID NO:83-96, published by Gasser, Steiger, & Mattanovich, Microb Cell Fact. 2015, 14: 196), or any other carbon source regulatable promoter, e.g., de-repressible promoters such as pG1-pG8 (pG1: SEQ ID NO:97, pG3: SEQ ID NO:105, pG4: SEQ ID NO:106, pG5: SEQ ID NO:107, pG7: SEQ ID NO:108, pG8: SEQ ID NO:109, and functional variants of any of the foregoing, such as fragments, e.g., fragments of pG1, designated pG1a-pG1f: SEQ ID NO:110-115), and the functional variants designated pG1-x, in particular pG1-3 (e.g., SEQ ID NO:98, such as referred to as pG1-D1240, or pG1-4 (e.g., SEQ ID NO:99, such as referred to as pG1-D1427), published in WO2013050551 and WO2017021541, or a functional variant of any of the foregoing with a length of at least 300, 400, or 500 bp (in particular including the 3′-end), or a functional variant of any of the foregoing.

Specifically, a functional variant of a promoter described herein comprises at least any one of 80%, 85%, 90%, 95%, or 100% sequence identity to the promoter from which it is derived, over the full-length or the part at the 3′-end of the promoter sequence which part has a length of at least 300, 400, or 500 bp, and is functional to operatively control expression of the polynucleotide to be expressed, in particular with about the same promoter activity (e.g. +/−any one of 50%, 40%, 30%, 20%, or 10%), although the promoter activity may be improved as compared to the promoter from which it is derived. Specific functional promoter variants of pG1-3 or pG1-4 are those comprising at least two main regulatory regions and/or at least two core regulatory regions, and/or at least two T motifs, as indicated in FIG. 1.

Further examples of suitable promoter sequences are described in Prielhofer et al. (BMC Syst Biol. 2017. 11(1):123) and Mattanovich et al. (Methods Mol. Biol. (2012) 824:329-58) and include glycolytic enzymes like triosephosphate isomerase (TPI), phosphoglycerate kinase (PGK), glyceraldehyde-3-phosphate dehydrogenase (GAPDH or GAP) and variants thereof, lactase (LAC) and galactosidase (GAL), P. pastoris glucose-6-phosphate isomerase promoter (PPGI), the 3-phosphoglycerate kinase promoter (pPGK), the glycerol aldehyde phosphate dehydrogenase promoter (pGAP), translation elongation factor promoter (PTEF), and the promoters of P. pastoris enolase 1 (pEN01), triose phosphate isomerase (pTPI), ribosomal subunit proteins (pRPS2, pRPS7, pRPS31, pRPL1), alcohol oxidase promoter (pAOX1, pAOX2) or variants thereof with modified characteristics, the formaldehyde dehydrogenase promoter (pFLD), isocitrate lyase promoter (pICL), alpha-ketoisocaproate decarboxylase promoter (pTHI), the promoters of heat shock protein family members (pSSA1, pHSP90, pKAR2), 6-phosphogluconate dehydrogenase (pGND1), phosphoglycerate mutase (pGPM1), transketolase (pTKL1), phosphatidylinositol synthase (pPIS1), ferro-02-oxidoreductase (pFET3), high affinity iron permease (pFTR1), repressible alkaline phosphatase (pPH08), N-myristoyl transferase (pNMT1), pheromone response transcription factor (pMCM1), ubiquitin (pUBI4), single-stranded DNA endonuclease (pRAD2), the promoter of the major ADP/ATP carrier of the mitochondrial inner membrane (pPET9) (WO2008/128701) and the formate dehydrogenase (FMD) promoter.

Further examples of suitable promoters include S. cerevisiae enolase (ENO1), S. cerevisiae galactokinase (GAL1), S. cerevisiae alcohol dehydrogenase and S. cerevisiae glyceraldehyde-3-phosphate dehydrogenase (ADH1, ADH2, GAP), S. cerevisiae triose phosphate isomerase (TPI), S. cerevisiae metallothionein (CUP1), and S. cerevisiae 3-phosphoglycerate kinase (PGK), and the maltase gene promoter (MAL).

The term “nucleotide sequence” or “nucleic acid sequence” used herein refers to either DNA or RNA. “Nucleic acid sequence” or “polynucleotide sequence” or simply “polynucleotide” refers to a single or double-stranded polymer of deoxyribonucleotide or ribonucleotide bases read from the 5′ to the 3′ end. It includes expression cassettes, self-replicating plasmids, infectious polymers of DNA or RNA, and non-functional DNA or RNA.

The term “protein of interest (POI)” as used herein refers to a polypeptide or a protein that is produced by means of recombinant technology in a host cell. More specifically, the protein may either be a polypeptide not naturally-occurring in the host cell, i.e. a heterologous protein, or else may be native to the host cell, i.e. a homologous protein to the host cell, but is produced, for example, by transformation or transfection with a self-replicating vector containing the nucleic acid sequence encoding the POI, or upon integration by recombinant techniques of one or more copies of the nucleic acid sequence encoding the POI into the genome of the host cell, or by recombinant modification of one or more regulatory sequences controlling the expression of the gene encoding the POI, e.g., of the promoter sequence. In some cases, the term POI as used herein also refers to any metabolite product by the host cell as mediated by the recombinantly expressed protein.

The term “sequence identity” of a variant, homologue or orthologue as compared to a parent nucleotide or amino acid sequence indicates the degree of identity of two or more sequences. Two or more amino acid sequences may have the same or conserved amino acid residues at a corresponding position, to a certain degree, up to 100%. Two or more nucleotide sequences may have the same or conserved base pairs at a corresponding position, to a certain degree, up to 100%.

Sequence similarity searching is an effective and reliable strategy for identifying homologs with excess (e.g., at least 50%) sequence identity. Sequence similarity search tools frequently used are e.g., BLAST, FASTA, and HMMER.

Sequence similarity searches can identify such homologous proteins or genes by detecting excess similarity, and statistically significant similarity that reflects common ancestry. Homologues may encompass orthologues, which are herein understood as the same protein in different organisms, e.g., variants of such protein in different different organisms or species.

“Percent (%) amino acid sequence identity” with respect to an amino acid sequence, homologs and orthologues described herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

For purposes described herein, the sequence identity between two amino acid sequences is determined using the NCBI BLAST program version BLASTP 2.8.1 with the following exemplary parameters: Program: blastp, Word size: 6, Expect value: 10, Hitlist size: 100, Gapcosts: 11.1, Matrix: BLOSUM62, Filter string: F, Compositional adjustment: Conditional compositional score matrix adjustment.

For pairwise protein sequence alignment of two amino acid sequences along their entire length the EMBOSS Needle webserver (https://www.ebi.ac.uk/Tools/psa/emboss_needle/) was used with default settings (Matrix: EBLOSUM62; Gap open:10; Gap extend: 0.5; End Gap Penalty: false; End Gap Open: 10; End Gap Extend: 0.5). EMBOSS Needle uses the Needleman-Wunsch alignment algorithm to find the optimum alignment (including gaps) of the two input sequences and writes their optimal global sequence alignment to file.

“Percent (%) identity” with respect to a nucleotide sequence e.g., of a promoter or a gene, is defined as the percentage of nucleotides in a candidate DNA sequence that is identical with the nucleotides in the DNA sequence, after aligning the sequence and introducing gaps, if necessary, to achieve the maximum percent sequence identity, and not considering any conservative substitutions as part of the sequence identity. Alignment for purposes of determining percent nucleotide sequence identity can be achieved in various ways that are within the skill in the art, for instance, using publicly available computer software. Those skilled in the art can determine appropriate parameters for measuring alignment, including any algorithms needed to achieve maximal alignment over the full length of the sequences being compared.

For purposes described herein (unless indicated otherwise), the sequence identity between two amino acid sequences is determined using the NCBI BLAST program version BLASTN 2.8.1 with the following exemplary parameters: Program: blastn, Word size: 11, Expect threshold: 10, Hitlist size: 100, Gap Costs: 5.2, Match/Mismatch Scores: 2,−3, Filter string: Low complexity regions, Mark for lookup table only.

The term “isolated” or “isolation” as used herein with respect to a POI shall refer to such compound that has been sufficiently separated from the environment with which it would naturally be associated, in particular a cell culture supernatant, so as to exist in “purified” or “substantially pure” form. Yet, “isolated” does not necessarily mean the exclusion of artificial or synthetic mixtures with other compounds or materials, or the presence of impurities that do not interfere with the fundamental activity, and that may be present, for example, due to incomplete purification. Isolated compounds can be further formulated to produce preparations thereof, and still for practical purposes be isolated—for example, a POI can be mixed with pharmaceutically acceptable carriers or excipients when used in diagnosis or therapy.

The term “purified” as used herein shall refer to a preparation comprising at least 50% (mol/mol), preferably at least 60%, 70%, 80%, 90% or 95% of a compound (e.g., a P01). Purity is measured by methods appropriate for the compound (e.g., chromatographic methods, polyacrylamide gel electrophoresis, HPLC analysis, and the like). An isolated, purified POI as described herein may be obtained by purifying the cell culture supernatants to reduce impurities.

As isolation and purification methods for obtaining a recombinant polypeptide or protein product, methods, such as methods utilizing difference in solubility, such as salting out and solvent precipitation, methods utilizing difference in molecular weight, such as ultrafiltration and gel electrophoresis, methods utilizing difference in electric charge, such as ion-exchange chromatography, methods utilizing specific affinity, such as affinity chromatography, methods utilizing difference in hydrophobicity, such as reverse phase high performance liquid chromatography, and methods utilizing difference in isoelectric point, such as isoelectric focusing may be used.

The following standard methods are preferred: cell (debris) separation and wash by Microfiltration or Tangential Flow Filter (TFF) or centrifugation, POI purification by precipitation or heat treatment, POI activation by enzymatic digest, POI purification by chromatography, such as ion exchange (IEX), hydrophobic interaction chromatography (HIC), affinity chromatography, size exclusion (SEC) or HPLC chromatography, POI precipitation, concentration and washing, such as by ultrafiltration steps.

A highly purified product is essentially free from contaminating proteins, and preferably has a purity of at least 90%, more preferred at least 95%, or even at least 98%, up to 100%. The purified products may be obtained by purification of the cell culture supernatant or else from cellular debris.

An isolated and purified POI can be identified by conventional methods such as Western blot, HPLC, activity assay, or ELISA.

The term “recombinant” as used herein shall mean “being prepared by or the result of genetic engineering. A “recombinant cell” or “recombinant host cell” is herein understood as a cell or host cell that has been genetically engineered or modified to comprise a nucleic acid sequence which was not native to said cell. A recombinant host may be engineered to delete and/or inactivate one or more nucleotides or nucleotide sequences, and may specifically comprise an expression vector or cloning vector containing a recombinant nucleic acid sequence, in particular employing nucleotide sequence foreign to the host. A recombinant protein is produced by expressing a respective recombinant nucleic acid in a host. The term “recombinant” with respect to a POI as used herein, includes a POI that is prepared, expressed, created or isolated by recombinant means, such as a POI isolated from a host cell transformed or transfected to express the POI. In accordance with the present invention conventional molecular biology, microbiology, and recombinant DNA techniques within the skill of the art may be employed. Such techniques are explained fully in the literature. See, e.g., Maniatis, Fritsch & Sambrook, “Molecular Cloning: A Laboratory Manual, Cold Spring Harbor, (1982).

Certain recombinant host cells are “engineered” host cells which are understood as host cells which have been manipulated using genetic engineering, i.e. by human intervention. When a host cell is engineered to express, co-express or overexpress a given gene or the respective protein, the host cell is manipulated such that the host cell has the capability to express such gene and protein, respectively, to a higher extent compared to the host cell under the same condition prior to manipulation, or compared to the host cells which are not engineered such that said gene or protein is expressed, co-expressed or overexpressed. As herein described, the yield of a protein of interest (POI) can be increased by co-expressing or overexpressing the TIF(s) described herein, when compared to the same cell expressing the same POI under the same culturing conditions, however, without the polynucleotides encoding the TIF(s) being co-expressed or overexpressed or without being engineered to co-express or overexpress the polynucleotide encoding the TIF(s).

It has surprisingly turned out that overexpression of TIFs which are part of the mRNP, but not of subunits of eIF3, was leading to increased production and secretion of several recombinant POIs.

According to a specific example as described herein, TIF overexpression enhanced translational capacity of the engineered cells and also correlated with higher levels of POI transcripts and endogenous transcripts.

It was even more surprising that the yield of POI production was increased by overexpression of single TIFs such as eIF4A, eIF4G, eIF4E, PAB1 and RLI1 as well as combinations thereof in different modes of cultivation (screening, fed batch, and continuous cultivation).

The foregoing description will be more fully understood with reference to the following examples. Such examples are, however, merely representative of methods of practicing one or more embodiments of the present invention and should not be read as limiting the scope of invention.

### EXAMPLES

### Example 1: Construction of Translation Factor (TIF) Overexpression Strains

a) Host Strains and Expression Vectors:

P. pastoris strains CBS7435 or CBS2612 (CBS-KNAW Fungal Biodiversity Centre, Centraalbureau voor Schimmelcultures, Utrecht, The Netherlands) were used as background strains. To evaluate the impact of translation factor overexpression, different secretory model proteins were used as reporters. For this purpose, CBS2612_P_{G1-3}_vHH #4 (described in WO2020/144313A1) was used for expression. To generate additional host strains, the expression cassette for P_{G1-3}_HSA was transformed into CBS2612 and the P_{G1-3}_vHH expression cassette was transformed into CBS7435 as described in WO2020144313A1. MutS P_{AOX1}-vHH (Zavec et al. 2020, Biotechnol Bioeng. 117(5):1394-1405) was used to evaluate the effect in methanol conditions.

b) Generation of TIF Overexpression Vectors

Targets were overexpressed by homologous recombination of their respective expression cassettes into the host strains. Plasmids for this were generated by usage of two different cloning strategies. The chosen target translation factors are shown in Tables 2 and 7.

Construction and Selection of P. pastoris Translation Factor Overexpression Strains by pPuzzle Based Expression

The pPM2aK21 plasmid, a derivative of the pPuzzle_ZeoR vector backbone described in WO2008/128701A2, consisting of an AOX terminator sequence (for integration into the native AOX1 terminator locus), an origin of replication for E. coli (pUC19), an antibiotic resistance cassette (kanMX conferring resistance to Kanamycin and G418) for selection in E. coli and yeast, an expression cassette for the gene of interest (GOI) consisting of a GAP promoter, a multiple cloning site (MCS) and the S. cerevisiae CYC1 transcription terminator, was used. The chosen overexpression genes were amplified by PCR (Q5® High-Fidelity DNA Polymerase, New England Biolabs) from start to stop codon using the primers shown in Table 8. The sequences were cloned into the MCS of the pPM2a expression vector with the two restriction enzymes SbfI and SfiI. Gene sequences were verified by Sanger sequencing.

Construction and Selection of P. pastoris Translation Factor Overexpression Strains with the GoldenPiCS System

The genes selected for overexpression were amplified by PCR (Q5® High-Fidelity DNA Polymerase, New England Biolabs) from start to stop codon or split into two to several fragments. The GoldenPiCS system (Prielhofer et al. 2017. BMC Systems Biol. 11, 123, doi: 10.1186/s12918-017-0492-3) requires the introduction of silent mutations in some coding sequences. This was performed by amplifying several fragments from one coding sequence by usage of the primers in Tables 3 and 9. Alternatively, gBlocks were obtained (Integrated DNA Technology IDT). Genomic DNA from P. pastoris strain CBS2612 was used as PCR templates. The resulting fragments, after amplification with the primers in Tables 3 and 9, were introduced into BB1 of the GoldenPiCS system by using the restriction enzyme BsaI. The GoldenPiCS system consists of the backbones BB1, BB2 and BB3. The assembled BB1s carrying the respective coding sequence were combined with promoter and terminator regions in BB2s and then further processed to create the required BB3 integration plasmids as described in Prielhofer et al. 2017. All promoters and terminators used to assemble expression cassettes in BB2 or BB3 backbones are described in Prielhofer et al. 2017. The used BB3rN contains the 5″-RGI1 genome integration region and the NatMX selection marker cassette for selection on nourseothricin. All plasmids contain an origin of replication for E. coli (pUC19).

c) Generation of TIF Overexpressing Transformants

Plasmids were linearized using AscI restriction enzyme prior to electroporation (using a standard transformation protocol described in Gasser et al. 2013 (Future Microbiol. 8(2):191-208) into P. pastoris. Selection of positive transformants was performed on YPD plates (per liter: 10 g yeast extract, 20 g peptone, 20 g glucose, 20 g agar-agar) containing 500 μg mL⁻¹ of G418 or 100 μg mL⁻¹ nourseotricin. Colony PCR was used to ensure the presence of the transformed plasmid in the correct locus. For this, genomic DNA was obtained by cooking P. pastoris colonies in 0.04 M NaOH which was directly applied for PCR with the appropriate primers.

d) Determination of Gene Copy Number (GCN) of Overexpression Targets

Expression strength is often correlated to the number of expression cassettes integrated into the P. pastoris genome. Therefore, the gene copy number of each of the overexpression targets was determined. Genomic DNA was isolated using the Wizard® Genomic DNA Purification Kit (Promega Corporation, Cat. No. A1120). Then, gene copy numbers were determined using quantitative real-time PCR (qPCR). For this, the Blue S′Green qPCR Kit (Biozym), was used. The Blue S′Green qPCR master mix was mixed with primers and samples and applied for real time analysis in a real-time PCR cycler (Rotor Gene, Qiagen). A list of used primers is shown in Table 1. All samples were analysed in triplicates. The Rotor Gene software was used for data analysis. As a calibrator, the ACT1 gene was used. GCN was determined by usage of P_{GAP} primers for the single overexpression constructs (see Example 4), and TIF2 primers for combined overexpression constructs (see Example 5). The results are shown in Table 10 and Table 12, respectively. As the chosen overexpression targets are endogenous genes of P. pastoris, a GCN of 2 shows successful integration of one additional gene copy, one of the genes being the original gene and the second being the overexpression cassette.

TABLE 1
qPCR Primers used for GCN determination. ACT1 was used as
calibrator. PGAP was used to determine GCN for the single overexpression
constructs, TIF2 for combined overexpression constructs.

| | | | Product |
|---|---|---|---|
| Name | Target | Sequence | Length |
| qPCR_PpACT1_fwd | ACT1 | CCTGAGGCTTTGTTCCACCCATCT | 148 bp |
| | | SEQ ID NO: 116 | |
| qPCR_PpACT1_rev | ACT1 | GGAACATAGTAGTACCACCGGACATAACGA | |
| | | SEQ ID NO: 117 | |
| qPCR_pGAP_fwd | P_{GAP} | TAAAATTCTCCGGGGTAAAAC | 116 bp |
| | | SEQ ID NO: 118 | |
| qPCR_pGAP_rev | P_{GAP} | CTCTCCAGCAGAGTAAAATTTC | |
| | | SEQ ID NO: 119 | |
| qPCR_TIF2_fwd | TIF2 | CACAGAATCGGTAGAGGTG | 139 bp |
| | | SEQ ID NO: 120 | |
| qPCR_TIF2_rev | TIF2 | CAGTGATGGAAGATGGC | |
| | | SEQ ID NO: 121 | |
| qPCR_HSA_fwd | HSA | AGACTTTCACTTTCCACGCT | 153 bp |
| | | SEQ ID NO: 122 | |
| qPCR_HSA_rev | HSA | CAACGAAAGCAGCGAAGTC | |
| | | SEQ ID NO: 123 | |

### Example 2: Analysing the Effects of TIF Overexpression on Recombinant Protein Production

To determine the effect of translation factor overexpression on recombinant protein secretion, engineered overexpression strains were cultivated in suitable screening conditions such as glucose limiting conditions when using pG-promoters for the GOI (Prielhofer et al. 2013. Microb Cell Fact 12, 5) or methanol induction in case of promoters derived from the methanol-utilization pathway (Gasser et al. 2015. Microb. Cell Fact 14:196). The engineering of the P. pastoris host strains were done as described in Example 1, by integrating either the pPuzzle-based or the GoldenPiCS BB3rN-based TIF expression vectors into the P. pastoris genome. The engineered P. pastoris strains were then cultivated in small scale (screening procedure), thereby simulating a fed-batch cultivation. The recombinant protein secreted into the supernatant was quantified and the titers and yields of the different engineered strains were compared to the parental host strain.

Media: synthetic screening medium ASMv6 per liter: 6.30 g (NH₄)₂HPO₄, 0.8 g (NH₄)₂SO₄, 0.49 MgSO₄*7H₂O, 2.64 g KCl, 0.0535 g CaCl₂*2H₂O, 22.0 g citric acid monohydrate, 1470 μL PTM₀ trace salt stock solution, 20 mL NH₄OH (25%), 4 mL Biotin (0.1 g L⁻¹). Solid KOH was added to set the pH to 6.4-6.6.

PTM₀ trace salt stock solution per liter: 5.0 ml H₂SO₄ (95-98%), 65.0 g FeSO₄*7H₂O, 20 g ZnCl₂, 6.00 g CuSO₄*5H₂O, 3.36 g MnSO₄*H₂O, 0.82 g CoCl₂*6H₂O, 0.20 g Na₂MoO₄*2H₂O, 0.08 g NaI, 0.02 g H₃BO₃

a) Screening of Engineered P. pastoris Strains with GOI Expression Under Control of pG-Promoters

For screening of model protein secretion, single colonies, with PCR verified gene integration into the correct locus, were inoculated in 2 mL liquid YPG medium (per liter: g peptone, 10 g yeast extract, 12.6 g glycerin 100%, pH 7.4-7.6) containing 50 pg mL⁻¹ Zeocin and 500 μg mL⁻¹ G418 or 100 μg mL⁻¹ nourseothricin (if appropriate). Additionally, on each plate the host strain was cultivated in quadruplicate for comparison. This preculture was grown for approximately 24 h at 25° C. in 24-DWP at 280 rpm. The precultures were then used to inoculate 2 mL of synthetic screening medium ASMv6 to a starting-OD₆₀₀ of 8. The media contained 50 g L⁻¹ polysaccharide (EnPump200 polysaccharide, Enpresso) and 0.4% of glucose-releasing enzyme (Reagent A, Enpresso) as carbon source. Cultivation conditions were similar to pre-culture conditions. After 48 hours, 1 mL of cell suspension was transferred to a pre-weighted 1.5 mL centrifugation tube and centrifuged at 16,000 g for 5 min at room temperature. Supernatants were carefully transferred to a new vial and stored at −20° C. until further use. Centrifugation tubes containing the pellets were weighted again to determine the wet cell weight (WCW). Quantification of the recombinant secreted protein in the supernatant was done by microfluidic capillary electrophoresis as described below.

b) Screening of Engineered P. pastoris Strains with GOI Expression Under Control of Methanol-Inducible Promoters

2 mL YPD medium (per liter: 20 g peptone, 10 g yeast extract, 22 g D(+)-glucose monohydrat, pH 7.4-7.6) containing 50 μg mL⁻¹ Zeocin and 500 μg mL⁻¹ G418 or 100 pg mL⁻¹ nourseothricin (if appropriate) were inoculated with a single colony of a P. pastoris clone and grown overnight at 25° C. in 24-DWP at 280 rpm. The precultures were then used to inoculate 2 mL of synthetic screening medium ASMv6 to a starting-OD₆₀₀ of 8. The media contained 25 g L⁻¹ polysaccharide (EnPump200 polysaccharide, Enpresso) and 0.35% of glucose-releasing enzyme (Reagent A, Enpresso) as carbon source. These cultures were incubated for 48 h at 25° C. in 24-DWP at 280 rpm. After the first 3 hours the cells were fed with 10 μL (0.5%) pure methanol. Then the cells were fed again after 19 h, 27 h and 43 h cultivation time with 20 μL (1%) pure methanol. After 48 hours, 1 mL of cell suspension was transferred to a pre-weighted 1.5 mL centrifugation tube and centrifuged at 16,000 g for 5 min at room temperature. Supernatants were carefully transferred to a new vial and stored at −20° C. until further use. Centrifugation tubes containing the pellets were weighted again to determine the wet cell weight (WCW). Quantification of the recombinant secreted protein in the supernatant was done by microfluidic capillary electrophoresis as described below.

c) Quantification of Secreted Recombinant Protein by Microfluidic Capillary Electrophoresis (mCE)

The ‘LabChip GX/GXII System’ (Perkin Elmer) was used for quantitative analysis of secreted protein titer in culture supernatants. The consumables ‘Protein Express Lab Chip’ (760499, PerkinElmer) and ‘Protein Express Reagent Kit’ (CLS960008, PerkinElmer) were used. Chip and sample preparation were done according to the manufacturer's recommendations. A brief description of the procedure is given below.

Chip preparation: After the reagents came to room temperature 520 and 280 μL of Protein Express Gel Matrix were transferred to spin filters. 20 μL of Protein Express Dye solution was added to the 520 μL Gel Matrix containing spin filter. After briefly vortexing the dye containing spin filter in the inverted orientation, both spin filters were centrifuged at 9300 g for 10 minutes. To wash the chip, 120 μL Milli-Q® water were added to all active chip wells and the chip was subjected to the instruments washing program. After two further rinsing steps with Milli-Q® water, remaining fluids were fully aspirated and appropriate amounts of the filtered Gel Matrix solutions as well as the Protein Express Lower Marker solution were added to the appropriate chip wells.

Sample and ladder preparation: For sample preparation 6 μL sample were mixed with 21 μL of sample buffer in a 96-microtiter plate. Samples were denatured at 100° C. for 5 min and centrifuged at 1,200 g for 2 min. Subsequently, 105 μL of Milli-Q® water were added. Sample solutions were briefly mixed by pipetting and centrifuged again at 1,200 g for 2 min before measurement. To prepare the ladder 12 μL of Protein Express Ladder were denatured at 100° C. for 5 min in a PCR tube. Subsequently, 120 μL of Milli-Q® water were added and the ladder solution was briefly vortexed before spinning the tube for 15 seconds in a minicentrifuge and starting the measurement.

Quantitation was done by employing the LabChip software provided by the manufacturer and comparison against BSA standards.

### Example 3: Effect of Translation Initiation Factor 3 (eIF3) Subunit Overexpression on Recombinant Protein Secretion in P. pastoris

First, the subunits of the translation initiation factor 3, eIF3, were overexpressed. This factor consists of 6 subunits in yeast, which were overexpressed on their own and in different combinations. For overexpression, the eIF3 subunits were cloned into GoldenPiCS vectors and transformed into the host strain CBS2612 P_{G1-3} vHH #4, as described in Example 1. The engineered strains were then screened as described in Example 2 and yields were compared to the host strain.

a) Overexpressing Single Subunits of eIF3.

The subunits of eIF3, shown in Table 2, were amplified by using the primers shown in Table 3 and cloned into the host strain, CBS2612 P_{G1-3} vHH #4, as described in Example 1. No overexpression vectors were obtained for eIF3a.

TABLE 2
Chosen overexpression targets. All of the given
genes are known to be subunits of eIF3 in yeast.

| Translation factor subunit | Gene | Gene Identifier | Gene Length | |
|---|---|---|---|---|
| eIF3a | RPG1 | PP7435_Chr3-0875 | 2544 | bp |
| eIF3b | PRT1 | PP7435_Chr3-0499 | 2148 | bp |
| eIF3c | NIP1 | PP7435_Chr3-0419 | 2433 | bp |
| eIF3g | TIF35 | PP7435_Chr4-0549 | 867 | bp |
| eIF3i | TIF34 | PP7435_Chr1-0286 | 1032 | bp |
| eIF3j | HCR1 | PP7435_Chr3-0147 | 771 | bp |

TABLE 3
Primers used for cloning of elF3 subunits.

| Name | Sequence |
|---|---|
| HCR1_fragment1_fwd | GATCGGTCTCAC [symbol] TCTTGGGACGACG |
| | SEQ ID NO: 124 |
| HCR1_fragment1_rev | GATCGGTCTCAGTCCTGCTTTGTCTCTAG |
| | SEQ ID NO: 125 |
| HCR1_fragment2_fwd | GATCGGTCTCTGGACTATGTGAACCTCC |
| | SEQ ID NO: 126 |
| HCR1_fragment2_rev | GATCGGTCTCAAAGC [symbol] CATGAAATCGTCATCACC |
| | SEQ ID NO: 127 |
| PRT1_fragment1_fwd | GATCGGTCTCACATGACAAACGAACCAGAA |
| | SEQ ID NO: 128 |
| PRT1_fragment1_rev | GATGGGTCTCAGACCGGTTTGGAAATCC |
| | SEQ ID NO: 129 |
| PRT1_fragment2_fwd | GATCGGTCTCAGGTCTGCCTTGTAGAAC |
| | SEQ ID NO: 130 |
| PRT1_fragment2_rev | GATCGGTCTCAGAGCCTGCATTCAAAGTTG |
| | SEQ ID NO: 131 |
| PRT1_fragment3_fwd | GATCGGTCTCAGCTCTGGTGACCTTG |
| | SEQ ID NO: 132 |
| PRT1_fragment3_rev | GATCGGTCTCAAAGC [symbol] ATCCACAATTTCTTCTTTCTC |
| | SEQ ID NO: 133 |
| NIP1_fragment1_fwd | GATGGGTCTCAC [symbol] TCCCGTTTCTTTGCGTCAG |
| | SEQ ID NO: 134 |
| NIP1_fragment1_rev | GATCGGTCTCTAAGC [symbol] TTTACTATAGATCTTCTTTTGGTCTTTGA |
| | CATTGGAGGACTG, SEQ ID NO: 135 |
| RPG1_fragment1_fwd | GATCGGTCTCAC [symbol] GCTCCAAACTACAAC |
| | SEQ ID NO: 136 |
| RPG1_fragment1_rev | GATCGGTCTCAGTGAAGAATTCGTAGATTGTCTC |
| | SEQ ID NO: 137 |
| RPG1_fragment2_fwd | GATCGGTCTCATCACCTCCAAAAGGGTTAG |
| | SEQ ID NO: 138 |
| RPG1_fragment2_rev | GATCGGTCTCAGTTATGTGTCTCGACCTTAC |
| | SEQ ID NO: 139 |
| RPG1_fragment3_fwd | GATCGGTCTCATAACAGGCTAAAGAGAATGG |
| | SEQ ID NO: 140 |
| RPG1_fragment3_rev | GATCGGTCTCTAAGC [symbol] TATTCTTCCTTGACGCTTTAG |
| | SEQ ID NO: 141 |
| TIF34_fragment1_fwd | GATCGGTCTCAC [symbol] AGGCCAATTTTACTGAAG |
| | SEQ ID NO: 142 |
| TIF34_fragment1_rev | GAAGGGTCTCATTGGACACCGAAAATAGC |
| | SEQ ID NO: 143 |
| TIF34_fragment2_fwd | GATCGGTCTCTCCAAGGATTCGGTAGC |
| | SEQ ID NO: 144 |
| TIF34_fragment2_rev | GATCGGTCTCAAAGC [symbol] AGAGGCAGTCTGTAAAG |
| | SEQ ID NO: 145 |
| TIF35_fragment1_fwd | GATCGGTCTCAC [symbol] GCAACAGCAGTAG |
| | SEQ ID NO: 146 |
| TIF35_fragment1_rev | GATCGGTCTCACCTTGACACAATCTACACACC |
| | SEQ ID NO: 147 |
| TIF35_fragment2_fwd | GATCGGTCTCAAAGGAGATCATTTCACTACC |
| | SEQ ID NO: 148 |
| TIF35_fragment2_rev | GAAGGGTCTCACTCCGAGTCCAGAGC |
| | SEQ ID NO: 149 |
| TIF35_fragment3_fwd | GATCGGTCTCAGGAGGCTCTGGAAGCTC |
| | SEQ ID NO: 150 |
| TIF35_fragment3_rev | GATCGGTCTCTAAGC [symbol] CACCTTAGGCTTTGGCTTG |
| | SEQ ID NO: 151 |

Table 4 shows the results of the single overexpression of the eIF3 subunits. Each target gene was overexpressed with a different promoter to achieve approximately 10-fold overexpression. These approximate overexpression strengths are shown in column OE and were calculated as described in Example 4a. The screening results are shown as fold change of the vHH yield compared to the host strain. The results in Table 4 clearly show that overexpression of single eIF3 subunits has no effect on recombinant protein secretion in Pichia pastoris (fold changes of the vHH yield between the engineered strains and the control are all around 1, meaning that there is no difference in protein production between the engineered strains and the parental control strain). This was unexpected as Roobol et al. (Metabolic Engineering 2020, 59:98-105) reported increased growth rate and increased protein synthetic capacity upon transient and stable overexpression of the eIF3i and eIF3v subunits in the mammalian HEK and CHO cell lines.

TABLE 4
Single overexpression of translation initiation factor eIF3 subunits
in strain CBS2612 P_{G1-3} VHH #4. The column FC vHH yield shows
the fold change of the vHH yield compared to the host strain.

| Name | Promoter | Gene | OE | FC vHH yield | Number of clones |
|---|---|---|---|---|---|
| eIF3b | P_{GAP} | PRT1 | 10 | 1.04 ± 0.16 | 10 |
| eIF3c | P_{MDH3} | NIP1 | 10 | 0.93 ± 0.12 | 10 |
| eIF3g | P_{RPP1B} | TIF35 | 10 | 1.15 ± 0.14 | 10 |
| eIF3i | P_{SPI1} | TIF34 | 10 | 1.12 ± 0.06 | 10 |
| eIF3j | P_{POR1} | HCR1 | 10 | 0.82 ± 0.11 | 10 |

a) Overexpressing Combinations of eIF3 Subunits.

Next, different combinations of eIF3 subunits were chosen for overexpression, described in Table 5. Cloning and transformation were done as described in Example 1 and the resulting strains were screened as described in Example 2. The promoters were chosen, as described in Example 4a, to keep the transcript concentration ratios in the cell the same as in the native strain. Column OE shows the calculated overexpression strengths.

TABLE 5
Chosen overexpression combinations for eIF3, shown also with the selected promoters. OE
shows the estimated increase in TIF transcript levels in the engineered strains, compared to
the parental strain according to the gene expression data from Rebnegger et al. 2014.
Biotech J. 9(4):511-25. CBS2612 P_{G1-3} vHH #4 was used as host strain.

| Name | Gene 1 | OE | Gene 2 | OE | Gene 3 | OE | Gene 4 | OE | Gene 5 | OE |
|---|---|---|---|---|---|---|---|---|---|---|
| C21 | P_{GAP} | 10 | P_{MDH3} | 10 | | | | | | |
| | PRT1 | | NIP1 | | | | | | | |
| C22 | P_{GAP} | | P_{POR1} | | | 10 | | | | |
| | PRT1 | | HCR1 | | | | | | | |
| C23 | P_{GAP} | | P_{MDH3} | | P_{SPI1} | | P_{RPP1B} | 10 | | |
| | PRT1 | | NIP1 | | TIF34 | | TIF35 | | | |
| C24 | P_{GAP} | | P_{MDH3} | | P_{SPI1} | | P_{RPP1B} | | P_{POR1} | 10 |
| | PRT1 | | NIP1 | | TIF34 | | TIF35 | | HCR1 | |

TABLE 6
Combined overexpression of eIF3 subunits in strain CBS2612 P_{G1-3}
vHH #4. FC yield is the fold change of the yield of secreted vHH

| Name | FC vHH yield | Number of clones |
|---|---|---|
| C21 | 1.01 ± 0.13 | 10 |
| C22 | 0.98 ± 0.10 | 10 |
| C23 | 1.05 ± 0.04 | 10 |
| C24 | 0.93 ± 0.02 | 10 |

Table 6 shows the fold change of the vHH yield in comparison to the host strain. Even combinations overexpressing several subunits of eIF3 did not increase vHH production in the screenings. As in Example 3a, the fold change values are all around 1, meaning that there is no significant increase in recombinant protein secretion when eIF3 subunits are overexpressed either alone or in combinations.

### Example 4: Effect of Overexpression of Single TIFs of the mRNP on Recombinant Protein Production

Translation factors acting on translation initiation and being part of the mRNP and the closed loop complex were selected for overexpression purposes: eIF4A, eIF4E, eIF4G, PAB1 and RLI1 (Table 7) and overexpression vectors were constructed as in Example 1 b using the primers shown in Tables 8 and 9. CBS2612_P_{G1-3}_vHH #4 (described in WO2020/144313A1) was used as parental host strain and transformed with the single TIF overexpressing vectors described in Example 1.

TABLE 7
Translation initiation factors (TIFs) chosen for overexpression

| | Translation | | Gene | Gene |
|---|---|---|---|---|
| Name | factor | Gene | identifier | length |
| CDC33 | eIF4E | CDC33 | PP7435_Chr3-0197 | 609 bp |
| TIF2 a | eIF4A | TIF2 | PAS_chr3_0595 | 1212 bp |
| TIF2 b | eIF4A | TIF2 | PP7435_Chr3-0610 | 1461 bp |
| TIF4632 | eIF4G | TIF4632 | PP7435_Chr1-0352 | 3297 bp |
| PAB1 | PAB1 | PAB1 | PP7435_Chr2-1212 | 1881 bp |
| RLI1 | RLI1 | RLI1 | PP7435_Chr2-1213 | 1821 bp |

a) Generation of Single OE Vectors and Determination of OE Strength

TABLE 8
Primers used for construction of P. pastoris translation factor
overexpression strains by pPuzzle based expression. The start and
stop codons of each respective gene are shown in italic and bold.

| Name | Sequence |
|---|---|
| TIF2a_fwd | ACGCCCTGCAGG [symbol] TCTGAAGGTATTATTGAAATCGACACT |
| | AACTTAATCG, SEQ ID NO: 152 |
| TIF2a_rev | GACTGGCCGAGGCGGCC [symbol] AGACTCATTAACTTCCTCAGT |
| | CTCAAACAAGTC, SEQ ID NO: 153 |
| TIF4632_fwd | GTCTCCTGCAGG [symbol] TCCAATAAGAACGTGGATACAGCTCCA, |
| | SEQ ID NO: 154 |
| TIF4632_rev | GACTGGCCGAGGCGGCC [symbol] AACTTCCTGTTCCTCTTCTTGC |
| | TCTC, SEQ ID NO: 155 |
| PAB1_fwd | GAGGCCTGCAGG [symbol] TCTGTCGATACCAAGGAAGTTCAAG, |
| | SEQ ID NO: 156 |
| PAB1_rev | GAGCGGCCGAGGCGGCC [symbol] GTTTGCTTGTGCATCCGCTT, |
| | SEQ ID NO: 157 |

TABLE 9
Primers used for construction of P. pastoris translation factor
overexpression strains with the GoldenPICS system. The Bsal restriction sites are
shown underlined. The start and stop codons of each respective gene are shown
in italic and bold. Silent mutations are shown in bold and underlined.

| Name | Sequence |
|---|---|
| CDC33_fragment1_fwd | GATCGGTCTCCC [symbol] TCAGAGACTGAAAACG |
| | SEQ ID NO: 158 |
| CDC33_fragment1_rev | GATCGGTCTCTATTCAGGTTTGATTCCATCTC |
| | SEQ ID NO: 159 |
| CDC33_fragment2_fwd | GATCGGTCTCAGAATGGGAGGACGAG |
| | SEQ ID NO: 160 |
| CDC33_fragment2_rev | GATCGGTCTCACGCGGGACCAC |
| | SEQ ID NO: 161 |
| CDC33_fragment3_fwd | GATCGGTCTCTCGCGGTCTGTTGAG |
| | SEQ ID NO: 162 |
| CDC33_fragment3_rev | GATCGGTCTCACATCCTTGGACTTGGTC |
| | SEQ ID NO: 163 |
| CDC33_fragment4_fwd | GATCGGTCTCAGATGAGGCAGTTTTAAGACC |
| | SEQ ID NO: 164 |
| CDC33_fragment4_rev | GATCGGTCTCAAAGC [symbol] AATGCTGAAAGAAGGTACG |
| | SEQ ID NO: 165 |
| TIF2a_fragment1_fwd | GATCGGTCTCAC [symbol] TCTGAAGGTATTATTGAAATCGACACTAA |
| | CTTAATCGAGACAAACTAC, SEQ ID NO: 166 |
| TIF2a_fragment1_rev | GATCGGTCTCAAAGC [symbol] AGACTCATTAACTTCCTCAGTCTCAAA |
| | CAAGTCAGTG, SEQ ID NO: 167 |
| TIF2b_fragment1_fwd | GACTGGTCTCAC [symbol] CATCCATACACCG |
| | SEQ ID NO: 168 |
| TIF2b_fragment1_rev | GATCGGTCTCAGGTCGTAGTTTGTCTCG |
| | SEQ ID NO: 169 |
| TIF2b_fragment2_fwd | GATCGGTCTCAGACCAGGTTGTCAGC |
| | SEQ ID NO: 170 |
| TIF2b_fragment2_rev | GATCGGTCTCAAAGC [symbol] AGACTCATTAACTTCCTCAG |
| | SEQ ID NO: 171 |
| TIF4632_fragment1_fwd | GATCGGTCTCAC [symbol] TCCAATAAGAACGTGG |
| | SEQ ID NO: 172 |
| TIF4632_fragment1_rev | GATCGGTCTCACCAGCGTCCTCAGAT |
| | SEQ ID NO: 173 |
| TIF4632_fragment2_fwd | GATCGGTCTCACTGGCAAGACTAGAGATG |
| | SEQ ID NO: 174 |
| TIF4632_fragment2_rev | GACTGGTCTCACTTAACGAACGAGGTACC |
| | SEQ ID NO: 175 |
| TIF4632_gBlock | CCATTGGGTCTCATAAGAAATAAGGAGGCTGAAGTCAAGACTGC |
| | TCCAGACGGATCTATAATAGTATCAGAAGAGGACATCAAAAGGA |
| | AAACTAAATCCCTTTTGAATAAGTTGACGTTGGAATTCTTTGATG |
| | ATATCTCAAACGATATAATTGCTTTGACCAAGCAAGCTCAATGGG |
| | AAGATGACGTCAAGACTTTGAAACAAGTTATTGAGTCTATATTTG |
| | CAAAGGCTTGTGACGAACCCTACTGGTCCTCTATGTACGCTAAA |
| | TTATGCGCCAAAATGTGCAAGGACACCCCACCTGAGATCAAGGA |
| | AACTAATGAGAAGGGAAATACTTTCACCGGTGGTGATTTGGTGA |
| | GAAGAGTGTTGATTAATAGATGTCATGAGACCGATTCG |
| | SEQ ID NO: 176 |
| TIF4632_fragment3_fwd | AGCTGGTCTCAGTCAAACCGAATATCAGAAAG |
| | SEQ ID NO: 177 |
| TIF4632_fragment3_rev | GATCGGTCTCAAAGC [symbol] AACTTCCTGTTCCTCTTC |
| | SEQ ID NO: 178 |
| PAB1_fragment1_fwd | GTACGGTCTCAC [symbol] TCTGTCGATACCAAG |
| | SEQ ID NO: 179 |
| PAB1_fragment1_rev | GATCGGTCTCAGAAGCCAATGTCTCGG |
| | SEQ ID NO: 180 |
| PAB1_fragment2_fwd | GATCGGTCTCTCTTCATTGTATGTTGGTGAG |
| | SEQ ID NO: 181 |
| PAB1_fragment2_rev | GATCGGTCTCACACGTTGGGACCAC |
| | SEQ ID NO: 182 |
| PAB1_fragment3_fwd | GATCGGTCTCACGTGACCCTTCCTTG |
| | SEQ ID NO: 183 |
| PAB1_fragment3_rev | GATCGGTCTCAGTTCTTGACAAAGACATTGG |
| | SEQ ID NO: 184 |
| PAB1_fragment4_fwd | GATCGGTCTCTGAACTTTGACACTGAGTCC |
| | SEQ ID NO: 185 |
| PAB1_fragment4_rev | GATCGGTCTCTAAGC [symbol] GTTTGCTTGTGCATCC |
| | SEQ ID NO: 186 |
| RLI1_fragment1_fwd | GATCGGTCTCGC [symbol] AGTGAGAAAAACACACG |
| | SEQ ID NO: 187 |
| RLI1_fragment1_rev | GATCGGTCTCAAAGC [symbol] TAACTCAGTGTTCTCAAGG |
| | SEQ ID NO: 188 |

For all the described single TIF overexpressions the strong and constitutive pGAP promoter was used and the TIF expression cassette was either integrated into the 5″-RGII locus or into the AOX1 transcription terminator. Based on gene expression data described in Rebnegger et al. 2014. Biotech J. 9(4):511-25, the expected degree of overexpression with the chosen promoter was calculated. The estimated increase in expression strength compared to the parent strain is given in Table 10 in the column “OE”.

Table 10 also shows the measured GCN of the chosen clones and the results of the screening procedure. All clones shown in Table 10 contained one additional copy of the respective TIF gene (indicated by GCN=2).

b) Effect of Single TIF Overexpression on Recombinant Protein Production Using vHH Under Control of pG1-3 as Reporter

The strains were cultivated as described in Example 2a and secreted vHH titers were determined after 48 h of cultivation by mCE (Example 2c). Titer (mg vHH L⁻¹), WCW (g L⁻¹) and biomass specific product yield (mg vHH g⁻¹ WCW) were calculated for each clone and then averaged for all clones overexpressing one factor as well as for the replicates of the parental strain. The fold changes (FC) of titers and yields were determined in comparison to the mean of the parental host strain cultivated in the same 24-DWP.

TABLE 10
Effect of single overexpression of translation initiation factors on recombinant protein
production in strain CBS2612 P_{G1-3} vHH #4. FC vHH yield is the foldchange of the yield
of secreted vHH. The results shown were measured after 48 hours of screening cultivation.
OE shows the estimated increase in TIF transcript levels in the engineered strains,
compared to the parental strain

| Name | Promoter | Gene | Clone # | OE | GCN | FC vHH yield | Mean ± STDEV |
|---|---|---|---|---|---|---|---|
| eIF4E | pGAP | CDC33 | 1 | 36 | 2 | 1.22 | 1.18 ± 0.08 |
| | | | 2 | | 2 | 1.25 | |
| | | | 3 | | 2 | 1.06 | |
| eIF4A | pGAP | TIF2 a | 2 | 5 | 2 | 1.61 | 1.59 ± 0.11 |
| | | | 6 | | 2 | 1.45 | |
| | | | 7 | | 2 | 1.72 | |
| eIF4A | pGAP | TIF2 b | 16 | 5 | 2 | 1.27 | 1.31 ± 0.06 |
| | | | 17 | | 2 | 1.40 | |
| | | | 18 | | 2 | 1.26 | |
| eIF4G | pGAP | TIF4632 | 4 | 20 | 2 | 1.97 | 2.02 ± 0.21 |
| | | | 1 | | 2 | 1.79 | |
| | | | 3 | | 2 | 2.30 | |
| PAB1 | pGAP | PAB1 | 6 | 11 | 2 | 2.15 | 1.95 ± 0.18 |
| | | | 8 | | 2 | 1.71 | |
| | | | 7 | | 2 | 1.98 | |
| RLI1 | pGAP | RLI1 | 4 | 13 | 2 | 1.46 | 1.40 ± 0.13 |
| | | | 14 | | 2 | 1.53 | |
| | | | 16 | | 2 | 1.23 | |

Unexpectedly, even single overexpression of the chosen TIFs of the mRNP clearly increased recombinant protein production and secretion (Table 10). The highest improvement can be seen with TIF4632 (eIF4G) and PAB1 overexpression, which both increased recombinant protein secretion by approx. 2.0-fold.

### Example 5: Effect of Overexpressing Combinations of TIFs of the mRNP on Recombinant Protein Secretion

In order to analyse if there is an effect of overexpressing several translation initiation factors in complexes, different combinations of the translation factors tested in Example 4 were chosen and compared for their impact on recombinant protein production. This combinatorial engineering was done using the GoldenPiCS toolbox, as described in Example 1 b. The resulting plasmids were transformed into the host strain CBS2612 P_{G1-3} vHH #4. The engineered P. pastoris strains were then cultivated in small scale as described in Example 2. The protein secreted into the supernatant was measured as in Example 2c and the titers and yields of different engineered strains were compared to the parental host.

a) Generated Combinations for Translation Initiation Factor Overexpression.

Different combinational overexpressions, described in Table 11, were tested. The described promoters were chosen to overexpress each gene approximately 10-fold. This was done to balance the transcript concentration ratios of the different target genes in the cell. However, also stronger or weaker overexpression could be chosen which still leads to increases in recombinant protein production as can be seen in Table 10.

TABLE 11
Chosen overexpression combinations, shown also with selected promoters. OE shows the
estimated increase in TIF transcript levels in the engineered strains, compared to the
parental strain according to the gene expression data from Rebnegger et al. 2014. Biotech J.
9(4):511-25.

| Name | Gene 1 | OE | Gene 2 | OE | Gene 3 | OE | Gene 4 | OE | Gene 5 | OE |
|---|---|---|---|---|---|---|---|---|---|---|
| C1 | P_{MDH3} | 13 | P_{POR1} | 4 | | | | | | |
| | TIF4632 | | TIF2 b | | | | | | | |
| C2 | P_{MDH3} | | P_{POR1} | | P_{PDC1} | 12 | | | | |
| | TIF4632 | | TIF2 b | | CDC33 | | | | | |
| C3a | P_{MDH3} | | P_{POR1} | | P_{PDC1} | | P_{GPM1} | 8 | | |
| | TIF4632 | | TIF2 a | | CDC33 | | PAB1 | | | |
| C3b | P_{MDH3} | | P_{POR1} | | P_{PDC1} | | P_{GPM1} | | | |
| | TIF4632 | | TIF2 b | | CDC33 | | PAB1 | | | |
| C13 | P_{MDH3} | | P_{POR1} | | P_{PDC1} | | P_{GPM1} | | P_{FBA1-1} | 9 |
| | TIF4632 | | TIF2 b | | CDC33 | | PAB1 | | RLI1 | |

a) Effect of Combined Overexpressions on Recombinant Protein Secretion.

The engineered strains were screened and the data analysed as described in Example 2.

TABLE 12
Combined overexpression of translation initiation
factors in strain CBS2612 P_{G1-3} vHH #4. FC yield
is the foldchange of the yield of secreted vHH.

| Name | Clone # | GCN | FC vHH yield | Mean ± STDEV |
|---|---|---|---|---|
| C1 | 4 | 2 | 1.92 | 1.76 ± 0.11 |
| | 9 | 2 | 1.69 | |
| | 15 | 2 | 1.68 | |
| C2 | 4 | 2 | 2.27 | 2.27 ± 0.04 |
| | 18 | 2 | 2.33 | |
| | 31 | 2 | 2.23 | |
| C3a | 2 | 2 | 2.25 | 2.23 ± 0.02 |
| | 4 | 2 | 2.21 | |
| | 5 | 2 | 2.24 | |
| C3b | 1 | 2 | 2.56 | 2.52 ± 0.03 |
| | 13 | 2 | 2.50 | |
| | 16 | 2 | 2.49 | |
| C13 | 8 | 2 | 2.60 | 2.42 ± 0.16 |
| | 19 | 2 | 2.43 | |
| | 21 | 2 | 2.21 | |

Table 12 shows the effect of overexpressing different TIF combinations on recombinant protein production. All clones shown in Table 12 were verified to have the overexpression cassette only inserted once, meaning they showed a GCN of 2. The fold change of the vHH yield is shown in comparison to the host strain CBS2612 P_{G1-3} vHH #4. While all combinations showed increased recombinant vHH secretion compared to the parent, the combinations C3 and C13 clearly show the biggest effect. C3a and C3b contain different versions of TIF2, which differ in length according to different annotations in the P. pastoris genome sequences. Independent of the TIF2 version, both of the combinations increased the vHH yield by more than 2-fold. C3b increased vHH yield by 2.5-fold.

b) Comparison of C3b Overexpression on Recombinant Protein Secretion in Different Background Strains.

To compare effects of different background strains the P_{G1_3}_vHH expression cassette was transformed into CBS7435 as described in Example 1a. Then the construct C3b was integrated into the genome of the resulting CBS7435 P_{G1-3}vHH production host strain, as described in Example 1c. The effect of C3b overexpression was then screened as described in Example 2a and the secreted vHH titer was determined as described in Example 2c. For comparison, 9 different clones of CBS7435 P_{G1-3} vHH C3b were screened and compared to a biological quadruplicate of the host strain CBS7435 P_{G1-3} vHH.

TABLE 13
Screening result of background strain comparison. The
fold change of the vHH yield is shown for the overexpression
construct, in comparison to the host strain.

| | Mean FC vHH yield | Clones screened |
|---|---|---|
| CBS7435 P_{G1-3} vHH C3b | 2.11 ± 0.28 | 9 |

Table 13 shows the fold change of the vHH yield of the C3b overexpression in the host strain CBS7435 P_{G1-3} vHH. These results show that independent of the choice of background strain, a strong beneficial effect on recombinant vHH secretion can be seen in all strains with C3b overexpression.

c) Effect of TIF Overexpression on Methanol Inducible Recombinant Protein Secretion.

To determine the effect of C3b overexpression on methanol inducible recombinant protein secretion, CBS7435 MutS containing the pAOX1-vHH expression cassette (Zavec et al. 2020, Biotechnol Bioeng. 117(5):1394-1405) was used as the host strain for C3b overexpression. The strains were screened as described in Example 2b using methanol shots for P_{AOX1} induction and the protein titers were determined as described in Example 2c.

The screening with ten C3b overexpression clones showed an average increase of vHH yield by 1.39±0.05-fold in comparison to the parental strain. This confirms that increases in recombinant protein secretion can be achieved with TIF overexpression, regardless of the applied carbon source or promoter system.

### Example 6: Characterization of the Impact of Translation Initiation Factor Overexpression on Cellular Processes

To assess which cellular processes were impacted upon translation initiation factor overexpression, additionally to the observed differences in recombinant protein secretion, two different approaches were followed: On the one hand, gene transcript levels were measured to determine a potential impact on transcript abundance (Example 6a). On the other hand, cellular translation activity was directly measured after setting up a puromycin based method (Example 6b) in P. pastoris.

a) Spike-In Method for Comparative Measurement of Transcript Levels.

First, the strains were cultivated in the 24-DWP screening procedure as described in Example 2 for 30 h. This corresponds to a growth rate of approximately 0.025 h⁻¹ at the point of harvest. 1 mL of culture was harvested and centrifuged for 5 minutes at 16,000 g at 4° C. The supernatant was discarded and the pellets stored at −80° C. until further use.

To be able to measure also potential changes of transcript concentration for common housekeeping genes the pellets were dissolved in PBS and pelleted again according to the WCW, to have the same amount of yeast mass in each sample. Then each of the pellets was spiked with 1 mL of S. cerevisiae S288c suspension (aliquots from a single shake flask culture). The resulting mixed P. pastoris-S. cerevisiae pellet was used for RNA isolation.

For RNA isolation 1 mL of TRI Reagent (Sigma-Aldrich) and 500 μL acid washed glass beads were added to the cells which were then disrupted in a FastPrep-24 (mpbio) at speed 5.5 m/s for 40 seconds. Afterwards, 200 μL of chloroform were added. Subsequently, samples were shaken vigorously and then allowed to stand for 5-10 min at room temperature. After centrifugation for 10 min at 16,000 g and 4° C. to promote phase separation, the upper colourless aqueous phase containing the RNA was transferred into a fresh tube and 500 μL of isopropanol were added to precipitate the RNA. After 10 minutes of incubation samples were centrifuged for 10 min at 16,000 g and 4° C. and the supernatant was discarded. The RNA pellet was washed once with 70% ethanol, air-dried and re-suspended in RNAse free water.

To remove residual DNA, the RNA samples were treated with the DNA-Free™-kit (Ambion) according to the manufacturer's manual. Subsequently, RNA quality, purity and concentration were analysed by gel electrophoresis as well as spectrophotometric analysis using a NanoDrop 2000 (Thermo Scientific).

Synthesis of cDNA was done with the Biozym cDNA Synthesis Kit according to the manufacturer's manual. Briefly, 1 μg of total RNA were added to the master mix containing reverse transcriptase, dNTPs, RNase inhibitor and synthesis buffer. As the priming oligo d(T)23 VN (NEB) was used. Incubation of the reaction mix was done for 45 min at 55° C. Subsequently, inactivation of the enzymes was achieved by incubation of the reaction mix at 99° C. for 5 min.

For quantitative real-time PCR (qPCR) P. pastorisACT1, TDH3 and vHH specific primers were used (see Table 14). Normalization was done by comparing to S. cerevisiae ACT1 expression levels (see Table 14). Transcript levels of the engineered strains were compared to the host strain transcript levels. Both sets of ACT1 primers were tested and verified to only bind to the cDNA of the desired organism. For qPCR 1 μL of cDNA, water and primers were mixed with Blue S′Green qPCR master mix (Biozym) and analysed in a real-time PCR cycler (Rotor-Gene, Qiagen). All samples were measured in technical triplicates. Data analysis was performed with the Rotor-Gene software employing the Comparative Quantitation (QC) method.

TABLE 14
Quantitative real-time PCR primers for transcript analysis

| | | | Product | Product |
|---|---|---|---|---|
| Primer name | Target | Sequence | length | name |
| qPCR_ScACT1_fwd | S. | AGCGGTGATTTCCTTTTGCATTCTTTCG, | 160 bp | ScACT1 |
| | cerevisiae | SEQ ID NO: 189 | | |
| qPCR_ScACT1_rev | ACT1 | TTTGGGTTTGGAATCTGCCGGTA, | | |
| | | SEQ ID NO: 190 | | |
| qPCR_PpACT1_fwd | P. | CCTGAGGCTTTGTTCCACCCATCT, | 148 bp | PpACT1 |
| | pastoris | SEQ ID NO: 191 | | |
| qPCR_PpACT1_rev | ACT1 | GGAACATAGTAGTACCACCGGACATAACGA, | | |
| | | SEQ ID NO: 192 | | |
| qPCR_GAPDH_fwd | P. | CGAGAGATCCTCCATCTTCGACGC, | 124 bp | TDH3 |
| | pastoris | SEQ ID NO: 193 | | |
| qPCR_GAPDH_rev | TDH3 | GTGTTGCAACAAGTCGACGACTCTG, | | |
| | | SEQ ID NO: 194 | | |
| qPCR_vHH_fwd | vHH | TGTAACGTGAATGTCGGATTTG, | 86 bp | vHH |
| | | SEQ ID NO: 195 | | |
| qPCR_vHH_rev | | TAGTGATGGTGGTGGTGATG, | | |
| | | SEQ ID NO: 196 | | |

b) Impact of TIF Overexpression on Transcript Abundance

The TIF overexpression strains shown in Table 15 and the host strain, CBS2612 P_{G1-3} vHH #4, in triplicate, were cultivated in the 24-DWP screening procedure as described in Example 2a for 30 h. Transcript abundance of two endogenous genes and the recombinant GOI were determined as described in Example 6a.

Table 15 shows the obtained results of the transcript level measurements. The measurements show, that the transcript levels of vHH are strongly affected by the TIF overexpressions. Especially high values can be seen for the overexpression combinations that also already showed higher recombinant protein secretion in Example 4 and 5. The highest transcript levels were found in the strains overexpressing C3b. This overexpression increased vHH transcript levels by 5.5-fold. Surprisingly, expression of TDH3 appears to be also to be increased in all of the overexpression strains, while expression of ACT1 appears to be increased especially in the combined overexpression strains. The increase of transcript level for both housekeeping genes, ACT1 and TDH3, indicates an increase of all transcripts in the cell. These results indicate that TIF overexpression has a positive and/or a stabilizing effect on cellular mRNA levels, which could be one factor leading to increased productivity.

TABLE 15
Relative transcript levels of the different overexpression strains compared to the host strain. The
measurement was taken after 30 h of the screening cultivation described in Example 2a.

| | Clone | | PpACT | | vHH | | TDH3 |
|---|---|---|---|---|---|---|---|
| | # | PpACT1 | 1 mean | vHH | mean | TDH3 | mean |
| Single | | | | | | | |
| over- | | | | | | | |
| expressions | | | | | | | |
| CDC33 | 1 | 0.84 ± 0.02 | 0.87 ± 0.04 | 1.03 ± 0.03 | 1.05 ± 0.04 | 2.54 ± 0.00 | 1.92 ± 0.44 |
| | 2 | 0.91 ± 0.03 | | 1.08 ± 0.03 | | 1.61 ± 0.05 | |
| TIF2a | 1 | 0.95 ± 0.03 | 0.93 ± 0.03 | 1.15 ± 0.00 | 1.18 ± 0.05 | 2.55 ± 0.07 | 2.57 ± 0.18 |
| | 6 | 0.91 ± 0.00 | | 1.20 ± 0.07 | | 2.59 ± 0.14 | |
| TIF2b | 16 | 0.77 ± 0.02 | 0.82 ± 0.05 | 1.01 ± 0.07 | 1.02 ± 0.06 | 2.54 ± 0.19 | 2.51 ± 0.16 |
| | 17 | 0.86 ± 0.02 | | 1.03 ± 0.03 | | 2.47 ± 0.11 | |
| TIF4632 | 4 | 1.34 ± 0.00 | 1.28 ± 0.08 | 3.21 ± 0.09 | 3.15 ± 0.17 | 2.68 ± 0.15 | 2.49 ± 0.26 |
| | 1 | 1.21 ± 0.06 | | 3.10 ± 0.22 | | 2.37 ± 0.24 | |
| PAB1 | 6 | 1.61 ± 0.04 | | 3.53 ± 0.19 | | 2.70 ± 0.26 | |
| RLI1 | 4 | 0.94 ± 0.03 | 0.93 ± 0.04 | 0.99 ± 0.05 | 0.93 ± 0.08 | 3.29 ± 0.53 | 3.14 ± 0.56 |
| | 14 | 0.91 ± 0.04 | | 0.87 ± 0.04 | | 2.54 ± 0.00 | |
| Combined | | | | | | | |
| over- | | | | | | | |
| expressions | | | | | | | |
| C1 | 4 | 1.29 ± 0.04 | 1.25 ± 0.05 | 2.25 ± 0.11 | 2.35 ± 0.14 | 1.68 ± 0.05 | 1.62 ± 0.31 |
| | 9 | 1.22 ± 0.03 | | 2.44 ± 0.11 | | 1.58 ± 0.39 | |
| C2 | 4 | 1.29 ± 0.03 | 1.30 ± 0.04 | 3.74 ± 0.10 | 3.72 ± 0.10 | 1.49 ± 0.22 | 1.54 ± 0.18 |
| | 18 | 1.31 ± 0.04 | | 3.70 ± 0.10 | | 1.62 ± 0.05 | |
| C3a | 2 | 1.91 ± 0.10 | 1.89 ± 0.10 | 5.42 ± 0.14 | 5.37 ± 0.23 | 2.43 ± 0.12 | 2.28 ± 0.25 |
| | 4 | 1.87 ± 0.09 | | 5.32 ± 0.28 | | 2.06 ± 0.24 | |
| C3b | 1 | 1.80 ± 0.05 | 1.67 ± 0.13 | 4.92 ± 0.00 | 5.49 ± 0.58 | 2.48 ± 0.25 | 2.30 ± 0.26 |
| | 13 | 1.55 ± 0.00 | | 6.07 ± 0.16 | | 2.12 ± 0.23 | |
| C13 | 8 | 1.96 ± 0.11 | 2.00 ± 0.09 | 5.53 ± 0.43 | 4.82 ± 0.78 | 2.27 ± 0.23 | 2.09 ± 0.28 |
| | 19 | 2.03 ± 0.05 | | 4.12 ± 0.19 | | 1.91 ± 0.20 | |

c) Measurement of Overall Translation Activity.

The measurement of overall translation activity with 0-propargyl labelled puromycin was done similarly to Nagelreiter et al. 2018. Biotechnol J 13, e1700492 after optimizing the procedure for use in yeast cells. Briefly, cells from the same cultivation as in Example 6a were pipetted into a 96-well microtiter plate with an end-OD₆₀₀ of 0.4 in 90 μL “Incubation Solution”. The “Incubation Solution” consisted of ASMv6 media (see Example 2) with 0.6 mM 0-propargyl puromycin (Jena Bioscience, NU-931-05), dissolved in 10% DMSO and PBS (2 mM KH₂PO₄, 10 mM Na₂HPO₄·2 H₂O, 2.7 mM g KCl, 8 mM NaCl, pH 7.4), and 1.5 g L⁻¹ Imipramine. The suspension was incubated for 2 h at 25° C. on a shaker, transferred into ice-cold Eppendorf tubes and centrifuged at 16,000 g for 5 min at 4° C. After washing the pelleted cells with 120 μL PBS, the again pelleted cells were fixed with 1 mL of ice-cold 70% ethanol. These fixed samples were stored between 1 day and 2 weeks at 4° C.

For the click chemistry reaction, the fixed samples were harvested by centrifugation at 16,000 g and 4° C. for 5 min. The pellet was transferred to a 96-well microtiter plate and washed with 100 μL “Click Chemistry Buffer” (115 mM Tris/HCl pH=8.5, 0.1% Triton X-100). Then the samples were incubated in “Click Chemistry Mix” (101 mM Click-it Click Chemistry Buffer, 1.9 mM CuSO4, 1.9 mg/mL ascorbic acid, 20 μM Alexa Fluor™ 488 azide (Invitrogen)) for 30 min at RT. Afterwards, the cells were harvested as before, washed in 150 μL PBS and dissolved in 150 μL fresh PBS. To measure the resulting fluorescence intensity, the cells were analysed by flow cytometry with an excitation wavelength of 488 nm and an emission wavelength of 525 nm. 40,000 events were measured for each sample. For data analysis, the geometric mean of each sample was used and a blank (cells treated without O-propargyl puromycin addition) was subtracted of each.

d) Impact of TIF Overexpression on Global Cellular Translation

Table 16 shows the fold change of the obtained fluorescence values, therefore the mean fold change of overall translation activity, in comparison to the host strain CBS2612 P_{G1-3} vHH #4. For measurement of the translation activity the same clones as shown in Table 15 were used. Translation activity was determined as described in Example 6c.

TABLE 16
Translation activity of TIF overexpression strains
relative to the host strain CBS2612 P_{G1-3} vHH#4 (set
to 1.0). The measurement was taken after 30 h of
the screening cultivation described in Example 2a.

| Single overexpressions | Relative translation activity per cell |
|---|---|
| CDC33 | 0.97 ± 0.005 |
| TIF2a | 1.24 ± 0.01 |
| TIF2b | 1.03 ± 0.01 |
| TIF4632 | 1.68 ± 0.14 |
| PAB1 | 1.65 |
| RLI1 | 1.39 ± 0.09 |
| Combined overexpressions | Relative translation activity per cell |
| C1 | 1.58 ± 0.17 |
| C2 | 1.98 ± 0.10 |
| C3a | 2.21 ± 0.06 |
| C3b | 2.29 ± 0.06 |
| C13 | 1.73 ± 0.03 |

Table 16 clearly shows that overexpression of single translation initiation factors led to enhanced translation activity in each cell. Overexpression of combinations of the chosen TIFs shows an even stronger increase in translational activity. This is also reflected by the increased recombinant protein secretion observed in these strains (Examples 4 and 5). The highest translation activity, 2.3-fold higher than in the host strain, could be achieved by overexpressing C3b. These results show, that the overexpression of selected translation initiation factors, or combinations thereof, increases overall cellular translation activity, not only translation of specific proteins such as the recombinant protein. Surprisingly, there is a clear correlation between the improvement of vHH yield (Tables 10 and 12) and the relative translational activity (correlation coefficient R2=0.84), indicating that the formation of the mRNP during translation initiation is a rate-limiting step for recombinant protein production.

### Example 7: Effect of Translation Factor Overexpression in Fed-Batch Cultivations

To further validate the observations made in the screenings, fed-batch cultivations similar to standard production processes were done with selected overexpression targets.

a) Effect of TIF Overexpression on vHH Production in Fed Batch Cultivations:

For this example, CBS2612_P_{G1-3}_vHH #4 overexpressing either C3b or RLI1 were chosen for cultivation. These strains showed a strong beneficial effect on recombinant protein secretion in screenings (Examples 4, 5 and 9). For the fed batch cultivations, different feeding profiles, while using the same media, were applied which resulted in the following calculated growth rates at the respective sampling points (Table 17). The media composition can be found below.

TABLE 17
Growth rates at the different sampling points in the fed-batches.

| Reactor Run# | Sample | μ [h⁻¹] |
|---|---|---|
| B289-B293 | F3 | 0.071 |
| | F20 | 0.056 |
| | F46 | 0.037 |
| B365-B368 | F1 | 0.010 |
| | F4 | 0.011 |
| | FEnd | 0.011 |
| C037-C040 | F3 | 0.065 |
| | F46 | 0.044 |
| C041-C043 | F3 | 0.100 |
| | F31 | 0.020 |
| | F54 | 0.016 |

Media:

PTM₀ trace salt stock solution per liter:

5.0 mL H₂SO₄ (95-98%), 65.0 g FeSO₄*7H₂O, 20 g ZnCl₂, 6.00 g CuSO₄*5H₂O, 3.0 g MnSO₄*H₂O, 0.5 g CoCl₂*6H₂O, 0.20 g Na₂MoO₄*2H₂O, 0.08 g NaI, 0.02 g H₃BO₃

Glycerol Batch medium contained per liter:

2 g Citric acid monohydrate (C₆H₈O₇*H₂O), 45 g Glycerol, 12.6 g (NH₄)₂HPO₄, 0.5 g MgSO₄*7H₂O, 0.9 g KCl, 0.022 g CaCl₂*2H₂O, 13.2 mL Biotin stock solution (0.1 g L⁻¹) and 4.6 mL PTM0 trace salts stock solution. HCl (conc.) was added to set the pH to 5.

Glucose feed media contained per liter:

495 g glucose monohydrate, 4.6 g MgSO_{4*7}H₂O, 8.4 g KCl, 0.28 g CaCl₂*2H₂O, 23.6 mL biotin stock solution (0.1 g L⁻¹) and 10.1 mL PTM0 trace salts stock solution.

b) Fed Batch Cultivations with Linear Feed with Minimum Growth Rates Reaching 0.04 h⁻¹.

Fed-batch cultivations were done with the host strain CBS2612 PG1-3vHH #4 and the corresponding overexpression strains CBS2612 P_{G1_3} vHH C3b #13, CBS2612 P_{G1-3} vHH C3b #16 and CBS2612 P_{G1-3} vHH P_{GAP} RLI1 #4 in 1 L benchtop bioreactors (SR07000DLS, Dasgip, Germany; reactor runs #A-B) or 1.8 L benchtop bioreactors (SR15000DLS, Dasgip, Germany; reactor runs #C). For pre-cultures 100 mL YPG media containing 50 μg mL⁻¹ Zeocin and 100 μg mL⁻¹ nourseothricin (if appropriate) in a 1 L shake flask were inoculated with a 1.0 mL cryostock and incubated for around 24 h at 180 rpm and 25° C. Batch cultures were operated at a working volume of 0.5 L and were inoculated to a starting OD₆₀₀ of 1.5. Glycerol batch media composition is given above. During the entire process the temperature was controlled at 30° C., the DO was kept at 30% by automated adjustment of stirrer speed (between 400 and 1200 rpm) and air flow (between 9.5 and 30 sL h⁻¹), and the pH was regulated to be at 5.0 by automated addition of 12.5% NH₄OH. After a sudden spike in DO, indicating batch-end (BE), a linear incremental glucose feed (media composition detailed above) resulting in fast initial growth rates (μ) followed by an extended phase of gradually decreasing p was applied. The linear increase of the feed was set to follow the equation: F[mL h⁻¹]=0.1431*t+2.0499. The same fed-batch cultivations were done twice to confirm the obtained results.

Yeast dry mass (YDM) and secreted recombinant proteins were analysed at various time points throughout the process (shown in Table 18). For YDM analysis 1 mL of culture broth was transferred to a 2 mL pre-dried (at 105° C. for at least 24 h) and pre-weighted centrifugation tube. After centrifugation at 16,000 g and 4° C. for 5 min the supernatant was carefully transferred to a fresh vial and stored at −20° C. until further use. Cell pellets were washed twice with 0.1 M HCl and dried at 105° C. for at least 24 h before the weight was measured again.

Supernatants were analyzed by microfluidic capillary electrophoresis (GXII, Perkin-Elmer) as described in Example 2c.

TABLE 18
YDM of the two fed-batch cultivation runs, B289-B292 and
C037-C040 and the FC of titer and yield can be seen here. Samples
were taken at 2 or 3 different timepoints. FC vHH titer/yield is the
fold change of the overexpression construct titer/yield compared to the
host strain titer/yield, at the same timepoint.

| | | | time after | | FC | FC |
|---|---|---|---|---|---|---|
| | Reactor | | feed start | YDM | vHH | vHH |
| | # | Sample | [h] | [g L⁻¹] | titer | yield |
| P_{G1-3} vHH | B289 | F3 | 3.2 | 30.2 ± 0.2 | — | — |
| | C037 | F3 | 3.1 | 30.4 ± 0.1 | — | — |
| | B289 | F20 | 20.0 | 78.2 ± 0.3 | — | — |
| | B289 | F46 | 46.0 | 140.6 ± 0.7 | — | — |
| | C037 | F46 | 45.8 | 148.7 ± 0.8 | — | — |
| P_{G1-3} vHH | B290 | F3 | 3.2 | 30.0 ± 0.1 | 1.2 | 1.2 |
| P_{GAP} RLI1 | C038 | F3 | 3.1 | 29.2 ± 0.5 | 1.2 | 1.3 |
| #4 | B290 | F20 | 20.0 | 77.7 ± 0.2 | 2.5 | 2.5 |
| | B290 | F46 | 46.0 | 143.2 ± 0.5 | 2.1 | 2.1 |
| | C038 | F46 | 45.8 | 137.8 ± 1.0 | 2.0 | 2.2 |
| P_{G1-3} vHH | C039 | F3 | 3.1 | 29.9 ± 0.2 | 1.3 | 1.4 |
| C3b #13 | B291 | F20 | 20.0 | 79.4 ± 0.3 | 2.4 | 2.3 |
| | B291 | F46 | 46.0 | 140.2 ± 0.2 | 2.9 | 2.9 |
| | C039 | F46 | 45.8 | 144.3 ± 0.2 | 2.4 | 2.5 |
| P_{G1-3} vHH | B292 | F3 | 3.2 | 31.7 ± 0.1 | 1.5 | 1.4 |
| C3b #16 | C040 | F3 | 3.1 | 31.0 ± 0.4 | 2.0 | 1.9 |
| | B292 | F20 | 20.0 | 80.2 ± 0.3 | 2.9 | 2.9 |
| | B292 | F46 | 46.0 | 142.2 ± 0.0 | 2.7 | 2.6 |
| | C040 | F46 | 45.8 | 144.4 ± 0.3 | 2.5 | 2.6 |

In Table 18 can be seen that overexpression of the TIFs had no impact on biomass concentration in fed batch cultivations. In contrast, product titers and yields were increased compared to the parental control during the whole fed batch course. Especially at the later time points F20 and F46, the clear positive effect of RLI1 overexpression on product titers and yields can be seen, exceeding the parental host strain by 2.2-fold at the end of the fermentation. Overexpression of C3b led to an even higher increase, of 2.7-fold higher product yields and titers on average.

c) Cultivation at Constant Feed with a Minimum Growth Rate of 0.02 h⁻¹.

Another fed-batch cultivation was done with host strain CBS2612 P_{G1-3} vHH #4 and the corresponding overexpression strains CBS2612 P_{G1-3} vHH C3 #16 and CBS2612 PG1-3vHH P_{GAP} RLI1 #4 as described in Example 7a and 7b. In this cultivation, however, a different feed profile was chosen, which applied a constant glucose feed instead of the linear incremental glucose feed described in Example 7b. The constant feed was held at 4 mL h⁻¹ during the whole fed-batch cultivation. This resulted in a faster decrease of growth rates in the beginning and a longer cultivation at slow growth. Sampling was done as described above. Additionally to YDM and supernatant, 1 mL of cell suspension was collected, pelleted and frozen at −80° C. for transcript level determination.

TABLE 19
YDM of the fed-batch cultivation run, C041-C044, and the FC of titer
and yield can be seen here. Samples were taken 3 different timepoints.
FC titer/yield is the fold change of the overexpression construct
titer/yield compared to the host strain titer/yield, at the same timepoint.

| | | | time after | | FC | FC |
|---|---|---|---|---|---|---|
| | Reactor | | feed start | YDM | vHH | vHH |
| | # | Sample | [h] | [g L⁻¹] | titer | yield |
| P_{G1-3} vHH | C041 | F3 | 3.1 | 35.0 ± 0.4 | — | — |
| | | F31 | 30.6 | 97.6 ± 0.4 | — | — |
| | | F54 | 54.0 | 120.8 ± 0.5 | — | — |
| P_{G1-3} vHH | C042 | F3 | 3.1 | 34.3 ± 0.2 | 1.2 | 1.2 |
| P_{GAP} RLI1 | | F31 | 30.6 | 97.0 ± 0.4 | 2.9 | 2.8 |
| #4 | | F54 | 54.0 | 121.5 ± 0.6 | 2.2 | 2.2 |
| P_{G1-3} vHH | C044 | F3 | 3.1 | 36.9 ± 0.2 | 0.7 | 0.6 |
| C3b #16 | | F31 | 30.6 | 94.7 ± 0.4 | 5.5 | 5.6 |
| | | F54 | 54.0 | 117.2 ± 0.2 | 2.9 | 3.0 |

Table 19 shows that also with this feeding strategy, both RLI1 and C3b overexpression resulted in increased product titers and yields in comparison to the host strain while producing the same amount of YDM. As in Example 7b, C3b overexpression proves to be highly beneficial for recombinant protein production, reaching 4-fold higher product yields and titers on average.

Together, this indicates that overexpression of single TIFs or combinations thereof have a strong positive effect on recombinant protein production independent of the applied feeding strategy.

d) Effect of Translation Factor Overexpression on Transcript Level in Fed Batch.

In order to assess if the effect of TIF overexpression on transcript abundance seen in Example 6b was also persistent when cultivating the cells in fed batch, the transcript levels of PpACT1, vHH and TDH3 were analysed in samples from the fed-batch runs C041-0044 (Example 7b). The procedure was done as described in Example 6a. As described above, the transcript levels were normalised to S. cerevisiae ACT1. Additionally, they were then normalised to the host strain, reactor C041, at the corresponding sampling point. Table 20 shows the fold change of the relative transcript levels of PpACT1, vHH and TDH3.

TABLE 20
Relative transcript levels of the two P. pastoris housekeeping genes
ACT1 and TDH3, and of the secreted recombinant protein, vHH.

| | P_{G1-3} vHH P_{GAP} RLI1 #4 | | | P_{G1-3} vHH C3b #16 | | |
|---|---|---|---|---|---|---|
| | Reactor # C042 | | | Reactor # C044 | | |
| Sample | PpACT1 | vHH | TDH3 | PpACT1 | vHH | TDH3 |
| F3 | 1.04 ± | 0.96 ± | 1.13 ± | 1.11 ± | 1.89 ± | 1.82 ± |
| | 0.03 | 0.04 | 0.09 | 0.05 | 0.11 | 0.23 |
| F31 | 1.22 ± | 1.16 ± | 1.36 ± | 1.47 ± | 2.85 ± | 2.22 ± |
| | 0.03 | 0.11 | 0.10 | 0.11 | 0.46 | 0.13 |
| F54 | 1.23 ± | 0.89 ± | 1.02 ± | 1.47 ± | 2.44 ± | 1.63 ± |
| | 0.00 | 0.15 | 0.03 | 0.00 | 0.17 | 0.16 |

While overexpression of RLI1 led to a small increase of transcript levels of the three analysed genes (on average 1.2 for the two native P. pastoris genes at the later timepoints F31 and F54), C3b overexpression led to an increase of up to 1.5 for PpACT1, up to 2.9-fold for vHH and up to 2.2-fold for TDH3 (Table 20). The vHH transcript level increase in C3b appears to correlate to the increase in titer seen in the same fed-batch cultivation (Table 19). The increase of transcript level for both housekeeping genes, ACT1 and TDH3, indicates an increase of all transcripts in the cell, independent of the mode of cultivation.

e) Fed-Batch Cultivation with Methanol Inducible Recombinant Protein Secretion.

Fed batch cultivation of clones requiring methanol induction was performed according to standard processes and media for MutS strains as described in Zavec et al. 2020. For pre-cultures, 100 mL YPG media containing 50 μg mL⁻¹ Zeocin and 100 pg mL⁻¹ nourseothricin (if appropriate) in a 1 L shake flask was inoculated with a 1.0 mL cryostock and incubated for around 24 h at 180 rpm and 25° C.

Batch cultures were operated at a working volume of 0.4 L BSM medium (Mellitzer et al., 2014) and were inoculated to a starting OD₆₀₀ of 2.5. The temperature was controlled and kept at 25° C., the DO was kept at 20% by automated adjustment of stirrer speed (between 200 and 1250 rpm),air flow (between 9.5 and 50 sL h⁻¹), and oxygen supplementation. The pH was regulated to be at 5.0 by automated addition of 25% NH₄OH. After a sudden spike in DO, indicating batch-end (BE), glycerol feeding followed by glycerol/methanol co-feeding was initiated. The glycerol feed (60% w/w+12 mL/L PTM1) with a linearly increasing (y=0.225x+1.95) glycerol feed lasted for 8 hours. This was followed by an 18 h co-feed of 60% glycerol and 100% methanol. In the co-feed, the 60% glycerol feed was linearly decreasing (y=3.75-0.111x) and the methanol feed was linearly increasing (y=0.028x+0.6). Finally, in the methanol only feed phase a linearly increasing methanol feed (y=0.028x+1.10) was applied for 72h. Sampling, YDM determination and protein quantification were performed as described in Example 7b.

TABLE 21
YDM of the methanol-based fed-batch cultivation runs B365-B368, and
the FC of titer and yield. Samples were taken 3 different timepoints.
The time after feed start corresponds to the time after the pure glycerol
feed start. FC titer/yield is the fold change of the overexpression
construct titer/yield compared to the average of the two host strain
titers/yields, at the same timepoint.

| | | | time after | | FC | FC |
|---|---|---|---|---|---|---|
| | Reactor | | feed start | YDM | vHH | vHH |
| | # | Sample | [h] | [g L⁻¹] | titer | yield |
| MutS P_{AOX} | B365 | F1 | 32.7 | 100.4 ± 1.4 | — | — |
| vHH | B365 | F4 | 72.3 | 123.0 ± 0.2 | — | — |
| | B365 | FEnd | 120.3 | 137.8 ± 0.2 | — | — |
| MutS P_{AOX} | B366 | F1 | 32.7 | 101.8 ± 0.9 | — | — |
| vHH | B366 | F4 | 72.3 | 128.0 ± 0.8 | — | — |
| | B366 | FEnd | 120.3 | 145.0 ± 0.4 | — | — |
| MutS P_{AOX} | B367 | F1 | 32.7 | 91.7 ± 0.6 | 1.6 | 1.7 |
| vHH C3b | B367 | F4 | 72.3 | 110.5 ± 0.9 | 1.7 | 1.7 |
| | B367 | FEnd | 120.3 | 123.5 ± 0.5 | 1.7 | 1.8 |
| MutS P_{AOX} | B368 | F1 | 32.7 | 90.9 ± 0.1 | 1.7 | 1.8 |
| vHH C3b | B368 | F4 | 72.3 | 111.7 ± 0.6 | 1.7 | 1.8 |
| | B368 | FEnd | 120.3 | 125.0 ± 0.1 | 1.7 | 1.8 |

Table 21 shows that also when using a methanol-based recombinant protein production strategy, C3b overexpression resulted in increased product titers and yields in comparison to the host strain and therefore shows a clear beneficial effect. The increases are around 1.7-fold, starting shortly after the initiation of the pure methanol feed and continuing until the end of cultivation.

### Example 8: Effect of TIF Overexpression in Chemostat Cultivations

As continuous cultivation is getting more attention in the field of biopharmaceutical production, the effect of C3b overexpression on recombinant protein secretion was also analysed at a fixed growth rate in chemostat cultivations. This method offers the possibility of continuous cultivation during a production process and allows tight control of the growth rate.

a) Effect of C3b Overexpression on Recombinant Protein Secretion at a Fixed Growth Rate in Chemostat.

Media:

Trace element solution for chemostat per liter:

g EDTA, 4.5 g ZnSO₄*7H₂O, 1.03 g MnCl₂*4H₂O, 0.3 CoClo₂*6H₂O, 0.3 g CuSO₄, 0.4 g Na₂MoO₄*2H₂O, 4.5 g CaCl₂*2H₂O, 3 g FeSO₄*7H₂O, 1 g H₃BO₃, 0.1 KI

EDTA and ZnSO₄*7H₂O were dissolved in H₂O, the pH set to 6 with solid NaOH and then the other salts dissolved one by one. Then the pH was set to 4 with solid NaOH and conc. HCl.

Glucose media for chemostat per liter (to achieve a YDM of 10 g L⁻¹): 22 g glucose monohydrate, 10 g (NH₄)₂SO₄, 6 g KH₂PO₄, 1 MgSO₄*7H₂O, 0.5 g Pluronic® PE 6100, 3 mL trace element solution for chemostat, 1.6 ml biotin stock solution (0.1 g L⁻¹)

The pH was set to 5 by addition of solid KOH.

For the chemostat the strain CBS2612 P_{G1-3} vHH #4 and the corresponding C3b overexpression strain, CBS2612 P_{G1-3} vHH C3b #13, were cultivated in duplicate in 1.8 L benchtop bioreactors (SR15000DLS, Dasgip, Germany). For pre-cultures 100 mL YPG media containing 50 μg mL⁻¹Zeocin and 100 μg mL⁻¹ nourseothricin (if appropriate) in a 1 L shake flask were inoculated with a 1.0 mL cryostock and incubated for ca. 24 h at 180 rpm and 25° C. The batch cultures were operated at a working volume of 0.6 L and were inoculated to a starting OD₆₀₀ of 0.4. The media described above was used for batch and continuous cultivation. For the batch cultivation the DO was kept at 30% by automated adjustment of stirrer speed (between 400 and 1200 rpm) and air flow (between 9.5 and 30 sL h⁻¹). For the continuous cultivation, the stirrer speed was set to 700 rpm and the airflow to 30 sL h⁻¹. During the whole process, the temperature was kept at 30° C. and the pH at 5 by automated addition of 12.5% NH₄OH. The media was designed so that the YDM reached a concentration of approximately 10 g/L in batch and continuous cultivation. After a sudden spike in DO, indicating batch-end, the continuous culture was started. The chosen feed rate was 9 mL h⁻¹ for μ=D=0.015 h⁻¹ and the culture volume was kept constant at 0.6 L. This was done by using a level sensor and automatic pumping of additional culture out of the reactor, whenever the volume exceeded 0.6 L. The samples were taken after 333 h, corresponding to 5 volume changes of reactor volume, which was accepted as steady-state condition.

Yeast dry mass (YDM) and secreted recombinant protein were analysed at the chosen sampling point as described in Example 7b. Also, samples for transcript level analysis were taken as described in Example 6a. Additionally, cell pellets, made by centrifugation of 1 mL culture at 16,000 g for 5 min, discarding the supernatant and storing the pellets at −20° C., were collected. These were used for the total protein measurement described in Example 8c.

Table 22 shows the results of above described chemostat. Even at this fixed and slow growth rate and in steady-state conditions, titers were up to 1.75-fold higher upon TIF overexpression while the biomass concentration (YDM) was similar to the control host strain. Specific productivity was increased by 1.5 to 1.8-fold with the C3b overexpression strain compared to the host strain. This verifies that the positive effect of TIF overexpression on recombinant protein production seen in fed batch cultures (Example 7) can be also achieved in continuous cultivation.

TABLE 22
vHH Titer, YDM and specific productivity of vHH for the host
strain CBS2612 P_{G1-3} vHH #4 and the C3b overexpression strain.

| | Reactor | μ | Titer vHH | YDM | specific productivity |
|---|---|---|---|---|---|
| | # | [h⁻¹] | [mg L⁻¹] | [g L⁻¹] | of vHH [mg g⁻¹ h⁻¹] |
| P_{G1-3} | C049 | 0.015 | 97.1 ± 2.6 | 12.1 ± 0.1 | 0.12 |
| vHH | C050 | 0.015 | 98.5 ± 6.9 | 12.1 ± 0.2 | 0.12 |
| P_{G1-3} | C051 | 0.015 | 171.0 ± 1.5 | 11.3 ± 0.1 | 0.22 |
| vHH | C052 | 0.015 | 144.9 ± 5.8 | 11.6 ± 0.2 | 0.18 |
| C3b | | | | | |
| #13 | | | | | |

b) Effect of Translation Factor Overexpression on Transcript Level in a Continuous Cultivation.

To further elucidate the effect of C3b overexpression transcript levels were measured with the procedure described in Example 6a. As described above, the transcript levels were normalised to S. cerevisiae ACT1. Additionally, they were then normalised to the host strain, reactors C050 to receive the fold change of relative transcript levels for PpACT1 and vHH.

The overexpression of C3b led to 1.40±0.04-fold higher relative transcript level of vHH. PpACT1 showed similar fold change of 1.46±0.04. This confirms the results obtained in screenings and fed batch cultivation, showing that transcript levels are in general increased in cells overexpressing the selected translation initiation factor(s) independent of the applied cultivation mode.

c) Determination of Total Protein Concentration.

To determine, if this effect of increased transcript level has an impact on the concentration of total protein in the cells, the Biuret method was used.

Briefly, the collected cell pellets, described in Example 8a, were washed three times with water. Then they were diluted with water to receive 8 mg mL⁻¹ YDM in each tube. Two times 240 μL of cell suspension per sample were mixed with 125 μL of 3M NaOH and boiled at 99° C. for 5 min. After cooling, 125 μL 2.5% CuSO4 were added and the samples centrifuged at 16,000 g for 5 min. Of the obtained supernatant, two times 200 μL per tube were used for measurement in a Tecan Reader (Tecan Infinite M200) at a wavelength of 555 nm. For the calibration curve, dilutions of bovine serum albumin (Albumin Fraction V 98% for Molecular biology) (13, 12, 10, 8, 6, 4, 2, 1 and 0 g L⁻¹) were treated the same way as the samples.

The two reactors with the host strain, CBS2612 P_{G1-3} vHH #4, produced 0.24±0.00 mg protein per mg dry mass, whereas the two reactors with the overexpression strain, CBS2612 P_{G1-3} vHH C3b #13, produced 0.29±0.00 mg protein per mg dry mass. The 1.2-fold increase of total protein observed in the overexpression strain indicates that the increase of transcript level results in an increase of total protein in the cells. However, the effect on the recombinant POI is significantly stronger (1.8-fold) than for the overall cellular proteins (1.2-fold), once again highlighting our surprising findings that the TIFs of the mRNP are limiting during recombinant protein production and that their overexpression results in higher productivity.

### Example 9: Effect of the Overexpression of TIFs on the Secretion of Other Model Proteins

Human serum albumin (HSA) was chosen as another model protein to confirm the effects of TIF overexpression.

a) Generation of HSA Producer Strains

As described in Example 1a, the expression cassette for P_{G1-3}_HSA was transformed into CBS2612 to generate a HSA producing strain. The resulting strains were screened as described in Example 2a and the titers determined as described in Example 2c, by microfluidic capillary electrophoresis (mCE). Two HSA producing clones with different productivity were chosen as host for TIF overexpression. CBS2612 PG1-3 HSA #15 was chosen as the average producer host strain, whereas CBS2612 PG1-3 HSA #10 was chosen as the high producer host strain. These two strains were rescreened in quadruplicate and the results can be seen in Table 23. Additionally, the GCN of these strains was determined to explain the difference in productivity. GCN determination was done according to Example 1d.

TABLE 23
Two clones were chosen in the first screening to be used
as host strains for subsequent TIF overexpression. Titer,
WCW and yield were obtained in the rescreening of the
two chosen clones in quadruplicate. Additionally shown
is the GCN determined for the recombinant protein expression
cassette in these two host strains.

| | | | Yield | |
|---|---|---|---|---|
| | Titer HSA | WCW | HSA | |
| | [mg L⁻¹] | [g L⁻¹] | [mg g⁻¹] | GCN |
| CBS2612 P_{G1-3} | 208.4 ± 19.1 | 90.4 ± 1.4 | 2.3 ± 0.2 | 6 |
| HSA #10 | | | | |
| CBS2612 P_{G1-3} | 38.9 ± 3.5 | 97.3 ± 2.1 | 0.4 ± 0.0 | 1 |
| HSA #15 | | | | |

The titer seen in Table 23 shows the mean of the quadruplicate. The high producer, produces over 5 times more than the average producer, which correlates nicely to the higher GCN. Both strains were used for overexpression of the chosen translation initiation factor constructs.

b) Generation of TIF Overexpression Strains and their Effects on HSA Secretion

The combined overexpression C3b was chosen to be tested in the two HSA production strains described in Example 9a. Cloning was done as described in Example 1, followed by screening and titer determination as described in Example 2a and 2c.

TABLE 24
Screening results of C3b overexpression in the two different
chosen HSA producer strains. Additionally to HSA titer, WCW
and HSA yield, also the fold change of the HSA yield is shown. The
fold change was calculated in comparison to each respective host
strain, CBS2612 P_{G1-3} HSA #10 or #15. Also, the number
of clones used in the screening is shown.

| | Titer | | Yield | FC | Number |
|---|---|---|---|---|---|
| | HSA | WCW | HSA | HSA | of |
| | [mg L⁻¹] | [g L⁻¹] | [mg g⁻¹] | yield | clones |
| CBS2612 P_{G1-3} | 298.4 ± | 89.0 ± | 3.3 ± | 1.44 | 9 |
| HSA #10 C3b | 37.95 | 1.24 | 0.42 | | |
| CBS2612 P_{G1-3} | 52.9 ± | 90.8 ± | 0.6 ± | 1.42 | 10 |
| HSA #15 C3b | 0.03 | 2.35 | 0.03 | | |

Table 24 shows the results of the screening. Despite the difference in absolute HSA titers between the two producer host strains, the impact of TIF C3b overexpression is approximately the same, with an increase of 1.4-fold. This shows that the TIF overexpression has an effect of increasing recombinant protein secretion in high producer strains, as well as in average ones. Additionally, the results in Table 24 show that C3b overexpression increases recombinant protein secretion not only for vHH, as verified in the Examples above, but also for HSA, thus enforcing the notion of the general positive impact of TIF overexpression on recombinant protein production.

c) Fed-Batch Cultivations of HSA Producer Strains Overexpressing TIFs

Next. the HSA host strains and the corresponding C3b overexpression strains were used for fed-batch cultivations. The fed-batch cultivations were done as described in Example 7. In this case, the following equation for the linear incremental glucose feed was used: F[mL h⁻¹]=0.01*t+2. This resulted in an approximate growth rate of 0.029 h⁻¹ at sampling point F9.

TABLE 25
YDM of the fed-batch cultivation run, A499-A502, and the FC of titer
and yield can be seen here. Samples were taken at 2 different timepoints.
FC titer/yield is the fold change of the overexpression construct
titer/yield compared to the host strain titer/yield, at the same timepoint.

| | | | time after | | FC | FC |
|---|---|---|---|---|---|---|
| | Reactor | | feed start | YDM | HSA | HSA |
| | # | Sample | [h] | [g L⁻¹] | titer | yield |
| P_{G1-3} HSA | A499 | BE | 0 | 26.8 ± 0.2 | — | — |
| #10 | | F9 | 8.9 | 32.4 ± 0.8 | — | — |
| P_{G1-3} HSA | A500 | BE | 0 | 25.9 ± 0.1 | — | — |
| #10 C3b | | F9 | 8.9 | 33.0 ± 0.2 | 1.9 | 1.9 |
| P_{G1-3} HSA | A501 | BE | 0 | 26.3 ± 0.2 | — | — |
| #15 | | F9 | 8.9 | 32.6 ± 0.5 | — | — |
| P_{G1-3} HSA | A502 | BE | 0 | 25.9 ± 0.3 | — | — |
| #15 C3b | | F9 | 8.9 | 34.2 ± 0.2 | 1.2 | 1.2 |

As for the other model proteins and strains, C3b overexpression increased recombinant HSA secretion yields in both producer host strains, as can be seen in Table 25. In particular, the increase in secreted protein is much more pronounced for the high producer strain, CBS2612 P_{G1-3} HSA #10, with an increase of 1.9-fold. This leads to the conclusion that TIFs of the mRNP pose a stronger bottleneck on cells with higher capability for recombinant protein expression (e.g. by higher transcription due to higher GCN and/or promoters with high expression strength), and that such cells benefit even more by TIF overexpression.

1. A recombinant eukaryotic host cell expressing a gene of interest (GOI) which is engineered by genetic modifications to increase expression of two or more genes encoding translation initiation factors (TIF genes) of the messenger ribonucleoprotein (mRNP), compared to the host cell prior to said one or more genetic modifications, wherein said TIF genes comprise at least a gene encoding eIF4A and a gene encoding eIF4G, and wherein expression of at least one of said TIF genes is under transcriptional control of a promoter different from the promoter controlling expression of said GOI.

2. The host cell of claim 1, wherein
a) said eIF4A comprises at least 60% sequence identity to any one of SEQ ID NO:12-33; and
b) said eIF4G comprises at least 60% sequence identity to any one of SEQ ID NO: 34-44.

3. The host cell of claim 1 or 2, wherein said TIF genes further comprise any one or more of
a) a gene encoding eIF4E,
b) a gene encoding PAB1; or
c) a gene encoding RLI1.

4. The host cell of claim 3, wherein
a) said eIF4E comprises at least 60% sequence identity to any one of SEQ ID NO:1-11;
b) said PAB1 comprises at least 60% sequence identity to any one of SEQ ID NO:45-55; and
c) said RLI1 comprises at least 60% sequence identity to any one of SEQ ID NO:56-65.

5. The host cell of any one of claims 1 to 4, wherein one or more of said TIF genes are optimized for expressing said TIF gene(s) in the host cell.

6. The host cell of any one of claims 1 to 5, overexpressing at least
a) genes encoding eIF4A and eIF4G,
b) genes encoding eIF4A, eIF4G, and eIF4E,
c) genes encoding eIF4A, eIF4G, eIF4E, and PAB1;
d) genes encoding eIF4A, eIF4G, and PAB1.

7. The host cell of claim 6, wherein the host cell is additionally engineered to overexpress a gene encoding RLI1.

8. The host cell of any one of claims 1 to 7, wherein said genetic modifications comprise a knockin, substitution, disruption, deletion or knockout of (i) one or more polynucleotides, or a part thereof; or (ii) an expression control sequence, preferably an expression control sequence selected from the group consisting of a promoter, a ribosomal binding site, transcriptional or translational start and stop sequences, an enhancer and activator sequence, preferably wherein said one or more genetic modifications comprise the integration of a heterologous polynucleotide or expression cassette into the host cell genome.

9. The host cell of any one of claims 1 to 8, wherein said genetic modifications include an increase in the number of said TIF gene(s) or the number of expression cassettes comprising said TIF gene(s), and/or a gain-of-function alteration in said TIF gene(s), resulting in increasing the level or activity of said TIF gene(s).

10. The host cell of any one of claims 1 to 9, wherein said TIF genes are endogenous or heterologous to the host cell.

11. The host cell of any one of claims 1 to 10, comprising
a) an expression system to express one or more of said TIF genes in one or more heterologous TIF expression cassettes, each comprising one or more expression control sequences operably linked to said TIF gene(s); and
b) a GOI expression cassette comprising a GOI and one or more expression control sequences operably linked to said GOI;
wherein the expression system of a) and the expression cassette of b) are engineered to express the respective TIF gene(s) and GOI when culturing the host cell in a cell culture.

12. The host cell of claim 11, wherein
a) at least one of the TIF expression cassettes comprises a constitutive promoter; and/or
b) the GOI expression cassette comprises an inducible, de-repressible or otherwise regulatable promoter, or a constitutive promoter.

13. The host cell of any one of claims 1 to 12, which is:
a) a yeast cell of a genus selected from the group consisting of Pichia, Hansenula, Komagataella, Saccharomyces, Kluyveromyces, Candida, Ogataea, Yarrowia, and Geotrichum, preferably Pichia pastoris, Komagataella phaffii, Komagataella pastoris, Komagataella pseudopastoris, Saccharomyces cerevisiae, Ogataea minuta, Kluyveromces lactis, Kluyveromes marxianus, Yarrowia lipolytica or Hansenula polymorphs;
b) a cell of filamentous fungi, such as Aspergillus awamori or Trichoderma reesei;
c) a non-human primate, human, rodent or bovine cell, such as mouse myeloma (NSO)-cell lines, Chinese hamster ovary (CHO)-cell lines, HT1080, H₉, HepG2, MCF7, MDBK Jurkat, MDCK, NIH3T3, PC12, BHK (baby hamster kidney cell), VERO, SP2/0, YB2/0, Y0, C127, L cell, COS, e.g., COS1 and COS7, QC1-3, HEK-293, VERO, PER.C6, HeLA, EBI, EB2, EB3, oncolytic or hybridoma-cell lines;
d) an insect cell, such as Sf9, Mimic™ Sf9, Sf21, High Five (BT1-TN-5B1-4), or BT1-Ea88 cells;
e) an algae cell, such as of the genus Amphora, Bacillariophyceae, Dunaliella, Chlorella, Chlamydomonas, Cyanophyta (cyanobacteria), Nannochloropsis, Spirulina, or Ochromonas); or
f) a plant cell, such as cells from monocotyledonous plants, preferably maize, rice, wheat, or Setaria, or from a dicotyledonous plant, preferably cassava, potato, soybean, tomato, tobacco, alfalfa, Physcomitrella patens or Arabidopsis.

14. A method for producing a host cell of any one of claims 1 to 13, comprising genetically engineering a host cell to comprise within one or more heterologous expression cassettes one or more of said TIF genes and a gene of interest (GOI).

15. A method for producing a protein of interest (POI) encoded by a gene of interest (GOI) by culturing the host cell of claim 14 under conditions to produce said POI.

16. A method of claim 15, wherein the host cell is cultured in a culture medium under conditions to co-express one or more of said TIF genes and to secrete said POI into the host cell culture, and the POI is recovered from the host cell culture.

17. The method of claim 15 or 16, wherein said host cell is modified to co-express one or more of said TIF genes at a level that increases the host cell's specific productivity for said POI (μg/g yeast dry mass (YDM) per hour and/or volumetric productivity for said POI (μg/L per hour).

18. The method of any one of claims 15 to 17, wherein the POI is a therapeutic or diagnostic product, preferably a peptide, polypeptide or protein selected from the group consisting of an antigen-binding protein, a therapeutic protein, an enzyme, a peptide, a protein antibiotic, a toxin fusion protein, a carbohydrate—protein conjugate, a structural protein, a regulatory protein, a vaccine antigen, a growth factor, a hormone, a cytokine, a process enzyme, and a metabolic enzyme.

19. A method of increasing the yield of a protein of interest (POI) when produced by a host cell expressing a gene of interest (GOI) encoding said POI, by co-expressing one or more heterologous expression cassettes expressing one or more TIF gene(s) of the messenger ribonucleoprotein (mRNP) in a cell culture.

20. A polypeptide expression system comprising one or more heterologous expression cassettes expressing one or more TIF gene(s) of the messenger ribonucleoprotein (mRNP).

21. The expression system of claim 20, which comprises an expression cassette comprising a gene of interest (GOI) encoding a protein of interest (POI) with and one or more expression control sequences operably linked to said GOI.


SEQUENCE LISTING (196 sequences)

SEQ ID NO: 1 - protein, 202 aa, Komagataella phaffii
MSETENAPHEKSLLDDQVDFTVKHPLNSEWTLWYTKPAVHEQESWSDLLRPIISFNSVEEFWGIFNSIPKASDLPVKSDYHLFRDGIKPEWEDERNANGGKLTYQFAYRKVDINELWSRGLLSVIGETIQDDDGETEVNGIVLSIRRAALKIALWTKSKDEAVLRPIGERFKKVLKLTARDHIDFQPHKSGAGSKNVPSFSI

SEQ ID NO: 2 - protein, 202 aa, Komagatealla pastoris; identical to SEQ ID NO: 1

SEQ ID NO: 3 - protein, 217 aa, Homo sapiens
MATVEPETTPTPNPPTTEEEKTESNQEVANPEHYIKHPLQNRWALWFFKNDKSKTWQANLRLISKFDTVEDFWALYNHIQLSSNLMPGCDYSLFKDGIEPMWEDEKNKRGGRWLITLNKQQRRSDLNRFWLETLLCLIGESFDDYSDDVCGAVVNVRAKGDKIAIWTTECENREAVTHIGRVYKERLGLPPKIVIGYQSHADTATKSGSTTKNRFVV

SEQ ID NO: 4 - protein, 247 aa, Aspergillus niger
MAAVAENGHAPVADENAAPVAAAVEQKEQTEKTNGDSVTVFHDPENFNVKHPLMHEWTLWFTKPPSGKGDNWNDLLKEVVTFNSVEEFWGIYNNISPTSELGLKADYHLFKKGVRPEWEDPQNKHGGKWSYSFKDKRSVPIDDLWLHAQLAAIGETLENDGDNEVMGVVVNVRKGFYRVGLWTRTVGKSIPGDKNGRTPAQGKEILENIGRRFKEVLRLKEADAVEFSGHTDSAHSGSTRAKAKYTV

SEQ ID NO: 5 - protein, 212 aa, Cricetulus griseus
METTPTTNPPPVEEEKTESNQEVANPEHYIKHPLQNRWALWFFKNDKSKTWQANLRLISKFDTVEDFWALYNHIQLSSNLMPGCDYSLFKDGIEPMWEDEKNKRGGRWLITLNKQQRRSDLDRFWLETLLCLIGESFDDYSDDVCGAVVNVRAKGDKIAIWTTECENRDAVTHIGRVYKERLGLPPKIVIGYQSHADTATKSGSTTKNRFVV

SEQ ID NO: 6 - protein, 210 aa, Kluyveromyces lactis
MSVEEVTQKTQDLTVDEAKPATVLSDSKEFQLKHPLSSKWTLWYTKPPVDSSESWSDLLRPVTSLDTVEEFWAIQNGIPKPFDLPIKSDYHLFRNDIRPEWEDEANSEGGKLSYQFKNKRHNIDDLWLRTLLAVIGETIDEEESEINGCVLNVRKAVFKIALWTRTTNLNALTNIATKFKQVLQLGEGEKLEFYNHRTSNSGHAPPTALL

SEQ ID NO: 7 - protein, 211 aa, Ogataea polymorpha
MSEELNQAAKDLSLDEKKDVTVLDSREEFNVKHPLNSKWTLWYTKPAVDSSESWSDLLKPVVAFDTVEEFWGIFNSIPKADELPLKSDYHLFRENIKPEWEDPANSKGGRFSFQFKGKRLEHGINEIWTRALLSVIGETIEDDQNEVNGVVLNVRKVGYRVCLWTKSCDRAALVPIGERLKKILMLREGESVEFISHKDSNDRGAKPAFYV

SEQ ID NO: 8 - protein, 213 aa, Saccharomyces cerevisiae
MSVEEVSKKFEENVSVDDTTATPKTVLSDSAHFDVKHPLNTKWTLWYTKPAVDKSESWSDLLRPVTSFQTVEEFWAIIQNIPEPHELPLKSDYHVFRNDVRPEWEDEANAKGGKWSFQLRGKGADIDELWLRTLLAVIGETIDEDDSQINGVVLSIRKGGNKFALWTKSEDKEPLLRIGGKFKQVLKLTDDGHLEFFPHSSANGRHPQPSITL

SEQ ID NO: 9 - protein, 243 aa, Schizosaccharomyces pombe
MADAEDSRHSKNEGFPNTSLITEKLDLLDLFGSPKVKTEREGRPARLLEGLSAVNAETAFVKTHPLQHEWTLWFLKPPTQGLEWSDLLKEIISFKTVEEFWGIFKTISKASMLPAKSDYSYFLKGIRPEWEDPQNMNGGKWAYQSKHKGSNLDELWLYMVLAAIGETLDPTGKEVTGVVCNMRKGFYRIAVWTRNCNDKDVLEKIGLRFKEVLGISDKETIEYSAHEDSSKAGSMRAKTRMSL

SEQ ID NO: 10 - protein, 259 aa, Trichoderma reesei
MASAAVDGANMDQQVDLTSIPISPNGDEHAGETKENKDDGKPVTVFHDKDNFTVKHPLQSKWTLWFTKPPSGKGDNWNDLLKEVITFDSVEEFWGVYNNVAPVSELALKSDYHLFKAGVRPEWEDPQNKHGGKWSYQYKDKRNVDVDRLWLQVMMAAIGETLEEEEDGEVMGVVVNVRKGFYRIGVWTRTIGKSIPGRGDGDVAGGKGRSNEKGKEILMNIGRRFKETLELPANEQIEFSGHTDSAHAGSTRAKAKYVV

SEQ ID NO: 11 - protein, 278 aa, Yarrowia lipolytica
MSTMAIQTDVSVISTDIALQLSQMAATTDNVTEAVANLNIKEEAPVASEPAEVTETVEEEVTPAEPFSTVFDSKTEFATVHKLTTPWTLWYIKPPSPAVAWHDLIKEVVSFDTVEGFWAVYNNIPKLVELPIRSDYAFFRKGIRPEWECDSNKAGGKWFYQFKPKSANEFDEIWLHTVLSVIGESLETDEEIAKHEGKYNNVDGSASNVNGIFVNIRKAGVRFNVWTKTSEVTDELRQMGVKIKQVLRLPEKEEVQFQSHEESASKNVKSQGKTRLAV

SEQ ID NO: 12 - protein, 403 aa, Komagataella phaffii
MSEGIIEIDTNLIETNYDQVVSTFDELNLKEQILRAVFAYGYEFPSAIQSRAILPIVAGRDVLAQAQSGTGKTATFAISALQRLDEKLKAAQVLILAPTRELALQIKKVVLALGMHLNISVHACIGGVDPALDVKAFNSGVQIVVGTPGRVQDMIERGAFRTENVKMVILDEADEMLSTGFKEQIYTLFTLLPSTTQVVLLSATMPQEVLEVTTKFMNNPIRILVKKDQLTLEGIKQFYVDVGEEQYKFDCLCDLYDSISVTQAVIFCNTRRKVEELTERLTAQKFTVSAIHSELSQTERDTIMQEFRTGSSRILISTDLLARGIDVQQVSLVINYDLPSNKENYIHRIGRGGRFGRKGVAINFVTSKDVEMMKEIEKFYSTQIDELPSSITDLFETEEVNES

SEQ ID NO: 13 - protein, 403 aa, Komagataella pastoris
MSEGIIEIDTNLIETNYDQVVSTFDELNLKEQILRAVFAYGYEFPSAIQSRAILPIVAGRDVLAQAQSGTGKTATFAISALQRLDEKLKAAQVLILAPTRELALQIKKVVLALGMHLNISVHACIGGVDPALDVKAFNSGVQIVVGTPGRVQDMIERGAFRTENVKMVILDEADEMLSTGFKEQIYTLFTLLPSTTQVVLLSATMPQEVLEVTTKFMNNPIRILVKKDQLTLEGIKQFYVDVGEEQYKFDCLCDLYDSISVTQAVIFCNTRRKVEELTERLTAQKFTVSAIHSELSQSERDTIMQEFRTGSSRILISTDLLARGIDVQQVSLVINYDLPSNKENYIHRIGRGGRFGRKGVAINFVTSKDVEMMKEIEKFYSTQIDELPSSITDLFETEEVNES

SEQ ID NO: 14 - protein, 413 aa, Homo sapiens
MATTATMATSGSARKRLLKEEDMTKVEFETSEEVDVTPTFDTMGLREDLLRGIYAYGFEKPSAIQQRAIKQIIKGRDVIAQSQSGTGKTATFSISVLQCLDIQVRETQALILAPTRELAVQIQKGLLALGDYMNVQCHACIGGTNVGEDIRKLDYGQHVVAGTPGRVFDMIRRRSLRTRAIKMLVLDEADEMLNKGFKEQIYDVYRYLPPATQVVLISATLPHEILEMTNKFMTDPIRILVKRDELTLEGIKQFFVAVEREEWKFDTLCDLYDTLTITQAVIFCNTKRKVDWLTEKMREANFTVSSMHGDMPQKERESIMKEFRSGASRVLISTDVWARGLDVPQVSLIINYDLPNNRELYIHRIGRSGRYGRKGVAINFVKNDDIRILRDIEQYYSTQIDEMPMNVADLILE

SEQ ID NO: 15 - protein, 398 aa, Aspergillus niger
MASNDKGLEEIPEGQIETNYDEVTDSFDSMDLKPELLRGVYAYGFERPSAIQQRAIKPIIAGHDVIAQAQSGTGKTATFSISALQKIDQELKACQALIVAPTRELAQQIQKVVVAIGDFMNIECHACIGGTNVRDDMNALRAGPQVVVGTPGRIHDMIERRVLKTDQMKLFILDEADEMLSRGFTEQIYDIFQLLPQSTQVTLLSATMPQDVLEVTTKFMRDPIRILVKKQELTLEGIKQFYIAVEKEEWKLDTLSDLYETVTITQAVIFCNTRRKVDWLTDKLTARDFTVSAMHGDMEQGQRDVIMKEFRSGSSRVLIATDLLARGIDVQQVSLVINYDLPANRENYIHRIGRGGRFGRKGVAINFVTADDVRMMREIEQFYSTQIEEMPMNVADLI

SEQ ID NO: 16 - protein, 411 aa, Cricetulus griseus
MAAAATMATSGSARKRLLKEEDMTKVEFETSEEVDVTPTFDTMGLREDLLRGIYAYGFEKPSAIQQRAIKQIIKGRDVIAQSQSGTGKTATFSISVLQCLDIQVRETQALILAPTRELAVQIQKGLLALGDYMNVQCHACIGGTNVGEDIRKLDYGQHVVAGTPGRVFDMIRRRSLRTRAIKMLVLDEADEMLNKGFKEQIYDVYRYLPPATQVVLISATLPHEILEMTNKFMTDPIRILVKRDELTLEGIKQFFVAVEREEWKFDTLCDLYDTLTITQAVIFCNTKRKVDWLTEKMREANFTVSSMHGDMPQKERESIMKEFRSGASRVLISTDVWARGLDVPQVSLIINYDLPNNRELYIHRIGRSGRYGRKGVAINFVKNDDIRILRDIEQYYSTQIDEMPMNVADLI

SEQ ID NO: 17 - protein, 396 aa, Kluyveromyces lactis
MSDGITDIDENQIQSTYDKVVYSFDDLKLKEELLRGIFGYGFVEPSAIQQRAILPIIEGKDVLAQAQSGTGKTGTFSIAALQNIDEKIKAPQGLILAPTRELALQIQKVVMALAIHMDVKVHACIGGTSLQEDSEALRGGAQIIVGTPGRVFDMIDRRIFKTDNIKMFILDEADEMLSTGFKEQIYNIFTMLPPTSQVVLLSATMPGDVLEVTSKFMKDPVRILVKKDELTLEGIGQYYVNVEEEQYKYDCLTDLYDSISVTQAVIFCNTRRKVEELTERLRENNFTVSAIYSDLQQQERDTIMKEFRSGSSRILISTDLLARGIDVQQVSLVINYDLPSNKENYIHRIGRGGRFGRKGIAINFVTNKDIGAMRELERFYSTQIEELPSSISELFD

SEQ ID NO: 18 - protein, 397 aa, Ogataea polymorpha
MASENIQEVDSNLIESNYDNVVYTFDELNLKPEIIRGIFAYGYEQPSAIQQRAILPITEGRDVLAQAQSGTGKTATFTISALQRIDEKLKKTQALILAPTRELALQIQKVVLAIGMHLDLTVHASIGGKAVQEDMEALRNGAQIVVGTPGRVYDMIERGYFKTDAIKMFIMDEADEMLSSGFKEQIYNVFRFLPTTTQVVLLSATMPQDVLEVTTKFMRNPVRILVKKDELTLEGIKQFYIDVDEEQYKFDCLCDLYDSISVTQAVIFCNTRRKVETLTQQLTENNFTVSAIHSDLSQQDRDTIMNEFRTGSSRILISTDLLARGIDVQQVSLVINYDLPSNKENYIHRIGRGGRFGRKGVAINLITREDTEMLREIEMFYSTQIVEMPSSINDLFD

SEQ ID NO: 19 - protein, 395 aa, Saccharomyces cerevisiae
MSEGITDIEESQIQTNYDKVVYKFDDMELDENLLRGVFGYGFEEPSAIQQRAIMPIIEGHDVLAQAQSGTGKTGTFSIAALQRIDTSVKAPQALMLAPTRELALQIQKVVMALAFHMDIKVHACIGGTSFVEDAEGLRDAQIVVGTPGRVFDNIQRRRFRTDKIKMFILDEADEMLSSGFKEQIYQIFTLLPPTTQVVLLSATMPNDVLEVTTKFMRNPVRILVKKDELTLEGIKQFYVNVEEEEYKYECLTDLYDSISVTQAVIFCNTRRKVEELTTKLRNDKFTVSAIYSDLPQQERDTIMKEFRSGSSRILISTDLLARGIDVQQVSLVINYDLPANKENYIHRIGRGGRFGRKGVAINFVTNEDVGAMRELEKFYSTQIEELPSDIATLLN

SEQ ID NO: 20 - protein, 392 aa, Schizosaccharomyces pombe
MVDQLEDSVIETNYDEVIDTFDDMNLKPELLRGIYAYGFERPSAIQQRAIMPILGERDVLAQAQSGTGKTATFSISVLQKIDTSLKQCQALILAPTRELAQQIQKVVVALGDLMNVECHACIGGTLVRDDMAALQAGVHVVVGTPGRVHDMIQRRALPTDAVQMFVLDEADEMLSRGFKDQIYDIFQLLPPTAQVVLLSATMPQDVLEVTTKFMRDPIRILVKKDELTLEGIKQFYVAVEKEEWKLDTLCDLYETVTVTQAVIFCNTRRKVDWLTEQLTERDFTVSSMHGDMDQAQRDTLMHEFRTGSSRILITTDLLARGIDVQQVSLVINYDLPANRENYIHRIGRGGRFGRKGVSINFVTNDDVRMMREIEQFYNTHIEEMPMNIADLI

SEQ ID NO: 21 - protein, 396 aa, Trichoderma reesei
MADKGLEDVPEGQIESNYDETVDSFDDMNLKSELLRGVYAYGFERPSAIQQRAIMPVIKGHDVIAQAQSGTGKTATFSISVLQKIDPSVKQCQALILAPTRELAQQIQKVVVAIGDFMNVECHACIGGTSVREDMKALQDGPQVVVGTPGRVHDMIQRRFLKTDAMKMFVLDEADEMLSRGFTDQIYDIFQLLPQSTQVVLLSATMPQDVLEVTTRFMRDPVRILVKKDELTLEGIKQFYIAVEKEEWKLDTLSDLYETVTITQAVIFCNTRRKVDWLTDKLTARDFTVSAMHGDMDQAQRDLIMKEFRSGSSRVLIATDLLARGIDVQQVSLVINYDLPANRENYIHRIGRGGRFGRKGVAINFVTADDVRMMREIEQFYSTQIEEMPMNVADLI

SEQ ID NO: 22 - protein, 395 aa, Yarrowia lipolytica
MADGLTDIDSSQITTNYDEVVTSFDDLGLKDELLRGIYGYGFENPSSIQQRAILPVIKGNDVLAQAQSGTGKTATFSISALQNIDEKIKKPQALIIAPTRELAHQIQKVVLAFGEYMKIECHACIGGTSVAEDIRVIQEGVHVIVGTPGRIHDMIERRILKTDLIKMFILDEADEMLSREFKDPIYDIFTTLPETTQTVLLSATMPAEVLDITGKFMRDPVRILVKKDELTLEGIKQFYIDVEQESYKFEVLCDLYETINVSQAVIFCNTRRKVDYLTQALTEADFTVSSMHGETEQSQRDVIMKAFRTGSSRILITTDLLARGIDVQQVSLVINFDLPSNRENYIHRIGRGGRFGRKGVAINFVTSEDHGMLKELERFYSTEIVEMPTNIADLI

SEQ ID NO: 23 - protein, 486 aa, Komagataella phaffii
MHPYTVVFARLLLGVFSTAAPNSPPPFCTSCHLGDQRLWNLAQNLACTLPPQKNFPCLVARRSKIKSFSTTFVPNKSPFYLPIMSEGIIEIDTNLIETNYDQVVSTFDELNLKEQILRAVFAYGYEFPSAIQSRAILPIVAGRDVLAQAQSGTGKTATFAISALQRLDEKLKAAQVLILAPTRELALQIKKVVLALGMHLNISVHACIGGVDPALDVKAFNSGVQIVVGTPGRVQDMIERGAFRTENVKMVILDEADEMLSTGFKEQIYTLFTLLPSTTQVVLLSATMPQEVLEVTTKFMNNPIRILVKKDQLTLEGIKQFYVDVGEEQYKFDCLCDLYDSISVTQAVIFCNTRRKVEELTERLTAQKFTVSAIHSELSQTERDTIMQEFRTGSSRILISTDLLARGIDVQQVSLVINYDLPSNKENYIHRIGRGGRFGRKGVAINFVTSKDVEMMKEIEKFYSTQIDELPSSITDLFETEEVNES

SEQ ID NO: 24 - protein, 403 aa, Komagatealla pastoris; identical to SEQ ID NO: 13

SEQ ID NO: 25 - protein, 411 aa, Homo sapiens [sequence omitted]

SEQ ID NO: 26 - protein, 398 aa, Aspergillus niger; identical to SEQ ID NO: 15

SEQ ID NO: 27 - protein, 411 aa, Cricetulus griseus; identical to SEQ ID NO: 16

SEQ ID NO: 28 - protein, 396 aa, Kluyveromyces lactis; identical to SEQ ID NO: 17

SEQ ID NO: 29 - protein, 397 aa, Ogataea polymorpha; identical to SEQ ID NO: 18

SEQ ID NO: 30 - protein, 395 aa, Saccharomyces cerevisiae; identical to SEQ ID NO: 19

SEQ ID NO: 31 - protein, 392 aa, Schizosaccharomyces pombe; identical to SEQ ID NO: 20

SEQ ID NO: 32 - protein, 396 aa, Trichoderma reesei; identical to SEQ ID NO: 21

SEQ ID NO: 33 - protein, 395 aa, Yarrowia lipolytica; identical to SEQ ID NO: 22

SEQ ID NO: 34 - protein, 1098 aa, Komagataella phaffii
MSNKNVDTAPDNGSGGSNSPASVPHYPQQHRANYNPHKNHTNHGGNNGGAQGYMQHQQQQQMYYNQMGYPMPYPNAGGNFNQNVYLVPYNTMVPGTPYGAGAPGVPPSYPVSPSPAFASPQKTHVPVSLRTPSGEPINLVPAGQTSSSSSNASPHVKSTSIPPSAVTSPAASIAPTVASPTKAAAGSEDAGKTRDAFKQMVQALAAKKKAAAAAKAEGKTDDKKESVESKPKSAVQPETKPKVEVKLEPKPKVEVKLEPKPKVEIKLEPKPVSEAKPETKTVPEVEEPSTVATIKSESTPIEELKPKVEVTPEVASKPEQVESAQEPANDVKPSEVSAKEVQESKEESLKQPKEALNDIKSTEESNKKFSLPEEESNDHQTLSQIQGVKDTEQSQEFEEPQGTQETQETQETQETAKPLEAQAPQEIQETQEPEEVQEQTPETSETVSSVTEEVSPAPAPGLPLSKFLDRIRKAPAISNPFKHEYSGDFHGPDESLSGLKTIKYDPGFLIQFATFNFHIDEDFINDVGSKVVIPVNTRRDNFSKSMSTSNFKGSGRNMGSNFRNDFGSRNNSKQGSKKKNKSQKSQSRRGPKYSNEEEPAEPAVPAELPNRAASKWVPRSLRNKEAEVKTAPDGSIIVSEEDIKRKTKSLLNKLTLEFFDDISNDIIALTKQAQWEDDVKTLKQVIESIFAKACDEPYWSSMYAKLCAKMCKDTPPEIKETNEKGNTFTGGDLVRRVLINRCQTEYQKGWIDQLPTNEDGSPLEPELMSDEYYKQAAAKRRGLGLVKFIGQLYQLNLLSDNVIFHCVLGQSKETDNPSEDTMENLVQLLEAVGYRMEMSSEPKIRRVLDLAFANIKAIVDANKIPSRIRFMLMDLMDLRRNGWATTEENAGPKTIQEIHLEAEIKRSEEEREKERDRHSRRGRYNDSKPNSRQKSSWGSSVVSPNDIRNAGYVRNSDKSLGPVNSFSRGKSSRQASKQSNSDFTSVPITSSGSRTALNHSGDSSTSLNEPQSREYSKKSESRPAPSNIFAALEDHDHDHEHGHEQEENKHSKDLQNGEAEAPEDDNVGEIAQADENVQGQEQEEEREEEREQEEEQEV

SEQ ID NO: 35 - protein, 1101 aa, Komagataella pastoris
MSNKNVDTAPDNGSGGSNSPASVPHYPQQHRSNYNPHKNHTNHGGNNGGAQGYMQHQQQQQMYYNQMGYPMPYPNAGGNFNQNVYLVPYNTMVPGTPYGAGAPGVPPSYPVSPTPAFASPQKTHVPVSLRTPSGEPINLVPAGQTSSSSSNASPHVKSTSIPASAVTSPAASIAPTVASPAKPAAGSEDAGKTRDAFKQMVQALAAKKKAAAAKAEGKTDDKKESVESKPKAAVQPEAKPKVEVKLEPKPKVEVKLEPKPKVEIKLEPKPVSEAKLETKTAPEVEEPSTVATIKSETKPIEELKPTVEVSLQVEAEPKQFESAQEPAADLNSSEVSAKEVKETKEEALNQPKEALNDIKSAEELNEKSTPQEEESNDQQTLSQSQEAKETEQSQGSEEPQETQEAQEVQVTQTTPEGQEGKETQEPQEAQEAQEVQEQTPETSETVSSVTEEVSPAPAPGLPLSTFLDRIRKSPAISNPFKHQYSGDFHGPDESLSGLKTIKYDPGFLIQFASFNFHIDEDFINDVGSKVVIPVNTRRDNFSKSMSTSNFKGSGRNMGSNFRNDFGSRNNSKQGSKKKNKSQKSQSRRGPKYSNEEEPAEPAVPAELPNRAASKWVPRSLRNKEAEVKTAPDGSVIVSEEDIKRKTKSLLNKLTLEFFDDISNDIIALTKQAQWEDDVKTLKQVIESIFAKACDEPYWSSMYAKLCAKMCKDTPPEIKETNEKGNTFTGGDLVRRVLINRCQTEYQKGWIDQLPTNEDGSPLEPELMSDEYYKQAAAKRRGLGLVKFIGQLYQLNLLSDNVIFHCVLGQSKETDNPSEDTMENLVQLLEAVGYRMEMSSEPKIRRVLDLAFANIKAIVDANKIPSRIRFMLMDLMDLRRNGWATTEENAGPKTIQEIHLEAEIKRSEEEREKERDRHSRRGRYNDSKPNSRQKSSWGSSVVSPNDIRNAGYVRNSDKSLGPVNSFSRGKSSRQASKQSNSDFTSVPITSSGSRTALNHSGDSSTSLNEPQSREYSKKSESRPAPSNIFAALEDHDHDHEHGHEQEENKHSKDLQNGEAETPENVNVEEIAQADENVQGQEQDEEQDEEQDEEREQEEEQEV

SEQ ID NO: 36 - protein, 1780 aa, Homo sapiens
VKKIHRHRLSPATEKGAASSGEGDKDPPPPAHEDIAVKGTATAAGTGPGTGAAAAAAAAVPPPHPAAAAAAAAAAAAAAAAVPPPHPNIRALQTQAPQQIPRGPVQQPLEDRIFTPAVSAVYSTVTQVARQPGTPTPSPYSAHEINKGHPNLAATPPGHASSPGLSQTPYPSGQNAGPTTLVYPQTPQTMNSQPQTRSPGGFRPIQFFQRPQIQPPRATIPNSSPSIRPGAQTPTAVYQANQHIMMVNHLPMPYPVPQGPQYCIPQYRHSGPPYVGPPQQYPVQPPGPGPFYPGPGPGDFPNAYGTPFYPSQPVYQSAPIIVPTQQQPPPAKREKKTIRIRDPNQGGKDITEEIMSGGGSRNPTPPIGRPTSTPTPPQLPSQVPEHSPVVYGTVESAHLAASTPVTAASDQKQEEKPKPDPVLKSPSPVLRLVLSGEKKEQEGQTSETTAIVSIAELPLPPSPTTVSSVARSTIAAPTSSALSSQPIFTTAIDDRCELSSPREDTIPIPSLTSCTETSDPLPTNENDDDICKKPCSVAPNDIPLVSSTNLINEINGVSEKLSATESIVEIVKQEVLPLTLELEILENPPEEMKLECIPAPITPSTVPSFPPTPPTPPASPPHTPVIVPAAATTVSSPSAAITVQRVLEEDESIRTCLSEDAKEIQNKIEVEADGQTEEILDSQNLNSRRSPVPAQIAITVPKTWKKPKDRTRTTEEMLEAELELKAEEELSIDKVLESEQDKMSQGFHPERDPSDLKKVKAVEENGEEAEPVRNGAESVSEGEGIDANSGSTDSSGDGVTFPFKPESWKPTDTEGKKQYDREFLLDFQFMPACIQKPEGLPPISDVVLDKINQPKLPMRTLDPRILPRGPDFTPAFADFGRQTPGGRGVPLLNVGSRRSQPGQRREPRKIITVSVKEDVHLKKAENAWKPSQKRDSQADDPENIKTQELFRKVRSILNKLTPQMFNQLMKQVSGLTVDTEERLKGVIDLVFEKAIDEPSFSVAYANMCRCLVTLKVPMADKPGNTVNFRKLLLNRCQKEFEKDKADDDVFEKKQKELEAASAPEERTRLHDELEEAKDKARRRSIGNIKFIGELFKLKMLTEAIMHDCVVKLLKNHDEESLECLCRLLTTIGKDLDFEKAKPRMDQYFNQMEKIVKERKTSSRIRFMLQDVIDLRLCNWVSRRADQGPKTIEQIHKEAKIEEQEEQRKVQQLMTKEKRRPGVQRVDEGGWNTVQGAKNSRVLDPSKFLKITKPTIDEKIQLVPKAQLGSWGKGSSGGAKASETDALRSSASSLNRFSALQPPAPSGSTPSTPVEFDSRRTLTSRGSMGREKNDKPLPSATARPNTFMRGGSSKDLLDNQSQEEQRREMLETVKQLTGGVDVERNSTEAERNKTRESAKPEISAMSAHDKAALSEEELERKSKSIIDEFLHINDFKEAMQCVEELNAQGLLHVFVRVGVESTLERSQITRDHMGQLLYQLVQSEKLSKQDFFKGFSETLELADDMAIDIPHIWLYLAELVTPMLKEGGISMRELTIEFSKPLLPVGRAGVLLSEILHLLCKQMSHKKVGALWREADLSWKDFLPEGEDVHNFLLEQKLDFIESDSPCSSEALSKKELSAEELYKRLEKLIIEDKANDEQIFDWVEANLDEIQMSSPTFLRALMTAVCKAAIIADSSTFRVDTAVIKQRVPILLKYLDSDTEKELQALYALQASIVKLDQPANLLRMFFDCLYDEEVISEDAFYKWESSKDPAEQNGKGVALKSVTAFFTWLREAEEESEDN

SEQ ID NO: 37 - protein, 1536 aa, Aspergillus niger
MSSIPPKSGLQPQGQSTSTQAASSAHTSSPSVGGKSPTPSSTSTTAPRSYANATKKSATDSTAAPVTVGGPSQHGKSTTASPVSGKPMQQSQTPGVTIVNGAPAPASQGDHTRKPSVTITSAGTSGFIPNGGQTGRPNSLQFGFAANQQSSPNMGNPAVLANQPQSGLGVTPPMNPRVTSPQTSPSPIPQPASSGGRPPPSSYQSQGNVPNFGSFGDAGDNMRPAPQAPLGPGAQSTHLRRESSQSTHSDMSNHMGGGPGGRGGYHHQGGRGRGFSQSSYQGQMPYSPNASFRSTPNQPRGGPNMAPQFPNQPRPMPFPNSPHQASRSPALANAHPATPQMNQVPMAHPQMAPQPYYGQPMPPQPVRPHPLQKPARRGGALNRKKGPSRTPITPRSSVTPNPHITLPPNLAPESGHFERYLTMMKSKQGYPTFDPAYGFYPNYNMGGVPYMPPSPQPRPGIPYNAQAPYMQNQYPVQPAAQATALSRSPSHSNDRPGSSLGQGQPPAGAPGPAHAHNASRSSNSPAPQKSSFVIPTAKKNAVVIKDPGSGAVKTFEKAPASPARVTPSPVKIATPTSTPPPRTGSSADHTRTDSKSTKTDEEKKKELRDAVHQKIEQDRIEKEEAERRKAEEEAAQKKKEEEEEAARKKAEEDAAQKKKEEEEAAQKKAADEEAARKGLEDLSLKDKPAETKAEEPSKPAAAPAPADDDDDIDYDAIEREMAEIEAKERAAEEAYYARKKAEKEEKERKEKEEREAYEANMKNAEREAEALEEARAKKREAGVEEESKDKDLFASLKKGGFPATEASTPGDSGAATPASDMSMGPPSKPASANKREKPAALKLETTKAVEPPQPSAAMKALHSARFVDDLSKITYPASIVSPNPALNASAPADRKFHYNKEFLLQFQSVFKEKPSIDWDARVRETVGDSDSSRPQSARTPMMGGRNSSRPGAAPFQMGNFGQTPSRASNLPPGTTSAERFAMSNAARTASMGNPFGNFGRPGLGIGAPGISRTNSASAMHPGMPSSPRVGSSNRSGTRTGSKRDKQNAKKEEEMAKSMPLTAGMEVKALQVSSTGWKPRSIGQAPAAAAGPTPGGATHMPPDMVQRKVKAALNKMTPENFDRIAGQLLEIVSQSKDETDGRTLRQVIQLTFEKATDEAHWASIYAKFCKRMLESMSAEIKDENIHDKNGNVVAGGSLFRKYLLNRCQEEFERGWKVNLPPKPEGETEEAAMMSDEYYKAAAAKRRGLGLVKFIGELYKLGMLTERIMHECVKKLVDYEGMPDEAEVESLTSLLRTIGASLDVSEKGHAFMNAYFHRIGLMIQTPGLPSRLRFMLMDIVDLRAARWQSKDADKGPKTIQQIREEAARAQQEAEMERLRQQANRGSRPGLGRGDARSYSGYGNQAPPQDFASSKVGSDDLRRLRTSRQTNQPMSFGPSSMLGSRSNSGRKNLGPGGNLVRGSDDSAASSRTGTPTGKKEDKEAASSINAFSALASLEDRDNMATSPPSNPTSPLLTKSQPAVERRPSKTPSKDGEEAS

SEQ ID NO: 38 - protein, 1725 aa, Cricetulus griseus
MGRPQGFRIPRGPVQQPLEDRIFPPTVSAVYSTVTQVARQPGPPTPSPYSAHEINKGHPNLAATPPGHASSPGLSQTPYPSGQNAGPTTLVYPQAPQTMNSQPQTRSPPSRTVPIHCTDNWKRRKVLEQTPVYRSLAGRGWIKYCIFAAGPRPAHHQFFQRPQIQPPRATIPNSSPSIRPGAQTPTAVYQANQHIMMVNHLPMPYPVPQGPQYCIPQYRHSGPPYVGPPQQYPVQPPGPGPFYPGPGPGDFANAYGTPFYPSQPVYQSAPIIVPTQQQPPPAKREKKTIRIRDPNQGGKDITEEIMSGGGSRNPTPPIGRPTSTPTPPQQLPSQVPEHSPVVYGTVESAHLAASTPVTAASDQKQEEKPKPDPVFQSPSTVLRLVLSGEKKEQASPVPETPAGEPTPELPRTSSPTTLAPLARSSVPSPTCAALSSQPLFTTALEDKRDLSSSQEDALPVPSPTSCTVTSDPSLTDDKNICKKPCSVAPHDIQLISTPNLMNEMNGVGEKLPAQESIVDMAKQETLPLTLELEILEHAQEEMKVECTPTAITPSMLTSFSPAPLTPPSSPCAPVISATTARSPVAATVVQRVAEEGESIRTCLSEDANETQYKTEAEADGQTEEIVDSQNLNSRRSPGPVQTAIIAPKTWKKAKDRSRTPDEVLEAEAEPKADEELPDDRVLEPETDKMSQGFHFERDSSALKRVKAGEENGEEVEPLRNGAESPSEGEGGDANSGSTDSSGDGIPFPFKAESWKPADTEGKKQYDREFLLDIQFMPACIQKPEGLPPISDVVLDKINQPRLPMRTLDPRILPRGPDFTPAFADFGRQTPGGRGVPLLNVGPRRSQPGQRREPRKIITVSVKEDVHLRKAENAWKPSQKRDSHADDPESIKTQELFRKVRSILNKLTPQMFNQLMKQVSGLTVDTEERLKGVIDLVFEKAIDEPSFSVAYANMCRCLVTLKVPMAEKPGNTVNFRKLLLNRCQKEFEKDKADDDVFEKKQKELEAASAPEERTRLHDELEEAKDKARRRSIGNIKFIGELFKLKMLTEAIMHDCVVKLLKNHDEESLECLCRLLTTIGKDLDFEKAKPRMDQYFNQMEKIVKERKTSSRIRFMLQDVIDLRLCNWVSRRADQGPKTIEQIHKEAKIEEQEEQRKVQQLMTKEKRRPGVQRVDEGGWNTVQGAKNSRVLDPSKFLKITKPTIDEKIQLVPKAQLGSWGKGSSGGAKASETDALRSSASSLNRFSPLQPPAPSGSPSTTPLEFDSRRALTSRGSMGREKSDKPLPTGAARPNTFLRGSSKDLLDNQSQEEQRREMLETVKQLTGGVDTERASSEADRSKTRELVKSEMCAVSPPDKPALSEEEVERKSKSIIDEFLHINDFKEATQCIEELSAQGPLHVFVKVGVEFTLERSQITRDHMGHLLYQLVQSEKLSKQDFFKGFSETLELADDMAIDIPHIWLYLAELVTPMLKEGGISMRELIVEFSKPLLPVGRAGVLLSEILRLLCKQMSHKKVGALWREADLSWKDFLPEGEDVHHFLLEQKLDFIETESSCSSEALSKRELSAEELSQRLEKLIMEDKADDERIFDWVEANLDESQMSSPTFLRALMTAVCKAAIIADCPTFRVDTAVIKQRVPILLKYLDSDTEKELQALYALQASIVKLDQPANLLRMFFDCLYDEEVISEDAFYKWESSKDPAEQNGKGVALKSVTAFFTWLREAEEESEDN

SEQ ID NO: 39 - protein, 1017 aa, Kluyveromyces lactis
MGEPTSEQQPAVEAPVVQEETISSPQKNSGYVKNTAGSGAPRNGKYDGNRKNSRPYNQRGNNNNNNNGSSSNKHYQKYNQPAYGVPAGYIPNYAVSAEYNPLYYNQYQQQQQLYAAAYQTPMSGQGYVAPVVSPAAVSAKPAKVEITNKSGEHIDIASIAHPHTHSHSQSHSRAVPVVSPPANAAVTAAASSSVSPSASPAVKVQSPAANDKEQSPAKPEEPKKDTLIVNDFLEQVKRRKAALAAKKAVEERGSEEQKEPVVGADTDANVDAKTGSTAADSTKSEEAQSESQEKTKEEAPAEPKPLTLAEKLRLKRMEAAKQASAKTEELKTEESKPEELKTEEPKAEEPKAEEPKLEEPKPEEPKTEEPKTEEPKTEEPKSEEPKSEEPKSDEPKSDEPKAEEPKTEELKTEEPKTEELKTEEPKTEETKPEEPKTEESATEANAKEGEPAPAGSVETPADVETKPQEETEVEDDGKITMTDFLQKLKEVSPVDDIYSFQYPSDITPPNDRYKKTSIKYAYGPDFLYQFKDKVDVKYDPAWMAEMTSKIVIPPKKPGSSGRGEDRFSKGKVGSLRSEGRSGSRSNSKKKSKRDDRKSNRSYTSRKDRERFREEEVEEPKVEVAPLVPSANRWVPKSKMKKTEVKLAPDGTELYDAEEASRKMKSLLNKLTLEMFEPISDDIMKIANQSKWEEKGETLKIVIQQIFNKACDEPHWSSMYAQLCGKVVKDLDDSIKDSETPDKTGSHLVLHYLVQRCQTEFQTGWTDQLPTNEDGTPLQPEMMSDEYYKMAAAKRRGLGLVRFIGFLYRSNLLTSRMVFFCFKRLMKDIQNSPTEDTLESVCELLETIGEQFEGARIQVTAEAVIEGSSLLDTLFDQIKNVIENGDISSRIKFKLIDIVELREKRNWNSKNKNDGPKTIAQIHEEEALKRALEERERERDRHGSRGGSRRMNSERNSSRRDFSSHSHSHNQNRDGFTTTRSSSVRYSEPKKEEQAPTPTKSSAGAANMFDALMDAEDD

SEQ ID NO: 40 - protein, 1013 aa, Ogataea polymorpha
MSNPNSVPPTGNAHHHYNSNYHKNRRNNGSYSNYSQSHPQPPYYQQQGWTGYMTQPYQAAAPGYAYSYYAPSPQGVSGGAYYAPPPPGAIAISPTLPNSATHTPVTKPVNLSIKNKNGEAVQFNKGHSKTASLSSSSSSSTPVLSKVNASLPAVPPSTSSTASVPMSPSKKDSSPAKPAQPEVKKANEEFKAKFLEQLRLKNSKATSEKKEEKSQPTKEVEKESKPDASSKKSEPPKELPQEISKEAPKPAEKPETSKEEPKEPAESAKPAKPAVPSASIEQQKEETKEIEKTQPNSKQAEKPEELQAVPESPVESIQPKPVAASAPSVDEVKEEAQPTPVKEEPINADSEQTEGPKSSPVADSQEDTKDETAEPLEAASPGIDKTFDMTRFFAQISKATPIKDIYAFPYKEGQEKPDLRLQNSPKNKYSPPFLLQFQPVCQFPVDDEWDAKVRSIIDLVGAKKESRDRGFSRSGSQFGRGGAGSSNAMPRSATLRGEFGSRNGSRGGSKKKMGLGSREKSQRQGSKKPRRLDDEKDNKDEETEAPKQVVEVKPLEKSANRWVPRSQRNKEKEVRYAPDGVTVILEEDDVEAKVKSHLNKLTLDNFDTISAEIIQIANQARWEDDVKTLKQVIELTFAKACDEHLWSSVYAKLCLTLCTDISEDVEDKSQPRPEQVPKRVYSSGLVRKLLLSRCQAEYEKGWSDKLPTNEDGTPLEPEMMSDEYYKLAAAKRRGLGLVRFIGELYYLNLLSEKIIIKCLVDQLSDSDSPSEDTLETLTQLLTTTGAKLDSSNRGPLDFAFEKINHIINNCQLSSRIKFKLMDLCDLRANRWADAKINAGPKTIAEIHQEAEAKRLQEERDAIRRRKADPRSGSSRSNSSRSNSKWGNSNQISSNDISKVGVVRNSSERSLGPFNKQKSKLSSSKLSQQQFTTVPSAGSANSSGASSGQKRAGTPPDEPREASRNIFAALGDHEDEDSDREAEHHEPEPEHEGADISSAHHEGVSAVADSESQA

SEQ ID NO: 41 - protein, 952 aa, Saccharomyces cerevisiae
MTDETAHPTQSASKQESAALKQTGDDQQESQQQRGYTNYNNGSNYTQKKPYNSNRPHQQRGGKFGPNRYNNRGNYNGGGSFRGGHMGANSSNVPWTGYYNNYPVYYQPQQMAAAGSAPANPIPVEEKSPVPTKIEITTKSGEHLDLKEQHKAKLQSQERSTVSPQPESKLKETSDSTSTSTPTPTPSTNDSKASSEENISEAEKTRRNFIEQVKLRKAALEKKRKEQLEGSSGNNNIPMKTTPENVEEKGSDKPEVTEKTKPAEEKSAEPEVKQETPAEEGEQGEKGQIKEESTPKVLTFAERLKLKKQQKEREEKTEGKENKEVPVQEETKSAIESAPVPPSEQVKEETEVAETEQSNIDESATTPAIPTKSDEAEAEVEAEAGDAGTKIGLEAEIETTTDETDDGTNTVSHILNVLKDATPIEDVFSFNYPEGIEGPDIKYKKEHVKYTYGPTFLLQFKDKLNVKADAEWVQSTASKIVIPPGMGRGNRSRDSGRFGNNSSRGHDFRNTSVRNMDDRANSRTSSKRRSKRMNDDRRSNRSYTSRRDRERGSYRNEEKREDDKPKEEVAPLVPSANRWVPKFKSKKTEKKLAPDGKTELLDKDEVERKMKSLLNKLTLEMFDAISSEILAIANISVWETNGETLKAVIEQIFLKACDEPHWSSMYAQLCGKVVKELNPDITDETNEGKTGPKLVLHYLVARCHAEFDKGWTDKLPTNEDGTPLEPEMMSEEYYAAASAKRRGLGLVRFIGFLYRLNLLTGKMMFECFRRLMKDLTDSPSEETLESVVELLNTVGEQFETDSFRTGQATLEGSQLLDSLFGILDNIIQTAKISSRIKFKLIDIKELRHDKNWNSDKKDNGPKTIQQIHEEEERQRQLKNNSRSNSRRTNNSSNRHSFRRDAPPASKDSFITTRTYSQRNSQRAPPPKEEPAAPTSTATNMFSALMGESDDEE

SEQ ID NO: 42 - protein, 1403 aa, Schizosaccharomyces pombe
MSSKPPSNTPKFSYARALASSQSNKSNSTKASENNTATAEKQAVKPSGVEPTNTSRANAQKKTESTGKITSEADTEKYNSSKSPVNKEGSVEKKSSEKSSTNNKPWRGDNTSKPSANSSAERTSSQHQKPETSSQIGKDNAAPVENVNEKSTSQETAPPVSTVPIQFGSITRNAAIPSKPKVSGNMQNKSGVSSYSSKSQSVNSSVTSNPPHTEEPVAAKPEASSTATKGPRPTTSASNTNTSPANGAPTNKPSTDINTTDPATQTTQVSASNSPALSGSSTPSNTSSRSNRQNHGNFSEKRHYDRYGNSHPSYNKYSHYQHGFNYNNSGNNRNESGHPRFRNSRRNYNNQGAYPTYMSNGRSANQSPRNNPQNVNNGSTPIQIPVSLQTPYGQVYGQPQYIVDPNMVQYGPILQPGYVPQYYPVYHQTPYTQNFPNMSRSGSQVSDQVVESPNSSTLSPRNGFAPIVKQQKKSSALKIVNPVTHTEVVVPQKNASSPNPSETNSRAETPTAAPPQISEEEASQRKDAIKLAIQQRIQEKAEAEAKRKAEEKARLEAEENAKREAEEQAKREAEEKAKREAEEKAKREAEEKAKREAEENAKREAEEKAKREAEEKAKREAEEKAKREAEEKAKREAEEKAKREAEEKAKREAEEKAKREAEENAKREAEEKAKREAEENAKREAEEKVKRETEENAKRKAEEEGKREADKNPEIKSSAPLASSEANVDTSKQTNATEPEVVDKTKVEKLKASEGKSTSSLSSPSHSTSSKRDLLSGLESLSLKTNPKSEQCLESLLNSQFITDFSALVYPSTIKPPSTEEALKAGKYEYDVPFLLQFQSVYTDKPMKGWDERMKETVASAFSDKSSRGMYSSSRQSSRSGSNTHSHAGPGFGGPSERKGISRLGIDRGFSSSGAGFGSGSNYKSAPSRGVSHHGHGGMSGSHRGSQRGSRRGGGERDKPDPSSLTIPVDQVAPLQLSANRWQPKKLTEKPAETKGEDEEALLPPEVVQRKVKGSLNKMTLEKFDKISDQILEIAMQSRKENDGRTLKQVIQLTFEKATDEPNFSNMYARFARKMMDSIDDSIRDEGVLDKNNQPVRGGLLFRKYLLSRCQEDFERGWKANLPSGKAGEAEIMSDEYYVAAAIKRRGLGLVRFIGELFKLSMLSEKIMHECIKRLLGNVTDPEEEEIESLCRLLMTVGVNIDATEKGHAAMDVYVLRMETITKIPNLPSRIKFMLMDVMDSRKNGWAVKNEVEKGPKTIAEIHEEAERKKALAESQRPSSGRMHGRDMNRGDSRMGGRGSNPPFSSSDWSNNKDGYARLGQGIRGLKSGTQGSHGPTSLSSMLKGGSVSRTPSRQNSALRREQSVRAPPSNVAVTSANSFELLEEHDHDNDGGQKDSNSKTSS

SEQ ID NO: 43 - protein, 1603 aa, Trichoderma reesei
MTSPANQQTPIPATTTNAPTAPSYASAAGAPKKPAQAPVVASGSNAPLVVGSSAPTASAQNAKAASSPSPVNGKQPVPPAVPTVARSSFNGSGPDHFRKGSVTMAANGPSGFVPNGGSIAGAKSGIQFGYDSPAMGHSTPHVGHSAPIPVPDANNHHRVPSPAHSPAPIPQPSASGGRPPSSLQQPTNQMTFGSLGSDGDRHMRQGSVPPNPNLANQSGAHFRRESSHSVQSDSSRGNFGPQNNRNRNSFNPHATQFNNQMGFPPQNQFRTPGQNRGMPPSFQPNPRGYPNSPQPARSPALANLVPSLPGTPNMPPATMQPNMAMPNAPAYHYPPPIPASHQQQVQYPFPAVAAKMHSKPSYKKSKSRREYDRHFSQGSPLDTAHDAPTSAAWPQQQQQQQHHQRRFGSQGQRADTQWRAHDKNNMRPISSGIQKDGPSSPSLPLVDAPLSHPNSLDLSPENGGFELWLTWKNQTYGFPPQQMNQYGQPMTVPYYGGAMPYMGPPQPGAQAYNQQFAPPTFHQQSHSMSRTPSQPERPASASQPSQPAIVSSNPKALNAQANPNSFVKPRKSAAVQIKNLAGEVVDTSTFKQPASPVPAQTRTPPVIAASTPPQQKLDAPSHARTDSQSPSKTAKEIQEELKAKIQAEIRAASEKAAKQEAGAKAGAAEDASAKKAEAAEAKVEEKTEAKAEAQPEVKAQPEATPDVKPEEPKAEPAETKAEAKAEPQPEAAAKAEAPAPEKAAEKPAEEPAEKATEKAAEKPAETPAEAPKAEEPPAKSEEPAKSDEPEEDELERIIREMEEAEAKREAQEEEHRKKREAEKAAQKAAEEANQKKNAEEADRKLREQEREMERLEEEKERRREAAESSGKTLSVAEALAQMRSGNAPDKKDEPASVEAVADKLADFSIAGDKSGDAAGQKEKRVAKPAALNLAPIKTSAVEPPQPSAALQSLKSARFLQVMDQDIYPEGIKSPNPALNAAVAKKGKTFKYDAAFLLQFQKVFTEQPSLQFHQQIKQLIGDGDGNRSASRGQTPSSARQGSRGGGGFPGGSFGVPGGRTLPPGTTSADRMAIASGALPRPQVNPMASFQRPGAAFPASSSMSRTSSQNMRNNLPNSPRQSSRSTRGSRRNDMAAKEAQAAKTMPLTAGQEVKPITVSASGWKASSLGKAPSAATPQPGQLMDPEMVQRKVKAALNKMTPENFERISEQILAIASQSKDETDGRTLRQVIQLTFEKATDEAHWASMYAKFCKRMLETMSPDIRDENIKDKNGNVVSGGNLFRKYLLNRCQKEFERGWTTNLPEAPKDTEEGEKKTGEAAMLSDEYYAAAAAKRRGLGLVQFIGELFKLGMLTERIMHECVTKLVDYKGMPDEAEIESLCKLLRTIGANLDETDKGRPLMDIYFQRIQGMADLPELQSRMRFMLMDVIDLRKARWVSKEGNKGPKTLEEVRAEAEAQSAAKAQEAARTSQRGGPGGGRPPAGRGDARGFSGSFNQPSNQVGMDDLRRLKGSSVNRTSSSNVTLGPTSMLSSRSNSGRRFGPGGALGRGGDDSNSSSRAGTPGPTSSNAFSLLATMDDGHPASPPSAGPSPLMAKAVPATDKKDNE

SEQ ID NO: 44 - protein, 904 aa, Yarrowia lipolytica
MSYTYNNGAYSPRNNQPGTPNQGQQHMAYSPAAMYNGGYGYVDMTQMQGQYYPPQEGMSPGAQYAQPYGSPHHTHSPLTIMDPVTHRPIVTPGSNTGTPTGPNATLASPKPNSPRVAHRRTDSSNPAGDSIKANFMAQVLAAKAKKEAAEKGTEKKEEVKEEKKEEPKPETKPEEEVKKEEPKKEEPKEDQKEEVKEELKETPKEDAKPVEAEKPKEDVKEEAKPVEAEKPAETKLEEESSAPAQEEKVEKKVEIVEPPKEPSAEGEPTEDASESTPNPVEKIAETMRLKKRFAHSWNNATPLTAEEITTKVTYPESVTKSLMEVSPAPTFRYSLDFLLQFQPLVTEQPPSGLEGAKAITEDCKGSKAPRASSRGSGGGPNQQMGSFSNFGRGGPGGPSGRGMPRMGSSSNMRGDHGGHHQSHRGGDRGDREGGGRSTRNNSSRNRRNPSNRERYPPQGANQEIIEYVPPPPKSENSWAAKRLAAKKETSTPPPAESEPAEEETPRIPPDVVQKKVNSMLNKMTLEKFDKISDQILEIASQSKHEHDGRTLRQVLELTFAKATDEAHWASMYARFCKKTMVTVDPDIYDDDVKDKNGEYVRGGALYRKYLLNRCQDEFEKGWEDKLPTNEDGSPMDPDNATPEYDKAVVAKRRGLGLMVFIGELFMLNMLSENIMRSCLTRLISPKDGASEETIESLCKLTSTVGLRMDQSPNSKEFMDMFFQRLTDTKNSGTLPSRLKFKILDLLEMRAGGWKQSNDQGPKTISEIQDEAAAAKEKQQQQQDRDRSHRGGNRRSGHPMGGRSESYRGGNERDRNTNSLNTGDLKQFGKIRSDSSNTFGPPSSMMGRGGSNASRNSSQTSLQQQGRQNSSSPGIPLSKDSSRASSKRGNIFEHLMGDGEGEEEN

SEQ ID NO: 45 - protein, 626 aa, Komagataella phaffii
MSVDTKEVQESLDQLNISESPATAVTEETPATSTEAAEESNESSTQASETLASLYVGELDPTVTESDLYEFFSPIGSVNSIRVCRDAVTKRSLGYGYVNFHSQAAGERALEELNYAEIKGVRCRLMWSQRDPSLRRSGSGNIFIKNLDPAIENKTLHDTFSSFGKVLSCKVATDENGNSKGFGFVHYESDEAAQAAIENINGMLLNGREIYVGPHLAKKDRESRFQEMIKNYTNVFVKNFDTESTEDELRELFESYGPITSIHLQVDSEGHNKGFGFVNFAEHDDAVKAVEALNDKEYKGKPLYVGRAQKKNERVHELTKKYEADRLEKLQKYQSVNLFIKNLDESIDDARLEEEFKPFGTITSAKVMLDENGKSRGFGFVCLSTPEEATKAISEMNQRMVANKPLYVALAQPKAIRRSQLAQQIQARNQMRMQQQAGPGIPNQFVQPIFYGQQPGMLPPGARVPPMGNQIPQFAGMPRPGPFPQGQFPRMAPNGQPMPVYGQPVFNGNGPQQRGYYPNNRQNKNRNQKEEGNSLAAILPQLPLEQQKRVLGEELYPKVVATNKTQDPEAAGKITGMILDLDNQEILQLLEDEELFNTHFNEALQAYEEYKSKTVEQQQQADAQAN

SEQ ID NO: 46 - protein, 626 aa, Komagataella pastoris
MSVDTKEVQESLDQLNISESPATAVTEETPATSTEAAEESNESSTQASETLASLYVGELDPTVTESDLYEFFSPIGSVNSIRVCRDAVTKRSLGYGYVNFHSQAAGERALEELNYAEIKGVRCRLMWSQRDPSLRRSGSGNIFIKNLDPAIENKTLHDTFSSFGKVLSCKVATDENGNSKGFGFVHYESDEAAQAAIENINGMLLNGREIYVGPHLAKKDRESRFQEMIKNYTNVFVKNFDTESTEDELRELFESYGPITSIHLQVDSEGHNKGFGFVNFAEHDDAVKAVEALNDKEFKGKPLYVGRAQKKNERVHELTKKYEADRLEKLQKYQSVNLFIKNLDESIDDTRLEEEFKPFGTITSAKVMLDENGKSRGFGFVCLSTPEEATKAISEMNQRMVANKPLYVALAQPKAIRRSQLAQQIQARNQMRMQQQAGPGIPNQFVQPIFYGQQPGMLPPGARVPPMGNQIPQFAGMPRPGPFPQGQFPRMAPNGQPMPVYGQPVFNGNGPQQRGYYPNNRQNKNRNQKEEGNSLAAILPQLPIEQQKRVLGEELYPKVVATNKAQDPEAAGKITGMILDLDNQEILQLLEDEELFNTHFNEALQAYEEYKSKTVEQQQQADAQAN

SEQ ID NO: 47 - protein, 636 aa, Homo sapiens
MNPSAPSYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKEREAELGARAKEFTNVYIKNFGEDMDDERLKDLFGKFGPALSVKVMTDESGKSKGFGFVSFERHEDAQKAVDEMNGKELNGKQIYVGRAQKKVERQTELKRKFEQMKQDRITRYQGVNLYVKNLDDGIDDERLRKEFSPFGTITSAKVMMEGGRSKGFGFVCFSSPEEATKAVTEMNGRIVATKPLYVALAQRKEERQAHLTNQYMQRMASVRAVPNPVINPYQPAPPSGYFMAAIPQTQNRAAYYPPSQIAQLRPSPRWTAQGARPHPFQNMPGAIRPAAPRPPFSTMRPASSQVPRVMSTQRVANTSTQTMGPRPAAAAAAATPAVRTVPQYKYAAGVRNPQQHLNAQPQVTMQQPAVHVQGQEPLTASMLASAPPQEQKQMLGERLFPLIQAMHPTLAGKITGMLLEIDNSELLHMLESPESLRSKVDEAVAVLQAHQAKEAAQKAVNSATGVPTV

SEQ ID NO: 48 - protein, 764 aa, Aspergillus niger
MSADVSTTPAAENVNGAAEASPAAAAPSATTPEVTAVENSTPAPAANQPHSASLYVGELDPSVTEAMLYELFSSIGQVASIRVCRDAVTRRSLGYAYVNYNNTADGERALEDLNYTLIKGKPCRIMWSQRDPALRKTGQGNVFIKNLDSAIDNKALHDTFAAFGNILSCKVAQDEFGNSKGYGFVHYETAEAANNAIKHVNGMLLNDKKVFVGHHISKKDRQSKFEEMKANFTNVYIKNLDSEIDDDEFRKMFEKFGEITSATLSRDQEGKSRGFGFVNFSTHESAQAAVEEMNDKEIRSQKLYVGRAQKKHEREEELRKQYEAARLEKASKYQGVNLYVKNLTDDIDDEKLRELFGPYGTITSAKVMRDTNVERESSPESAGKEKEANKENDKEATPEAEKAEEKAEEKPAESSEEKDKEAKKSDKKPFGKSKGFGFVCFSSPDEASKAVTEMNQRMVNGKPLYVALAQRKDVRRSQLEASIQARNTIRQQQAAAAAGMPQPYMQPAVFYGPGQQGFIPGGQRGMAFPPQPGMVMGIPGGRPGQYPGPFPGQQGGRGMGPNQQIPPNFAQGIPMGMQGPGAIPNGMGYPQMAQVQFGRGAGGRGQVPGMPMGQGMRGGPGYGQGRGGVPVQQGQIRPGQGGRGQNAAAPAGPQEGAAGGVNAQTLGAAPPAQQKQMLGEALYPKIQAQQPELAGKITGMLLEMDNTELLSLLEDEEALRAKVDEALSVYDEYMKNKGTESEAPAAEAAKPEAEKEASTEENKA

SEQ ID NO: 49 - protein, 636 aa, Cricetulus griseus
MNPSAPSYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKEREAELGARAKEFTNVYIKNFGEDMDDERLKDLFGKFGPALSVKVMTDESGKSKGFGFVSFERHEDAQKAVDEMNGKELNGKQIYVGRAQKKVERQTELKRKFEQMKQDRITRYQGVNLYVKNLDDGIDDERLRKEFSPFGTITSAKVMMEGGRSKGFGFVCFSSPEEATKAVTEMNGRIVATKPLYVALAQRKEERQAHLTNQYMQRMASVRAVPNPVINPYQPAPPSGYFMAAIPQTQNRAAYYPPSQIAQLRPSPRWTAQGARPHPFQNMPGAIRPAAPRPPFSTMRPASSQVPRVMSTQRVANTSTQTMGPRPAAAAAAATPAVRTVPQYKYAAGVRNPQQHLNAQPQVTMQQPAVHVQGQEPLTASMLASAPPQEQKQMLGERLFPLIQAMHPTLAGKITGMLLEIDNSELLHMLESPESLRSKVDEAVAVLQAHQAKEAAQKAVNSAAGVPSV

SEQ ID NO: 50 - protein, 592 aa, Kluyveromyces lactis
MSDITEKTAEQLENLQINDDQQPAQSASAPSTSASESEASSVSKVENNNASLYVGELDPNITEALLYDVFSPLGPISSIRVCRDAVTKASLGYAYVNYTDYEAGKKAIQELNYAEINGRPCRIMWSERDPAIRKKGSGNIFIKNLHPAIDNKALHETFSTFGEVLSCKVALDENGNSRGFGFVHFKEESDAKDAIEAVNGMLMNGLEVYVAMHVPKKDRISKLEEAKANFTNIYVKNIDVETTDEEFEQLFSQYGEIVSAALEKDAEGKPKGFGFVNFVDHNAAAKAVEELNGKEFKSQALYVGRAQKKYERAEELKKQYEQYRLEKLAKFQGVNLFIKNLDDSIDDEKLKEEFAPYGTITSARVMRDQEGNSKGFGFVCFSSPEEATKAMTEKNQQIVAGKPLYVAIAQRKDVRRSQLAQQIQARNQIRFQQQQQQQAAAAAAGMPGQYMPQMFYGVMAPRGFPGPNPGMNGPMGAGIPKNGMVPPPQQFAGRPNGPMYQGMPPQNQFPRHQQQHYIQQQKQRQALGEQLYKKVSAKIDDENAAGKITGMILDLPPQQVIQLLDNDEQFEQQFQEALAAYENFKKEQEAQA

SEQ ID NO: 51 - protein, 629 aa, Ogataea polymorpha
MSSAEINQITEQAEKLSVADPAAAETPATTAEESSTAESTQNSETLASLYVGELDPSVTESDLFEVFSPIGQVSTIRVCRDAVSKQSLGYAYVNFQSHADGEKALEELNYTPIKGKACRIMWSQRDPSLRRNGSGNIFIKNLHPAIDNKTLHDTFSAFGKILSCKIATDENGNSKGFGFVHYEDSESAKAAIENVNGMLLNDHEVYVGPHLAKKDRQSKMRELIANFTNVYVKNINLNWDEDKLRETFSPFGAISSIFLSKDESGKSRGFGFVNFEKHEDAVKAVEELNNKEIDGQKLYVGRAQKKSERMESLKHQYEAARQEQLNKYQGYNLFVKNLDDSIDDAKLEEEFKPYGTITSAKVMLDDAGKSKGFGFVCYSSPEEATKAITEMHQRMVAGKPLYVALAQRKEVRRSQLSQQIQARNQMRMQQAAAQGGMGQFVAPMFYGQNPGFLPPGARGAPFAAPGQQMMMQQGIPRPGQGVPVSPGQFRVGPNGQPVPMYMQPVFNEYPQNGRMPQQRYYQGGRPGQQPPQAGPRGKSEDGAVPSLAQVLPQFPPEQQKRMLGEELYPLIVATGKAQDPEAAGKITGMMLEMDNQQILALLEDRDLFNSQFEEALSAYEEYKNKEASA

SEQ ID NO: 52 - protein, 581 aa, Saccharomyces cerevisiae
GPDSMADITDKTAEQLENLNIQDDQKQAATGSESQSVENSSASLYVGDLEPSVSEAHLYDIFSPIGSVSSIRVCRDAITKTSLGYAYVNFNDHEAGRKAIEQLNYTPIKGRLCRIMWSQRDPSLRKKGSGNIFIKNLHPDIDNKALYDTFSVFGDILSSKIATDENGKSKGFGFVHFEEEGAAKEAIDALNGMLLNGQEIYVAPHLSRKERDSQLEETKAHYTNLYVKNINSETTDEQFQELFAKFGPIVSASLEKDADGKLKGFGFVNYEKHEDAVKAVEALNDSELNGEKLYVGRAQKKNERMHVLKKQYEAYRLEKMAKYQGVNLFVKNLDDSVDDEKLEEEFAPYGTITSAKVMRTENGKSKGFGFVCFSTPEEATKAITEKNQQIVAGKPLYVAIAQRKDVRRSQLAQQIQARNQMRYQQATAAAAAAAAGMPGQFMPPMFYGVMPPRGVPFNGPNPQQMNPMGGMPKNGMPPQFRNGPVYGVPPQGGFPRNANDNNQFYQQKQRQALGEQLYKKVSAKTSNEEAAGKITGMILDLPPQEVFPLLESDELFEQHYKEASAAYESFKKEQEQQTEQA

SEQ ID NO: 53 - protein, 653 aa, Schizosaccharomyces pombe
MPSTDLKKQADAAVESDVNTNNEAVESSTKEESSNTPSTETQPEKKAEEPEAAAEPSESTSTPTNASSVATPSGTAPTSASLYVGELDPSVTEAMLFELFNSIGPVASIRVCRDAVTRRSLGYAYVNFHNMEDGEKALDELNYTLIKGRPCRIMWSQRDPSLRKMGTGNVFIKNLDPAIDNKALHDTFSAFGKILSCKVAVDELGNAKGYGFVHFDSVESANAAIEHVNGMLLNDKKVYVGHHVSRRERQSKVEALKANFTNVYIKNLDTEITEQEFSDLFGQFGEITSLSLVKDQNDKPRGFGFVNYANHECAQKAVDELNDKEYKGKKLYVGRAQKKHEREEELRKRYEQMKLEKMNKYQGVNLFIKNLQDEVDDERLKAEFSAFGTITSAKIMTDEQGKSKGFGFVCYTTPEEANKAVTEMNQRMLAGKPLYVALAQRKEVRRSQLEAQIQARNQFRLQQQVAAAAGIPAVQYGATGPLIYGPGGYPIPAAVNGRGMPMVPGHNGPMPMYPGMPTQFPAGGPAPGYPGMNARGPVPAQGRPMMMPGSVPSAGPAEAEAVPAVPGMPERFTAADLAAVPEESRKQVLGELLYPKVFVREEKLSGKITGMLLEMPNSELLELLEDDSALNERVNEAIGVLQEFVDQEPGFTE

SEQ ID NO: 54 - protein, 745 aa, Trichoderma reesei
MAANTTSAAVDQLASDLNNTSLNGSGDVKAPAIDTSVSAGAEDASAPTPTAAPHPQNSASLYVGELDPSVTEAMLFELFSQIGAVASIRVCRDAVTRRSLGYAYVNYNSTPDGEKALEELNYTLIKGRPCRIMWSQRDPALRKTGQGNVFIKNLDVAIDNKALHDTFAAFGNILSCKVAQDENGNSKGYGFVHYETDEAAAQAIKHVNGMLLNEKKVYVGYHIPKKDRQSKFEEMKANFTNVYVKNIAPDVTDEDFRQLFEKFGDVTSSSLARDQEGKTRGFGFVNFTTHEAAFKAVEELNGKDFRGQDLYVGRAQKKHEREEELRKSYEAARLEKANKYQGVNLYIKNLDDDVDDEKLRQMFAEFGPITSAKVMRDVPQEGEEEAKDQEKDKENQKEGEKEGESAEGAEKKTEKKSDKKLGKSKGFGFVCFSNPDDATKAVAEMNQRMINNKPLYVALAQRKDVRKSQLEASIQARNQLRMQQAAAAAGMPQQYMQPPVFYAPGQQPGFIPQGGRGIPFPQPGMPMPNVQGGRPGQFPGYPQQGGRNMPQGLPPNMYSIPGQFPPQFGQPGTPQFMAAMQQAQQAAALAGGRGGAPQGGRGAMAGMPPNVQGGMPGYPPNNRQMGRNNNGGNGNNAGRNGNFPNQAPRDSNTASMLQNQLAAAQPAQQKQILGELIFPKIQAINSELAGKITGMLLEMDNSELVNLIEDEAALKAKVDEALAVYDEYVKSQATGSEKKEEETKA

SEQ ID NO: 55 - protein, 645 aa, Yarrowia lipolytica
MSADASLVAAEKALEKMTLENKAEASPATKEETTTEAAPAEGEAKTESSEEKGSKEDQGDNASLYVGELDPSVTEAMLFEIFNPIGPVTSVRVCRDAITRRSLGYAYVNFHNQADGIRALEELNYSPIKERPCRIMWSQRDPALRKTGAGNIYIKNLDPAIDNKALHDTFSAFGQILSCKIATDEFGNSRGFGFVHYESAESAESAIQHVNGMLLNDKKVFVGPHVPKSDRMQSFEEQKNSFTNVFIKNLGTEITEAEFEELVNKFGETSSVHLSTNDEGKPTGFGFVDYKEHDVAVKAIDGLSETEFKGNKLFAGRAKKKYERADELRKQYEASRLEKLNKYQGVNLYIKNLDDTIDDDKLRAEFAPHGTITSAKVMVDEAGKSKGFGFVCYSSPEEATKAVTEMNHRLVAGKPLYVVLAQRKDVRRSQLQQQIQAKNQMRLQQQAAAGGLPGQYMGNPGVFYPGQPGFMPPGRGGMPFGANPQMMMRPPMPPQNQFPPRGVPGGPNMYGAPPQGYQQGGFPPQGPMRGGQPPRSGQPGPQGQFRGAPRRKDGESRVADSISNALENAPEEQHKQLVGEALYPKVLAEKAIDGNAEFAGKITGMLLEMPIKEILEVIDDEEGLQAQINDAITAYNEYLNSQKEE

SEQ ID NO: 56 - protein, 606 aa, Komagataella phaffii
MSEKNTRIAIVSDDRCKPKKCRQECRKSCPVVKTGKLCIEVEAYSKIAFISETLCIGCGICVKKCPFGAINIINLPTNLEKEVTHRYSANSFKLHRLPTPRPGQVLGLVGTNGIGKSTALKILAGKQKPNLGRYDAPPDWEEILKYFRGSELQNYFTKVLEDNIKAIIKPQYVDNLPRAIKGPVNKVGELLKAKLERKEEGKHIIQVLELKHVLNREVAKLSGGELQRFAIGMTCVQDANVYMFDEPSSYLDVKQRLNAAQIIRSLLHATCYVIVVEHDLSVLDYLSDFVCILYGVPSVYGVVTLPASVREGINIFLDGHIPTENLRFRTESLKFKLTDASDSVLIEQSRAFEYPKMVKTQGDFKLTVNAGDFSDSEIIVMMGENGTGKTTLCKLLAGAIEGDEGTKVPKLNVSMKPQKIAPKFTGTVRQLFLKKIRSSFLHPQFNTDVMKPLKVDDVIDQEVQHLSGGELQRVAIVLALGKEADIYLIDEPSAYLDSEQRIICSKVIRRFILHSKKTAFIVEHDFIMATYLADKVMVFEGQPSKNAVANRPETLLTGCNRFLKNLNVTFRRDPNSFRPRINKLDSQMDREQKLSGNYFFLENTEL

SEQ ID NO: 57 - protein, 606 aa, Komagataella pastoris; identical to SEQ ID NO: 56

SEQ ID NO: 58 - protein, 599 aa, Homo sapiens
MADKLTRIAIVNHDKCKPKKCRQECKKSCPVVRMGKLCIEVTPQSKIAWISETLCIGCGICIKKCPFGALSIVNLPSNLEKETTHRYCANAFKLHRLPIPRPGEVLGLVGTNGIGKSTALKILAGKQKPNLGKYDDPPDWQEILTYFRGSELQNYFTKILEDDLKAIIKPQYVDQIPKAAKGTVGSILDRKDETKTQAIVCQQLDLTHLKERNVEDLSGGELQRFACAVVCIQKADIFMFDEPSSYLDVKQRLKAAITIRSLINPDRYIIVVEHDLSVLDYLSDFICCLYGVPSAYGVVTMPFSVREGINIFLDGYVPTENLRFRDASLVFKVAETANEEEVKKMCMYKYPGMKKKMGEFELAIVAGEFTDSEIMVMLGENGTGKTTFIRMLAGRLKPDEGGEVPVLNVSYKPQKISPKSTGSVRQLLHEKIRDAYTHPQFVTDVMKPLQIENIIDQEVQTLSGGELQRVALALCLGKPADVYLIDEPSAYLDSEQRLMAARVVKRFILHAKKTAFVVEHDFIMATYLADRVIVFDGVPSKNTVANSPQTLLAGMNKFLSQLEITFRRDPNNYRPRINKLNSIKDVEQKKSGNYFFLDD

SEQ ID NO: 59 - protein, 587 aa, Aspergillus niger
MKKCRQECKKSCPVVRTGKLCIEVTPESKIAFISERLCIGCGICPKKCPFGAIHIINLPTNLETQVTHRYSANSFKLHRLPMPRPGQVLGLVGTNGIGKSTALKILSGKLKPNLGRFDNPPDWEEILKYFRGSELQNYFTKVLEDDLKAVVKPQYVDQIPRAVKGPVQNVGALIKARAQLDNLDHILDTLELRQVQDRDIGHLSGGELQRFAIGLVCVQKADVYMFDEPSSYLDVKQRLAAARSIRELLRPDDYVIVVEHDLSVLDYLSDFVCVLYGRPALYGVVTLPASVREGINIFLDGHIPTENLRFREESLTFRLAEAGDDFMVDRARAFAYPSMEKTLGNFHLKIDAGSFTDSEIIVMMGENGTGKTTFCKMLAGAEKPDGGKSVPRLNISMKPQKITPKFQGTVRQLFFKRIKAAFLSPQFQTDVYKPLKIDDFIDQEVQNLSGGELQRVAIVLALGIPADIYLIDEPSAYLDSEQRIVASRVIKRFIMHTKKTAFIVEHDFIMATYLADRVIVFDGQPSVDAHANAPESLVTGCNTFLKNLDVTFRRDPNSYRPRINKYQSQMDQEQKLAGNYFFLEEEK

SEQ ID NO: 60 - protein, 599 aa, Cricetulus griseus
MADKLTRIAIVNHDKCKPKKCRQECKKSCPVVRMGKLCIEVTPQSKIAWISETLCIGCGICIKKCPFGALSIVNLPSNLEKETTHRYCANAFKLHRLPIPRPGEVLGLVGTNGIGKSTALKILAGKQKPNLGKYDDPPDWQEILTYFRGSELQNYFTKILEDDLKAIIKPQYVDQIPKAAKGTVGSILDRKDETKTQAIVCQQLDLTHLKERNVEDLSGGELQRFACAVVCIQKADIFMFDEPSSYLDVKQRLKAAITIRSLINPDRYIIVVEHDLSVLDYLSDFICCLYGVPSAYGVVTMPFSVREGINIFLDGYVPTENLRFRDASLVFKVAETANEEEVKKMCMYKYPEMKKKMGEFELAIVAGEFTDSEIMVMLGENGTGKTTFIRMLAGRLKPDEGGAVPILNVSYKPQKISPKSTGSVRQLLHEKIRDAYTHPQFVTDVMKPLQIENIIDQEVQTLSGGELQRVALALCLGKPADVYLIDEPSAYLDSEQRLMAARVVKRFILHAKKTAFVVEHDFIMATYLADRVIVFDGVPSKNTVANSPQTLLAGMNKFLSQLEITFRRDPNNYRPRINKLNSIKDVEQKKSGNYFFLDD

SEQ ID NO: 61 - protein, 607 aa, Kluyveromyces lactis
MSSKNTRIAIVSEDKCKPKKCRQECKRSCPVVKTGKLCIEVMPTSKIAFISENLCIGCGICVKKCPFDAIQIINLPTNLDSQVTHRYSANSFKLHRLPTPRPGQVLGLVGTNGIGKSTALKILAGKQKPNLGRFEDPPEWQEIIKYFRGSELQNYFTKMLEDDIKAIIKPQYVDNIPRAIKGPVQKVGELLKLRLEKSKDDAKNYIDILQLKNVLNREVSALSGGELQRFAIGMSCVQEADVYMFDEPSSYLDVKQRLNAALIIRSLLKPTTYVICVEHDLSVLDYLSDFVCIIYGVPSVYGVVTLPSSVREGINIFLDGHIPSENLRFRTEALQFRMADAGDELEADASRAFTYPDMKRTQGDFSLTVEAGEFSDSQILVMMGENGTGKTTLIKLLAGAIPADDGQDIPKLNVSMKPQKIAPKFTGTVRQLFFKRIRAQFLNPQFQTDVVKPLKIDDIIDQEVQHLSGGELQRVAIVLSLGMNADIYLIDEPSAYLDSEQRIICSKVIRRFILHNKKTAFIVEHDFIMATYLADKVIVFDGTPSKQAFARAPESLLTGCNKFLKNLNVTFRRDPTSFRPRINKLDSQMDKEQKSSGNYFFLDHNGV

SEQ ID NO: 62 - protein, 608 aa, Saccharomyces cerevisiae
MSDKNSRIAIVSADKCKPKKCRQECKRSCPVVKTGKLCIEVTPTSKIAFISEILCIGCGICVKKCPFDAIQIINLPTNLEAHVTHRYSANSFKLHRLPTPRPGQVLGLVGTNGIGKSTALKILAGKQKPNLGRFDDPPEWQEIIKYFRGSELQNYFTKMLEDDIKAIIKPQYVDNIPRAIKGPVQKVGELLKLRMEKSPEDVKRYIKILQLENVLKRDIEKLSGGELQRFAIGMSCVQEADVYMFDEPSSYLDVKQRLNAAQIIRSLLAPTKYVICVEHDLSVLDYLSDFVCIIYGVPSVYGVVTLPASVREGINIFLDGHIPAENLRFRTEALQFRIADATEDLQNDSASRAFSYPSLKKTQGDFVLNVEEGEFSDSEILVMMGENGTGKTTLIKLLAGALKPDEGQDIPKLNVSMKPQKIAPKFPGTVRQLFFKKIRGQFLNPQFQTDVVKPLRIDDIIDQEVQHLSGGELQRVAIVLALGIPADIYLIDEPSAYLDSEQRIICSKVIRRFILHNKKTAFIVEHDFIMATYLADKVIVFEGIPSKNAHARAPESLLTGCNRFLKNLNVTFRRDPNSFRPRINKLDSQMDKEQKSSGNYFFLDNTGI

SEQ ID NO: 63 - protein, 593 aa, Schizosaccharomyces pombe
MSESLTRIAIVSEDKCRPKKCRQECRRSCPVVRTGKLCIEVNPTDRIAFISETLCIGCGICVKKCPFGAINIINLPTNLESEVTHRYSANSFKLHRLPTPRPGQVLGLVGTNGIGKSTALKILSGKMKPNLGRYDNPPDWAEVVKYFRGSELQNFFTKVVEDNIKALIKPQYVDHIPRAIKTGDKTVSGLIKARANNNNFEEVMDHTDLQNLLNREVGHLSGGELQRFAIAAVATQKADVYMFDEPSSYLDIKQRLKAGRVIRSLLATTNYVIVVEHDLSVLDYLSDFVCVLYGVPSMYGVVTLPYSVREGINIFLDGHIPTENLRFRSEALTFRLADASDEITADRTAEYNYPDHVIEQGDFKLTIKSGGFSDAEIIVLLGENGTGKTTFCKWMAKNSDLKISMKPQTIAPKFQGTVRMLFLKKIRAAFLNGKFQSEVCKPLSIDNIIDQEVLNLSGGELQRVAICLALGMPADVYLIDEPSAYLDSEQRIIASKVIRRFIVNSRKTAFIVEHDFIMATYLADRVILFEGQPSRDARCNPPQSLLTGMNTFLKNLDVTFRRDPNTLRPRINKFDSQMDQEQKNAGNYFFLEN

SEQ ID NO: 64 - protein, 253 aa, Trichoderma reesei
MEKTLGNFRLRIDAGEFTDSEIIVMMGENGTGKTTFCRLLAGALKPDSKRSVPDMKISMKPQTITPKFEGTVRQLFFKKIKAAFLSPQFQTDVVKPLKLDDFIDQEVKNLSGGELQRVAIVLALGMPADIYLIDEPSAYLDSEQRIIASRVIKRFIMHSKKTAFVVEHDFIMATYLADRVIVFDGKPGIDAHANRPESLLTGCNTFLKNLDVTFRRDPTNYRPRINKLGSQLDQEQKLSGNFFFLEEKPEETA

SEQ ID NO: 65 - protein, 616 aa, Yarrowia lipolytica
MPPKNKNEVKDGDKNTRIAIVNVDKCKPKKCRQECKKSCPVVRTGKLCIEVTPDSKIAFISETLCIGCGICPKKCPFGALQIINLPTNLDSEVTHRYSANSFKLHRLPTPRPGQVLGLVGTNGIGKSTALKILAGKQKPNLGRYEDPPTWQEILTHFRGSELQNYFTRVLEDNLKAIIKPQYVDNLPRAIKGPLQKVGELLKAKLERKDDFENIVNTLELKGVWKREVSLLSGGELQRFAIGMSCVQLAQVYMFDEPSSYLDVKQRLHAAAMIRSLIDSTGYIIVVEHDLSVLDYLSDFVCILYGVPSVYGVVTLPSSVREGINIFLDGHIPTENLRFRTESLQFRLADATEIIPLDQSRSFKYPTMSRTQGDFKLNVESGEFTDSEIIVMMGENGTGKTTLCRLLAGAVAPDEGQKAPKLNVSMKPQKIAPKFTGTVRQLFLKKIRSSMHHPQFQTDVYKPLRIDDIIDQEVQNLSGGELQRVAIVLALGQPADIYLIDEPSAYLDSEQRIICAKVIRRFILHSKKTAFIVEHDFIMATYLADRVIVFDGQPSVEAFARRPESLLTGCNRFLKSLNVTFRRDPTSFRPRINKKDSQMDKEQKAHGNYFFLDTAEA

SEQ ID NO: 66 - DNA, 609 bp, Komagataella phaffii
atgtcagagactgaaaacgctcctcacgaaaagtcactgcttgacgaccaagttgatttcactgttaagcatccgctcaactctgagtggacactatggtatacgaagccagccgttcatgaacaagaaagttggtcagatttgctacgtcctatcatttcattcaactcggtagaggagttctggggaatcttcaactccattccaaaggcctccgatttgccagtcaagtcggactatcatctttttagagatggaatcaaacctgaatgggaagacgagcgcaatgctaacggaggaaaactgacataccagtttgcctacagaaaggttgatatcaatgaattgtggtctcgcggtctgttgagtgtgattggtgaaacgattcaggatgatgacggagaaactgaggtgaacggtattgttctgagcattcgtcgtgccgctttgaagattgccctctggaccaagtccaaggatgaggcagttttgagaccaattggcgagagattcaagaaagttttgaagttgacagcccgtgatcatattgactttcaacctcataagagtggtgctggtagcaagaacgtaccttctttcagcatttag

SEQ ID NO: 67 - DNA, 609 bp, artificial
  eIF4E gene mutant
atgtcagagactgaaaacgctcctcacgaaaagtcactgcttgacgaccaagttgatttcactgttaagcatccgctcaactctgagtggacactatggtatacgaagccagccgttcatgaacaagaaagttggtcagatttgctacgtcctatcatttcattcaactcggtagaggagttctggggaatcttcaactccattccaaaggcctccgatttgccagtcaagtcggactatcatctttttagagatggaatcaaacctgaatgggaggacgagcgcaatgctaacggaggaaaactgacataccagtttgcctacagaaaggttgatatcaatgaattgtggtcccgcggtctgttgagtgtgattggtgaaacgattcaggatgatgacggagaaactgaggtgaacggtattgttctgagcattcgtcgtgccgctttgaagattgccctctggaccaagtccaaggatgaggcagttttaagaccaattggcgagagattcaagaaagttttgaagttgacagcccgtgatcatattgactttcaacctcataagagtggtgctggtagcaagaacgtaccttctttcagcatttag

SEQ ID NO: 68 - DNA, 1212 bp, Komagataella phaffii
atgtctgaaggtattattgaaatcgacactaacttaatcgagaccaactacgaccaggttgtcagcaccttcgatgagctgaacctgaaggagcaaatcctgagagccgtttttgcttacggttacgagttcccatctgccattcaatctcgtgccattttgccaattgttgctggtcgtgatgtcttggcccaagcccagtccggtaccggtaagactgctacctttgccatctccgctttgcagagattggatgagaagctgaaggctgcccaagttttgatcttggccccaaccagagagttggccctgcagatcaagaaggttgttttggccctcggtatgcacttgaacatctctgtccatgcctgtatcggtggtgttgacccagctttggatgtcaaggccttcaactccggtgttcaaatcgtcgttggtaccccaggtagagtccaagacatgatcgaacgtggagctttcagaactgagaacgtcaagatggtcattttggatgaggccgatgagatgttgtccactggattcaaggagcaaatctacaccttgttcacattgttaccatccactacacaggttgttcttctatccgccaccatgcctcaagaggtgttggaggttaccaccaagttcatgaacaaccctattcgtatcttggtcaagaaggaccaattgaccttggaaggtattaagcaattctacgtcgacgtcggtgaagaacaatacaagttcgactgtctgtgtgatctgtacgactccatctccgtcacccaggccgttatcttctgtaacaccagacgaaaggtcgaggagttgactgagcgtttgaccgcccagaagttcaccgtctctgctatccactctgagctgtcacaaaccgagagagacaccatcatgcaagagttcagaaccggttcttccagaatcttaatctccaccgacttgctggccagaggtatcgatgtccaacaagtctcattggttatcaactacgacttaccatctaacaaggaaaactacatccacagaatcggtagaggtggtcgtttcggtagaaagggtgttgctatcaacttcgtcaccagcaaggacgttgagatgatgaaggagatcgagaagttctactctacccaaatcgacgagttgccatcttccatcactgacttgtttgagactgaggaagttaatgagtcttag

SEQ ID NO: 69 - DNA, 1212 bp, artificial
  eIF4A gene mutant
atgtctgaaggtattattgaaatcgacactaacttaatcgagacaaactacgaccaggttgtcagcaccttcgatgagctgaacctgaaggagcaaatcctgagagccgtttttgcttacggttacgagttcccatctgccattcaatctcgtgccattttgccaattgttgctggtcgtgatgtcttggcccaagcccagtccggtaccggtaagactgctacctttgccatctccgctttgcagagattggatgagaagctgaaggctgcccaagttttgatcttggccccaaccagagagttggccctgcagatcaagaaggttgttttggccctcggtatgcacttgaacatctctgtccatgcctgtatcggtggtgttgacccagctttggatgtcaaggccttcaactccggtgttcaaatcgtcgttggtaccccaggtagagtccaagacatgatcgaacgtggagctttcagaactgagaacgtcaagatggtcattttggatgaggccgatgagatgttgtccactggattcaaggagcaaatctacaccttgttcacattgttaccatccactacacaggttgttcttctatccgccaccatgcctcaagaggtgttggaggttaccaccaagttcatgaacaaccctattcgtatcttggtcaagaaggaccaattgaccttggaaggtattaagcaattctacgtcgacgtcggtgaagaacaatacaagttcgactgtctgtgtgatctgtacgactccatctccgtcacccaggccgttatcttctgtaacaccagacgaaaggtcgaggagttgactgagcgtttgaccgcccagaagttcaccgtctctgctatccactctgagctgtcacaaaccgagagagacaccatcatgcaagagttcagaaccggttcttccagaatcttaatctccaccgacttgctggccagaggtatcgatgtccaacaagtctcattggttatcaactacgacttaccatctaacaaggaaaactacatccacagaatcggtagaggtggtcgtttcggtagaaagggtgttgctatcaacttcgtcaccagcaaggacgttgagatgatgaaggagatcgagaagttctactctacccaaatcgacgagttgccatcttccatcactgacttgtttgagactgaggaagttaatgagtcttag

SEQ ID NO: 70 - DNA, 1461 bp, Komagataella phaffii
atgcatccatacaccgtagtatttgcgcgcctcctcctgggtgttttctcaactgccgcccccaactcgcccccccccttttgcacttcttgccaccttggagatcagagattgtggaaccttgctcaaaatctggcatgcacactacctcctcaaaaaaattttccttgccttgttgcaaggcgctcaaaaatcaaaagtttttctacaacttttgttcccaacaaatcacctttttacttacccattatgtctgaaggtattattgaaatcgacactaacttaatcgagaccaactacgaccaggttgtcagcaccttcgatgagctgaacctgaaggagcaaatcctgagagccgtttttgcttacggttacgagttcccatctgccattcaatctcgtgccattttgccaattgttgctggtcgtgatgtcttggcccaagcccagtccggtaccggtaagactgctacctttgccatctccgctttgcagagattggatgagaagctgaaggctgcccaagttttgatcttggccccaaccagagagttggccctgcagatcaagaaggttgttttggccctcggtatgcacttgaacatctctgtccatgcctgtatcggtggtgttgacccagctttggatgtcaaggccttcaactccggtgttcaaatcgtcgttggtaccccaggtagagtccaagacatgatcgaacgtggagctttcagaactgagaacgtcaagatggtcattttggatgaggccgatgagatgttgtccactggattcaaggagcaaatctacaccttgttcacattgttaccatccactacacaggttgttcttctatccgccaccatgcctcaagaggtgttggaggttaccaccaagttcatgaacaaccctattcgtatcttggtcaagaaggaccaattgaccttggaaggtattaagcaattctacgtcgacgtcggtgaagaacaatacaagttcgactgtctgtgtgatctgtacgactccatctccgtcacccaggccgttatcttctgtaacaccagacgaaaggtcgaggagttgactgagcgtttgaccgcccagaagttcaccgtctctgctatccactctgagctgtcacaaaccgagagagacaccatcatgcaagagttcagaaccggttcttccagaatcttaatctccaccgacttgctggccagaggtatcgatgtccaacaagtctcattggttatcaactacgacttaccatctaacaaggaaaactacatccacagaatcggtagaggtggtcgtttcggtagaaagggtgttgctatcaacttcgtcaccagcaaggacgttgagatgatgaaggagatcgagaagttctactctacccaaatcgacgagttgccatcttccatcactgacttgtttgagactgaggaagttaatgagtcttag

SEQ ID NO: 71 - DNA, 1461 bp, Artificial Sequence
  eIF4A gene mutant
atgcatccatacaccgtagtatttgcgcgcctcctcctgggtgttttctcaactgccgcccccaactcgcccccccccttttgcacttcttgccaccttggagatcagagattgtggaaccttgctcaaaatctggcatgcacactacctcctcaaaaaaattttccttgccttgttgcaaggcgctcaaaaatcaaaagtttttctacaacttttgttcccaacaaatcacctttttacttacccattatgtctgaaggtattattgaaatcgacactaacttaatcgagacaaactacgaccaggttgtcagcaccttcgatgagctgaacctgaaggagcaaatcctgagagccgtttttgcttacggttacgagttcccatctgccattcaatctcgtgccattttgccaattgttgctggtcgtgatgtcttggcccaagcccagtccggtaccggtaagactgctacctttgccatctccgctttgcagagattggatgagaagctgaaggctgcccaagttttgatcttggccccaaccagagagttggccctgcagatcaagaaggttgttttggccctcggtatgcacttgaacatctctgtccatgcctgtatcggtggtgttgacccagctttggatgtcaaggccttcaactccggtgttcaaatcgtcgttggtaccccaggtagagtccaagacatgatcgaacgtggagctttcagaactgagaacgtcaagatggtcattttggatgaggccgatgagatgttgtccactggattcaaggagcaaatctacaccttgttcacattgttaccatccactacacaggttgttcttctatccgccaccatgcctcaagaggtgttggaggttaccaccaagttcatgaacaaccctattcgtatcttggtcaagaaggaccaattgaccttggaaggtattaagcaattctacgtcgacgtcggtgaagaacaatacaagttcgactgtctgtgtgatctgtacgactccatctccgtcacccaggccgttatcttctgtaacaccagacgaaaggtcgaggagttgactgagcgtttgaccgcccagaagttcaccgtctctgctatccactctgagctgtcacaaaccgagagagacaccatcatgcaagagttcagaaccggttcttccagaatcttaatctccaccgacttgctggccagaggtatcgatgtccaacaagtctcattggttatcaactacgacttaccatctaacaaggaaaactacatccacagaatcggtagaggtggtcgtttcggtagaaagggtgttgctatcaacttcgtcaccagcaaggacgttgagatgatgaaggagatcgagaagttctactctacccaaatcgacgagttgccatcttccatcactgacttgtttgagactgaggaagttaatgagtcttag

SEQ ID NO: 72 - DNA, 3297 bp, Komagataella phaffii
atgtccaataagaacgtggatacagctccagataatggttctggtggctccaattccccagcttctgtgccgcattatccacagcaacaccgtgctaattataatcctcataagaatcatactaatcatgggggcaataatggaggcgcccagggttacatgcagcatcagcaacagcaacaaatgtattacaatcagatgggctaccctatgccgtatccgaatgctggtggcaatttcaaccagaatgtatacttggttccttataatactatggttccaggtactccatatggtgcaggtgccccgggcgtgcctccaagctacccagtgtctccttctccagcattcgcctctccacaaaagactcatgtgccagtaagtctgcgtaccccttctggagaacctataaatttggttcctgctgggcaaacgagttccagttcatccaatgcctctcctcatgttaagtctacctctattcctccttctgctgtcactagtcctgctgcttccattgctccaactgttgcatcaccgactaaggccgcggctggatctgaagacgctggcaagactagagatgcattcaagcagatggttcaggctttagctgctaaaaagaaggctgcagctgctgccaaggctgaaggtaaaacagatgataaaaaggagtctgtggagtctaagcctaagtctgctgtacagcccgagacaaagccaaaagttgaggttaagttagagccaaaaccaaaagttgaggtcaaattagagccaaagccaaaggttgagattaaactagagcccaaaccagtatcggaggctaaaccagagacaaaaacagtacctgaggttgaagaaccttcaacagttgcaacgatcaaatcagaatccacaccaattgaggagctcaaacccaaagttgaggtaaccccagaagttgcatccaagcctgaacaggttgagtctgcacaggagccggctaacgatgtaaaaccatctgaagtctcagctaaagaggttcaagaatccaaggaggagtctttgaaacaacctaaggaagctttgaatgacattaaatccaccgaggagtcaaataaaaagttttctcttccagaagaggagtctaacgatcaccagacactttcccaaattcaaggagttaaagatacagaacaatcgcaagaatttgaagaacctcaaggaacccaggaaactcaggaaactcaggaaacgcaggaaaccgcaaagcctttggaagctcaagcacctcaagaaattcaagaaacccaagaacctgaggaagttcaggaacaaactcctgaaacatctgagactgtttcttctgtcacagaagaagtgtctccagctccagcacctggattgcccctttcaaagttcttggacagaatcagaaaggctcccgccatttcaaacccattcaagcatgagtactcaggtgatttccatggtccagacgaaagtttaagtggtctgaaaaccatcaaatatgatccgggtttcttgatccaattcgccaccttcaacttccatattgatgaagatttcatcaacgatgtcggttctaaggttgtgatccctgtcaacaccagaagagacaatttttccaagagcatgtctaccagcaatttcaaaggttctggtagaaatatgggttctaatttcagaaatgattttggatctagaaacaactccaagcaaggttccaagaaaaagaataaatctcaaaagagccaatctaggaggggtccaaagtactccaatgaagaagaacctgctgaaccagccgtgcctgctgaattgcctaatagagctgcaagcaagtgggtacctcgttcgttaagaaataaggaggctgaagtcaagactgctccagacggatctataatagtatcagaagaagacatcaaaaggaaaactaaatcccttttgaataagttgacgttggaattctttgatgatatctcaaacgatataattgctttgaccaagcaagctcaatgggaagatgacgtgaagactttgaaacaagttattgagtctatatttgcaaaggcttgtgacgaaccctactggtcttctatgtacgctaaattatgcgccaaaatgtgcaaggacaccccacctgagatcaaggaaactaatgagaagggaaatactttcaccggtggtgatttggtgagaagagtgttgattaatagatgtcaaaccgaatatcagaaaggatggattgaccagctaccaaccaacgaggatggttcacctctagagccagaactgatgtctgacgaatattacaaacaagctgctgctaaacgtagaggtcttggtctggttaagttcattggtcaattgtaccagttgaacttattatcagacaatgtcattttccactgtgtccttggtcagtccaaagaaacagacaatccatcagaagatactatggagaacctggttcaattattggaagctgtaggttatagaatggagatgtccagtgaacctaagattagaagggttcttgatctcgcctttgccaacatcaaggccattgttgacgctaacaagattccttcaagaataagatttatgctcatggatttgatggatctaagaagaaatggctgggctacaacagaagagaacgctggtccaaagaccattcaagagattcacttagaggctgagatcaagagatcagaggaggagagagaaaaagaaagagacaggcattctcgtagaggacgttacaacgactccaaacctaactctcgtcaaaagtcatcttggggaagttcagtggtttcacctaatgatattagaaatgcaggctatgttagaaactctgacaaatctctggggcctgtcaacagtttttccaggggcaagtcatcccgtcaagcttcaaagcaatcaaacagtgattttaccagtgttcctatcacttccagtggtagtagaactgctctaaaccactctggcgacagctcaacgtctctgaacgaacctcaatcaagagaatattcaaaaaaatctgaatctaggcccgctccttccaacatttttgctgcattagaggatcacgatcatgatcatgaacatggccacgaacaagaagaaaacaagcattccaaggatctccaaaacggagaggccgaagcacctgaggacgacaatgtgggagaaatagcccaagctgacgaaaatgtgcaagggcaagagcaagaggaagaacgagaggaagagcgagagcaagaagaggaacaggaagtttaa

SEQ ID NO: 73 - DNA, 3297 bp, Artificial Sequence
  eIF4G gene mutant
atgtccaataagaacgtggatacagctccagataatggttctggtggctccaattccccagcttctgtgccgcattatccacagcaacaccgtgctaattataatcctcataagaatcatactaatcatgggggcaataatggaggcgcccagggttacatgcagcatcagcaacagcaacaaatgtattacaatcagatgggctaccctatgccgtatccgaatgctggtggcaatttcaaccagaatgtatacttggttccttataatactatggttccaggtactccatatggtgcaggtgccccgggcgtgcctccaagctacccagtgtctccttctccagcattcgcctctccacaaaagactcatgtgccagtaagtctgcgtaccccttctggagaacctataaatttggttcctgctgggcaaacgagttccagttcatccaatgcctctcctcatgttaagtctacctctattcctccttctgctgtcactagtcctgctgcttccattgctccaactgttgcatcaccgactaaggccgcggctggatctgaggacgctggcaagactagagatgcattcaagcagatggttcaggctttagctgctaaaaagaaggctgcagctgctgccaaggctgaaggtaaaacagatgataaaaaggagtctgtggagtctaagcctaagtctgctgtacagcccgagacaaagccaaaagttgaggttaagttagagccaaaaccaaaagttgaggtcaaattagagccaaagccaaaggttgagattaaactagagcccaaaccagtatcggaggctaaaccagagacaaaaacagtacctgaggttgaagaaccttcaacagttgcaacgatcaaatcagaatccacaccaattgaggagctcaaacccaaagttgaggtaaccccagaagttgcatccaagcctgaacaggttgagtctgcacaggagccggctaacgatgtaaaaccatctgaagtctcagctaaagaggttcaagaatccaaggaggagtctttgaaacaacctaaggaagctttgaatgacattaaatccaccgaggagtcaaataaaaagttttctcttccagaagaggagtctaacgatcaccagacactttcccaaattcaaggagttaaagatacagaacaatcgcaagaatttgaagaacctcaaggaacccaggaaactcaggaaactcaggaaacgcaggaaaccgcaaagcctttggaagctcaagcacctcaagaaattcaagaaacccaagaacctgaggaagttcaggaacaaactcctgaaacatctgagactgtttcttctgtcacagaagaagtgtctccagctccagcacctggattgcccctttcaaagttcttggacagaatcagaaaggctcccgccatttcaaacccattcaagcatgagtactcaggtgatttccatggtccagacgaaagtttaagtggtctgaaaaccatcaaatatgatccgggtttcttgatccaattcgccaccttcaacttccatattgatgaagatttcatcaacgatgtcggttctaaggttgtgatccctgtcaacaccagaagagacaatttttccaagagcatgtctaccagcaatttcaaaggttctggtagaaatatgggttctaatttcagaaatgattttggatctagaaacaactccaagcaaggttccaagaaaaagaataaatctcaaaagagccaatctaggaggggtccaaagtactccaatgaagaagaacctgctgaaccagccgtgcctgctgaattgcctaatagagctgcaagcaagtgggtacctcgttcgttaagaaataaggaggctgaagtcaagactgctccagacggatctataatagtatcagaagaggacatcaaaaggaaaactaaatcccttttgaataagttgacgttggaattctttgatgatatctcaaacgatataattgctttgaccaagcaagctcaatgggaagatgacgtcaagactttgaaacaagttattgagtctatatttgcaaaggcttgtgacgaaccctactggtcctctatgtacgctaaattatgcgccaaaatgtgcaaggacaccccacctgagatcaaggaaactaatgagaagggaaatactttcaccggtggtgatttggtgagaagagtgttgattaatagatgtcaaaccgaatatcagaaaggatggattgaccagctaccaaccaacgaggatggttcacctctagagccagaactgatgtctgacgaatattacaaacaagctgctgctaaacgtagaggtcttggtctggttaagttcattggtcaattgtaccagttgaacttattatcagacaatgtcattttccactgtgtccttggtcagtccaaagaaacagacaatccatcagaagatactatggagaacctggttcaattattggaagctgtaggttatagaatggagatgtccagtgaacctaagattagaagggttcttgatctcgcctttgccaacatcaaggccattgttgacgctaacaagattccttcaagaataagatttatgctcatggatttgatggatctaagaagaaatggctgggctacaacagaagagaacgctggtccaaagaccattcaagagattcacttagaggctgagatcaagagatcagaggaggagagagaaaaagaaagagacaggcattctcgtagaggacgttacaacgactccaaacctaactctcgtcaaaagtcatcttggggaagttcagtggtttcacctaatgatattagaaatgcaggctatgttagaaactctgacaaatctctggggcctgtcaacagtttttccaggggcaagtcatcccgtcaagcttcaaagcaatcaaacagtgattttaccagtgttcctatcacttccagtggtagtagaactgctctaaaccactctggcgacagctcaacgtctctgaacgaacctcaatcaagagaatattcaaaaaaatctgaatctaggcccgctccttccaacatttttgctgcattagaggatcacgatcatgatcatgaacatggccacgaacaagaagaaaacaagcattccaaggatctccaaaacggagaggccgaagcacctgaggacgacaatgtgggagaaatagcccaagctgacgaaaatgtgcaagggcaagagcaagaggaagaacgagaggaagagcgagagcaagaagaggaacaggaagtttaa

SEQ ID NO: 74 - DNA, 1881 bp, Komagataella phaffii
atgtctgtcgataccaaggaagttcaagaatcccttgatcaacttaacatttcagaatcaccagctactgctgtgactgaagagactccagctacctctaccgaggccgctgaagagtccaacgagtcatcgactcaagcttccgagaccttggcttcattgtatgttggtgagctggaccctaccgttactgagagtgacttgtatgagttcttctctccaatcggtagtgttaattccatccgtgtgtgtcgtgatgcagtcaccaagcgttcgttgggttatggatacgttaactttcacagccaagctgccggcgagcgtgccttggaggagctgaactacgctgagatcaaaggtgtccgttgtagattgatgtggtctcaacgtgacccttccttgcgtcgtagtggttccggtaacatcttcatcaagaacttagaccctgctattgagaacaagactttgcacgatactttctcctcttttggaaaagttttgtcctgtaaggttgccactgatgagaatggaaactcaaagggattcggattcgttcattacgaaagcgatgaagcagctcaagctgccattgagaacatcaacggtatgctgttgaatggtcgtgaaatctacgttggacctcacttggccaagaaggaccgtgaatctagattccaggagatgatcaagaactacaccaatgtcttcgtcaagaactttgacactgagtccactgaggatgaactgagagagctgtttgagtcatacggtccaatcacttcgatccacttgcaagttgattctgagggtcacaacaagggttttggtttcgtcaactttgctgaacacgacgatgctgttaaggccgttgaagccttgaacgacaaggagtacaagggcaagccattgtacgttggacgtgctcagaagaaaaacgagagagtccacgaacttaccaagaagtacgaggctgatcgtcttgaaaaattgcaaaagtatcaatcagttaacttgttcatcaagaacttggatgagtccattgatgatgcccgtcttgaggaagagttcaaaccatttggaaccatcacctctgccaaggtcatgttggacgaaaatggaaaatctagaggtttcggtttcgtttgtctctctactccagaggaagccactaaggctatcagtgagatgaaccaacgtatggttgccaacaagccattatacgttgctttggctcaaccaaaagccatcagacgctctcaattggctcagcagatccaagctagaaaccaaatgagaatgcaacaacaagctggtccaggtataccaaaccagttcgttcaacctatcttttacgggcaacagccaggtatgttgccaccaggtgctagagtcccaccaatgggtaaccagattcctcaatttgctggtatgccaagaccaggcccatttccccaaggccaattccctagaatggctcctaacggtcagccaatgcctgtgtatggccaacctgtattcaacggtaacggtcctcagcagcgtggctactacccaaacaacagacagaacaagaacaggaatcaaaaggaggaaggaaactctttggctgcaatcttgcctcagttgccattagagcaacaaaagagagtactgggtgaggagctttatccaaaggttgttgctactaacaagacccaagatccagaagctgcaggtaaaatcactggtatgatcttggatttggacaaccaagaaattttgcaactgttggaagatgaagaacttttcaacactcatttcaacgaagctctccaagcttacgaggagtacaagagtaagactgttgaacagcaacagcaagcggatgcacaagcaaactag

SEQ ID NO: 75 - DNA, 1881 bp, Artificial Sequence
  PAB1 gene mutant
atgtctgtcgataccaaggaagttcaagaatcccttgatcaacttaacatttcagaatcaccagctactgctgtgactgaagagactccagctacctctaccgaggccgctgaagagtccaacgagtcatcgactcaagcttccgagacattggcttcattgtatgttggtgagctggaccctaccgttactgagagtgacttgtatgagttcttctctccaatcggtagtgttaattccatccgtgtgtgtcgtgatgcagtcaccaagcgttcgttgggttatggatacgttaactttcacagccaagctgccggcgagcgtgccttggaggagctgaactacgctgagatcaaaggtgtccgttgtagattgatgtggtcccaacgtgacccttccttgcgtcgtagtggttccggtaacatcttcatcaagaacttagaccctgctattgagaacaagactttgcacgatactttctcctcttttggaaaagttttgtcctgtaaggttgccactgatgagaatggaaactcaaagggattcggattcgttcattacgaaagcgatgaagcagctcaagctgccattgagaacatcaacggtatgctgttgaatggtcgtgaaatctacgttggacctcacttggccaagaaggaccgtgaatctagattccaggagatgatcaagaactacaccaatgtctttgtcaagaactttgacactgagtccactgaggatgaactgagagagctgtttgagtcatacggtccaatcacttcgatccacttgcaagttgattctgagggtcacaacaagggttttggtttcgtcaactttgctgaacacgacgatgctgttaaggccgttgaagccttgaacgacaaggagtacaagggcaagccattgtacgttggacgtgctcagaagaaaaacgagagagtccacgaacttaccaagaagtacgaggctgatcgtcttgaaaaattgcaaaagtatcaatcagttaacttgttcatcaagaacttggatgagtccattgatgatgcccgtcttgaggaagagttcaaaccatttggaaccatcacctctgccaaggtcatgttggacgaaaatggaaaatctagaggtttcggtttcgtttgtctctctactccagaggaagccactaaggctatcagtgagatgaaccaacgtatggttgccaacaagccattatacgttgctttggctcaaccaaaagccatcagacgctctcaattggctcagcagatccaagctagaaaccaaatgagaatgcaacaacaagctggtccaggtataccaaaccagttcgttcaacctatcttttacgggcaacagccaggtatgttgccaccaggtgctagagtcccaccaatgggtaaccagattcctcaatttgctggtatgccaagaccaggcccatttccccaaggccaattccctagaatggctcctaacggtcagccaatgcctgtgtatggccaacctgtattcaacggtaacggtcctcagcagcgtggctactacccaaacaacagacagaacaagaacaggaatcaaaaggaggaaggaaactctttggctgcaatcttgcctcagttgccattagagcaacaaaagagagtactgggtgaggagctttatccaaaggttgttgctactaacaagacccaagatccagaagctgcaggtaaaatcactggtatgatcttggatttggacaaccaagaaattttgcaactgttggaagatgaagaacttttcaacactcatttcaacgaagctctccaagcttacgaggagtacaagagtaagactgttgaacagcaacagcaagcggatgcacaagcaaactag

SEQ ID NO: 76 - DNA, 1821 bp, Komagataella phaffii
atgagtgagaaaaacacacgtattgccattgtttcagacgatagatgcaaacctaagaaatgtcgtcaggaatgcagaaaatcctgtcctgttgttaagacaggaaagctttgtattgaagttgaagcatactccaaaattgccttcatcagtgagactctgtgtataggttgtggtatttgtgtgaaaaagtgtccatttggagcaattaacattatcaatctgcccactaacttggagaaggaggtcacccacagatacagtgccaactcattcaaattgcacagattgccaacacctaggccaggtcaagtcctaggtcttgttggaaccaatggtattgggaaatccaccgcattgaaaattcttgcaggtaagcagaaaccaaatttaggtagatacgacgcgccacctgactgggaagaaattttgaaatacttcagaggaagtgaattgcaaaattattttaccaaagttttggaggacaatattaaagcaattattaagcctcaatatgttgacaaccttccaagggcaatcaaaggtcctgttaataaggttggtgagcttttgaaagccaaactggaaagaaaggaagagggaaagcatatcatccaagttttggaattgaaacacgtattgaacagagaagtcgctaagctttctggtggtgagcttcaaagatttgcaattggtatgacttgtgtccaagatgctaatgtgtacatgtttgatgaaccttcatcctatttggatgttaagcaacgtttgaatgctgctcagattattagatctttgttgcatgccacctgttatgttattgttgttgagcacgatctgtctgttttggattatctttctgattttgtttgtattttgtatggtgtaccatcggtttacggtgttgtcactttgccagcctctgtcagagaaggtatcaatattttccttgatggtcatatccctaccgagaatttgaggttcagaactgagtctttaaaattcaaacttacagatgcttcggactctgtattaattgaacaaagtagagcttttgaataccccaaaatggttaagactcaaggtgatttcaagttaaccgtcaacgctggtgacttctcagactcggaaattattgtgatgatgggtgaaaatggtaccggtaagaccactttatgtaagctgttagctggtgccattgaaggtgatgaaggcaccaaggtacccaaattgaatgtttctatgaagccccaaaagattgctccaaaattcacaggaacagttagacaactgttcttgaaaaagattagatcatctttcttacatccacaattcaacaccgatgtgatgaaacctttaaaagttgacgatgtcattgatcaagaagtccagcatttgtctggtggtgaattgcaaagagttgccatcgtcttagctttaggaaaagaggcagacatctacttgattgacgagccatctgcttacttggactctgaacagcgtattatctgttcgaaggttattagaagattcatcctgcattccaagaaaactgctttcattgtggaacacgatttcatcatggccacatacttagccgataaagtgatggtatttgaaggtcagccttccaagaatgctgttgctaaccgcccagaaacccttttgactggttgtaacaggttcttgaaaaacctgaacgtcactttcagaagagatccaaactcttttagacccagaatcaacaaactggactcccagatggatagagagcaaaaactgagtggtaattacttcttccttgagaacactgagttataa

SEQ ID NO: 77 - protein, 362 aa, Artificial Sequence
  vHH expression construct
MRFPSIFTAVLFAASSALAAPVNTTTEDETAQIPAEAVIGYSDLEGDFDVAVLPFSNSTNNGLLFINTTIASIAAKEEGVSLEKRQVQLQESGGGLVQAGGSLRLSCAASGRTFTSFAMGWFRQAPGKEREFVASISRSGTLTRYADSAKGRFTISVDNAKNTVSLQMDNLNPDDTAVYYCAADLHRPYGPGTQRSDEYDSWGQGTQVTVSSGGGSGGGGSGGGGSGGGGSGGGGSGGGEVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGHHHHHH

SEQ ID NO: 78 - DNA, 1092 bp, Artificial Sequence
  vHH Nucleotide Sequence
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagacaggttcagctgcaggagtccggtggtggtctggttcaagccggtggttcattaagattgtcctgtgctgcctctggtagaactttcacttctttcgcaatgggttggtttagacaagcacctggaaaagagagagagtttgttgcttctatctccagatccggtactttaactagatacgctgactctgccaagggtagattcactatttctgttgacaacgccaagaacactgtttctttgcaaatggacaaccttaacccagatgacaccgcagtctattactgtgccgctgacttgcacagaccatacggtccaggaacccaaagatccgatgagtacgattcttggggtcagggaactcaagtcactgtctcttcaggtggtggatctggtggtggaggttcaggtggtggaggatccggtggtggtggttctggtggtggtggatctggtggaggtgaagttcaacttgtcgaatccggtggtgcacttgtccaacctggtggatctcttagactttcttgtgccgcctccggttttcctgttaaccgttactctatgcgttggtacagacaagcccctggaaaagaacgtgaatgggttgccggaatgtcctcagctggtgacagatcctcctacgaagattctgtgaagggacgtttcaccatctccagagatgacgcccgtaacaccgtttaccttcaaatgaactcccttaagcctgaggatactgccgtctactattgtaacgtgaatgtcggatttgaatactggggacagggaacccaagttactgtctcttccggtggacatcaccaccaccatcactaatag

SEQ ID NO: 79 - protein, 609 aa, Artificial Sequence [sequence omitted]

SEQ ID NO: 80 - DNA, 1830 bp, Artificial Sequence
  Human serum albumin expression construct
atgaagtgggttactttcatctccttgttgttcttgttctcctcagcttactccagaggtgttttcagaagagatgctcacaagtccgaggttgctcacagattcaaggacttgggtgaagagaacttcaaggctttggttttgatcgctttcgctcagtacttgcagcagtgtccattcgaggaccacgttaagttggttaacgaggttactgagttcgctaagacttgtgttgctgacgaatccgctgagaactgtgataagtccttgcacactttgttcggtgacaagttgtgtactgttgctactttgagagaaacttacggtgagatggctgactgttgtgctaagcaagagcctgagagaaacgagtgtttcttgcaacacaaggacgacaacccaaacttgccaagattggttagaccagaggttgacgttatgtgtactgctttccacgacaacgaagagactttcttgaagaagtacttgtacgagatcgctagaagacacccatacttctacgctccagagttgttgttcttcgctaagagatacaaggctgctttcactgagtgttgtcaggctgctgataaggctgcttgtttgttgccaaagttggacgagttgagagatgagggtaaggcttcttccgctaagcagagattgaagtgtgcttccttgcagaagttcggagagagagcttttaaggcttgggctgttgctagattgtcccagagattcccaaaggctgagttcgctgaggtttccaagttggttactgacttgactaaggttcacacagagtgttgtcacggtgacttgttggaatgtgctgatgacagagctgacttggctaagtacatctgtgagaaccaggattccatctcctccaagttgaaagaatgttgtgagaagcctttgttggagaagtcccactgtatcgctgaggttgaaaacgacgaaatgccagctgacttgccatctttggctgctgacttcgttgaatccaaggacgtctgcaagaactacgctgaggctaaggacgttttcttgggtatgttcttgtatgagtacgctagaagacatccagactactccgttgttttgttgttgagattggctaagacttacgagactactttggagaagtgttgtgctgctgctgacccacatgagtgttacgctaaggttttcgacgagttcaagccattggttgaggaaccacagaacttgatcaagcagaactgtgagttgttcgagcagttgggtgagtacaagttccagaacgctttgttggttagatacactaagaaggttccacaggtttccactccaactttggttgaggtttccagaaacttgggtaaggttggttccaagtgttgtaagcacccagaggctaagagaatgccatgtgctgaggactacttgtctgttgttttgaaccagttgtgtgtcttgcacgaaaagacaccagtttccgacagagttactaagtgttgtactgaatccttggttaacagaagaccttgtttctccgctttggaggttgacgagacttacgttccaaaagagttcaacgctgagactttcactttccacgctgacatctgtactttgtccgagaaagagagacagatcaagaagcagactgctttggttgagttggttaagcacaagccaaaggctacaaaagagcagttgaaggctgttatggacgacttcgctgctttcgttgagaaatgttgtaaggctgacgacaaagagacttgtttcgctgaagagggtaagaagttggttgctgcttcccaagctgctttgggtctgtaa

SEQ ID NO: 81 - DNA, 1001 bp, Komagataella phaffii
catgttggtattgtgaaatagacgcagatcgggaacactgaaaaataacagttattattcgagatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 82 - DNA, 1000 bp, Komagataella phaffii
gcttaaaggactccatttcctaaaatttcaagcagtcctctcaactaaatttttttccattcctctgcacccagccctcttcatcaaccgtccagccttctcaaaagtccaatgtaagtagcctgcaaattcaggttacaacccctcaattttccatccaagggcgatccttacaaagttaatatcgaacagcagagactaagcgagtcatcatcaccacccaacgatggtgaaaaactttaagcatagattgatggagggtgtatggcacttggcggctgcattagagtttgaaactatggggtaatacatcacatccggaactgatccgactccgagatcatatgcaaagcacgtgatgtaccccgtaaactgctcggattatcgttgcaattcatcgtcttaaacagtacaagaaactttattcatgggtcattggactctgatgaggggcacatttccccaatgattttttgggaaagaaagccgtaagaggacagttaagcgaaagagacaagacaacgaacagcaaaagtgacagctgtcagctacctagtggacagttgggagtttccaattggttggttttgaatttttacccatgttgagttgtccttgcttctccttgcaaacaatgcaagttgataagacatcaccttccaagataggctatttttgtcgcataaatttttgtctcggagtgaaaaccccttttatgtgaacagattacagaagcgtcctacccttcaccggttgagatggggagaaaattaagcgatgaggagacgattattggtataaaagaagcaaccaaaatcccttattgtccttttctgatcagcatcaaagaatattgtcttaaaacgggcttttaactacattgttcttacacattgcaaacctcttccttctatttcggatcaactgtattgactacattgatcttttttaacgaagtttacgacttactaaatccccacaaacaaatcaactgagaaaa

SEQ ID NO: 83 - DNA, 991 bp, Artificial Sequence
  promoter
gcaaggcaactgagaaattgaatagtggtttcaagcccgctgactttttgtattatctcaatgtcggtgtttcacagtccccagaagggggctttgccttcaagggagacggaagagacatcgtcaaccctggggagaagtatttcaaatggcgcaagttcgctaatttttacgattaagcagtgctgtatggggtagttaataaatcgggaatatccttctgacgtgactgtaacaaatctctttttacgtggtgcgcatactggacagaggcagagtctcaatttcttcttttgagacaggctactacagcctgtgattcctcttggtacttggatttgcttttatctggctccgttgggaactgtgcctgggttttgaagtatcttgtggatgtgtttctaacactttttcaatcttcttggagtgagaatgcaggactttgaacatcgtctagctcgttggtaggtgaaccgttttaccttgcatgtggttaggagttttctggagtaaccaagaccgtcttatcatcgccgtaaaatcgctcttactgtcgctaataatcccgctggaagagaagttcgaacagaagtagcacgcaaagctcttgtcaaatgagaattgttaatcgtttgacaggtcacactcgtgggctatgtacgatcaacttgccggctgttgctggagagatgacaccagttgtggcatggccaattggtattcagccgtaccactgtatggaaaatgagattatcttgttcttgatctagtttcttgccattttagagttgccacattcgtaggtttcagtaccaataatggtaacttccaaacttccaacgcagataccagagatctgccgatccttccccaacaataggagcttactacgccatacatatagcctatctattttcactttcgcgtgggtgcttctatataaacggttccccatcttccgtttcatactacttgaattttaagcactaaa

SEQ ID NO: 84 - DNA, 864 bp, Artificial Sequence
  promter
cttttctttgggcaaggaaaaatcaagaaaaagcagaggttaaagttttcaggggaatggcaattgctttatatatgggagaaagttaactacgtcggtgctgtaggcgtagagagcgactggagaatgcgtgatgaggtcgtctcttttcgcccccccttggcggggtaaaaattgcactactgcagaattactacacccctattccgaggagacggagtgcgacaaaaatggtaaagttcaccctagtctgcgacttttaattgacggacaccggcgtttacatgcgaaaaaaactaaagtgcgcgcatttcacggccgaggggggtcccacttgggactgagagggggtgggatctgaaatcgaggaggtatcaagaccccccgtttctcaactccctaatcaaaaattacgaagtcctcgttggaaaggagttaaaataattaagcggggtcggacgccataccgaggttatcttgcaggcattttactaatattggaattcggagctcaacttgcaaccaggcagggtttagctatgtaatcaatgtaatcaatataataaagcactaccacatcgaaggtttgggagggaggccaatagtgtcccccacagggtgctgatatcgcgattcttgggtgaggagacacatatttcactcctctcaccaaccaaccaagcggctcctcgcaagatgatttatccgattatccggacactatactcccatccagtttgatgccgatttcatcgattgtcctaaataatccttaaatatgtatagaacggtaccctggggttacataatccttatttaataatccctcccccaccgcttttctttttttttcttcttattgtc

SEQ ID NO: 85 - DNA, 1000 bp, Artificial Sequence
  promoter
aaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaaca

SEQ ID NO: 86 - DNA, 549 bp, Artificial Sequence
  promoter
aatgatataaacaacaattgagtgacaggtctactttgttctcaaaaggccataaccatctgtttgcatctcttatcaccacaccatcctcctcatctggccttcaattgtggggaacaactagcatcccaacaccagactaactccacccagatgaaaccagttgtcgcttaccagtcaatgaatgttgagctaacgttccttgaaactcgaatgatcccagccttgctgcgtatcatccctccgctattccgccgcttgctccaaccatgtttccgcctttttcgaacaagttcaaatacctatctttggcaggacttttcctcctgccttttttagcctcaggtctcggttagcctctaggcaaattctggtcttcatacctatatcaacttttcatcagatagcctttgggttcaaaaaagaactaaagcaggatgcctgatatataaatcccagatgatctgcttttgaaactattttcagtatcttgattcgtttacttacaaacaactattgttgattttatctggagaataatcgaacaaa

SEQ ID NO: 87 - DNA, 1000 bp, Artificial Sequence
  promoter
attactgttttgggcaatcctgttgataagacgcattctagagttgtttcatgaaagggttacgggtgttgattggtttgagatatgccagaggacagatcaatctgtggtttgctaaactggaagtctggtaaggactctagcaagtccgttactcaaaaagtcataccaagtaagattacgtaacacctgggcatgactttctaagttagcaagtcaccaagagggtcctatttaacgtttggcggtatctgaaacacaagacttgcctatcccatagtacatcatattacctgtcaagctatgctaccccacagaaataccccaaaagttgaagtgaaaaaatgaaaattactggtaacttcaccccataacaaacttaataatttctgtagccaatgaaagtaaaccccattcaatgttccgagatttagtatacttgcccctataagaaacgaaggatttcagcttccttaccccatgaacagaaatcttccatttaccccccactggagagatccgcccaaacgaacagataatagaaaaaagaaattcggacaaatagaacactttctcagccaattaaagtcattccatgcactccctttagctgccgttccatccctttgttgagcaacaccatcgttagccagtacgaaagaggaaacttaaccgataccttggagaaatctaaggcgcgaatgagtttagcctagatatccttagtgaagggttgttccgatacttctccacattcagtcatagatgggcagctttgttatcatgaagagacggaaacgggcattaagggttaaccgccaaattatataaagacaacatgtccccagtttaaagtttttctttcctattcttgtatcctgagtgaccgttgtgtttaatataacaagttcgttttaacttaagaccaaaaccagttacaacaaattataacccctctaaacactaaagttcactcttatcaaactatcaaacatcaaaa

SEQ ID NO: 88 - DNA, 1000 bp, Artificial Sequence
  promoter
gtcaactgcgtactcttttgtcgaatggactactgaatctgcctcgatagccactataggaaggtccatagaggccagtttttcaactagtcttggtggaaagaaaccgacaaagcctttcatggagtcaccgatactgaaaggttcaaacaaagaatgcttgggtagtctcttaatacccatggcaacgaaaaaggggtcttcattgttcaacatgaattcgtatccacctttaatgtagtcataaagctgctgaagttccgaatcagtgatggaactgtctacagtgacaatataggagttctcaatcaccttatatccagtcgaatatatctggatagggtcgggtctcactgtggaagattcaaatgggttagatccctgtaatttcagcgatggagactcagtatgatggggcaaggaaaacggcaattggatattcaattggtcaagagatggtatcaaaagcgagtgtgccagggtagccacggtagccactgatgctaatctgataattttcatttctggagtgtcaaaacagtagtgataaaaggctatgaaggaggttgtctaggggctcgcggaggaaagtgattcaaacagacctgccaaaaagagaaaaaagagggaatccctgttctttccaatggaaatgacgtaactttaacttgaaaaataccccaaccagaagggttcaaactcaacaaggattgcgtaattcctacaagtagcttagagctgggggagagacaactgaaggcagcttaacgataacgcggggggattggtgcacgactcgaaaggaggtatcttagtcttgtaacctcttttttccagaggctattcaagattcataggcgatatcgatgtggagaagggtgaacaatataaaaggctggagagatgtcaatgaagcagctggatagatttcaaattttctagatttcagagtaatcgcacaaaacgaaggaatcccaccaagcaaaaaaaaaaatctaag

SEQ ID NO: 89 - DNA, 704 bp, Artificial Sequence
  promoter
aaattaatccataagataaggcaaatgtgcttaagtaattgaaaacagtgttgtgattatataagcatggtatttgaatagaactactggggttaacttatctagtaggatggaagttgagggagatcaagatgcttaaagaaaaggattggccaatatgaaagccataattagcaatacttatttaatcagataattgtggggcattgtgacttgacttttaccaggacttcaaacctcaaccatttaaacagttatagaagacgtaccgtcacttttgcttttaatgtgatctaaatgtgatcacatgaactcaaactaaaatgatatcttttactggacaaaaatgttatcctgcaaacagaaagctttcttctattctaagaagaacatttacattggtgggaaacctgaaaacagaaaataaatactccccagtgaccctatgagcaggatttttgcatccctattgtaggcctttcaaactcacacctaatatttcccgccactcacactatcaatgatcacttcccagttctcttcttcccctattcgtaccatgcaacccttacacgccttttccatttcggttcggatgcgacttccagtctgtggggtacgtagcctattctcttagccggtatttaaacatacaaattcacccaaattctaccttgataaggtaattgattaatttcataaat

SEQ ID NO: 90 - DNA, 1000 bp, Artificial Sequence
  promoter
agctcagattggaaatgatttttgatcctaccaagaagcctttgatttccagaatctccgctaagtaagtaacccccgcaaacgcatgcatccatgcaaacaaaatactaacaattttagccccgttgttgagaaacccagaaaattgaatgttcaaccaatccagacgatcaataagaaaaaaggcccaaaggctacttccaaacctgctgccgccaaacctgctccttcaaaagccggtcccaagggaggtaagaaggtgagaaagccaaagaagacagttgaagaattggatcaggaaatggctgactactttgaaaataagaattagcccaacaaaatatgtacaagtattatataaatgaatctacatggtgtgttttatttagatcctccaaaccaaggaaagaaactaaacttatctccggacttacgagtcaaataactatccgcagttccttggaactcagactttcttccataagcggtcatatcatctttggactgtgggaatcctggacgaatctttgaaatgtcataatcttgctctctatctccaagcacagcgtccggtaaatgctggttcttctttctcagatgaatcttggatttaacaaataaagccgtgcctatggctaatgtactcaaaaacaaagtctgcttccagaatttcgcaaacgatggaatgccatttcctgtaaatgtactcattgaacctatgtttgattaaagttggtgtgaagtcatcaaacgagagtaaaatcagatactcgtgcaccggccaaaattgactgagctaatctctgcaggcttgacatccgaacacaacaaataggcgacaaatcttaactatctaatcgtaggctatggtagaactttgtgggggtagaggaagactacaacagcaagacaaaacaaaagagtcatagtttgactctctgcttttttcttctttctcttctttttcttcctccatattcgttatttatttcgaactgga

SEQ ID NO: 91 - DNA, 1000 bp, Artificial Sequence
  promoter
cagccattaatctcacctcagtttttgaatcagtagaatttttaatgaaacaaacggttggtatattatttgatagagttgccaaatttccaaagataaatttttcatcaggtaatatcctgaataccgtaacatagtgactattggaagacactgctatcatattatatttcggataaaaatccaaaccccagaccgacctcttgagtctcaactccaagtcagccgcaactttaattatccgtggattgggagctagtttggacaacgcatcagtataatataactttacggttccattatcagacgctattgcaagaacttcctttccattgatctcgccaatgcggcagtaattgatatcgtagggtaggtctggaaagacgctggcgcttgtgtcccattctgcaggaatctctggcacggtgctaatggtagttatccaacggagctgaggtagtcgatatatctggatatgccgcctataggataaaaacaggagagggtgaaccttgcttatggctactagattgttcttgtactctgaattctcattatgggaaactaaactaatctcatctgtgtgttgcagtactattgaatcgttgtagtatctacctggagggcattccatgaattagtgagataacagagttgggtaactagagagaataatagacgtatgcatgattactacacaacggatgtcgcactctttccttagttaaaactatcatccaatcacaagatgcgggctggaaagacttgctcccgaaggataatcttctgcttctatctcccttcctcatatggtttcgcagggctcatgccccttcttccttcgaactgcccgatgaggaagtccttagcctatcaaagaattcgggaccatcatcgatttttagagccttacctgatcgcaatcaggatttcactactcatataaatacatcgctcaaagctccaactttgcttgttcatacaattcttgatattcaca

SEQ ID NO: 92 - DNA, 1000 bp, Artificial Sequence
  promoter
tggttccctctcggtccaataccaaaaatattatcaccatacaggtctcccttcgataccagtgcaaagttgaaccgtgggattaccttggaatctacaaaaatagtgtcactcacaagtttgtcatcaaccacgctgccgcttgcaaaggagaactgaacatgaaggttgttagggtttgttatattggaataagtggtggatttgttgaaggcgaacgcaccaaagctacatccgtcctgagcacactgtgaatttgtcacggaattgaccaagaggtcagacgatcctgtatcccattgagccgttatgctttgtgggggaaaccctatttctatcgtactaagaaaaccaatggtgaactcatattcggtatcaatggcgacgattccagcatagcctgtagacagtaacaacactagggcaacagcaactaacatatcttcattgatgaaacgttgtgatcggtgtgacttttatagtaaaagctacaactgtttgaaataccaagatatcattgtgaatggctcaaaagggtaatacatctgaaaaacctgaagtgtggaaaattccgatggagccaactcatgataacgcagaagtcccattttgccatcttctcttggtatgaaacggtagaaaatgatccgagtatgccaattgatactcttgattcatgccctatagtttgcgtagggtttaattgatctcctggtctatcgatctgggacgcaatgtagaccccattagtggaaacactgaaagggatccaacactctaggcggacccgctcacagtcatttcaggacaatcaccacaggaatcaactacttctcccagtcttccttgcgtgaagcttcaagcctacaacataacacttcttacttaatctttgattctcgaattgtttacccaatcttgacaacttagcctaagcaatactctggggttatatatagcaattgctcttcctcgctgtagcgttcattccatctttcta

SEQ ID NO: 93 - DNA, 1001 bp, Artificial Sequence
  promoter
gatatcgatctacacttaatagtagatgacgaggcatctctccaataggtaccatatctggtgtttcttgtaatttaagaatctgttggtctatgaatgtagatttgtcatgaacaatgatatatgggtcaggaggacaagatggtttctctgagttgggttgttgaggtgcctggcaagacttcggagcgttgatatccccaagacttgtagtgaccgatagttgaagcgtgtgtttgcaggaacggcacatcaatgcaactttcgtaactttggaattgagagttgatgcactgatgacgatacccgaaattttgacgattttaccaatatgacttgaagacaagtctctcattgaaaccttattatcgttactaagcaaaacgagctgacaagaagggaaggtggtcggtatttcctcgttgttcaaatatatgattctcctggcaatatctgtgatggcctgttcaaaaagtggaatcatttctgcaggatcatctaccaactttttattgagctcctcattgaatacgattaagtggtcattttgaatcgtcagtaagtacttgtttacaagtaaattctgtctgagttgttctctgtagatgtactgattttccatacgaaactccaaaatgaacgaacggaatgccttaatgacctcactgaactggtcatcgttctgttctccgggaaggacacttgtgttaaagactgatgctctatcaaaggacattgcaacaaagtataaacggttgtgagcgggaaaaagatgtgtaggtaattgtcgtagatgagactgattcagtagaaaacgcgtcctgcactatttttttctttcttcattacatttcctaatcgggacaaaatgaatctaaagacgtggttatgtagtacacgcatcgataggctatccccataccaaaacactattttaccccatccttgacaggttataaatatgcgatagtatgagtatcttcaaattcagctgaaatatc

SEQ ID NO: 94 - DNA, 1000 bp, Artificial Sequence
  promoter
aataaaaaaacgttatagaaagaaattggactacgatatgctccaatccaaattgtcaaaattgaccaccgaaaaagaacaattggaatttgacaagaggaacaactcactagattctcaaacggagcgtcacctagagtcagtttccaagtcaattacagaaagtttggaaacagaagaggagtatctacaattgaattccaaacttaaagtcgagctgtccgaattcatgtcgctaaggctttcttacttggaccccatttttgaaagtttcattaaagttcagtcaaaaattttcatggacatttatgacacattaaagagcggactaccttatgttgattctctatccaaagaggattatcagtccaagatcttggactctagaatagataacattctgtcgaaaatggaagcgctgaaccttcaagcttacattgatgattagagcaatgatataaacaacaattgagtgacaggtctactttgttctcaaaaggccataaccatctgtttgcatctcttatcaccacaccatcctcctcatctggccttcaattgtggggaacaactagcatcccaacaccagactaactccacccagatgaaaccagttgtcgcttaccagtcaatgaatgttgagctaacgttccttgaaactcgaatgatcccagccttgctgcgtatcatccctccgctattccgccgcttgctccaaccatgtttccgcctttttcgaacaagttcaaatacctatctttggcaggacttttcctcctgccttttttagcctcaggtctcggttagcctctaggcaaattctggtcttcatacctatatcaacttttcatcagatagcctttgggttcaaaaaagaactaaagcaggatgcctgatatataaatcccagatgatctgcttttgaaactattttcagtatcttgattcgtttacttacaaacaactattgttgattttatctggagaataatcgaacaaa

SEQ ID NO: 95 - DNA, 1000 bp, Artificial Sequence
  promoter
attgttgtgaatactctccttcatttggatttcttggacttcggactctcttgatctctcttcgaaagttttaactctgttcatgtataattttacccgctgtaggtcgctcataataccatgagtatgcacatcttttactccattaactttcaggtatgcaaaatacaatgaagatagtatatagctcaaagaatttagcattttgcattgatctaattgtgacattttctctatgatatcatctagcttcttaaactcgagaatctcgtccaacgaggcagaaacattgtccagtcttacgtcaagattattcacgagtttctggaccgtatcaacgttttccatcttaagattacagtaagtatcgtccttttgaactgcaaaggtagaaaagttaatttttgatttggtagtacactatgaaacttgctcaccccaatctttcctcctgacaggttgatctttatccctctactaaattgccccaagtgtatcaagtagactagatctcgcgaaagaacagcctaataaactccgaagcatgatggcctctatccggaaaacgttaagagatgtggcaacaggagggcacatagaatttttaaagacgctgaagaatgctatcatagtccgtaaaaatgtgatagtactttgtttagtgcgtacgccacttattcggggccaatagctaaacccaggtttgctggcagcaaattcaactgtagattgaatctctctaacaataatggtgttcaatcccctggctggtcacggggaggactatcttgcgtgatccgcttggaaaatgttgtgtatccctttctcaattgcggaaagcatctgctacttcccataggcaccagttacccaattgatatttccaaaaaagattaccatatgttcatctagaagtataaatacaagtggacattcaatgaatatttcattcaattagtcattgacactttcatcaacttactacgtcttattcaacaat

SEQ ID NO: 96 - DNA, 1000 bp, Artificial Sequence
  promoter
tatacggtctatccactttggaaacgatgtagttgaaacggggaagtaatagtggttcccaaacgacatgaagaggttatataagtttgcaagagggtgacaccattttagttgtggttcccgggtatttttttaatctttttagtctaagatagcctccccagatattaccgagttgggccatttggggcggtatcggtggtatctgatggtagcgcgtttttacatgcctgtgcattgaactggcaaagagtatactatcgtggggccctgaaggaggcagcaaatggaccgtcaattggttgatcagggactcaagacaggtattgagcttttcaaacaaaaagagtataggcgctgctacaaggcatttacttctactatcaatttcattgagaatgatcccgagttggccgccagctgtgtatctcaactgatatctctgttagattgtagggcagcctgtttggaaaagctagatcaattgaatatggccttgaaagatggtcttaaaatgatcaagagagagtgccacaactgcaagggttatttgagaacttgcaaaattttagacctacaagggaagatcagtgaggctttgtctacagcaagagaagggatctccataatagaaactagaagagatcaggataatcaatttagatattccaaggttcttttggaacaattaaaggaactgaaaaatgcactgaaaatcaaattggacaagaaaaatcagctacacttcaaagttttaaagtttgacgcaccagtgccttgtacaaagaaactaagattagtcactccaagaacaatagatccttccatttttttgccgatagagctagtgaagctgatctttcgcctgttgaatttctcagacatgtatgcctgtttattggtctcaacaaaatggaactcaattatatcctcatcaccggaactgtttcgaaaacttcagttgaaatcccaactgtccaacaaggcgttaaacaattgt

SEQ ID NO: 97 - DNA, 951 bp, Artificial Sequence
  pG1 promoter
  misc_feature (578)..(578): n...(T)x wherein x is 14-16
caaacatttgctccccctagtctccagggaaatgtaaaatatactgctaatagaaaacagtaagacgctcagttgtcaggataattacgttcgactgtagtaaaacaggaatctgtattgttagaaagaacgagagttttttacggcgccgccatattgggccgtgtgaaaacagcttgaaaccccactactttcaaaggttctgttgctatacacgaaccatgtttaaccaacctcgcttttgacttgactgaagtcatcggttaacaatcaagtaccctagtctgtctgaatgctcctttccatattcagtaggtgtttcttgcacttttgcatgcactgcggaagaattagccaatagcgcgtttcatatgcgcttttaccccctcttttgtcaagcgcaaaatgcctgtaagatttggtgggggtgtgagccgttagctgaagtacaacaggctaattccctgaaaaaactgcagatagacttcaagatctcagggattcccactatttggtattctgatatgtttttcctgatatgcatcaaaactctaatctaaaacctgaatctccgctangatgaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 98 - DNA, 1238 bp, Artificial Sequence
  pG1-3 promoter
caaacatttgctccccctagtctccagggaaatgtaaaatatactgctaatagaaaacagtaagacgctcagttgtcaggataattacgttcgactgtagtaaaacaggaatctgtattgttagaaagaacgagagttttttacggcgccgccatattgggccgtgtgaaaacagcttgaaaccccactactttcaaaggttctgttgctatacacgaaccatgtttaaccaacctcgcttttgacttgactgaagtcatcggttaacaatcaagtaccctagtctgtctgaatgctcctttccatattcagtaggtgtttcttgcacttttgcatgcactgcggaagaattagccaatagcgcgtttcatatgcgcttttaccccctcttttgtcaagcgcaaaatgcctgtaagatttggtgggggtgtgagccgttagctgaagtacaacaggctaattccctgaaaaaactgcagctcagggattcccactatttggtattctgatatgtttttcctgatatgcatcaaaactctaatctaaaacctgaatctccgctattttttttttttttgatgaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgagatctcagggattcccactatttggtattctgatatgtttttcctgatatgcatcaaaactctaatctaaaacctgaatctccgctattttttttttttttgatgaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 99 - DNA, 1425 bp, Artificial Sequence
  pG1-4 promoter
caaacatttgctccccctagtctccagggaaatgtaaaatatactgctaatagaaaacagtaagacgctcagttgtcaggataattacgttcgactgtagtaaaacaggaatctgtattgttagaaagaacgagagttttttacggcgccgccatattgggccgtgtgaaaacagcttgaaaccccactactttcaaaggttctgttgctatacacgaaccatgtttaaccaacctcgcttttgacttgactgaagtcatcggttaacaatcaagtaccctagtctgtctgaatgctcctttccatattcagtaggtgtttcttgcacttttgcatgcactgcggaagaattagccaatagcgcgtttcatatgcgcttttaccccctcttttgtcaagcgcaaaatgcctgtaagatttggtgggggtgtgagccgttagctgaagtacaacaggctaattccctgaaaaaactgcagctcagggattcccactatttggtattctgatatgtttttcctgatatgcatcaaaactctaatctaaaacctgaatctccgctattttttttttttttgatgaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccacccttagatctcagggattcccactatttggtattctgatatgtttttcctgatatgcatcaaaactctaatctaaaacctgaatctccgctattttttttttttttgatgaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 100 - DNA, 491 bp, Komagataella phaffii
cttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcacctgcaggcc

SEQ ID NO: 101 - DNA, 488 bp, Komagataella phaffii
cttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 102 - DNA, 985 bp, Komagataella phaffii
agggcatcattgaggtttccacaaaaggaagaaacatggatccagagacatcaacagagaggaaagcgggtagtgaagccgaagccacaacacagcccgatttggaagggagttcacaatcaaggtgagtccagccattttttttcttttttttttttttattcaggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgtcactcgcttcactcaacaacaaaa

SEQ ID NO: 103 - DNA, 975 bp, Komagataella phaffii
gtagcttgggtaggacttgacaagtacggcttccgtggtcataccaaacgcctttgttaccgttggctatacctaatgaccaaggcatttgtggattataacggtatcgtagttgaaaaatatgacgtaaccactggtactagcccccacaaggttgatgctgaatacgggaatcaaggtgccgattttaaaggagtagccactgaagggtttggctgggtcaatgcctcttttattttgggattaacctacttagatgtccaaggcatccgtgcgataggcgccgttacgtcccctgatgtatttttcaggaagctcaaaccttgggaacgcgcaagttatggcctaaggccatgtaacgagatagtcaagtcaaactagaagtatacggtttccccgcagaaatagcagaaataggcgacaaatacatacaacattttcattgtgatagggggcggcggttcctaggagggacaacccccagaaaccttgtagactacgttttcacgacgatgggttattactgtaaaggaagaatatactacccaccagttgaatgtttgaacggatcaaaggtcgaagggagtacacggcccaaccaacgtagctaccggagaaagcaagactttcccaaaccaaatagctccgggtttcttctccggcaacccgtcagtttttgtgtggccggacaaaaattcgcaccctcagtctaattgaaaggtcgggctccgagctctaggcgtttgcgcatgtaatattgcatcccctcccatagataatactgcgcgaacacagggtgcaaattatgatgaccacacatgccagtgaccaaaacagttttttagtctttaaaaaccctcggaacttctgagtatataaaggcttctcatttcctacaagcaaacaaagaagaaacttccactttctaactttttatctatagactttagagttacaaccaacgaacaataacaa

SEQ ID NO: 104 - DNA, 699 bp, Komagataella phaffii
cgcattatttgggtcggctactgtcattggttatgatggccttaccgagcacgaaggtggttggatagaccacaactggaaacagttgtacaaacagattgcattcatttttgcttgtattggatactcgatggccatcaccgctcttatctgtttcatcctcaaccgtattccatttttgcaactgcgagcttcagaagaggctgaggagaaaggtatggatgaggatcagattggagagttcgcttatgactacgtggaagtacgtcgtgattttttggcttggggatcaggcccaaacaatggcttcaaggagccggaagttctggatcaggtagttccggttaatgatttcagcagtgaccagaatgtgactaatcagaccaacgaatctgagaagcagtagagtaaatatagagatgatatttagtgtattctaatgcttatgtaatgtattaagcaaaaagttgtgtttatgagttagcatttgtcttagcaaacataaaattatgtcgacatttgcaacccgcatgtctagtgtttttagatcgatcttcgatgtgtagaataatgcctccacgtgatgccccgcgattttgttgggtcgcaatgcctccaacataaacccatcacgtataaaaagccctcttaaccctcccccctgtttcgtttgcttcatcacttaacctgaactatcaa

SEQ ID NO: 105 - DNA, 1000 bp, Artificial Sequence
  pG3 promoter
gtaaatagcggcagcaatccagtaaccttttctgaatagcagagccttaactaaaataatggccagggtaaaaaattcgaaatttgacaccaaaaataaagacttgtcgttataagtcttaacaaagtccgcaattttggagctaacggtggcggttgctgggatattcaataatggtagaatgttgctgcgggtatatgacagagcgtgaaacacactgaacaaggtaaatggaacaacagcaattgcaatatgggggaggatagtcaagaacaaagcagcaatggcaaagtactgaatattctccaaagccaaaaggtccagtggtttcaacgacaaagtcttgttggtatagctttggaacaaaaggacaccgaaagactcgacagcgcccacaaatacagcgttgtagaagaacgaattgattgctccagagcttctaatagtcagaagataccccaaacctccgagcaacgttagcacatgacctaagaaccaggcgaagtgaagagtctggaataacgacacccagtcagtttttcctgagctcctggtgggattggtagaagcatttgatttgcttggagtggttttatttgaagatggtgttgaagccattgttgctaaagagtcggagttttgcttttagggtttgttaagcaaaggaggaaaaactgcgccgtttgaagtcccaggtagtttcgcgtgtgaggccagccagggaaagcttccttcggtacttttttttcttttgcaggttccggacggattaagcttcgggttatgaggggggcggtagccaattccggacacaatattgcgtcgcagctagtcaccccgccataaatatacgcaggattgaggtaataacatcgatagtcttagtaattaatacaattcagtggcgaatttggcaacatgacgtaaggcccactgttgtctataaaaggggatgaattttcatgtttttgaggcctcccggacaatttattgaactcaa

SEQ ID NO: 106 - DNA, 1000 bp, Artificial Sequence
  pG4 promoter
tggactgttcaatttgaagtcgatgctgacgatgtcaagagagatgctcaattatatttgtcatttgctggttacactggaaacgctacttttgttggcggaaactctaccagtttggccgtccatgtaaacgatgtcgttctgggccgtgaccgtttcaacacgaacataaccaatgacaaatccacttacaggtctagttcatatggaggcaattggtaccttacttctttggatgtcccaagtggggctttaacgtctggtactaacaatgtctcgtttgtcactacaaactccgaggtaaataaaggattcttgtgggattctctcaagtttgtttggaagttgtaacaggtttataagcatatcgtgcgcttgtccacaattgaatcatttattgttgcgagatacatgaacaaagtgtgaactgggacccattactacaattcccacgcaaccgttgtttcaaagcccatattttttgacaattgtttcgttacacccccagtttgatgtacatcgcttgcaatgatgtgtgtcccggagtattttccatattcagcttgaattcgtatactcaaccaatatctgggggtatacttttatgtaacctatacaaatcaactatactatttcacctttcgaccaatcatctcccatcttgttaagttttgcttcctatatccctgaccctgacatcacccatgattccgctcaacggttctcctctacatcgtccctcttttggagagggtgttcagtttgacattcaaattaccccccgccatcacgcgcaaccgagaccgcacccccgaattttcacaaattaccccacaccctatactccaccactatgagggttattagaactgatcacgtataaataccaccgcaagttcccaagggatcgtgttcttcttctccaattgcaatcatatttctgactctttctagttcagattaattcctttacacttgcttttttcccttacctttatcc

SEQ ID NO: 107 - DNA, 1001 bp, Artificial Sequence
  pG5 promoter
agaccagcagtttaactacgcaaatccacaggaatttctacatcacaataccaatggtaataccacgacgtcaaggaatggaaacgacgacttggaggaagacttcgtcaacctcttgcggagtacccgaggctaagacaataagaagaaaaaaaaaagaaaagcggtgggggagggattattaaataaggattatgtaaccccagggtaccgttctatacatatttaaggattatttaggacaatcgatgaaatcggcatcaaactggatgggagtatagtgtccggataatcggataaatcatcttgcgaggagccgcttggttggttggtgagaggagtgaaatatgtgtctcctcacccaagaatcgcgatatcagcaccctgtgggggacactattggcctccctcccaaaccttcgatgtggtagtgctttattatattgattacattgattacatagctaaaccctgcctggttgcaagttgagctccgaattccaatattagtaaaatgcctgcaagataacctcggtatggcgtccgaccccgcttaattattttaactcctttccaacgaggacttcgtaatttttgattagggagttgagaaacggggggtcttgatacctcctcgatttcagatcccaccccctctcagtcccaagtgggacccccctcggccgtgaaatgcgcgcactttagtttttttcgcatgtaaacgccggtgtccgtcaattaaaagtcgcagactagggtgaactttaccatttttgtcgcactccgtctcctcggaataggggtgtagtaattctgcagtagtgcaatttttaccccgccaagggggggcgaaaagagacgacctcatcacgcattctccagtcgctctctacgcctacagcaccgacgtagttaactttctcccatatataaagcaattgccattcccctgaaaactttaacctctgctttttcttgatttttccttgcccaaagaaaag

SEQ ID NO: 108 - DNA, 1000 bp, Artificial Sequence
  pG7 promoter
aattgattaagttcagtgaaatttcaaaccgctatacacaacaggacaactttgagtttagaaaaatccgatgtagtgtaacggctagcacggtccgctttcaccgggcagacccgggttcgactcccggcatcggagtttatttttccatttcgttctttagagtattctcctcagcatgcccccctgaatttttccttttttccatgtgtcccatttttccactttttttacagttttcctcgtgatgttaattggctacacaaaagctgccacacgaaaccttaatcacgaaaaactatacagccttcactaatccgtagccccataatatgttgtccacgtgctgttgggtactacctgtagactctcataccccactccgtctttctccaacaattaacgcagtaccgagatttatcagcagactcaaattgggcaaactctgtatttttccttgcccgcataatttatgggtctcaggcctccacgtttcctgtttacttgaagaatattggctgcggaaaaagtggtaaggacaacccccttttaattggatccagtttttccgaaatgttccgatccgtacgtcatctccgaagccgtacattttcactcaatctacgtagctttggactcagcgctcctggaattgccaggacagtttacttgagttgatattcccttgtagattgtgtgcttctttttccaaaatttgaggcttcgtttgaaaagtggaatctggtcgctagatcacttcatgcctatttttcacggaaaaataagtggtactatgcaccccttaaacctaaagaaaaacggaaaaattaccccaaaacctggtgatgtttttcgcccctttctttttatccgagtttttcttttttcttgtctgccaaattcctctcctgaccttagcgtccccggaaaaaattaactacttaaggaccgaatgagccccagcttttccccttctcttcattattccccataatataat

SEQ ID NO: 109 - DNA, 1000 bp, Artificial Sequence
  pG8 promoter
ctgcacaaccattgccagtaaggacgaagagaaggccccactacccaaaattcaggataacgtcttcataccatgcagcgacgcctacaagacgctgtcaagacatgccaacttcaacgaagtgaactttaacacattgatcgggaaattgaccaccaagggaatgctggttgaggctggaagcgttgccagtgtcctgagggaactggaccgaaagtttagtaatgcataagaggatatatataggaatgcagtaataatattagtacccattaagtgggctaagccattggaaggccgtctgactgatggtggtgttcttctcatttagatagtgcatttgcaactaccgtctgagattgagtttgatgtgaagctccagcgccaaaacagtataagaaccttatctccgcattattgttcttgcgtaaaagtttgtgtgaagaaacaggggtagttgcgcagattagttgtaatatgcgcataggatgggtcattgacttctttcctcgaaagagccacaccgttagctaaaaaaggacgcgcatctaccccaaaatagaatgtggggaaataggacgcgcaacttcctctcaatcactggacgtcagaaaaacaaatgcgcaatcgagtcaccctccgtgataccctccgtgataccccctctccgtctattctgacagcgtctccccatgacgtttcaatctacttagaaaagatttcgtttttttttccttcaattacacgatctcatcttctgcaagggtctggaggacatcaccaatctgcgactccataacttagtcctgagtttatatttacgcttcatctgatgagtaggaagaaaaagtttcacgaaattcccccgccaacttgcccttcggaataagcagccactctccttctgcccatagtaagcttgcgcgaggccccaacttggccagaaactttaaatatgccaaacaatctcccccaatctaagttctccctcttctaaaaa

SEQ ID NO: 110 - DNA, 859 bp, Artificial Sequence
  pG1a promoter
ggaatctgtattgttagaaagaacgagagttttttacggcgccgccatattgggccgtgtgaaaacagcttgaaaccccactactttcaaaggttctgttgctatacacgaaccatgtttaaccaacctcgcttttgacttgactgaagtcatcggttaacaatcaagtaccctagtctgtctgaatgctcctttccatattcagtaggtgtttcttgcacttttgcatgcactgcggaagaattagccaatagcgcgtttcatatgcgcttttaccccctcttttgtcaagcgcaaaatgcctgtaagatttggtgggggtgtgagccgttagctgaagtacaacaggctaattccctgaaaaaactgcagatagacttcaagatctcagggattcccactatttggtattctgatatgtttttcctgatatgcatcaaaactctaatctaaaacctgaatctccgctattttttttttttttttgatgaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 111 - DNA, 664 bp, Artificial Sequence
  pG1b promoter
ccatattcagtaggtgtttcttgcacttttgcatgcactgcggaagaattagccaatagcgcgtttcatatgcgcttttaccccctcttttgtcaagcgcaaaatgcctgtaagatttggtgggggtgtgagccgttagctgaagtacaacaggctaattccctgaaaaaactgcagatagacttcaagatctcagggattcccactatttggtattctgatatgtttttcctgatatgcatcaaaactctaatctaaaacctgaatctccgctattttttttttttttttgatgaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 112 - DNA, 493 bp, Artificial Sequence
  pG1c promoter
ctgcagatagacttcaagatctcagggattcccactatttggtattctgatatgtttttcctgatatgcatcaaaactctaatctaaaacctgaatctccgctattttttttttttttttgatgaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 113 - DNA, 370 bp, Artificial Sequence
  pG1d promoter
gaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 114 - DNA, 328 bp, Artificial Sequence
  pG1e promoter
ccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 115 - DNA, 283 bp, Artificial Sequence
  pG1f promoter
gcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 116 - DNA, 24 bp, Artificial Sequence
  primer
cctgaggctttgttccacccatct

SEQ ID NO: 117 - DNA, 30 bp, Artificial Sequence
  primer
ggaacatagtagtaccaccggacataacga

SEQ ID NO: 118 - DNA, 21 bp, Artificial Sequence
  primer
taaaattctccggggtaaaac

SEQ ID NO: 119 - DNA, 22 bp, Artificial Sequence
  primer
ctctccagcagagtaaaatttc

SEQ ID NO: 120 - DNA, 19 bp, Artificial Sequence
  primer
cacagaatcggtagaggtg

SEQ ID NO: 121 - DNA, 17 bp, Artificial Sequence
  primer
cagtgatggaagatggc

SEQ ID NO: 122 - DNA, 20 bp, Artificial Sequence
  primer
agactttcactttccacgct

SEQ ID NO: 123 - DNA, 19 bp, Artificial Sequence
  primer
caacgaaagcagcgaagtc

SEQ ID NO: 124 - DNA, 28 bp, Artificial Sequence
  primer
gatcggtctcacatgtcttgggacgacg

SEQ ID NO: 125 - DNA, 29 bp, Artificial Sequence
  primer
gatcggtctcagtcctgctttgtctctag

SEQ ID NO: 126 - DNA, 28 bp, Artificial Sequence
  primer
gatcggtctctggactatgtgaacctcc

SEQ ID NO: 127 - DNA, 36 bp, Artificial Sequence
  primer
gatcggtctcaaagcctacatgaaatcgtcatcacc

SEQ ID NO: 128 - DNA, 30 bp, Artificial Sequence
  primer
gatcggtctcacatgacaaacgaaccagaa

SEQ ID NO: 129 - DNA, 28 bp, Artificial Sequence
  primer
gatgggtctcagaccggtttggaaatcc

SEQ ID NO: 130 - DNA, 28 bp, Artificial Sequence
  primer
gatcggtctcaggtctgccttgtagaac

SEQ ID NO: 131 - DNA, 30 bp, Artificial Sequence
  primer
gatcggtctcagagcctgcattcaaagttg

SEQ ID NO: 132 - DNA, 26 bp, Artificial Sequence
  primer
gatcggtctcagctctggtgaccttg

SEQ ID NO: 133 - DNA, 39 bp, Artificial Sequence
  primer
gatcggtctcaaagcctaatccacaatttcttctttctc

SEQ ID NO: 134 - DNA, 34 bp, Artificial Sequence
  primer
gatgggtctcacatgtcccgtttctttgcgtcag

SEQ ID NO: 135 - DNA, 60 bp, Artificial Sequence
  primer
gatcggtctctaagcttatttactatagatcttcttttggtctttgacattggaggactg

SEQ ID NO: 136 - DNA, 30 bp, Artificial Sequence
  primer
gatcggtctcacatggctccaaactacaac

SEQ ID NO: 137 - DNA, 34 bp, Artificial Sequence
  primer
gatcggtctcagtgaagaattcgtagattgtctc

SEQ ID NO: 138 - DNA, 30 bp, Artificial Sequence
  primer
gatcggtctcatcacctccaaaagggttag

SEQ ID NO: 139 - DNA, 31 bp, Artificial Sequence
  primer
gatcggtctcagttatgtgtctcgaccttac

SEQ ID NO: 140 - DNA, 31 bp, Artificial Sequence
  primer
gatcggtctcataacaggctaaagagaatgg

SEQ ID NO: 141 - DNA, 39 bp, Artificial Sequence
  primer
gatcggtctctaagcttatattcttccttgacgctttag

SEQ ID NO: 142 - DNA, 33 bp, Artificial Sequence
  primer
gatcggtctcacatgaggccaattttactgaag

SEQ ID NO: 143 - DNA, 29 bp, Artificial Sequence
  primer
gaagggtctcattggacaccgaaaatagc

SEQ ID NO: 144 - DNA, 27 bp, Artificial Sequence
  primer
gatcggtctctccaaggattcggtagc

SEQ ID NO: 145 - DNA, 35 bp, Artificial Sequence
  primer
gatcggtctcaaagcctaagaggcagtctgtaaag

SEQ ID NO: 146 - DNA, 28 bp, Artificial Sequence
  primer
gatcggtctcacatggcaacagcagtag

SEQ ID NO: 147 - DNA, 32 bp, Artificial Sequence
  primer
gatcggtctcaccttgacacaatctacacacc

SEQ ID NO: 148 - DNA, 31 bp, Artificial Sequence
  primer
gatcggtctcaaaggagatcatttcactacc

SEQ ID NO: 149 - DNA, 26 bp, Artificial Sequence
  primer
gaagggtctcactccgagtccagagc

SEQ ID NO: 150 - DNA, 28 bp, Artificial Sequence
  primer
gatcggtctcaggaggctctggaagctc

SEQ ID NO: 151 - DNA, 36 bp, Artificial Sequence
  primer
atcggtctctaagcctacaccttaggctttggcttg

SEQ ID NO: 152 - DNA, 52 bp, Artificial Sequence
  ACGCCCTGCAGGATGTCTGAAGGTATTATTGAAATCGACACTAACTT AATCG
acgccctgcaggatgtctgaaggtattattgaaatcgacactaacttaatcg

SEQ ID NO: 153 - DNA, 53 bp, Artificial Sequence
  primer
gactggccgaggcggccctaagactcattaacttcctcagtctcaaacaagtc

SEQ ID NO: 154 - DNA, 42 bp, Artificial Sequence
  primer
gtctcctgcaggatgtccaataagaacgtggatacagctcca

SEQ ID NO: 155 - DNA, 46 bp, Artificial Sequence
  primer
gactggccgaggcggccttaaacttcctgttcctcttcttgctctc

SEQ ID NO: 156 - DNA, 40 bp, Artificial Sequence
  primer
gaggcctgcaggatgtctgtcgataccaaggaagttcaag

SEQ ID NO: 157 - DNA, 40 bp, Artificial Sequence
  primer
gagcggccgaggcggccctagtttgcttgtgcatccgctt

SEQ ID NO: 158 - DNA, 31 bp, Artificial Sequence
  primer
gatcggtctcccatgtcagagactgaaaacg

SEQ ID NO: 159 - DNA, 32 bp, Artificial Sequence
  primer
gatcggtctctattcaggtttgattccatctc

SEQ ID NO: 160 - DNA, 26 bp, Artificial Sequence
  primer
gatcggtctcagaatgggaggacgag

SEQ ID NO: 161 - DNA, 22 bp, Artificial Sequence
  primer
gatcggtctcacgcgggaccac

SEQ ID NO: 162 - DNA, 25 bp, Artificial Sequence
  primer
gatcggtctctcgcggtctgttgag

SEQ ID NO: 163 - DNA, 28 bp, Artificial Sequence
  primer
gatcggtctcacatccttggacttggtc

SEQ ID NO: 164 - DNA, 31 bp, Artificial Sequence
  primer
gatcggtctcagatgaggcagttttaagacc

SEQ ID NO: 165 - DNA, 37 bp, Artificial Sequence
  primer
gatcggtctcaaagcctaaatgctgaaagaaggtacg

SEQ ID NO: 166 - DNA, 63 bp, Artificial Sequence
  primer
gatcggtctcacatgtctgaaggtattattgaaatcgacactaacttaatcgagacaaactac

SEQ ID NO: 167 - DNA, 55 bp, Artificial Sequence
  primer
gatcggtctcaaagcctaagactcattaacttcctcagtctcaaacaagtcagtg

SEQ ID NO: 168 - DNA, 28 bp, Artificial Sequence
  primer
gactggtctcacatgcatccatacaccg

SEQ ID NO: 169 - DNA, 28 bp, Artificial Sequence
  primer
gatcggtctcaggtcgtagtttgtctcg

SEQ ID NO: 170 - DNA, 26 bp, Artificial Sequence
  primer
gatcggtctcagaccaggttgtcagc

SEQ ID NO: 171 - DNA, 38 bp, Artificial Sequence
  primer
gatcggtctcaaagcctaagactcattaacttcctcag

SEQ ID NO: 172 - DNA, 31 bp, Artificial Sequence
  primer
gatcggtctcacatgtccaataagaacgtgg

SEQ ID NO: 173 - DNA, 26 bp, Artificial Sequence
  primer
gatcggtctcaccagcgtcctcagat

SEQ ID NO: 174 - DNA, 29 bp, Artificial Sequence
  primer
gatcggtctcactggcaagactagagatg

SEQ ID NO: 175 - DNA, 29 bp, Artificial Sequence
  primer
gactggtctcacttaacgaacgaggtacc

SEQ ID NO: 176 - DNA, 393 bp, Artificial Sequence
  gBlock
ccattgggtctcataagaaataaggaggctgaagtcaagactgctccagacggatctataatagtatcagaagaggacatcaaaaggaaaactaaatcccttttgaataagttgacgttggaattctttgatgatatctcaaacgatataattgctttgaccaagcaagctcaatgggaagatgacgtcaagactttgaaacaagttattgagtctatatttgcaaaggcttgtgacgaaccctactggtcctctatgtacgctaaattatgcgccaaaatgtgcaaggacaccccacctgagatcaaggaaactaatgagaagggaaatactttcaccggtggtgatttggtgagaagagtgttgattaatagatgtcatgagaccgattcg

SEQ ID NO: 177 - DNA, 32 bp, Artificial Sequence
  primer
agctggtctcagtcaaaccgaatatcagaaag

SEQ ID NO: 178 - DNA, 36 bp, Artificial Sequence
  primer
gatcggtctcaaagcttaaacttcctgttcctcttc

SEQ ID NO: 179 - DNA, 30 bp, Artificial Sequence
  primer
gtacggtctcacatgtctgtcgataccaag

SEQ ID NO: 180 - DNA, 27 bp, Artificial Sequence
  primer
gatcggtctcagaagccaatgtctcgg

SEQ ID NO: 181 - DNA, 31 bp, Artificial Sequence
  primer
gatcggtctctcttcattgtatgttggtgag

SEQ ID NO: 182 - DNA, 25 bp, Artificial Sequence
  primer
gatcggtctcacacgttgggaccac

SEQ ID NO: 183 - DNA, 26 bp, Artificial Sequence
  primer
gatcggtctcacgtgacccttccttg

SEQ ID NO: 184 - DNA, 31 bp, Artificial Sequence
  primer
gatcggtctcagttcttgacaaagacattgg

SEQ ID NO: 185 - DNA, 30 bp, Artificial Sequence
  primer
gatcggtctctgaactttgacactgagtcc

SEQ ID NO: 186 - DNA, 34 bp, Artificial Sequence
  primer
gatcggtctctaagcctagtttgcttgtgcatcc

SEQ ID NO: 187 - DNA, 32 bp, Artificial Sequence
  primer
gatcggtctcgcatgagtgagaaaaacacacg

SEQ ID NO: 188 - DNA, 37 bp, Artificial Sequence
  primer
gatcggtctcaaagcttataactcagtgttctcaagg

SEQ ID NO: 189 - DNA, 28 bp, Artificial Sequence
  primer
agcggtgatttccttttgcattctttcg

SEQ ID NO: 190 - DNA, 23 bp, Artificial Sequence
  primer
tttgggtttggaatctgccggta

SEQ ID NO: 191 - DNA, 24 bp, Artificial Sequence
  primer
cctgaggctttgttccacccatct

SEQ ID NO: 192 - DNA, 30 bp, Artificial Sequence; identical to SEQ ID NO: 117
  primer

SEQ ID NO: 193 - DNA, 24 bp, Artificial Sequence
  primer
cgagagatcctccatcttcgacgc

SEQ ID NO: 194 - DNA, 25 bp, Artificial Sequence
  primer
gtgttgcaacaagtcgacgactctg

SEQ ID NO: 195 - DNA, 22 bp, Artificial Sequence
  primer
tgtaacgtgaatgtcggatttg

SEQ ID NO: 196 - DNA, 20 bp, Artificial Sequence
  primer
tagtgatggtggtggtgatg